(19) United States
(12) Patent Application Publication
(10) Pub. No.: US 2024/0425496 A1
(43) Pub. Date: Dec. 26, 2024

(54) AZETIDINYL PYRIMIDINES AND USES THEREOF

(71) Applicant: Aerie Pharmaceuticals, Inc., Bedminster, NJ (US)

(72) Inventors: David Archer Ellis, Bedminster, NJ (US); Heeren M. Gordhan, Durham, NC (US); Cynthia L. Lichorowic, Durham, NC (US); Jill M. Sturdivant, Chapel Hill, NC (US); Mitchell A. deLong, Chapel Hill, NC (US); Casey Kopczynski, Chapel Hill, NC (US); Jeffrey C. White, Chapel Hill, NC (US); Michelle Senchyna, Corona del Mar, CA (US); David Hollander, Beverly Hills, CA (US)

(21) Appl. No.: 18/573,305

(22) PCT Filed: Jul. 1, 2022
(86) PCT No.: PCT/US2022/073385

§ 371 Date: Dec. 21, 2023

Related U.S. Application Data
(60) Provisional application No. 63/217,607, filed on Jul. 1, 2021.

(51) Int. Cl. C07D 417/14; A61K 31/506; A61K 31/519; A61K 31/5377; A61K 31/5383; A61K 31/551; C07D 401/14; C07D 403/04; C07D 403/14; C07D 413/14; C07D 471/04; C07D 471/10; C07D 487/04; C07D 487/08; C07D 487/10; C07D 491/107; C07D 498/04

(52) U.S. Cl. CPC C07D 417/14; A61K 31/506; A61K 31/519; A61K 31/5377; A61K 31/5383; A61K 31/551; C07D 401/14; C07D 403/04; C07D 403/14; C07D 413/14; C07D 471/04; C07D 471/10; C07D 487/04; C07D 487/08; C07D 487/10; C07D 491/107; C07D 498/04

(57) ABSTRACT

Provided herein are azetidine pyrimidine compounds. In particular, provided herein are compounds that affect the function of kinases in a cell, and that are useful as therapeutic agents or with therapeutic agents. The compounds provided herein are useful in the treatment of a variety of diseases and conditions including inflammatory eye diseases such as uveitis, cardiovascular diseases, inflammatory diseases, and diseases characterized by abnormal growth, such as cancers. Also provided herein are compositions comprising azetidine pyrimidine compounds.

### RELATED APPLICATIONS

This application is a national stage entry under 35 U.S.C. § 371 of International Patent Application No. PCT/US2022/073385, filed Jul. 1, 2022, which claims priority of U.S. Provisional Patent Application No. 63/217,607, filed Jul. 1, 2021, the entire content of which is incorporated herein by reference.

### SEQUENCE LISTING

This application contains a sequence listing having the filename “Azetidinyl Pyrimidines and Uses Thereof.xml”, which is 2,845 bytes in size, and was created on Oct. 5, 2022. The entire content of this sequence listing is incorporated herein by reference.

### FIELD

The present disclosure relates to azetidinyl pyrimidine compounds that affect the function of kinases and other proteins in a cell and that are useful as therapeutic agents or with therapeutic agents. In particular, these compounds are useful in the treatment of eye diseases such as non-infectious uveitis or chorioretinitis, iritis, sterile conjunctivitis, keratitis, episcleritis, dry eye diseases, meibomian gland dysfunction, allergic conjunctivitis, glaucoma and retinal diseases, as anti-inflammatory agents, for the treatment of cardiovascular diseases, autoimmune related diseases and for diseases characterized by abnormal growth, such as cancers.

### BACKGROUND

A variety of hormones, neurotransmitters, and biologically active substances control, regulate or adjust the functions of living bodies via specific receptors located in cell membranes. Many of these receptors mediate the transmission of intracellular signals by activating guanine nucleotide-binding proteins (G proteins) to which the receptor is coupled. Such receptors are generically referred to as G-protein coupled receptors (GPCRs) and include, among others, α-adrenergic receptors, β-adrenergic receptors, interleukin receptors, opioid receptors, cannabinoid receptors and prostaglandin receptors. The biological effects of activating or inhibiting these receptors is not direct but is mediated by a host of intracellular proteins. The importance of these secondary proteins has been recognized and modulation of this class is now being investigated as intervention points in disease states. One of the most important classes of these downstream effectors is the “kinase” class.

The various kinases play important roles in the regulation of various physiological functions. For example, kinases have been implicated in a number of disease states, including, but not limited to: cardiac indications such as angina pectoris, essential hypertension, myocardial infarction, supraventricular and ventricular arrhythmias, congestive heart failure, atherosclerosis, renal failure, diabetes, respiratory indications such as asthma, chronic bronchitis, bronchospasm, emphysema, airway obstruction, upper respiratory indications such as rhinitis, seasonal allergies, inflammatory disease, autoimmune disease, inflammation in response to injury, rheumatoid arthritis. The importance of p38 MAPK inhibitors in particular as new drugs for rheumatoid arthritis is reflected by the large number of compounds that has been developed over the last years (J. Westra and P. C. Limburg Mini-Reviews in Medicinal Chemistry Volume 6, Number 8, August 2006). Other conditions include chronic inflammatory bowel disease, glaucoma, retinal geographic atrophy, age-related macular degeneration, hypergastrinemia, gastrointestinal indications such as acid/peptic condition, erosive esophagitis, gastrointestinal hypersecretion, mastocytosis, gastrointestinal reflux, peptic ulcer, Zollinger-Ellison syndrome, pain, obesity, bulimia nervosa, depression, obsessive-compulsive condition, organ malformations (e.g., cardiac malformations), neurodegenerative diseases such as Parkinson's Disease and Alzheimer's Disease, multiple sclerosis, Epstein-Barr infection and cancer (Nature Reviews Drug Discovery 2002, 1: 493-502). In other disease states, the role of kinases is only now becoming clear.

The retina is a complex tissue composed of multiple interconnected cell layers, highly specialized for transforming light and color into electrical signals that are perceived by the brain. Damage or death of the primary light-sensing cells, the photoreceptors, results in devastating effects on vision. Despite the identification of numerous mutations that cause inherited retinal degenerations, the cellular and molecular mechanisms leading from the primary mutations to photoreceptor apoptosis are not well understood but may involve the wnt pathway (AS Hackam “The Wnt Signaling Pathway in Retinal Degeneration” IUBMB Life Volume 57, Number 6/June 2005).

Janus Kinases (or JAK) are a family of cytoplasmic protein tyrosine kinases. The JAK family plays a role in the cytokine-dependent regulation of proliferation and function of cells involved in immune response. Currently, four JAK family members are known: JAK1, JAK2, JAK3, and TYK2. The JAKs usually associate with cytokine receptors in pairs as either homodimers or heterodimers. Specific cytokines are associated with specific JAK pairings. Each of the four members of the JAK family is implicated in the signaling pathways of at least one of the cytokines associated with inflammation. Binding of a cytokine to a JAK-dependent cytokine receptor induces receptor dimerization which results in phosphorylation of tyrosine residues on the JAK kinase, effecting JAK activation. Phosphorylated JAKs, in turn, bind and phosphorylate various STAT proteins which dimerize, translocate to the cell nucleus and directly modulate gene transcription, leading, among other effects, to the downstream effects associated with inflammatory and/or autoimmune disease. There is recent evidence that the kinases ROCK1 and ROCK2 may also participate in similar interactions with the STAT enzymes, resulting in the downregulation of IL-21 and IL-17 secretion. Inhibition of ROCK kinases has thus been noted to have an anti-inflammatory effect as well as the well-known effects of JAK inhibition.

In view of the role that kinases play in many disease states, there is an urgent and continuing need for small molecule ligands which inhibit or modulate the activity of kinases. Without wishing to be bound by theory, it is thought that modulation of the activity of kinases, in particular ROCK, JAK, or a combination of ROCK and JAK kinases, by the compounds of the present disclosure is, at least in part, responsible for their beneficial effects.

### SUMMARY

It has been found that the compounds described herein are useful as inhibitors of kinases, including JAK proteins.

Thus, in one aspect, provided herein are compounds, having a formula:

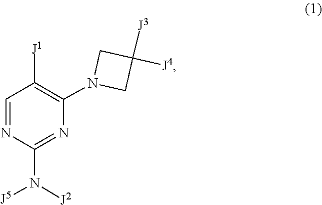

or a pharmaceutically acceptable salt thereof.

In one aspect, provided herein are compounds of the formula:

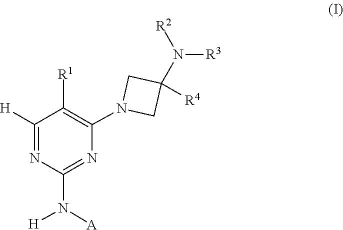

or a pharmaceutically acceptable salt thereof.

In another aspect, the present disclosure provides a pharmaceutical composition comprising a compound according to the present disclosure and a pharmaceutically acceptable excipient.

In another aspect, a pharmaceutical composition described herein may be housed in a package or container that is opaque, translucent or transparent with respect to visible light. Optionally, such a package or container can be formed that prevents light from a particular part of the electromagnetic spectrum, including UV light from penetrating into the interior of the container or package. Moreover, such a package or container can be formed of one or more polyolefin resins, one or more polyester resins, or any combination thereof when exposed to external effects such as light, particularly ultraviolet light, increased humidity of at least about 25%, at least about 40%, and at least about 65%, increased temperature of at least about 2° to about 8° C., at least about 25° C., at least about 30°, C and at least about 40° C., or any combination thereof for 1, 2, or 3 months or more, retards or slows the decrease of stability of the composition. As a result, the residual rate, i.e. the amount of an active contained in such a composition after exposure to an external effect may be at least 70%, at least 80%, at least 90%, at least 95% or at least 98% or more as compared to the amount of the active in the composition before such exposure to external effects.

In addition to the materials discussed above, a package or container for a pharmaceutical composition of the instant disclosure can be formed of glass or metal. A pharmaceutical composition of the instant disclosure may be housed in a primary package material, which itself may be housed in a secondary package material. The secondary package material can be made of paper, e.g. cardboard.

In another aspect, the present disclosure provides a method of treating an ocular disease or condition in a subject in need of treatment, comprising administering to the subject a compound or composition according to the present disclosure.

In another aspect, the present disclosure provides a method of reducing inflammation in a subject in need thereof, comprising administering to an eye of the subject a compound or composition according to the present disclosure.

In another aspect, the present disclosure provides a kit comprising a compound or composition according to the present disclosure and instructions for use.

### BRIEF DESCRIPTION OF THE DRAWINGS

FIG. 1 shows a general scheme for the synthesis of the azetidinyl pyrimidine compounds described herein.

FIG. 2 shows an exemplary synthetic scheme (Scheme 1) that may be used to prepare the compounds described herein.

FIG. 3 shows another exemplary synthetic scheme (Scheme 1′) that may be used to prepare the compounds described herein.

### DETAILED DESCRIPTION

Publications and patents are referred to throughout this disclosure. All U.S. Patents and published applications cited herein are hereby incorporated by reference in their entireties. All percentages, ratios, and proportions used herein are percent by weight unless otherwise specified.

Azetidinyl pyrimidine compounds are provided herein.

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

As used herein, the terms “alkyl” or “alkylene” refer to a saturated aliphatic hydrocarbon including straight chain and branched chain groups. “Alkyl” may be exemplified by groups such as methyl, ethyl, n-propyl, isopropyl, n-butyl and the like. “C₁₋₆ alkyl” refers to alkyl groups containing one to six carbon atoms. Alkyl groups may be substituted or unsubstituted, including substitution, independently, with alkenyl, alkynyl, alkoxyl, amino, amido, carboxyl, carboxamidyl, carboxhydroxamidyl, cycloalkyl, halo, heterocycloalkyl, hydroxyl, polycyclyl, aryl, heteroaryl, nitrile, boronyl, or cyano groups. Substituents may be themselves substituted.

The term “alkynyl” refers to an unsaturated hydrocarbon that includes at least one carbon-carbon triple bond.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “aryl” refers to a carbocyclic aromatic system comprising one, two, three, or more rings.

The term “Cₙ₋ₘ” refers to a moiety comprising n to m carbon atoms, wherein n and m are integers.

The terms “composition” and “pharmaceutical composition” refer to a mixture of at least one compound described herein with a pharmaceutically acceptable carrier. The pharmaceutical composition facilitates administration of the compound to a patient or subject. Multiple techniques of administering a compound exist including, but not limited to, intravenous, oral, aerosol, parenteral, ophthalmic, nasal, pulmonary, and topical administration.

As used herein, the term “contacting a cell” is used to mean contacting a cell in vitro or in vivo i.e., in a subject, such as a mammal, including humans, non-human primates, rabbits, cats and dogs.

As used herein, the term “controlling the disease or condition” is used to mean changing the activity of one or more kinases to affect the disease or condition.

The term “cycloalkyl” refers to a cyclic alkyl moiety comprising one, two, three, or more rings.

As used herein, the term “disease or condition associated with kinase activity” is used to mean a disease or condition treatable, in whole or in part, by inhibition of one or more kinases.

The terms “effective amount” and “therapeutically effective amount” refer to an amount of therapeutic compound, such as a compound described herein, administered to a subject, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

As used herein, the term “excipient” includes physiologically compatible additives useful in preparation of a pharmaceutical composition. A non-limiting number of examples of pharmaceutically acceptable carriers and excipients can for example be found in Remington Pharmaceutical Science, 16^{th} Ed.

As used herein, the phrase “ocular disease or condition” includes, but is not limited to, ocular inflammatory conditions such as non-infectious uveitis, chorioretinitis, iritis, sterile conjunctivitis, keratitis, episcleritis, dry eye diseases, meibomian gland dysfunction, allergic conjunctivitis, MGD, injury-related ocular inflammation or dry eye syndrome, Primary and Secondary Sjögren's syndrome, redness, blepharitis, keratoconjunctivitis sicca, ocular hyperemia, macular degeneration (wet and dry), diabetic retinopathy, DME, RVO, age-related macular degeneration, geographic atrophy, posterior uveitis, retinal inflammation, Inflammation due to gene therapy vectors (e.g. viral vectors), graft versus host disease, blepharitis, Thygeson superficial punctate keratitis (TSPK), or a combination thereof.

The terms “halo” or “halogen” refer to one or more atoms independently selected from F, Br, Cl, or I. In some embodiments, the halogen is fluoro, chloro, or bromo. In some embodiments, the halogen is fluoro.

The term “haloalkyl” refers to an alkyl moiety substituted with one or more halogens.

The term “haloaryl” refers to an aryl moiety substituted with one or more halogens.

The term “halocycloalkyl” refers to a cycloalkyl moiety substituted with one or more halogens.

As used herein, the term “heteroalkyl” refers to a non-aromatic carbocyclic radical having one or more heteroatoms in the carbocyclic ring.

The term “heteroaryl” refers to an aryl moiety comprising at least one ring heteroatom selected from O, S, or N, wherein each ring may comprise, independently, one, two, three, or four ring heteroatoms independently selected from O, S, or N.

The term “heterocycloalkyl” refers to a cycloalkyl moiety comprising at least one ring heteroatom selected from O, S, or N, wherein each ring may comprise, independently, one, two, three, or four ring heteroatoms independently selected from O, S, or N.

The term “heterocyclyl” refers to a ring system comprising at least one heteroatom selected from O, S, or N, one or more rings, wherein each ring may comprise, independently, one, two, three, or four ring heteroatoms independently selected from O, S, or N.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Non-limiting examples of other ingredients that may be included in a pharmaceutical composition described herein are known in the art and described, for example, in “Remington's Pharmaceutical Sciences” (Genaro (Ed.), Mack Publishing Co., 1985), the entire content of which is incorporated herein by reference.

As used herein, the term “polycyclic” means a non-aromatic radical that has two or more rings in the structure. Polycyclic rings include tri- and bicyclic rings, and rings that are spirocyclic. Polycyclic rings can contain heteroatoms or be entirely carbocyclic. Polycyclic rings can have substituents, including alkyl substituents, non-cyclic heteroatoms and halogens.

As used herein, a superscripted “R” and a subscripted “R” refer to the same moiety, thus R₁ and R¹ refer to the same group, as do R₂ and R², etc. In some depictions, a superscript might be used, and in other depictions, a subscript is used.

The term “refractory disease” refers to a disease that continues to progress during treatment with a pharmaceutical ingredient other than the compounds provided herein, partially responds to the other treatment, or transiently responds to the other treatment. The term may be applied to each of the diseases referred to herein.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability. When any such inclusion or exclusion occurs, the specification is deemed to contain the group as modified thus fulfilling the written description of all Markush groups used in the appended claims.

It is to be understood that the embodiments of this disclosure are illustrative. Accordingly, the present disclosure is not limited to that precisely as shown and described.

### Compounds

It has been found, surprisingly and unexpectedly, that the compounds described herein are useful as inhibitors of kinases, including JAK proteins.

Thus, in one aspect, provided herein are compounds, having a formula:

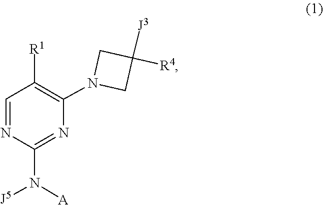

or a pharmaceutically acceptable salt thereof;
wherein
J⁵ is H or S(O)₂—(C₁₋₆ alkyl);
R¹ is H, CN, halogen, C₁₋₆ alkyl, O—(C₁₋₆ alkyl), or C₃₋₇ cycloalkyl;
A is C₆₋₁₆ aryl or C₂₋₁₅ heterocyclyl, each of which may be substituted with 1 or 2 groups selected, independently, from OH, halogen, C₁₋₆ alkyl, (C₁₋₆ alkylene)-OH, O—(C₁₋₆ alkyl), (C₁₋₆ alkylene)-O—(C₁₋₆ alkyl), S(O)₂—(C₁₋₆ alkyl), C₃₋₇ cycloalkyl, (C₁₋₁₆ alkylene)-C(O)OH, (C₁₋₁₆ alkylene)-C(O)N(H)—(C₁₋₆ alkyl), (C₁₋₁₆ alkylene)-C(O)N(H)—OH, or (C₁₋₆ alkylene)-N(C₁₋₆ alkyl)-(C₁₋₆ alkyl);
J³ is CN, OH, NH₂, C₁₋₆ alkyl, C₁₋₆ alkynyl, C₃₋₇ cycloalkyl, (C₁₋₃ alkylene)-CN, (C₁₋₃ alkylene)-NH₂, (C₁₋₃ alkylene)-N(H)C(O)—(C₃₋₇ cycloalkyl), (C₁₋₃ alkylene)-N(H)C(O)—(C₂₋₈ heterocycloalkyl), O—(C₃₋₇ cycloalkyl), O—(C₁₋₃ alkylene)-CN, N(H)C(O)—(C₃₋₇ cycloalkyl), N(H)C(O)—(C₂₋₈ heterocycloalkyl), N(H)C(O)—(C₂₋₅ heteroaryl), N(H)C(O)—(C₁₋₃ alkylene)-(C₂₋₅ heteroaryl), N(H)C(O)—(C₆₋₁₀ aryl), N(H)C(O)—(C₁₋₃ alkylene)-(C₆₋₁₀ aryl), N(H)C(O)NH₂, N(H)(C₁₋₆ alkyl), N(H)(C₃₋₇ cycloalkyl), N(C₁₋₆ alkyl)(C₃₋₇ cycloalkyl), N(H)(C₁₋₃ alkylene)-(C₂₋₈ heterocycloalkyl), N(H)(C₁₋₃ alkylene)-(C₂₋₅ heteroaryl), N(H)(C₁₋₃ alkylene)-(C₆₋₁₀ aryl), or C₂₋₁₅ heterocyclyl, each of which may be substituted with 1, 2, 3, or 4 groups selected, independently, from halogen, O, OH, O—(C₁₋₆ alkyl), S(O)₂—(C₁₋₆ alkyl), S(O)₂—(C₁₋₆ haloalkyl), C(NH₂)(NH), C₁₋₆ alkyl, C₁₋₆ haloalkyl, (C₁₋₆ alkylene)-OH, (C₁₋₆ alkylene)-O—(C₁₋₆ alkyl), (C₁₋₆ alkylene)-O—(C₁₋₆ haloalkyl), (C₁₋₆ alkylene)-S(O)₂—(C₁₋₆ alkyl), (C₁₋₆ alkylene)-NH₂, (C₁₋₆ alkylene)-N(H)(C₁₋₆ alkyl), (C₁₋₆ alkylene)-N(C₁₋₆ alkyl)(C₁₋₆ alkyl), (C₁₋₆ alkylene)-(C₃₋₇ cycloalkyl), (C₁₋₆ alkylene)-(C₃₋₇ halocycloalkyl), (C₁₋₆ alkylene)-(C₂₋₅ heteroaryl), (C₁₋₆ alkylene)-(C₆₋₁₀ haloaryl), NH₂, N(H)(C₁₋₆ alkyl), N(C₁₋₆ alkyl)(C₁₋₆ alkyl), N(H)—(C₁₋₃ alkylene)-(C₃₋₇ cycloalkyl), N(C₁₋₆ alkyl)-(C₁₋₃ alkylene)-(C₃₋₇ cycloalkyl), N(H)C(O)O—(C₁₋₆ alkyl), N(H)S(O)₂—(C₁₋₆ alkyl), C(O)—(C₁₋₆ alkyl), C(O)—(C₁₋₆ alkylene)-CN, C(O)—(C₁₋₆ alkylene)-NH₂, C(O)—(C₁₋₆ alkylene)-N(H)(C₁₋₆ alkyl), C(O)—(C₁₋₆ alkylene)-N(C₁₋₆ alkyl)(C₁₋₆ alkyl), C(O)—(C₁₋₆ alkylene)-N(H)[S(O)₂—C₁₋₆ alkyl], C(O)—(C₁₋₆ alkylene)-N(C₁₋₆ alkyl)[S(O)₂—C₁₋₆ alkyl], or C(O)O—(C₁₋₆ alkyl); and
R⁴ is H, OH, C₁₋₆ alkyl, (C₁₋₆ alkylene)-OH, C₁₋₆ haloalkyl, C₃₋₇ cycloalkyl, C₃₋₇ halocycloalkyl, (C₁₋₃ alkylene)-(C₃₋₇ cycloalkyl), (C₁₋₆ alkylene)CN, (C₁₋₆ alkylene)-C(O)O—(C₁₋₆ alkyl), (C₁₋₆ alkylene)-OC(O)—(C₁₋₆ alkyl), or C₂₋₈ heterocycloalkyl;
or J³ and R⁴, together with the atoms to which they are attached, combine to form C₃-7 cycloalkyl, C₂₋₈ heterocycloalkyl, CC(H)—(C₀₋₆ alkylene)CN, C₃₋₇ cycloalkyl substituted with 1 or 2 groups selected, independently, from halogen or (C₁₋₆ alkylene)CN, or C₂₋₈ heterocycloalkyl substituted with 1 or 2 groups selected, independently, from halogen or (C₁₋₆ alkylene)CN.

In some embodiments, J⁵ is H.

In one aspect, provided herein are compounds of the formula:

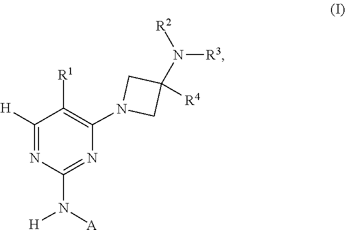

or a pharmaceutically acceptable salt thereof,
wherein
R¹ is H, F, Cl, Br, I, —CN, —NH₂, —OH, —OCF₃, —OCH₂CF₃, C₁₋₆ alkyl, C₃₋₇ cycloalkyl, or C₁₋₆ haloalkyl;
R² and R³ are independently, H, C₁₋₆ alkyl, C₁₋₆ heteroalkyl or C₁₋₆ haloalkyl, or R² and
R³ combine to form a heteroalkyl or polycyclic ring of at least 4 member atoms and at most 8 member atoms;
R⁴ is C₁₋₆ alkyl, C₁₋₆ heteroalkyl or C₁₋₆ haloalkyl, CH₂CN, CH₂CH₂CN, CH₂CH₂CH₂CN, C₃-C₇ cycloalkyl, alkylenecycloalkyl, alkylenecyclopropyl, or hydroxyl; and
A is phenyl, or a monocyclic C₃₋₅ heteroaryl having 1-3 heteroatoms, wherein each heteroatom is, independently, N, O, or S.

In some embodiments of the formulae provided herein, J³ is

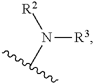

wherein R² and R³ are as defined herein.

In some embodiments of the formulae provided herein, J⁵ is H.

In some embodiments of the formulae provided herein, A is phenyl, thiazolyl, isothiazolyl, pyrazolyl, imidazolyl, pyridinyl, pyrimidinyl, indolinyl, isoindolinyl, indolyl, phenylmorpholinyl, phenyloxetanyl, phenylpiperazinyl, dihydrobenzoborolyl, or benzoborolyl, each of which may be substituted with 1 or 2 groups selected, independently, from OH, F, Cl, Br, C₁₋₃ alkyl, (C₁₋₃ alkyl)-OH, C₁₋₆ haloalkyl, O—(C₁₋₃ alkyl), (C₁₋₃ alkylene)-O—(C₁₋₃ alkyl), S(O)₂—(C₁₋₃ alkyl), C₃₋₅ cycloalkyl, (C₆₋₁₆ alkylene)-C(O)OH, (C₆₋₁₆ alkylene)-C(O)N(H)—(C₁₋₆ alkyl), (C₆₋₁₆ alkylene)-C(O)N(H)—OH, or (C₁₋₃ alkylene)-N(C₁₋₃ alkyl)-(C₁₋₃ alkyl).

In some embodiments of the formulae provided herein, J³ is CN, OH, NH₂, C₁₋₆ alkyl, C₁₋₆ alkynyl, C₃₋₇ cycloalkyl, (C₁₋₃ alkylene)-CN, or (C₁₋₃ alkylene)-NH₂,

In some embodiments of the formulae provided herein, J³ is OH, NH₂, C₁₋₃ alkyl, C₁₋₃ alkynyl, C₃₋₇ cycloalkyl, or (C₁₋₃ alkylene)-NH₂, each of which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments of the formulae provided herein, J³ is (C₁₋₃ alkylene)-N(H)C(O)—(C₃₋₇ cycloalkyl), (C₁₋₃ alkylene)-N(H)C(O)—(C₂₋₈ heterocycloalkyl), O—(C₃₋₇ cycloalkyl), or O—(C₁₋₃ alkylene)-CN, each of which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments of the formulae provided herein, J³ is N(H)C(O)—(C₃₋₇ cycloalkyl), N(H)C(O)—(C₂₋₈ heterocycloalkyl), N(H)C(O)—(C₂₋₅ heteroaryl), N(H)C(O)—(C₁₋₃ alkylene)-(C₂₋₅ heteroaryl), N(H)C(O)—(C₆₋₁₀ aryl), N(H)C(O)—(C₁₋₃ alkylene)-(C₆₋₁₀ aryl), N(H)C(O)NH₂, N(H)(C₁₋₆ alkyl), N(H)(C₃₋₇ cycloalkyl), N(C₁₋₆ alkyl)(C₃₋₇ cycloalkyl), N(H)(C₁₋₃ alkylene)-(C₂₋₈ heterocycloalkyl), N(H)(C₁₋₃ alkylene)-(C₂₋₅ heteroaryl), or N(H)(C₁₋₃ alkylene)-(C₆₋₁₀ aryl), each of which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments of the formulae provided herein, J³ is C₂₋₁₅ heterocyclyl, which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments of the formulae provided herein, J³ and R⁴, together with the atoms to which they are attached, combine to form C₃₋₇ cycloalkyl, C₂₋₈ heterocycloalkyl, CC(H)—(C₀₋₃ alkylene)CN, C₃₋₇ cycloalkyl substituted with 1 or 2 groups selected, independently, from halogen or (C₁₋₃ alkylene)CN, or C₂₋₈ heterocycloalkyl substituted with 1 or 2 groups selected, independently, from halogen or (C₁₋₃ alkylene)CN.

In one aspect, provided herein are compounds of the formula:

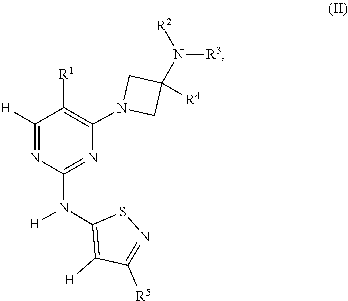

or a pharmaceutically acceptable salt thereof,
wherein
R¹ is H, F, Cl, Br, I, CN, NH₂, OH, OCF₃, OCH₂CF₃, C₁₋₆ alkyl, C₃₋₆ cycloalkyl, or C₁₋₆ haloalkyl;
R² and R³ are independently, H, C₁₋₆ alkyl, C₁₋₆ heteroalkyl or C₁₋₆ haloalkyl, or R² and R³ combine to form a heteroalkyl or polycyclic ring of at least 4 member atoms and at most 8 member atoms;
R⁴ is C₁₋₆ alkyl, C₁₋₆ heteroalkyl or C₁₋₆ haloalkyl, —CH₂CN, —CH₂CH₂CN, —CH₂CH₂CH₂CN, C₃-C₇ cycloalkyl, C₁₋₁₂ alkylenecycloalkyl, alkylenecyclopropyl, or hydroxyl;
R⁵ is C₁₋₁₂ alkyl, C₃₋₆ cycloalkyl, C₁₋₁₂ heteroalkyl or C₁₋₁₂ haloalkyl, —CH₂OH, —CH₂CH₂OH, —CH₂CH₂CH₂OH, alkylenecycloalkyl, alkylenecyclopropyl, or methoxy;

In some embodiments of the formulae provided herein, the compound is a compound of the formula selected from:

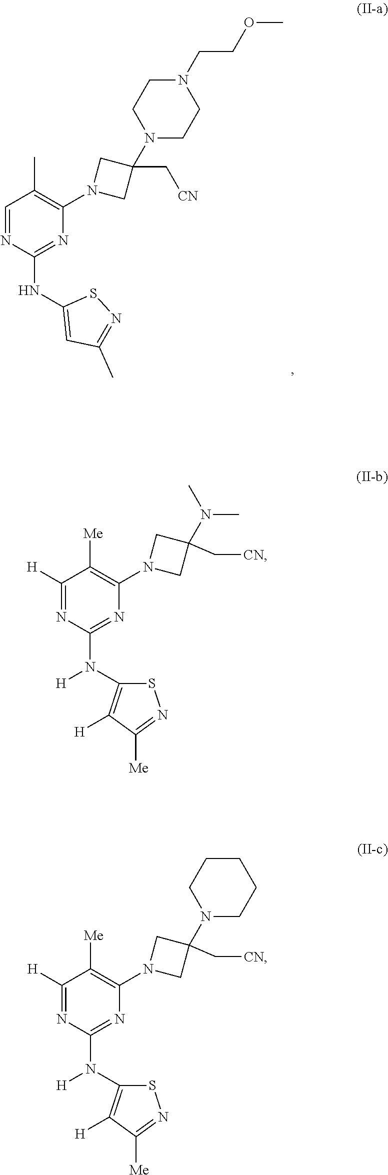 [SMILES: COCCN1CCN(C2(CC#N)CN(c3nc(Nc4cc(C)ns4)ncc3C)C2)CC1 ; Cc1cc(Nc2ncc(C)c(N3CC(CC#N)(N(C)C)C3)n2)sn1 ; Cc1cc(Nc2ncc(C)c(N3CC(CC#N)(N4CCCCC4)C3)n2)sn1]

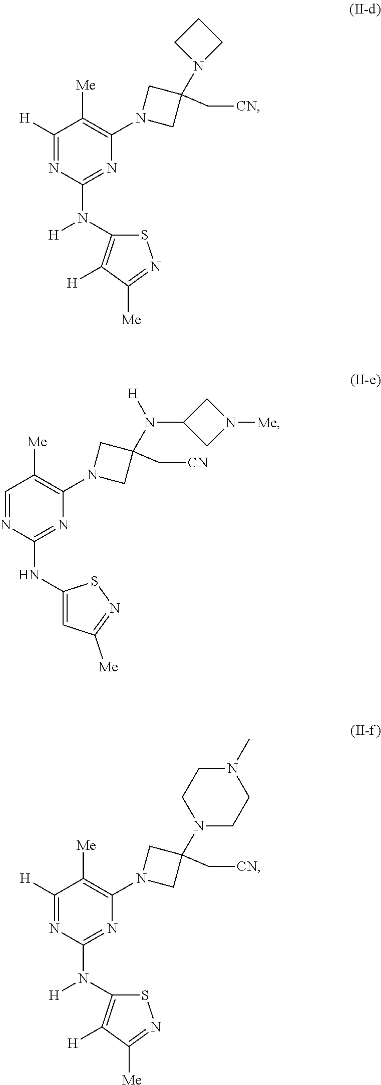 [SMILES: Cc1cc(Nc2ncc(C)c(N3CC(CC#N)(N4CCC4)C3)n2)sn1 ; Cc1cc(Nc2ncc(C)c(N3CC(CC#N)(NC4CN(C)C4)C3)n2)sn1 ; Cc1cc(Nc2ncc(C)c(N3CC(CC#N)(N4CCN(C)CC4)C3)n2)sn1]

or a pharmaceutically acceptable salt thereof.

In some embodiments of the formulae provided herein, the compound is of the formula:

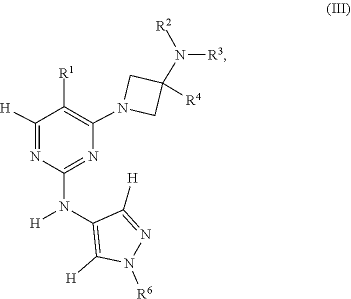

or a pharmaceutically acceptable salt thereof,
wherein
R¹ is H, F, Cl, Br, I, CN, NH₂, OH, OCF₃, OCH₂CF₃, C₁₋₆ alkyl, C₃₋₆ cycloalkyl, or C₁₋₆ haloalkyl;
R² and R³ are H, C₁₋₆ alkyl, C₁₋₆ heteroalkyl or C₁₋₆ haloalkyl, or R² and R³ combine to form a heteroalkyl or polycyclic ring of at least 4 member atoms and at most 10 member atoms;
R⁴ is C₁₋₆ alkyl, C₁₋₆ heteroalkyl or C₁₋₆ haloalkyl, CH₂CN, CH₂CH₂CN, CH₂CH₂CH₂CN, C₃-C₇ cycloalkyl, alkylenecycloalkyl, alkylenecyclopropyl, or hydroxyl; and
R⁶ is C₁₋₁₂ alkyl, C₁₋₁₂ heteroalkyl or C₁₋₁₂ haloalkyl, CH₂OH, CH₂CH₂OH, CH₂CH₂CH₂OH, alkylenecycloalkyl, alkylenecyclopropyl, or methoxy.

In some embodiments of the formulae provided herein, the compound is a compound of the formula selected from:

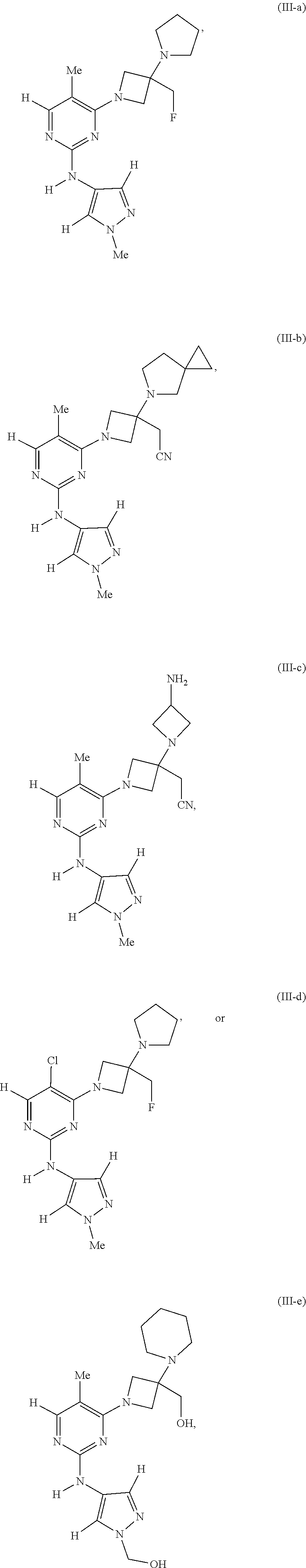

or a pharmaceutically acceptable salt thereof.

In some embodiments, the compound is of the formula:

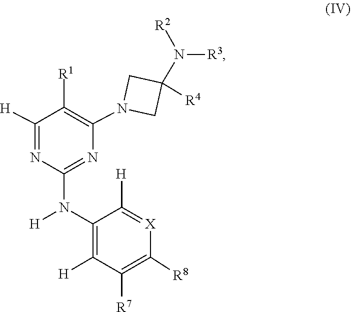

or a pharmaceutically acceptable salt thereof,
wherein
R¹ is H, F, Cl, Br, I, CN, NH₂, OH, OCF₃, OCH₂CF₃, C₁₋₆ alkyl, C₃₋₆ cycloalkyl, or C₁₋₆ haloalkyl;
R² and R³ are H, C₁₋₆ alkyl, C₁₋₆ heteroalkyl or C₁₋₆ haloalkyl, or R² and R³ combine to form a heteroalkyl or polycyclic ring of at least 4 member atoms and at most 10 member atoms;
R⁴ is C₁₋₆ alkylene, C₁₋₆ heteroalkyl or C₁₋₆ haloalkyl, CH₂CN, CH₂CH₂CN, CH₂CH₂CH₂CN, C₃-C₇ cycloalkyl, alkylenecycloalkyl, alkylenecyclopropyl, or hydroxyl;
X is CH or N or CR⁷
R⁷ & R⁸ are independently H, amino, aminoalkyl, C₁₋₁₂ alkyl, C₁₋₁₂ heteroalkyl or C₁₋₁₂ haloalkyl, halo, CH₂OH, CH₂CH₂OH, CH₂CH₂CH₂OH, alkylenecycloalkyl, alkylenecyclopropyl, or methoxy, sulfonyl, sulfonamyl, or R⁷ and R⁸ combine to form a heteroalkyl ring of at least 4 member atoms and at most 8 member atoms.

In some embodiments, the compound is of the formula:

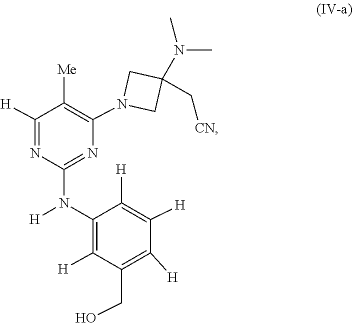 [SMILES: Cc1cnc(Nc2cccc(CO)c2)nc1N1CC(CC#N)(N(C)C)C1]

or a pharmaceutically acceptable salt thereof.

In some embodiments, the compound is of the formula:

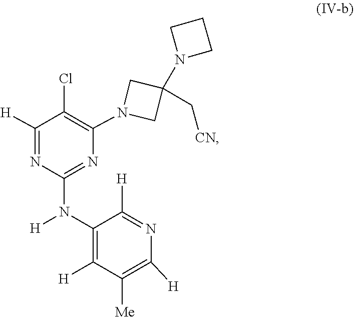 [SMILES: Cc1cncc(Nc2ncc(Cl)c(N3CC(CC#N)(N4CCC4)C3)n2)c1]

or a pharmaceutically acceptable salt thereof.

In some embodiments, the compound is of the formula:

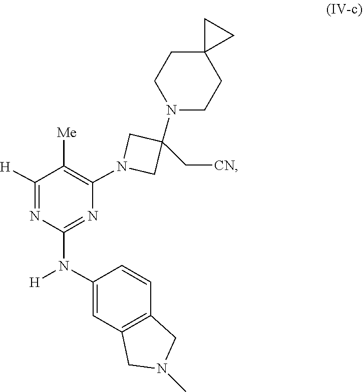 [SMILES: Cc1cnc(Nc2ccc3c(c2)CN(C)C3)nc1N1CC(CC#N)(N2CCC3(CC2)CC3)C1]

or a pharmaceutically acceptable salt thereof,

In some embodiments, the compound is selected from:

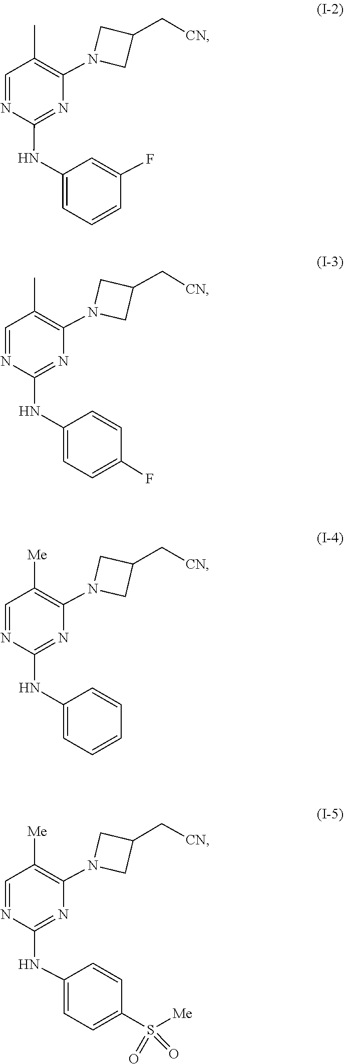 [SMILES: Cc1cnc(Nc2cccc(F)c2)nc1N1CC(CC#N)C1 ; Cc1cnc(Nc2ccc(F)cc2)nc1N1CC(CC#N)C1 ; Cc1cnc(Nc2ccccc2)nc1N1CC(CC#N)C1 ; Cc1cnc(Nc2ccc(S(C)(=O)=O)cc2)nc1N1CC(CC#N)C1]

or a pharmaceutically acceptable salt thereof.

In some embodiments, the compound is of the formula:

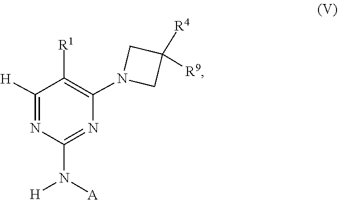

R¹ is H, F, Cl, Br, I, CN, OCF₃, OCH₂CF₃, C₁₋₃ alkyl, C₃₋₆ cycloalkyl, or C₁₋₆ haloalkyl;
R⁴ is C₁₋₆ alkyl, C₁₋₆ heteroalkyl or C₁₋₆ haloalkyl, CH₂CN, CH₂CH₂CN, CH₂CH₂CH₂CN, C₃-C₇ cycloalkyl, alkylenecycloalkyl, alkylenecyclopropyl, or hydroxyl;
R⁹ is H, C₁₋₆ alkyl, C₁₋₆ heteroalkyl or C₁₋₆ haloalkyl, or R⁴ and R⁹ combine to form a carbocyclic, polycyclic, or heteroalkyl ring of at least 4 member atoms and at most 10 member atoms;
A is phenyl, or a monocyclic C₃₋₅ heteroaryl having 1-3 heteroatoms, wherein each heteroatom is, independently, N, O, or S;
or a pharmaceutically acceptable salt thereof.

In some embodiments of the formulae provided herein:
R¹ is H, F, Cl, Me, CN, cyclopropyl, or C₁₋₃ alkyl;
R² is H, or C₁₋₃ alkyl; or
R⁷ is H, F, or C₁₋₃ alkyl.

In some embodiments of the formulae provided herein, R² and R³ combine to form a heteroalkyl ring of 6 member atoms.

In some embodiments of the formulae provided herein, R⁴ is —CH₂CN

In some embodiments of the formulae provided herein, R⁴ is —CH₂—CH₂CN

In some embodiments of the formulae provided herein, R⁴ is —OH.

In some embodiments of the formulae provided herein, R⁶ is substituted or unsubstituted C₁₋₁₄ alkyl, alkenyl, alkynyl, alkylenearyl, alkylenecycloalkyl.

In some embodiments of the formulae provided herein, A is pyridyl.

In some embodiments of the formulae provided herein, A is not phenyl.

In some embodiments of the formulae provided herein, R⁷ is alkyleneamino.

In some embodiments of the formulae provided herein, R¹ is H.

In some embodiments of the formulae provided herein, R⁸ is alkylene amino.

In some embodiments of the formulae provided herein, R⁸ is cyclopropyl.

In some embodiments of the formulae provided herein, R⁸ is a monocyclic C₃₋₅ heteroaryl having 1-3 nitrogen atoms.

In some embodiments the compound is:

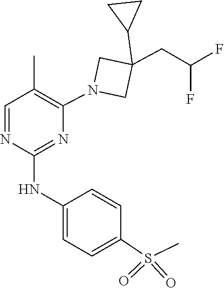 [SMILES: Cc1cnc(Nc2ccc(S(C)(=O)=O)cc2)nc1N1CC(CC(F)F)(C2CC2)C1]

or a pharmaceutically acceptable salt thereof.

In some embodiments, the compound is a compound of the formula selected from:

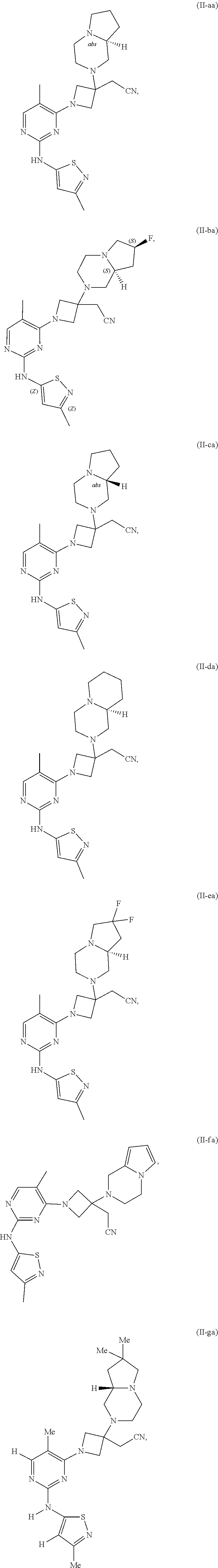

or a pharmaceutically acceptable salt thereof.

In some embodiments, the compound is of the formula:

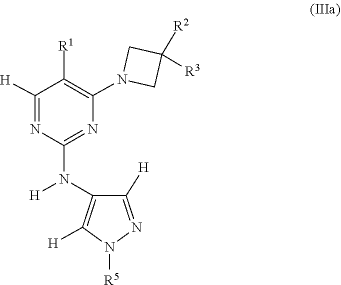

or a pharmaceutically acceptable salt thereof,
wherein
R¹ is H, F, Cl, CN, CF₃, C₁₋₆ alkyl, C₃₋₆ cycloalkyl, or C₁₋₆ haloalkyl;
R² is H, amino, amidyl, aminosulfonyl, hydroxyl, cycloalkyl, C₁₋₆ alkyl, C₁₋₆ heteroalkyl or C₁₋₆ haloalkyl, heterocyclyl, heteroaryl or can link to R³ to form a ring;
R³ is C₁₋₆ alkyl, C₁₋₆ heteroalkyl or C₁₋₆ haloalkyl, CH₂CCH, CH₂CN, CH₂CH₂CN, CH₂CH₂CH₂CN, CH₂CO₂R⁴, CH₂CO₂H, CH₂CH₂OH, CH₂CH₂F, CH₂CH₂Cl, CH₂CHF₂, CH₂CF₃ or can link to R³ to form a ring;
R⁴ is C₁₋₃ alkyl; and
R⁵ is C₁₋₁₂ alkyl, C₃₋₆ cycloalkyl, C₁₋₁₂ heteroalkyl or C₁₋₁₂ haloalkyl, CH₂OH, CH₂CH₂OH, CH₂CH₂CH₂OH, alkylenecycloalkyl, alkylenecyclopropyl, or methoxy.

In some embodiments, the compound is a compound of the formula selected from:

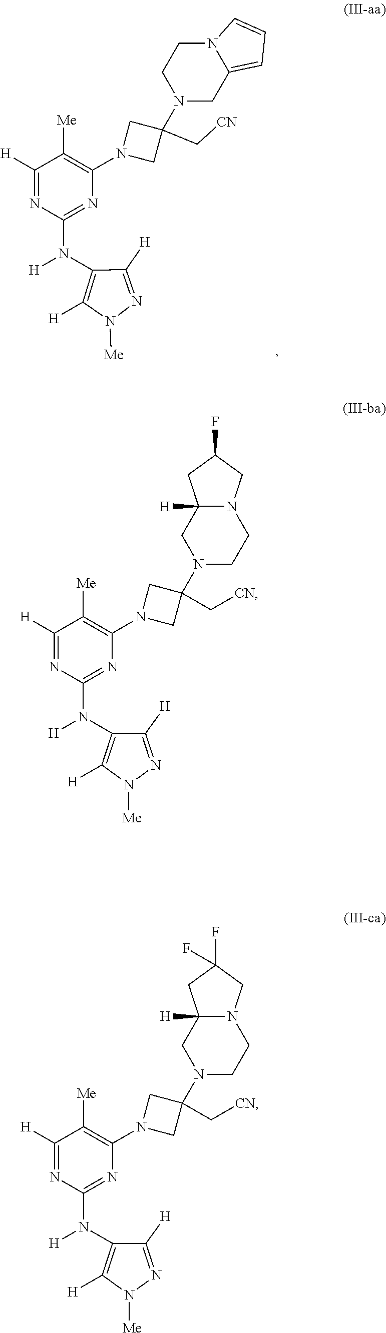 [SMILES: Cc1cnc(Nc2cnn(C)c2)nc1N1CC(CC#N)(N2CCn3cccc3C2)C1 ; Cc1cnc(Nc2cnn(C)c2)nc1N1CC(CC#N)(N2CCN3C[C@H](F)C[C@H]3C2)C1 ; Cc1cnc(Nc2cnn(C)c2)nc1N1CC(CC#N)(N2CCN3CC(F)(F)C[C@H]3C2)C1]

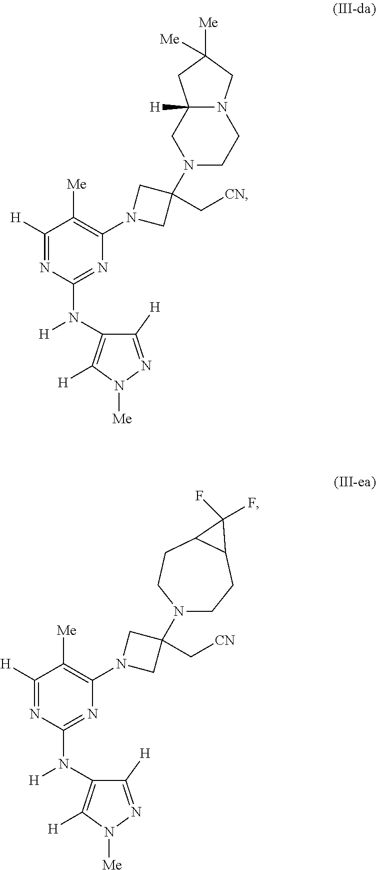 [SMILES: Cc1cnc(Nc2cnn(C)c2)nc1N1CC(CC#N)(N2CCN3CC(C)(C)C[C@H]3C2)C1 ; Cc1cnc(Nc2cnn(C)c2)nc1N1CC(CC#N)(N2CCC3C(CC2)C3(F)F)C1]

or a pharmaceutically acceptable salt thereof.

In some embodiments, the compound is of the formula:

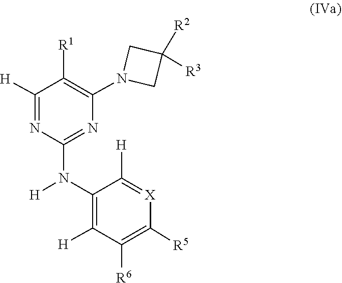

or a pharmaceutically acceptable salt thereof,
wherein
R¹ is H, F, Cl, CN, CF₃, C₁₋₆ alkyl, C₃₋₆ cycloalkyl, or C₁₋₆ haloalkyl;
R² is H, amino, amidyl, aminosulfonyl, cycloalkyl, C₁₋₆ alkyl, C₁₋₆ heteroalkyl or C₁₋₆ haloalkyl, heterocyclyl, heteroaryl or can link to R³ to form a ring;
R³ is C₁₋₆ alkyl, C₁₋₆ heteroalkyl or C₁₋₆ haloalkyl, CH₂CCH, CH₂CN, CH₂CH₂CN, CH₂CH₂CH₂CN, CH₂CO₂R⁴, CH₂CO₂H, CH₂CH₂OH, CH₂CH₂F, CH₂CH₂Cl, CH₂CHF₂, CH₂CF₃ or can link to R³ to form a ring;
R⁴ is C₁₋₃ alkyl;
R⁵ and R⁶ are each independently C₁₋₁₂ alkyl, C₃₋₆ cycloalkyl, C₁₋₁₂ heteroalkyl or C₁₋₁₂ haloalkyl, CH₂OH, CH₂CH₂OH, CH₂CH₂CH₂OH, alkylenecycloalkyl, alkylenecyclopropyl, methoxy, or R⁵ and R⁶ can combine to form a ring of 5 to 7 member atoms;
X is CH or N.

In some embodiments, R⁵ and R⁶ are each H.

In some embodiments, the compound is of the formula:

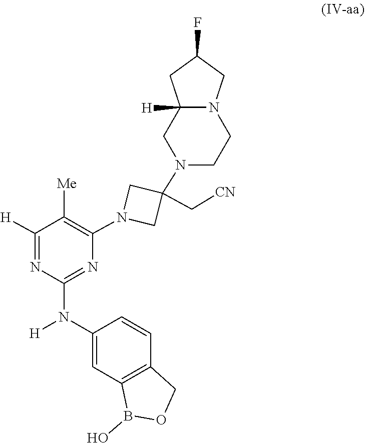 [SMILES: Cc1cnc(Nc2ccc3c(c2)B(O)OC3)nc1N1CC(CC#N)(N2CCN3C[C@H](F)C[C@H]3C2)C1]

or a pharmaceutically acceptable salt thereof.

In some embodiments, the compound is of the formula:

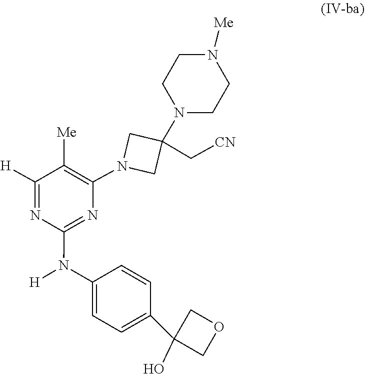 [SMILES: Cc1cnc(Nc2ccc(C3(O)COC3)cc2)nc1N1CC(CC#N)(N2CCN(C)CC2)C1]

or a pharmaceutically acceptable salt thereof.

In some embodiments, the compound is a compound of the formulae:

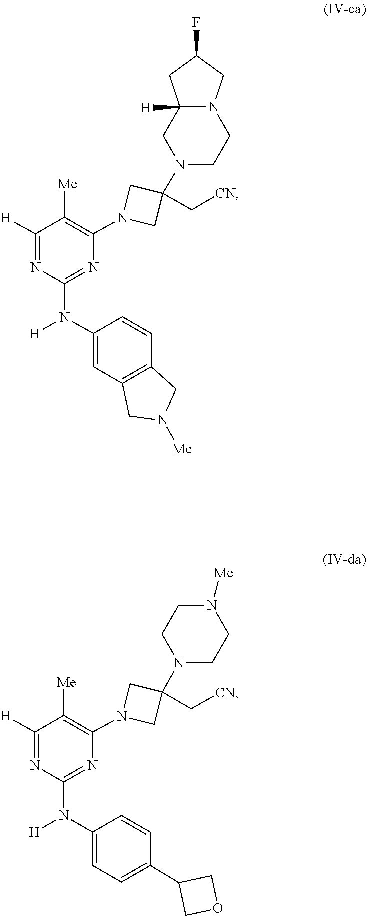 [SMILES: Cc1cnc(Nc2ccc3c(c2)CN(C)C3)nc1N1CC(CC#N)(N2CCN3C[C@H](F)C[C@H]3C2)C1 ; Cc1cnc(Nc2ccc(C3COC3)cc2)nc1N1CC(CC#N)(N2CCN(C)CC2)C1]

or a pharmaceutically acceptable salt thereof.

In one aspect, provided herein are compounds of the formula:

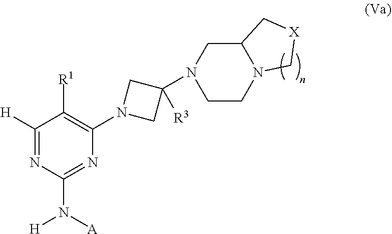

or a pharmaceutically acceptable salt thereof,
wherein
R¹ is H, F, Cl, CN, CF₃, C₁₋₆ alkyl, C₃₋₆ cycloalkyl, or C₁₋₆ haloalkyl;
R³ is C₁₋₆ alkyl, C₁₋₆ heteroalkyl or C₁₋₆ haloalkyl, CH₂CCH, CH₂CN, CH₂CH₂CN, CH₂CH₂CH₂CN, CH₂CO₂R⁴, CH₂CO₂H, CH₂CH₂OH, CH₂CH₂F, CH₂CH₂Cl, CH₂CHF₂, CH₂CF₃;
R⁴ is C₁₋₃ alkyl;
n is a whole number between 0 and 4;
X is CR⁵R⁶ for n=0 and n=1, and, when n>1, X can be either CR⁵R⁶ or O, or NR⁴;
R⁵ and R⁶ are each independently H, F, Cl, CN, OCH₃, CF₃, C₁₋₆ alkyl, C₃₋₆ cycloalkyl, or C₁₋₆ haloalkyl, C₁₋₆ heteroalkyl, or R⁵ and R⁶ can connect to form a ring of 3 to 5 members; and
A is heteroaryl.

In some embodiments, the compound is a compound of one of the formulae:

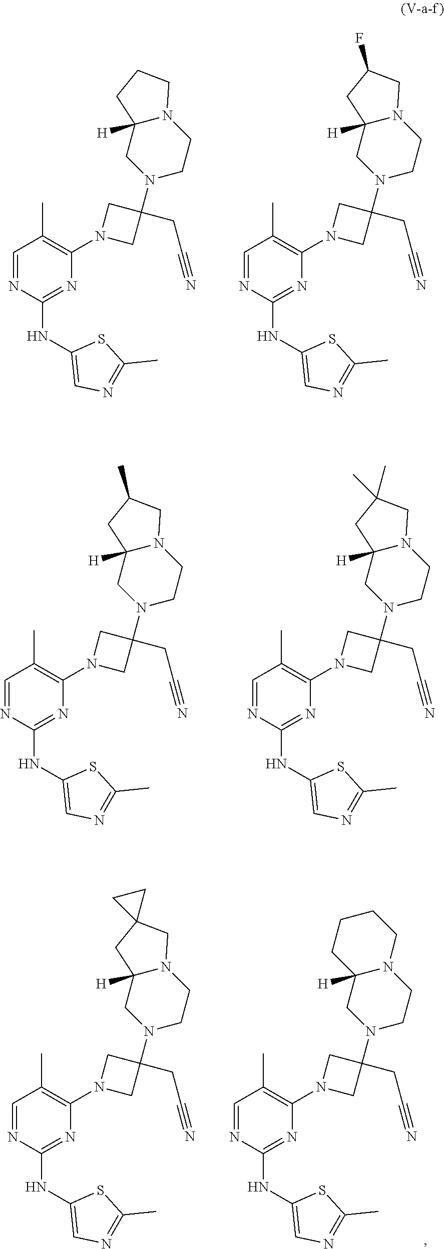

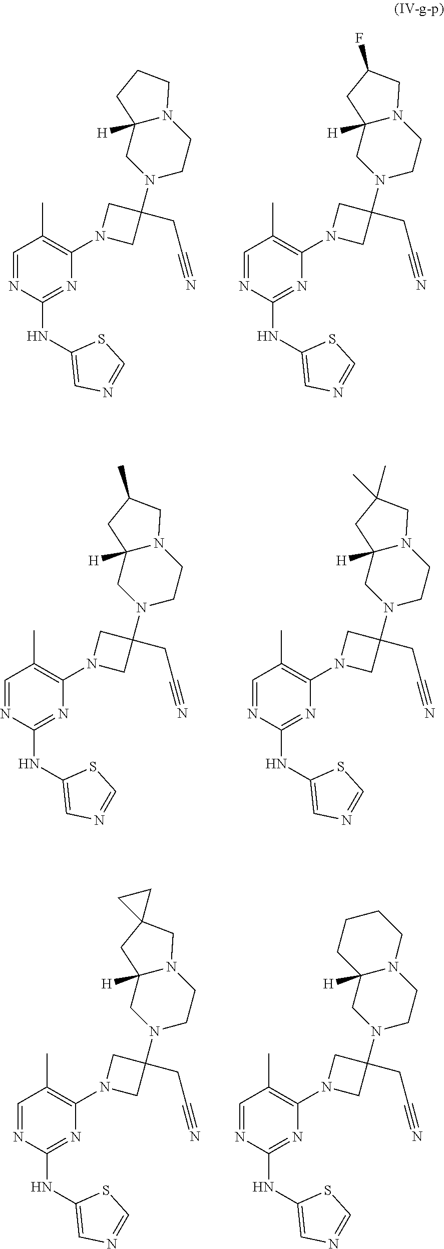

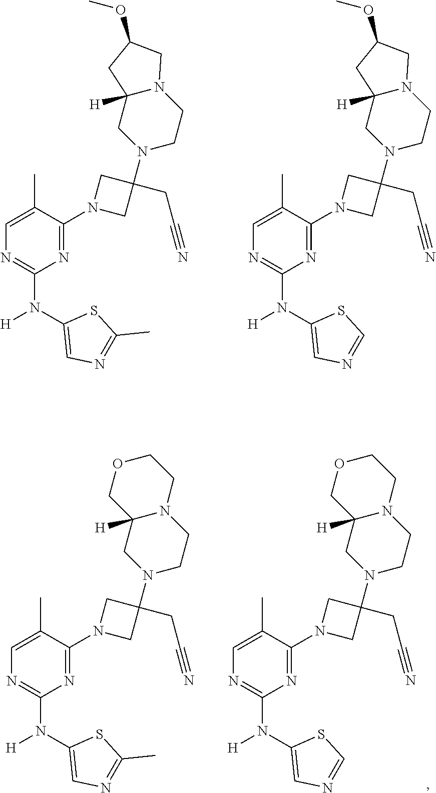

or a pharmaceutically acceptable salt thereof.

In some embodiments of the formulae provided herein:
R¹ is H, F, Cl, Me, CN, cyclopropyl, or C₁₋₃ alkyl;
R² is a substituted piperidyl; or
R⁵ is hydroxymethyl or C₁₋₃ alkyl.

In some embodiments of the formulae provided herein, R¹ is methyl.

In some embodiments of the formulae provided herein, R³ is —CH₂CN.

In some embodiments of the formulae provided herein, R³ is —CH₂CH₂CN.

In some embodiments of the formulae provided herein, R³ is —CH₂CH₂F.

In some embodiments of the formulae provided herein, R⁵ is SO₂Me.

In some embodiments of the formulae provided herein, X is a nitrogen atom.

In some embodiments of the formulae provided herein, R² is alkylene amino, e.g. (C₁₋₃ alkylene)-amino.

In some embodiments of the formulae provided herein, R¹ is H.

In some embodiments of the formulae provided herein, R³ is alkylene amino, e.g. (C₁₋₃ alkylene)-amino.

In some embodiments of the formulae provided herein, R³ is cyclopropyl.

In some embodiments of the formulae provided herein, R² is a monocyclic C₃₋₅ heteroaryl having 1-3 nitrogen atoms.

In some embodiments, the compound is:

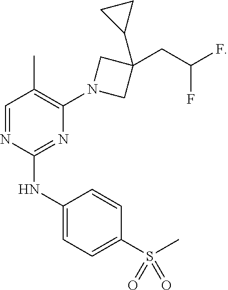 [SMILES: Cc1cnc(Nc2ccc(S(C)(=O)=O)cc2)nc1N1CC(CC(F)F)(C2CC2)C1]

### Synthesis

Methods of synthesizing the herein described compounds will be evident to those of ordinary skill in the art. Synthetic chemistry transformations and protecting group methodologies (protection and deprotection) useful in synthesizing the compounds are known in the art and include, for example, those such as described in R. LaRock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

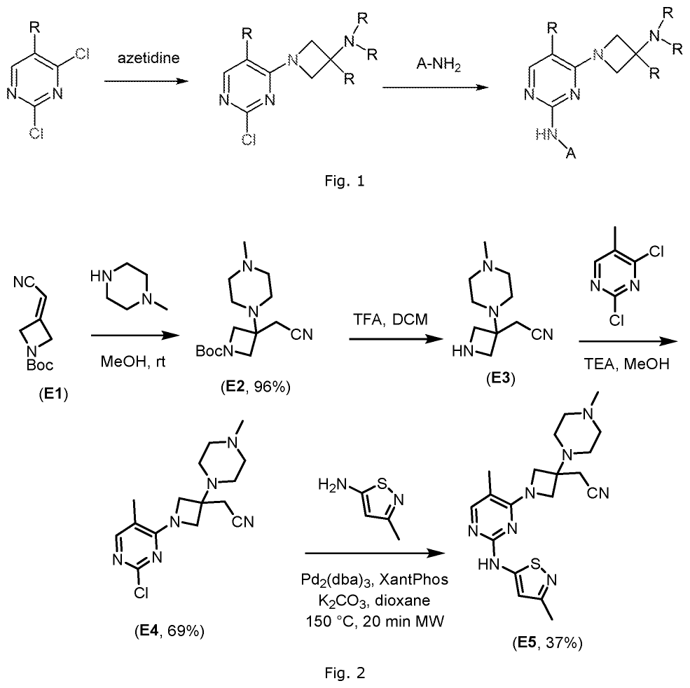

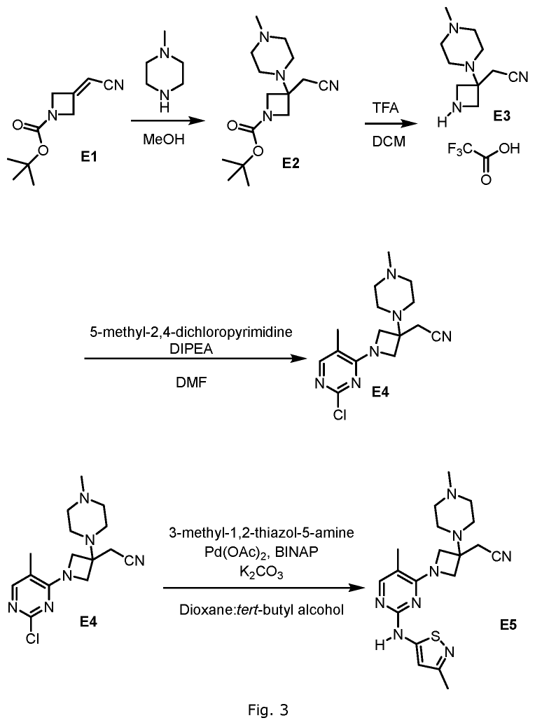

In general, the compounds provided herein may be prepared according to the general scheme shown in FIG. 1. For example, in some embodiments, the compounds are synthesized by first coupling the azetidine to the pyrimidine at the 4-position of the pyrimidine, and then the heteroaryl or aryl amine is added. FIG. 2 illustrates a typical synthesis using this route.

Preparation of tert-butyl 3-(cyanomethyl)-3-(4-methylpiperazin-1-yl)azetidine-1-carboxylate (E2): To a solution of tert-butyl 3-(cyanomethylene)azetidine-1-carboxylate (E1, 200 mg, 1.030 mmol) in MeOH (1.3 mL) was added 1-methylpiperazine (172 μL, 1.545 mmol). Reaction was stirred at room temperature until starting material was consumed by TLC (5% MeOH/DCM, iodine stain). The reaction was poured over saturated NaHCO₃ and extracted with EtOAc. The organic layer was dried over Na₂SO₄ then concentrated under reduced pressure. Compound E2 was purified by column chromatograph (0-5% MeOH:DCM) to give a white solid (E2, 96%).

Preparation of 2-(3-(4-methylpiperazin-1-yl)azetidin-3-yl)acetonitrile trifluoroacetate (E3): To a solution of tert-butyl 3-(cyanomethyl)-3-(4-methylpiperazin-1-yl)azetidine-1-carboxylate (E2, 110 mg, 0.374 mmol) in dichloromethane (3.7 mL) was added trifluoroacetic acid (0.4 mL). Reaction was stirred at room temperature until starting material was consumed based on TLC (5% MeOH/DCM, stained with KMnO₄). Solution was concentrated under reduced pressure and the clear, colorless, crude oil (E3) was carried on to the next step.

Preparation of 2-(1-(2-chloro-5-methylpyrimidin-4-yl)-3-(4-methylpiperazin-1-yl)azetidin-3-yl) acetonitrile (E4): To a solution of 2,4-dichloro-5-methylpyrimidine (73 mg, 0.449 mmol) in methanol (2 mL) was added triethylamine (0.5 mL, 3.74 mmol). E3 was added slowly to the solution and its vial rinsed with methanol (0.5 mL×3). Reaction was stirred at room temperature overnight or until E3 was consumed by LC/MS. The reaction was poured over saturated NaHCO₃ and extracted with EtOAc. The organic layer was dried over Na₂SO₄ then concentrated under reduced pressure. Compound E4 was purified by column chromatograph (0-5% MeOH:DCM) to give a white solid (69% over two steps).

Preparation of 2-(1-(5-methyl-2-((3-methylisothiazol-5-yl)amino)pyrimidin-4-yl)-3-(4-methyl-piperazin-1-yl)azetidin-3-yl)acetonitrile (E5): To a microwave vial was added E4 (100 mg, 0.312 mmol), 3-methylisothiazol-5-amine (39 mg, 0.343 mmol), Pd₂(dba)₃ (14 mg, 5 mol %), XantPhos (27 mg, 15 mol %), K₂CO₃ (172 mg, 1.25 mmol) and dioxane (3.1 mL). Reaction was heated to 150° C. for 20 minutes in an Anton Paar Monowave. Reaction mixture was filtered through celite into 1M HCl_{aq} in a separatory funnel. The celite was washed with ethyl acetate. Aqueous layer was washed with ethyl acetate to remove significant yellow color. Then sat NaHCO₃ was then added to the aqueous layer until basic and precipitate has formed. Aqueous layer was extracted with ethyl acetate. The organic layer was dried over Na₂SO₄ then concentrated under reduced pressure. Compound E5 was purified by column chromatograph (0-5% MeOH:DCM) to give a white solid (37%).

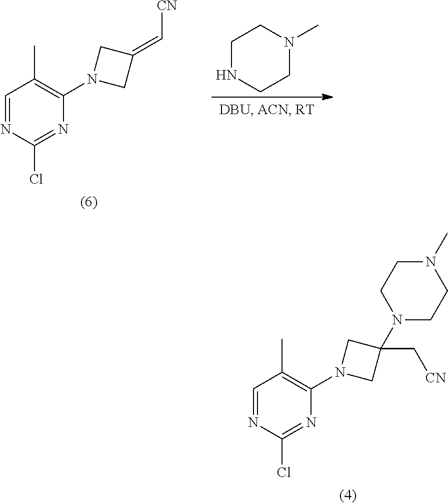

Alternate preparation of 2-(1-(2-chloro-5-methylpyrimidin-4-yl)-3-(4-methylpiperazin-1-yl)azetidin-3-yl)acetonitrile (E4): To a dry vial was charged intermediate (6) (100 mg; 0.45 mmol; 1.0 eq) and DBU (671 μL; 2.25 mmol; 10.0 eq) in dry ACN (0.6 mL). To the solution was added 1-methylpiperazine (76 μL; 0.68 mmol; 1.5 eq). The solution turned red. After 30 minutes, the reaction was complete by TLC and LC. The reaction mixture was poured over saturated sodium bicarbonate and sodium chloride and extracted with DCM:IPA (3:1). The combined organics were dried over sodium sulfate, filtered, then evaporated. The crude residue was purified by column chromatography (DCM:EtOH) to afford intermediate (E4) in 700/yield.

Using the schemes described above, making adjustments as necessary for protecting groups, etc., the compounds of Table 1 E5-E52 were synthesized.

TABLE 1

| Com- | | |
|---|---|---|
| pound | | |
| Num- | | |
| ber | Structure | IC₅₀ |
| E6 | 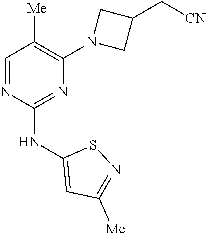 [SMILES: Cc1cc(Nc2ncc(C)c(N3CC(CC#N)C3)n2)sn1] | <10 nM TYK2 |
| E7 | 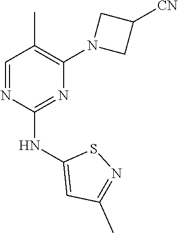 [SMILES: Cc1cc(Nc2ncc(C)c(N3CC(C#N)C3)n2)sn1] | <50 nM JAK2 |
| E8 | 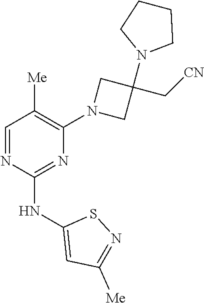 [SMILES: Cc1cc(Nc2ncc(C)c(N3CC(CC#N)(N4CCCC4)C3)n2)sn1] | <10 nM TYK2 <10 nM JAK1 |
| E9 | 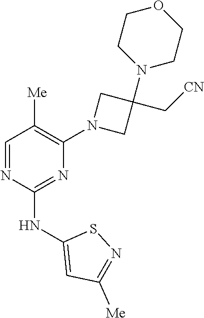 [SMILES: Cc1cc(Nc2ncc(C)c(N3CC(CC#N)(N4CCOCC4)C3)n2)sn1] | <10 nM TYK2 <10 nM JAK1 |
| E5 | 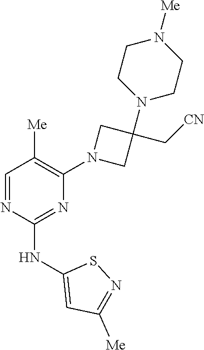 [SMILES: Cc1cc(Nc2ncc(C)c(N3CC(CC#N)(N4CCN(C)CC4)C3)n2)sn1] | <10 nM TYK2 <10 nM JAK1 |
| E10 | 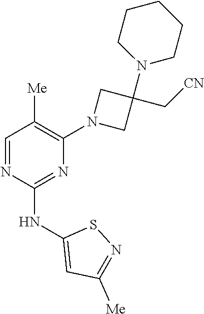 [SMILES: Cc1cc(Nc2ncc(C)c(N3CC(CC#N)(N4CCCCC4)C3)n2)sn1] | <10 nM TYK2 <10 nM JAK1 |
| E11 | 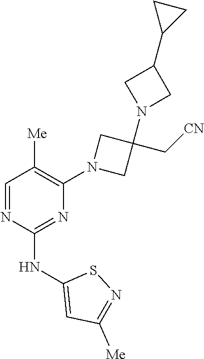 [SMILES: Cc1cc(Nc2ncc(C)c(N3CC(CC#N)(N4CC(C5CC5)C4)C3)n2)sn1] | <10 nM TYK2 <10 nM JAK1 |
| E12 | 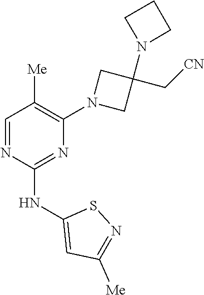 [SMILES: Cc1cc(Nc2ncc(C)c(N3CC(CC#N)(N4CCC4)C3)n2)sn1] | <10 nM TYK2 <10 nM JAK1 |
| E13 | 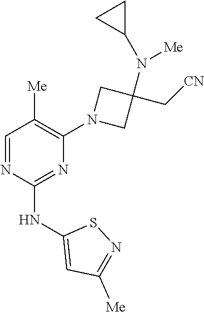 [SMILES: Cc1cc(Nc2ncc(C)c(N3CC(CC#N)(N(C)C4CC4)C3)n2)sn1] | <10 nM TYK2 <10 nM JAK1 |
| E14 | 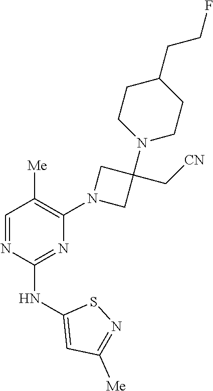 [SMILES: Cc1cc(Nc2ncc(C)c(N3CC(CC#N)(N4CCC(CCF)CC4)C3)n2)sn1] | <10 nM TYK2 <10 nM JAK1 |
| E15 | 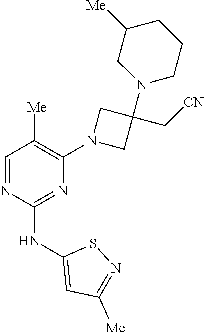 [SMILES: Cc1cc(Nc2ncc(C)c(N3CC(CC#N)(N4CCCC(C)C4)C3)n2)sn1] | <10 nM TYK2 <10 nM JAK1 |
| E16 | 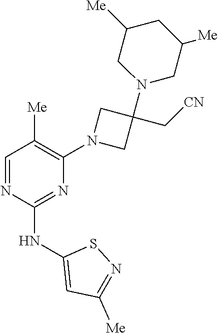 [SMILES: Cc1cc(Nc2ncc(C)c(N3CC(CC#N)(N4CC(C)CC(C)C4)C3)n2)sn1] | <5 nM TYK2 <5 nM JAK1 |
| E17 | 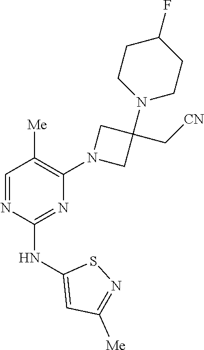 [SMILES: Cc1cc(Nc2ncc(C)c(N3CC(CC#N)(N4CCC(F)CC4)C3)n2)sn1] | <10 nM TYK2 <10 nM JAK1 |
| E18 | 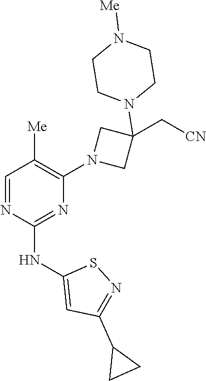 [SMILES: Cc1cnc(Nc2cc(C3CC3)ns2)nc1N1CC(CC#N)(N2CCN(C)CC2)C1] | <5 nM TYK2 <5 nM JAK1 |
| E19 | 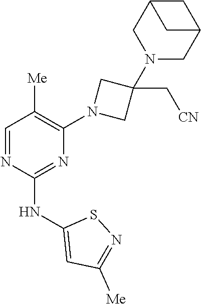 [SMILES: Cc1cc(Nc2ncc(C)c(N3CC(CC#N)(N4CC5CC(C5)C4)C3)n2)sn1] | <10 nM TYK2 <10 nM JAK1 |
| E20 | 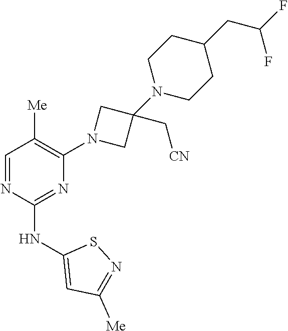 [SMILES: Cc1cc(Nc2ncc(C)c(N3CC(CC#N)(N4CCC(CC(F)F)CC4)C3)n2)sn1] | <10 nM TYK2 <10 nM JAK1 |
| E21 | 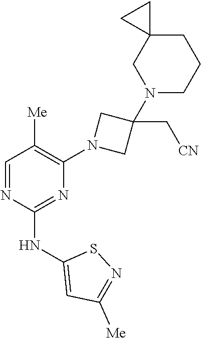 [SMILES: Cc1cc(Nc2ncc(C)c(N3CC(CC#N)(N4CCCC5(CC5)C4)C3)n2)sn1] | <10 nM TYK2 <10 nM JAK1 |
| E22 | 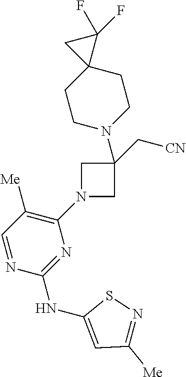 [SMILES: Cc1cc(Nc2ncc(C)c(N3CC(CC#N)(N4CCC5(CC4)CC5(F)F)C3)n2)sn1] | <10 nM TYK2 <10 nM JAK1 |
| E23 | 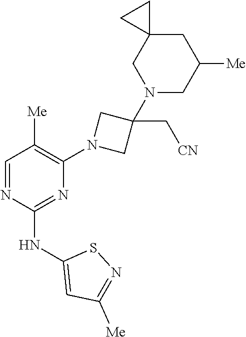 [SMILES: Cc1cc(Nc2ncc(C)c(N3CC(CC#N)(N4CC(C)CC5(CC5)C4)C3)n2)sn1] | <10 nM TYK2 <10 nM JAK1 |
| E24 | 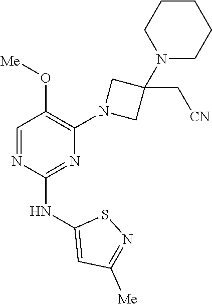 [SMILES: COc1cnc(Nc2cc(C)ns2)nc1N1CC(CC#N)(N2CCCCC2)C1] | <50 nM TYK2 <50 nM JAK1 |
| E25 | 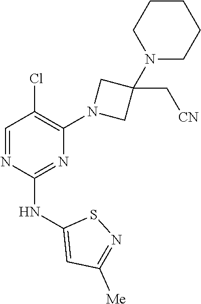 [SMILES: Cc1cc(Nc2ncc(Cl)c(N3CC(CC#N)(N4CCCCC4)C3)n2)sn1] | <10 nM TYK2 <10 nM JAK1 |
| E26 | 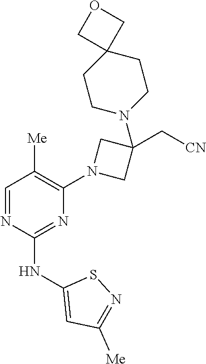 [SMILES: Cc1cc(Nc2ncc(C)c(N3CC(CC#N)(N4CCC5(CC4)COC5)C3)n2)sn1] | <10 nM TYK2 <10 nM JAK1 |
| E27 | 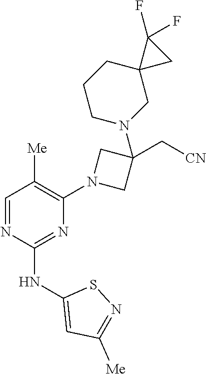 [SMILES: Cc1cc(Nc2ncc(C)c(N3CC(CC#N)(N4CCCC5(C4)CC5(F)F)C3)n2)sn1] | <10 nM TYK2 <10 nM JAK1 |
| E28 | 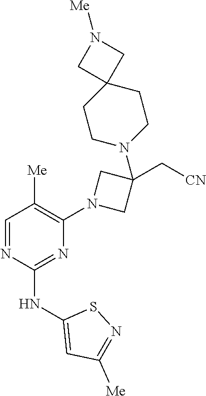 [SMILES: Cc1cc(Nc2ncc(C)c(N3CC(CC#N)(N4CCC5(CC4)CN(C)C5)C3)n2)sn1] | <50 nM TYK2 <50 nM JAK1 |
| E29 | 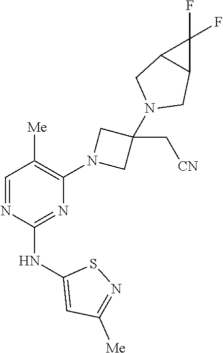 [SMILES: Cc1cc(Nc2ncc(C)c(N3CC(CC#N)(N4CC5C(C4)C5(F)F)C3)n2)sn1] | <10 nM TYK2 <10 nM JAK1 |
| E30 | 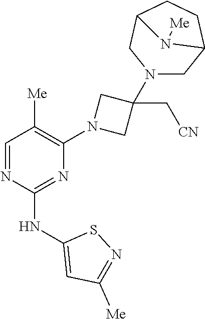 [SMILES: Cc1cc(Nc2ncc(C)c(N3CC(CC#N)(N4CC5CCC(C4)N5C)C3)n2)sn1] | <10 nM TYK2 <50 nM JAK1 |
| E31 | 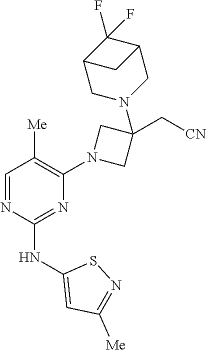 [SMILES: Cc1cc(Nc2ncc(C)c(N3CC(CC#N)(N4CC5CC(C4)C5(F)F)C3)n2)sn1] | <10 nM TYK2 <10 nM JAK1 |
| E32 | 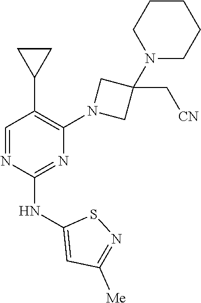 [SMILES: Cc1cc(Nc2ncc(C3CC3)c(N3CC(CC#N)(N4CCCCC4)C3)n2)sn1] | <10 nM TYK2 <50 nM JAK1 |
| E33 | 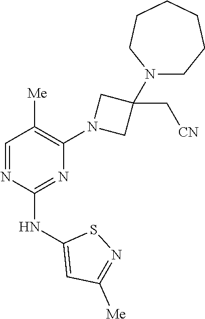 [SMILES: Cc1cc(Nc2ncc(C)c(N3CC(CC#N)(N4CCCCCC4)C3)n2)sn1] | <10 nM TYK2 <10 nM JAK1 |
| E34 | 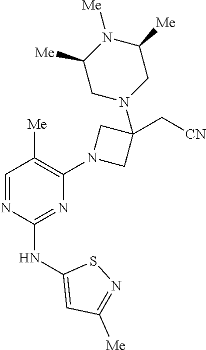 [SMILES: Cc1cc(Nc2ncc(C)c(N3CC(CC#N)(N4C[C@@H](C)N(C)[C@@H](C)C4)C3)n2)sn1] | <10 nM TYK2 <50 nM JAK1 |
| E35 | 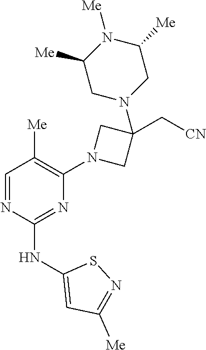 [SMILES: Cc1cc(Nc2ncc(C)c(N3CC(CC#N)(N4C[C@@H](C)N(C)[C@H](C)C4)C3)n2)sn1] | <10 nM TYK2 <50 nM JAK1 |
| E36 | 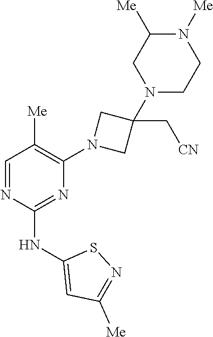 [SMILES: Cc1cc(Nc2ncc(C)c(N3CC(CC#N)(N4CCN(C)C(C)C4)C3)n2)sn1] | <10 nM TYK2 <10 nM JAK1 |
| E37 | 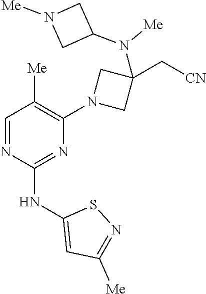 [SMILES: Cc1cc(Nc2ncc(C)c(N3CC(CC#N)(N(C)C4CN(C)C4)C3)n2)sn1] | <50 nM TYK2 <50 nM JAK1 |
| E38 | 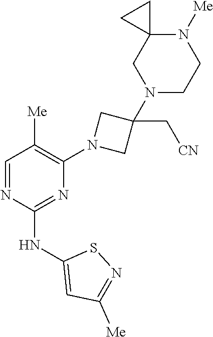 [SMILES: Cc1cc(Nc2ncc(C)c(N3CC(CC#N)(N4CCN(C)C5(CC5)C4)C3)n2)sn1] | <10 nM TYK2 <10 nM JAK1 |
| E39 | 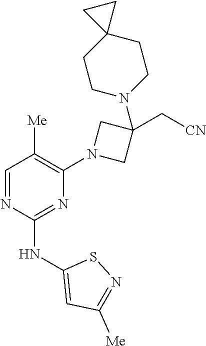 [SMILES: Cc1cc(Nc2ncc(C)c(N3CC(CC#N)(N4CCC5(CC4)CC5)C3)n2)sn1] | <10 nM TYK2 <10 nM JAK1 |
| E40 | 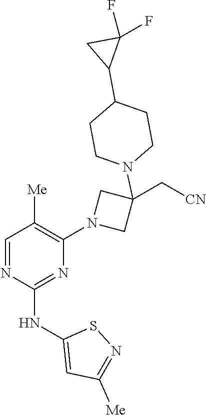 [SMILES: Cc1cc(Nc2ncc(C)c(N3CC(CC#N)(N4CCC(C5CC5(F)F)CC4)C3)n2)sn1] | <10 nM TYK2 <10 nM JAK1 |
| E41 | 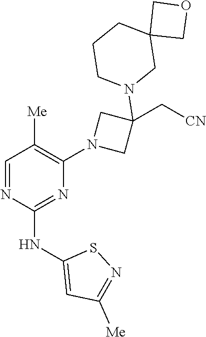 [SMILES: Cc1cc(Nc2ncc(C)c(N3CC(CC#N)(N4CCCC5(COC5)C4)C3)n2)sn1] | <10 nM TYK2 <10 nM JAK1 |
| E42 | 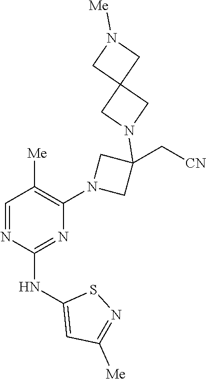 [SMILES: Cc1cc(Nc2ncc(C)c(N3CC(CC#N)(N4CC5(CN(C)C5)C4)C3)n2)sn1] | <1 μM TYK2 <1 μM JAK1 |
| E43 | 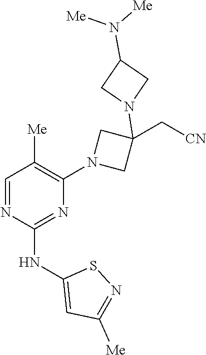 [SMILES: Cc1cc(Nc2ncc(C)c(N3CC(CC#N)(N4CC(N(C)C)C4)C3)n2)sn1] | <100 nM TYK2 <150 nM JAK1 |
| E44 | 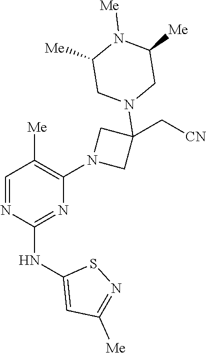 [SMILES: Cc1cc(Nc2ncc(C)c(N3CC(CC#N)(N4C[C@H](C)N(C)[C@@H](C)C4)C3)n2)sn1] | <20 nM TYK2 <50 nM JAK1 |
| E45 | 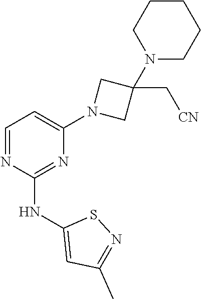 [SMILES: Cc1cc(Nc2nccc(N3CC(CC#N)(N4CCCCC4)C3)n2)sn1] | <20 nM TYK2 <200 nM JAK1 |
| E46 | 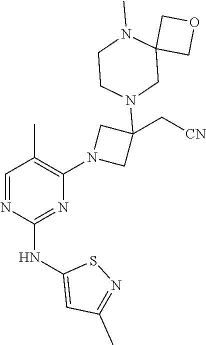 [SMILES: Cc1cc(Nc2ncc(C)c(N3CC(CC#N)(N4CCN(C)C5(COC5)C4)C3)n2)sn1] | <10 nM TYK2 <20 nM JAK1 |
| E47 | 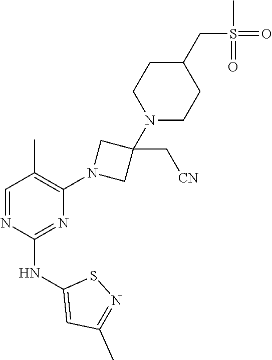 [SMILES: Cc1cc(Nc2ncc(C)c(N3CC(CC#N)(N4CCC(CS(C)(=O)=O)CC4)C3)n2)sn1] | <10 nM TYK2 <10 nM JAK1 |
| E48 | 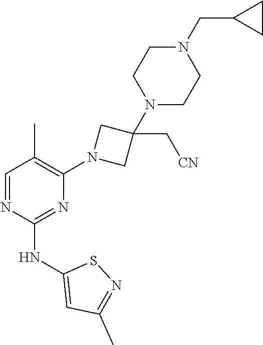 [SMILES: Cc1cc(Nc2ncc(C)c(N3CC(CC#N)(N4CCN(CC5CC5)CC4)C3)n2)sn1] | <10 nM TYK2 <10 nM JAK1 |
| E49 | 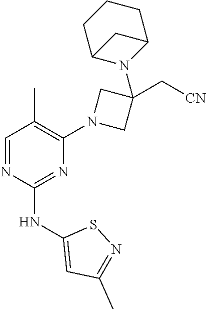 [SMILES: Cc1cc(Nc2ncc(C)c(N3CC(CC#N)(N4C5CCCC4C5)C3)n2)sn1] | <20 nM TYK2 <30 nM JAK1 |
| E50 | 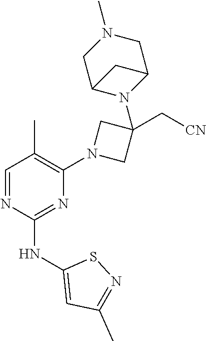 [SMILES: Cc1cc(Nc2ncc(C)c(N3CC(CC#N)(N4C5CC4CN(C)C5)C3)n2)sn1] | <400 nM TYK2 <250 nM JAK1 |
| E51 | 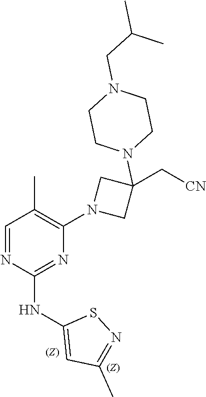 [SMILES: Cc1cc(Nc2ncc(C)c(N3CC(CC#N)(N4CCN(CC(C)C)CC4)C3)n2)sn1] | <20 nM TYK2 <15 nM JAK1 |
| E52 | 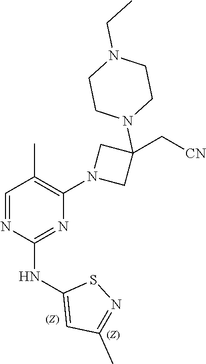 [SMILES: CCN1CCN(C2(CC#N)CN(c3nc(Nc4cc(C)ns4)ncc3C)C2)CC1] | <10 nM TYK2 <10 nM JAK1 |
| E52A | 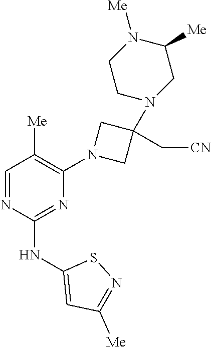 [SMILES: Cc1cc(Nc2ncc(C)c(N3CC(CC#N)(N4CCN(C)[C@@H](C)C4)C3)n2)sn1] | <10 nM TYK2 <20 nM JAK1 |
| E52B | 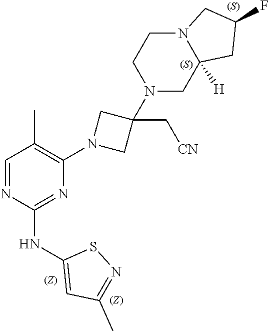 | <10 nM TYK2 <10 nM JAK1 |
| E52C | 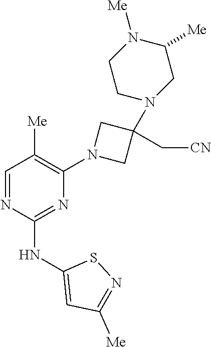 [SMILES: Cc1cc(Nc2ncc(C)c(N3CC(CC#N)(N4CCN(C)[C@H](C)C4)C3)n2)sn1] | <10 nM TYK2 <20 nM JAK2 |
| E52D | 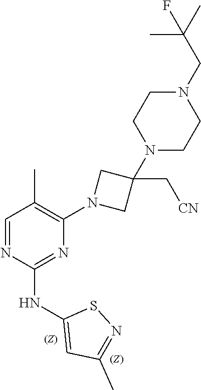 [SMILES: Cc1cc(Nc2ncc(C)c(N3CC(CC#N)(N4CCN(CC(C)(C)F)CC4)C3)n2)sn1] | <20 nM TYK2 <10 nM JAK1 |
| E52E | 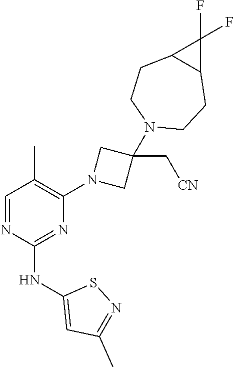 [SMILES: Cc1cc(Nc2ncc(C)c(N3CC(CC#N)(N4CCC5C(CC4)C5(F)F)C3)n2)sn1] | <10 nM TYK2 <10 nM JAK1 |
| E52F | 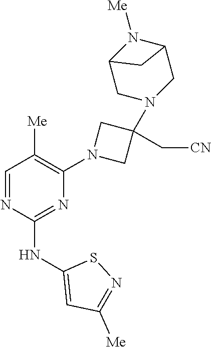 [SMILES: Cc1cc(Nc2ncc(C)c(N3CC(CC#N)(N4CC5CC(C4)N5C)C3)n2)sn1] | <50 nM TYK2 <100 nM JAK1 |
| E52G | 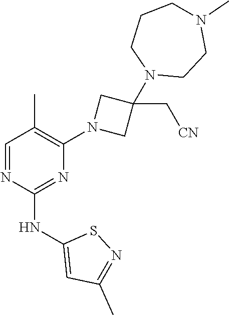 [SMILES: Cc1cc(Nc2ncc(C)c(N3CC(CC#N)(N4CCCN(C)CC4)C3)n2)sn1] | <50 nM TYK2 <100 nM JAK1 |
| E52H | 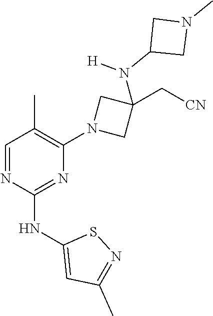 [SMILES: Cc1cc(Nc2ncc(C)c(N3CC(CC#N)(NC4CN(C)C4)C3)n2)sn1] | <100 nM TYK2 <100 nM JAK1 |

Using substantially the same procedures as described above, with appropriate modifications, compounds E53-E79 (Table 2 thru 5) can be prepared.

TABLE 2

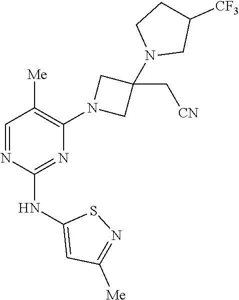 [SMILES: Cc1cc(Nc2ncc(C)c(N3CC(CC#N)(N4CCC(C(F)(F)F)C4)C3)n2)sn1]

E53

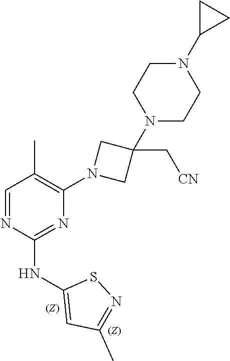 [SMILES: Cc1cc(Nc2ncc(C)c(N3CC(CC#N)(N4CCN(C5CC5)CC4)C3)n2)sn1]

E54
<10 nM TYK2
<10 nM JAK1

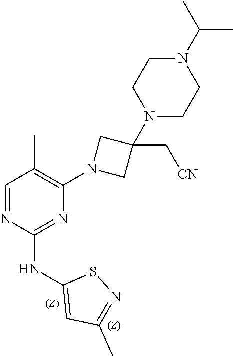 [SMILES: Cc1cc(Nc2ncc(C)c(N3CC(CC#N)(N4CCN(C(C)C)CC4)C3)n2)sn1]

E55
<50 nM TYK2
<50 nM JAK1

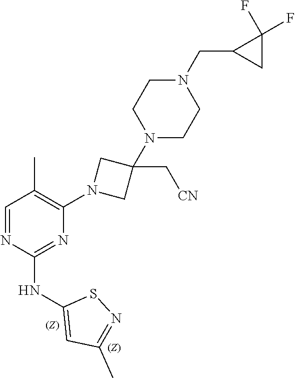 [SMILES: Cc1cc(Nc2ncc(C)c(N3CC(CC#N)(N4CCN(CC5CC5(F)F)CC4)C3)n2)sn1]

E56

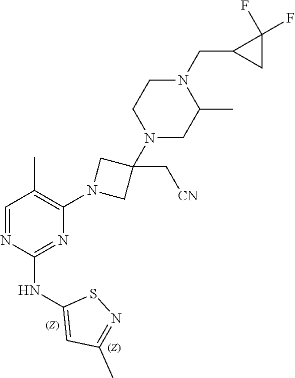 [SMILES: Cc1cc(Nc2ncc(C)c(N3CC(CC#N)(N4CCN(CC5CC5(F)F)C(C)C4)C3)n2)sn1]

E57

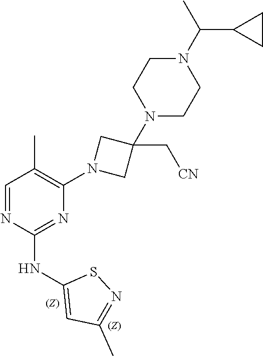 [SMILES: Cc1cc(Nc2ncc(C)c(N3CC(CC#N)(N4CCN(C(C)C5CC5)CC4)C3)n2)sn1]

E58

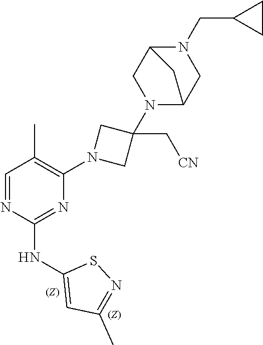 [SMILES: Cc1cc(Nc2ncc(C)c(N3CC(CC#N)(N4CC5CC4CN5CC4CC4)C3)n2)sn1]

E59

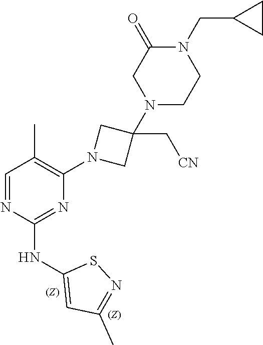 [SMILES: Cc1cc(Nc2ncc(C)c(N3CC(CC#N)(N4CCN(CC5CC5)C(=O)C4)C3)n2)sn1]

E60

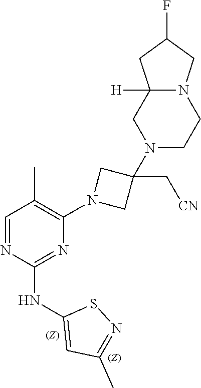 [SMILES: Cc1cc(Nc2ncc(C)c(N3CC(CC#N)(N4CCN5CC(F)CC5C4)C3)n2)sn1]

E61

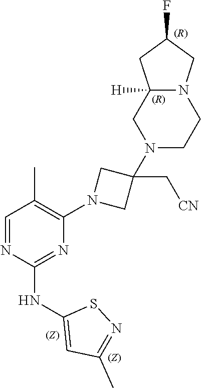

E62

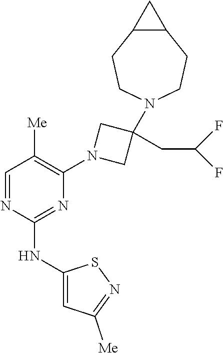 [SMILES: Cc1cc(Nc2ncc(C)c(N3CC(CC(F)F)(N4CCC5CC5CC4)C3)n2)sn1]

E63

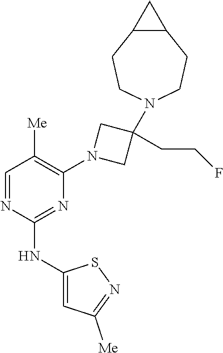 [SMILES: Cc1cc(Nc2ncc(C)c(N3CC(CCF)(N4CCC5CC5CC4)C3)n2)sn1]

E64

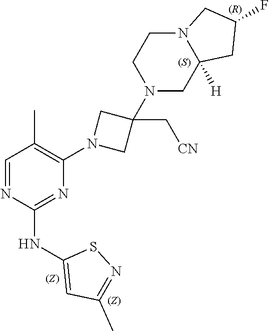

E65a

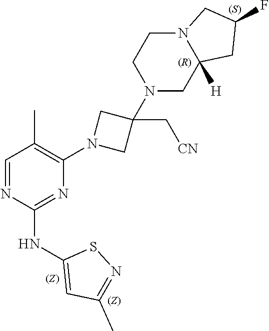

E66a

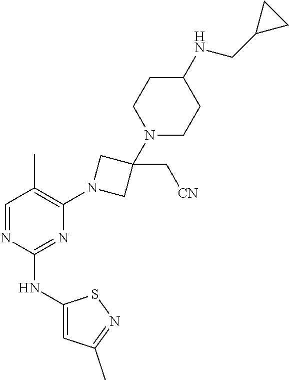 [SMILES: Cc1cc(Nc2ncc(C)c(N3CC(CC#N)(N4CCC(NCC5CC5)CC4)C3)n2)sn1]

E67

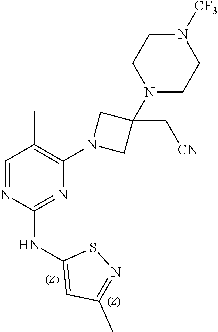 [SMILES: Cc1cc(Nc2ncc(C)c(N3CC(CC#N)(N4CCN(C(F)(F)F)CC4)C3)n2)sn1]

E68

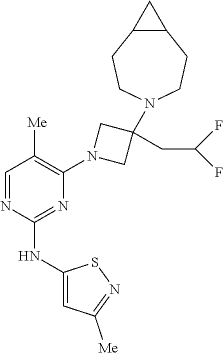 [SMILES: Cc1cc(Nc2ncc(C)c(N3CC(CC(F)F)(N4CCC5CC5CC4)C3)n2)sn1]

E69

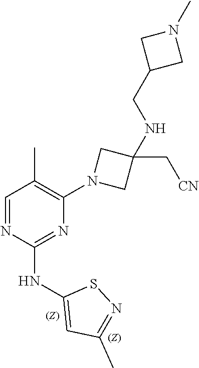 [SMILES: Cc1cc(Nc2ncc(C)c(N3CC(CC#N)(NCC4CN(C)C4)C3)n2)sn1]

E70

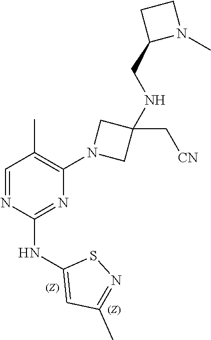 [SMILES: Cc1cc(Nc2ncc(C)c(N3CC(CC#N)(NC[C@H]4CCN4C)C3)n2)sn1]

E71

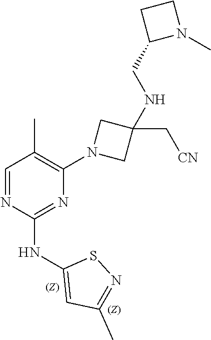 [SMILES: Cc1cc(Nc2ncc(C)c(N3CC(CC#N)(NC[C@@H]4CCN4C)C3)n2)sn1]

E72

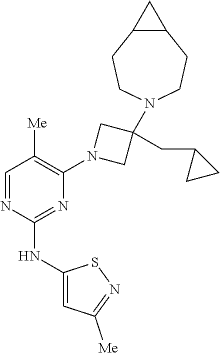 [SMILES: Cc1cc(Nc2ncc(C)c(N3CC(CC4CC4)(N4CCC5CC5CC4)C3)n2)sn1]

E73
E74

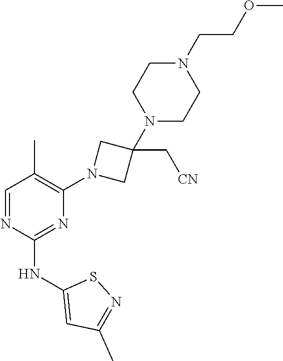 [SMILES: COCCN1CCN(C2(CC#N)CN(c3nc(Nc4cc(C)ns4)ncc3C)C2)CC1]

E75
<10 nM TYK2
<10 nM JAK1

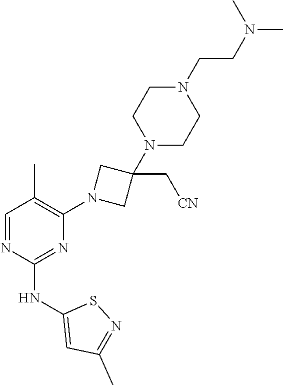 [SMILES: Cc1cc(Nc2ncc(C)c(N3CC(CC#N)(N4CCN(CCN(C)C)CC4)C3)n2)sn1]

E76

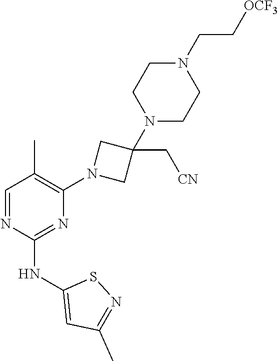 [SMILES: Cc1cc(Nc2ncc(C)c(N3CC(CC#N)(N4CCN(CCOC(F)(F)F)CC4)C3)n2)sn1]

E77

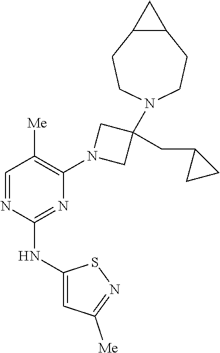 [SMILES: Cc1cc(Nc2ncc(C)c(N3CC(CC4CC4)(N4CCC5CC5CC4)C3)n2)sn1]

E78

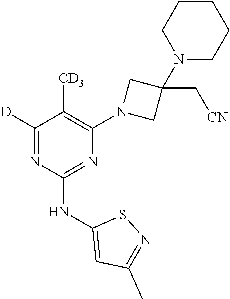 [SMILES: [2H]c1nc(Nc2cc(C)ns2)nc(N2CC(CC#N)(N3CCCCC3)C2)c1C([2H])([2H])[2H]]

E78A

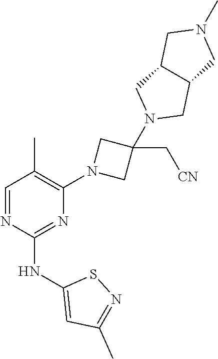 [SMILES: Cc1cc(Nc2ncc(C)c(N3CC(CC#N)(N4C[C@H]5CN(C)C[C@H]5C4)C3)n2)sn1]

E79

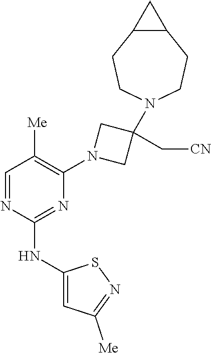 [SMILES: Cc1cc(Nc2ncc(C)c(N3CC(CC#N)(N4CCC5CC5CC4)C3)n2)sn1]

E79A

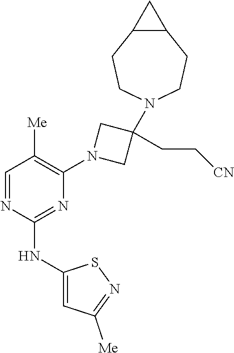 [SMILES: Cc1cc(Nc2ncc(C)c(N3CC(CCC#N)(N4CCC5CC5CC4)C3)n2)sn1]

E79B

TABLE 3

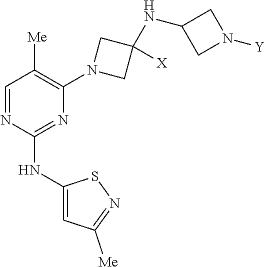

| Number | X | Y |
|---|---|---|
| S1-3-1 | methyl | methyl |
| S1-3-2 | ethyl | methyl |
| S1-3-3 | cyclobutyl | methyl |
| S1-3-4 | 3-fluorocyclobut-1-yl | methyl |
| S1-3-5 | cyclopentyl | methyl |
| S1-3-6 | tetrahydro-2H-pyran-4-yl | methyl |
| S1-3-7 | isopropyl | methyl |
| S1-3-8 | cyclohexyl | methyl |
| S1-3-9 | cycloheptyl | methyl |
| S1-3-10 | methylenecyclopropyl | methyl |
| S1-3-11 | 2-fluoroethyl | methyl |
| S1-3-12 | 2,2-difluoroethyl | methyl |
| S1-3-13 | 2-fluoroethyl | ethyl |
| S1-3-14 | —CH₂CN | isopropyl |

TABLE 4

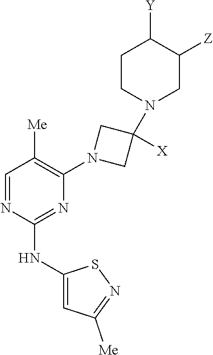

| Number | X | Y | Z |
|---|---|---|---|
| S1-4-1 | —CH₂CN | fluoro | methyl |
| S1-4-2 | —CH₂CH₂CN | fluoro | methyl |
| S1-4-3 | 2-fluoroethyl | fluoro | methyl |
| S1-4-4 | 3-fluorocyclobut-1-yl | fluoro | methyl |
| S1-4-5 | cyclopentyl | fluoro | methyl |
| S1-4-6 | tetrahydro-2H-pyran-4-yl | fluoro | methyl |
| S1-4-7 | isopropyl | fluoro | methyl |
| S1-4-8 | cyclohexyl | fluoro | methyl |
| S1-4-9 | cycloheptyl | fluoro | methyl |
| S1-4-10 | methylenecyclopropyl | fluoro | methyl |
| S1-4-11 | 2-fluoroethyl | fluoro | fluoro |
| S1-4-12 | 2,2-difluoroethyl | fluoro | methyl |
| S1-4-13 | 2-fluoroethyl | fluoro | ethyl |
| S1-4-14 | —CH₂CN | fluoro | isopropyl |

TABLE 5

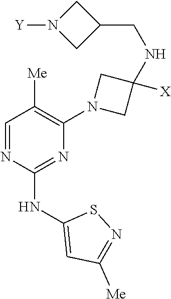

| Number | X | Y |
|---|---|---|
| S1-5-1 | methyl | methyl |
| S1-5-2 | ethyl | methyl |
| S1-5-3 | cyclobutyl | methyl |
| S1-5-4 | 3-fluorocyclobut-1-yl | methyl |
| S1-5-5 | cyclopentyl | methyl |
| S1-5-6 | tetrahydro-2H-pyran-4-yl | methyl |
| S1-5-7 | isopropyl | methyl |
| S1-5-8 | cyclohexyl | methyl |
| S1-5-9 | cycloheptyl | methyl |
| S1-5-10 | methylenecyclopropyl | methyl |
| S1-5-11 | 2-fluoroethyl | methyl |
| S1-5-12 | 2,2-difluoroethyl | methyl |
| S1-5-13 | 2-fluoroethyl | ethyl |
| S1-5-14 | —CH₂CN | isopropyl |
| S1-5-15 | —CH₂CN | methyl |

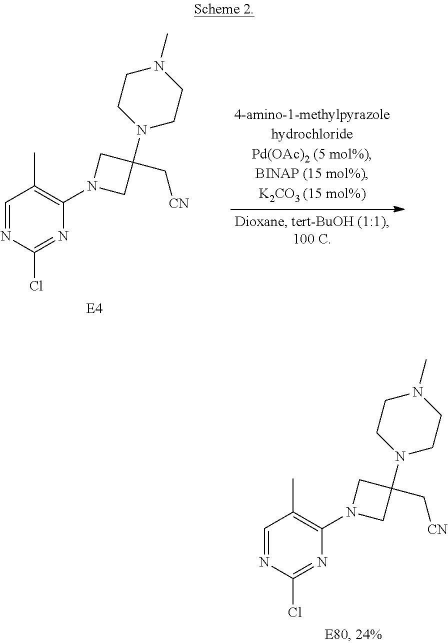 [SMILES: Cc1cnc(Cl)nc1N1CC(CC#N)(N2CCN(C)CC2)C1 ; Cc1cnc(Cl)nc1N1CC(CC#N)(N2CCN(C)CC2)C1]

Preparation of 2-(1-(5-methyl-2-((1-methyl-H-pyrazol-4-yl)amino)pyrimidin-4-yl)-3-(4-methyl-piperazin-1-yl)azetidin-3-yl)acetonitrile (E80): To 2-(1-(2-chloro-5-methylpyrimidin-4-yl)-3-(4-methylpiperazin-1-yl)azetidin-3-yl)acetonitrile (E4, 74 mg, 0.23 mmol, 1.0 eq) under nitrogen was charged 4-amino-1-methyl-pyrazole hydrochloride (41 mg, 0.24 mmol, 1.05 eq), palladium (II) acetate (2 mg, 0.01 mmol, 5 mol %), BINAP (19 mg, 0.03 mmol, 15 mol %), and potassium carbonate (127 mg, 0.92 mmol, 4.0 eq) followed by dioxane and tert-butyl alcohol (2 mL, 1:1). The reaction mixture was heated overnight at 100 deg C. The reaction mixture was poured into saturated sodium bicarbonate and ethyl acetate. The organic layer was separated, and the aqueous layer was extracted with ethyl acetate. The combined organic layers were dried over sodium sulfate, then filtered and evaporated. The crude residue was purified by column chromatography (DCM:EtOH) to afford 2-(1-(5-methyl-2-((1-methyl-1H-pyrazol-4-yl)amino)pyrimidin-4-yl)-3-(4-methylpiperazin-1-yl)azetidin-3-yl)acetonitrile (E80) in 24% yield (21 mg, 0.06 mmol).

Using procedures similar to those set forth above for Scheme 2 and substituting the appropriate starting materials, the compounds E80-82 (Table 6) were made and the compounds E83-109 and of Tables 7 thru 10 could be synthesized.

TABLE 6

| | | |
|---|---|---|
| E80 | 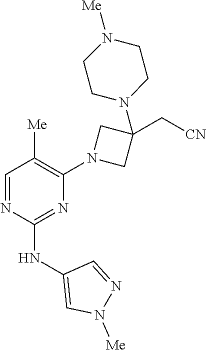 | <20 nM TYK2 <20 nM JAK1 |
| E81 | 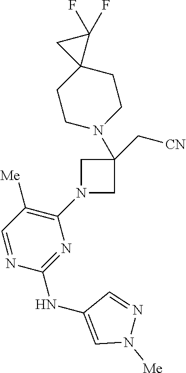 | <10 nM TYK2 <10 nM JAK1 |
| E82 | 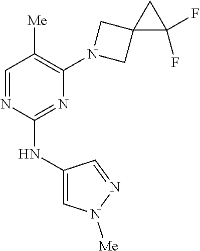 | <20 nM TYK2 <20 nM JAK1 |

TABLE 7

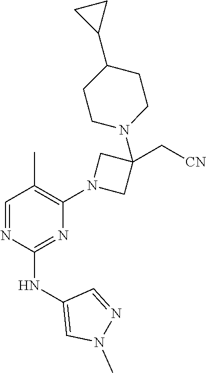 [SMILES: Cc1cnc(Nc2cnn(C)c2)nc1N1CC(CC#N)(N2CCC(C3CC3)CC2)C1]

E83

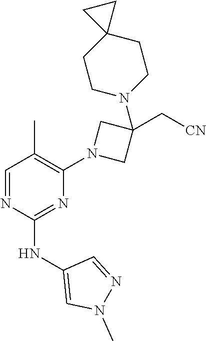 [SMILES: Cc1cnc(Nc2cnn(C)c2)nc1N1CC(CC#N)(N2CCC3(CC2)CC3)C1]

E84

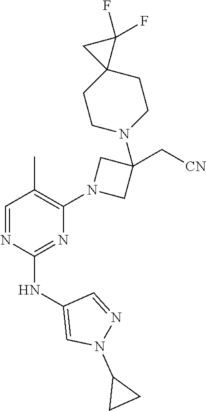 [SMILES: Cc1cnc(Nc2cnn(C3CC3)c2)nc1N1CC(CC#N)(N2CCC3(CC2)CC3(F)F)C1]

E85

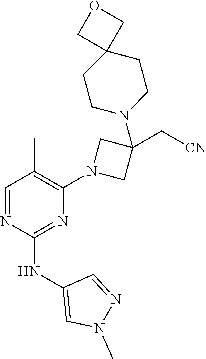 [SMILES: Cc1cnc(Nc2cnn(C)c2)nc1N1CC(CC#N)(N2CCC3(CC2)COC3)C1]

E86

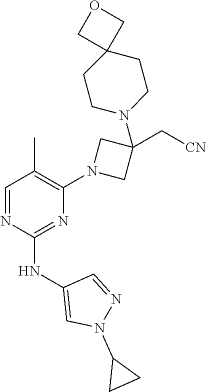 [SMILES: Cc1cnc(Nc2cnn(C3CC3)c2)nc1N1CC(CC#N)(N2CCC3(CC2)COC3)C1]

E87

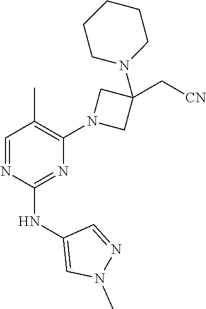 [SMILES: Cc1cnc(Nc2cnn(C)c2)nc1N1CC(CC#N)(N2CCCCC2)C1]

E88

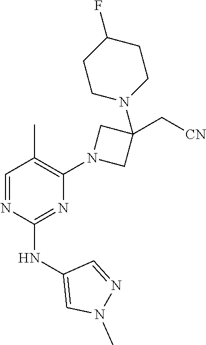 [SMILES: Cc1cnc(Nc2cnn(C)c2)nc1N1CC(CC#N)(N2CCC(F)CC2)C1]

E89

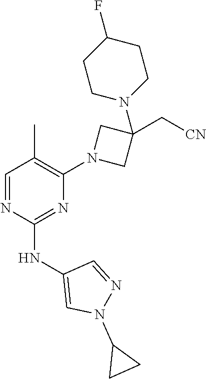 [SMILES: Cc1cnc(Nc2cnn(C3CC3)c2)nc1N1CC(CC#N)(N2CCC(F)CC2)C1]

E90

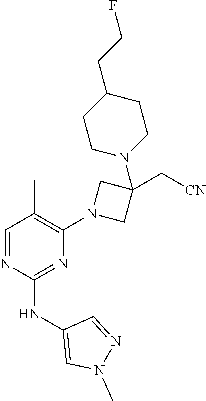 [SMILES: Cc1cnc(Nc2cnn(C)c2)nc1N1CC(CC#N)(N2CCC(CCF)CC2)C1]

E91

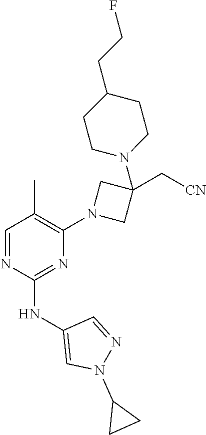 [SMILES: Cc1cnc(Nc2cnn(C3CC3)c2)nc1N1CC(CC#N)(N2CCC(CCF)CC2)C1]

E92

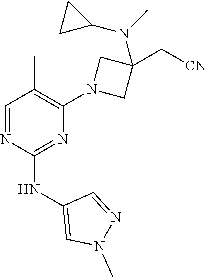 [SMILES: Cc1cnc(Nc2cnn(C)c2)nc1N1CC(CC#N)(N(C)C2CC2)C1]

E93

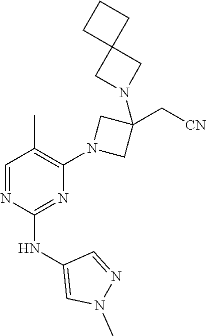 [SMILES: Cc1cnc(Nc2cnn(C)c2)nc1N1CC(CC#N)(N2CC3(CCC3)C2)C1]

E94

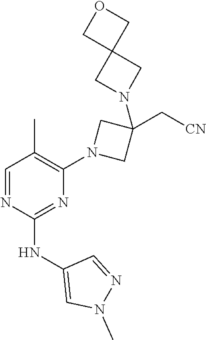 [SMILES: Cc1cnc(Nc2cnn(C)c2)nc1N1CC(CC#N)(N2CC3(COC3)C2)C1]

E95

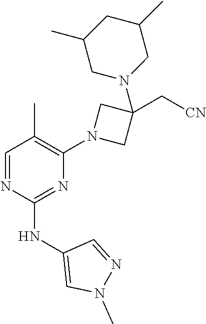 [SMILES: Cc1cnc(Nc2cnn(C)c2)nc1N1CC(CC#N)(N2CC(C)CC(C)C2)C1]

E96

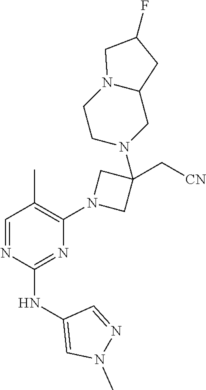 [SMILES: Cc1cnc(Nc2cnn(C)c2)nc1N1CC(CC#N)(N2CCN3CC(F)CC3C2)C1]

E97

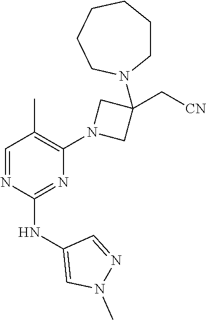 [SMILES: Cc1cnc(Nc2cnn(C)c2)nc1N1CC(CC#N)(N2CCCCCC2)C1]

E98

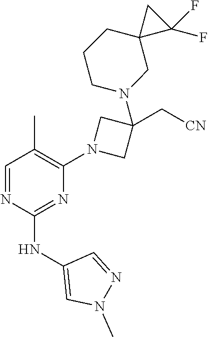 [SMILES: Cc1cnc(Nc2cnn(C)c2)nc1N1CC(CC#N)(N2CCCC3(C2)CC3(F)F)C1]

E99

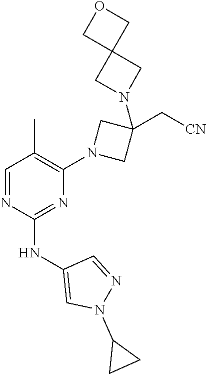 [SMILES: Cc1cnc(Nc2cnn(C3CC3)c2)nc1N1CC(CC#N)(N2CC3(COC3)C2)C1]

E100

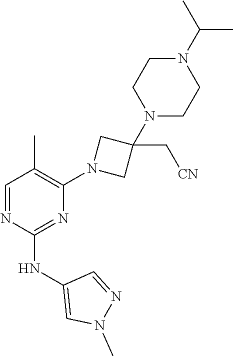 [SMILES: Cc1cnc(Nc2cnn(C)c2)nc1N1CC(CC#N)(N2CCN(C(C)C)CC2)C1]

E101

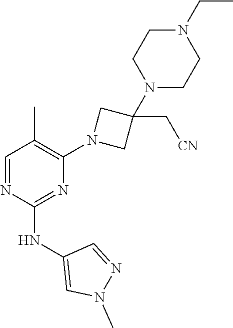 [SMILES: CCN1CCN(C2(CC#N)CN(c3nc(Nc4cnn(C)c4)ncc3C)C2)CC1]

E102

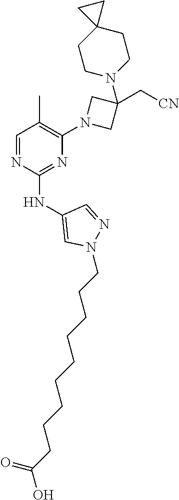 [SMILES: Cc1cnc(Nc2cnn(CCCCCCCCCC(=O)O)c2)nc1N1CC(CC#N)(N2CCC3(CC2)CC3)C1]

E103

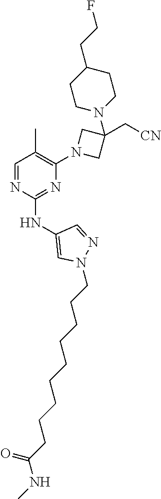 [SMILES: CNC(=O)CCCCCCCCCn1cc(Nc2ncc(C)c(N3CC(CC#N)(N4CCC(CCF)CC4)C3)n2)cn1]

E104

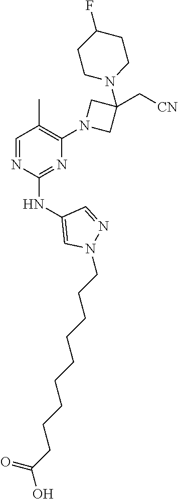 [SMILES: Cc1cnc(Nc2cnn(CCCCCCCCCC(=O)O)c2)nc1N1CC(CC#N)(N2CCC(F)CC2)C1]

E105

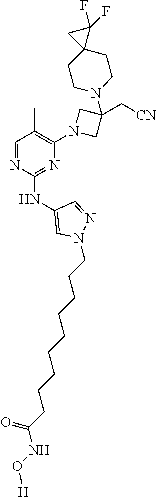 [SMILES: Cc1cnc(Nc2cnn(CCCCCCCCCC(=O)NO)c2)nc1N1CC(CC#N)(N2CCC3(CC2)CC3(F)F)C1]

E106

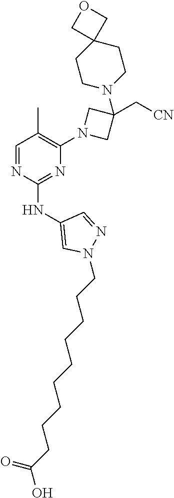 [SMILES: Cc1cnc(Nc2cnn(CCCCCCCCCC(=O)O)c2)nc1N1CC(CC#N)(N2CCC3(CC2)COC3)C1]

E107

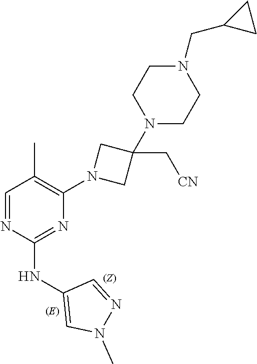 [SMILES: Cc1cnc(Nc2cnn(C)c2)nc1N1CC(CC#N)(N2CCN(CC3CC3)CC2)C1]

E108

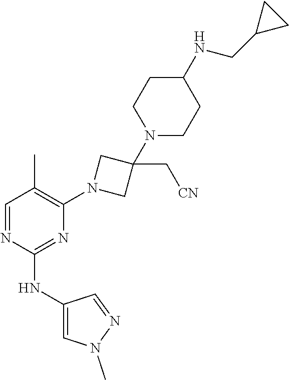 [SMILES: Cc1cnc(Nc2cnn(C)c2)nc1N1CC(CC#N)(N2CCC(NCC3CC3)CC2)C1]

E109

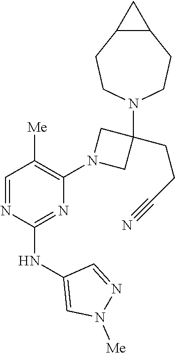 [SMILES: Cc1cnc(Nc2cnn(C)c2)nc1N1CC(CCC#N)(N2CCC3CC3CC2)C1]

E109A

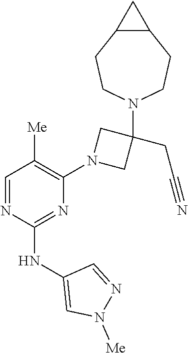 [SMILES: Cc1cnc(Nc2cnn(C)c2)nc1N1CC(CC#N)(N2CCC3CC3CC2)C1]

E109B

TABLE 8

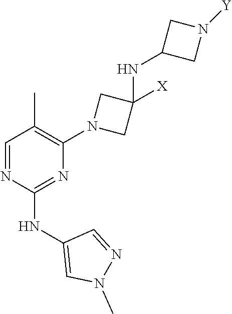

| Number | X | Y |
|---|---|---|
| S2-3-1 | methyl | methyl |
| S2-3-2 | ethyl | methyl |
| S2-3-3 | cyclobutyl | methyl |
| S2-3-4 | 3-fluorocyclobut-1-yl | methyl |
| S2-3-5 | cyclopentyl | methyl |
| S2-3-6 | tetrahydro-2H-pyran-4-yl | methyl |
| S2-3-7 | isopropyl | methyl |
| S2-3-8 | cyclohexyl | methyl |
| S2-3-9 | cycloheptyl | methyl |
| S2-3-10 | methylenecyclopropyl | methyl |
| S2-3-11 | 2-fluoroethyl | methyl |
| S2-3-12 | 2,2-difluoroethyl | methyl |
| S2-3-13 | 2-fluoroethyl | ethyl |
| S2-3-14 | —CH₂CN | isopropyl |

TABLE 9

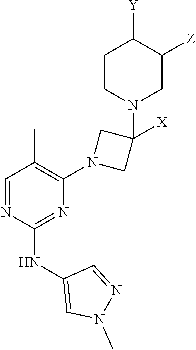

| Number | X | Y | Z |
|---|---|---|---|
| S2-4-1 | —CH₂CN | fluoro | methyl |
| S2-4-2 | —CH₂CH₂CN | fluoro | methyl |
| S2-4-3 | 2-fluoroethyl | fluoro | methyl |
| S2-4-4 | 3-fluorocyclobut-1-yl | fluoro | methyl |
| S2-4-5 | cyclopentyl | fluoro | methyl |
| S2-4-6 | tetrahydro-2H-pyran-4-yl | fluoro | methyl |
| S2-4-7 | isopropyl | fluoro | methyl |
| S2-4-8 | cyclohexyl | fluoro | methyl |
| S2-4-9 | cycloheptyl | fluoro | methyl |
| S2-4-10 | methylenecyclopropyl | fluoro | methyl |
| S2-4-11 | 2-fluoroethyl | fluoro | fluoro |
| S2-4-12 | 2,2-difluoroethyl | fluoro | methyl |
| S2-4-13 | 2-fluoroethyl | fluoro | ethyl |
| S2-4-14 | —CH₂CN | fluoro | isopropyl |

TABLE 10

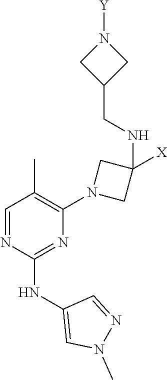

| Number | X | Y |
|---|---|---|
| S2-5-1 | methyl | methyl |
| S2-5-2 | ethyl | methyl |
| S2-5-3 | cyclobutyl | methyl |
| S2-5-4 | 3-fluorocyclobut-1-yl | methyl |
| S2-5-5 | cyclopentyl | methyl |
| S2-5-6 | tetrahydro-2H-pyran-4-yl | methyl |
| S2-5-7 | isopropyl | methyl |
| S2-5-8 | cyclohexyl | methyl |
| S2-5-9 | cycloheptyl | methyl |
| S2-5-10 | methylenecyclopropyl | methyl |
| S2-5-11 | 2-fluoroethyl | methyl |
| S2-5-12 | 2,2-difluoroethyl | methyl |
| S2-5-13 | 2-fluoroethyl | ethyl |
| S2-5-14 | —CH₂CN | isopropyl |

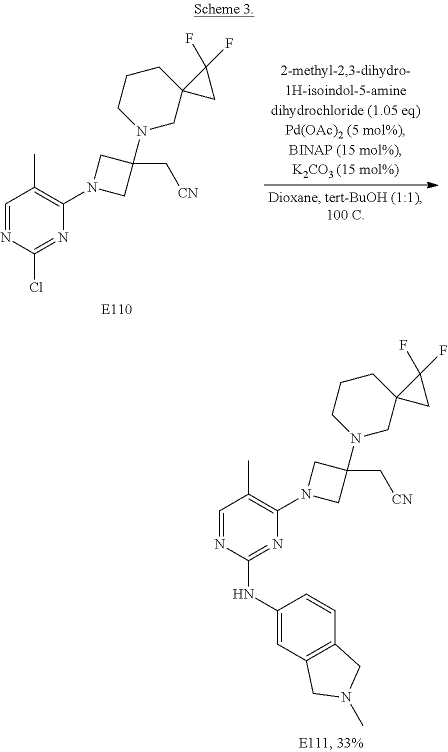 [SMILES: Cc1cnc(Cl)nc1N1CC(CC#N)(N2CCCC3(C2)CC3(F)F)C1 ; Cc1cnc(Nc2ccc3c(c2)CN(C)C3)nc1N1CC(CC#N)(N2CCCC3(C2)CC3(F)F)C1]

Preparation of 2-(3-(1,1-difluoro-5-azaspiro[2.5]octan-5-yl)-1-(5-methyl-2-((2-methylisoindolin-5-yl)amino)pyrimidin-4-yl)azetidin-3-yl)acetonitrile (E111): To 2-(1-(2-chloro-5-methylpyrimidin-4-yl)-3-(1,1-difluoro-5-azaspiro[2.5]octan-5-yl)azetidin-3-yl)acetonitrile (E110, 58 mg, 0.16 mmol, 1.0 eq) under nitrogen was charged 2-methyl-2,3-dihydro-1H-isoindol-5-amine dihydrochloride (37 mg, 0.17 mmol, 1.05 eq), palladium (II) acetate (2.2 mg, 0.01 mmol, 5 mol %), BINAP (15 mg, 0.02 mmol, 15 mol %), and potassium carbonate (231 mg, 0.63 mmol, 4.0 eq) followed by dioxane and tert-butyl alcohol (1.6 mL, 1:1). The reaction mixture was heated overnight at 100 deg C. The reaction mixture was poured into saturated sodium bicarbonate and ethyl acetate. The organic layer was separated, and the aqueous layer was extracted with 3:1 DCM/IPA. The combined organic layers were dried over sodium sulfate, then filtered and evaporated. The crude residue was purified by column chromatography (Hexanes:EtOAc, then DCM:EtOH) to afford 2-(3-(1,1-difluoro-5-azaspiro[2.5]octan-5-yl)-1-(5-methyl-2-((2-methylisoindolin-5-yl)amino)pyrimidin-4-yl)azetidin-3-yl)acetonitrile in 33% yield (E111, 25 mg, 0.05 mmol).

Using procedures to those set forth above for Schemes 1-3 and substituting the appropriate starting materials, the compounds E111-119 (Table 11) were made and compounds E120-144 and compounds in Tables 12 thru 15 could be synthesized.

TABLE 11

| | | |
|---|---|---|
| E112 | 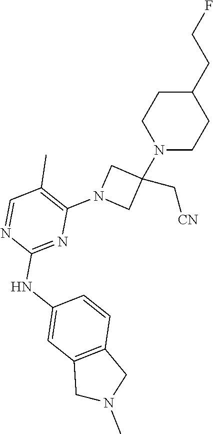 | <50 nM JAK2 <50 nM JAK1 |
| E113 | 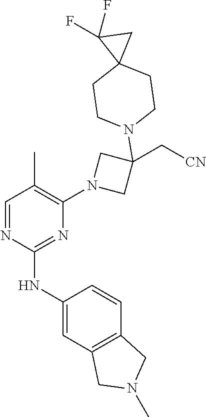 | <10 nM TYK2 <10 nM JAK1 |
| E111 | 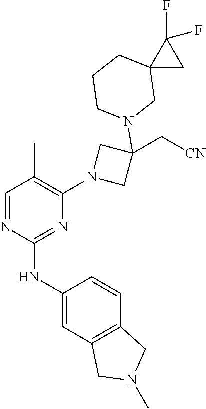 | <10 nM TYK2 <10 nM JAK1 |
| E114 | 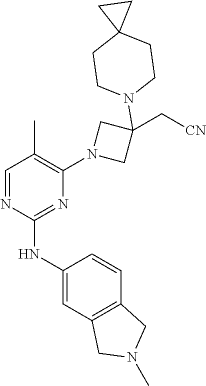 | <20 nM TYK2 <20 nM JAK1 |
| E115 | 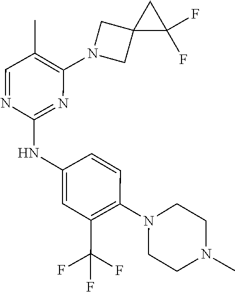 | <100 nM TYK2 <250 nM JAK1 |
| E116 | 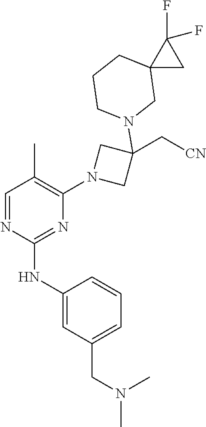 | <10 nM TYK2 <10 nM JAK1 |
| E117 | 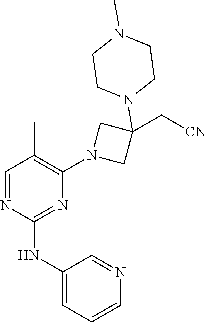 | <20 nM TYK2 <50 nM JAK1 |
| E118 | 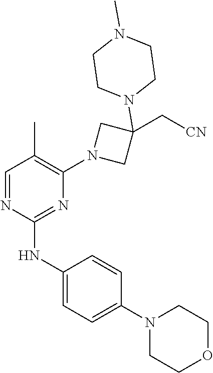 | <10 nM TYK2 <50 nM JAK1 |
| E119 | 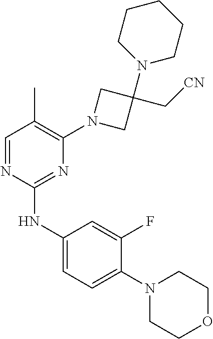 | <10 nM TYK2 <25 nM JAK1 |
| E119-A | 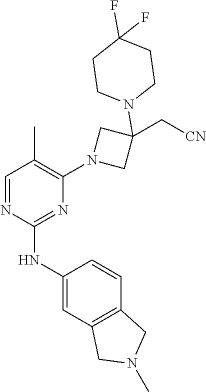 | <10 nM TYK2 <10 nM JAK1 |

TABLE 12

| | |
|---|---|
| 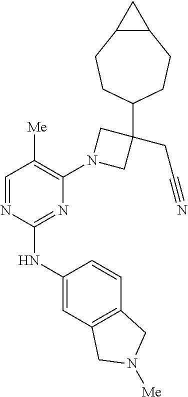 [SMILES: Cc1cnc(Nc2ccc3c(c2)CN(C)C3)nc1N1CC(CC#N)(C2CCC3CC3CC2)C1] | E120 |
| 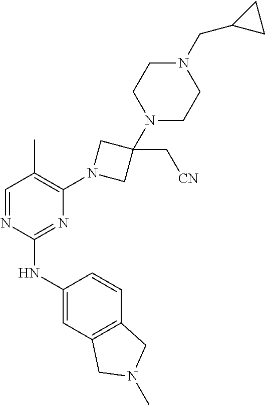 [SMILES: Cc1cnc(Nc2ccc3c(c2)CN(C)C3)nc1N1CC(CC#N)(N2CCN(CC3CC3)CC2)C1] | E121 |
| 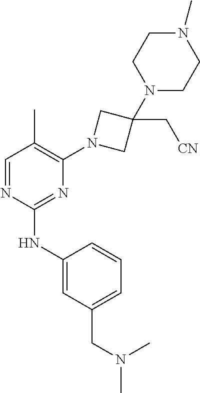 [SMILES: Cc1cnc(Nc2cccc(CN(C)C)c2)nc1N1CC(CC#N)(N2CCN(C)CC2)C1] | E122 |
| 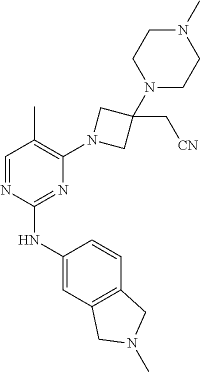 [SMILES: Cc1cnc(Nc2ccc3c(c2)CN(C)C3)nc1N1CC(CC#N)(N2CCN(C)CC2)C1] | E123 |
| 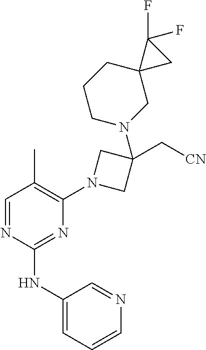 [SMILES: Cc1cnc(Nc2cccnc2)nc1N1CC(CC#N)(N2CCCC3(C2)CC3(F)F)C1] | E124 |
| 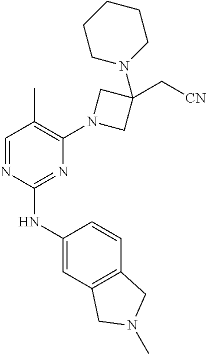 [SMILES: Cc1cnc(Nc2ccc3c(c2)CN(C)C3)nc1N1CC(CC#N)(N2CCCCC2)C1] | E125 |
| 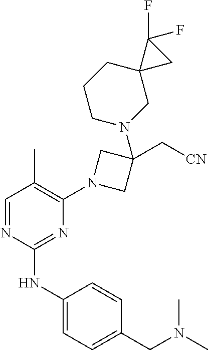 [SMILES: Cc1cnc(Nc2ccc(CN(C)C)cc2)nc1N1CC(CC#N)(N2CCCC3(C2)CC3(F)F)C1] | E126 |
| 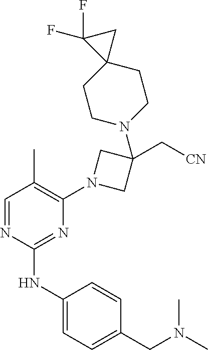 [SMILES: Cc1cnc(Nc2ccc(CN(C)C)cc2)nc1N1CC(CC#N)(N2CCC3(CC2)CC3(F)F)C1] | E127 |
| 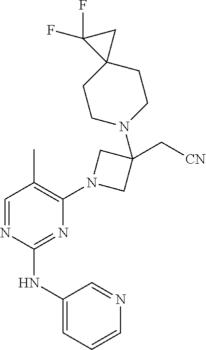 [SMILES: Cc1cnc(Nc2cccnc2)nc1N1CC(CC#N)(N2CCC3(CC2)CC3(F)F)C1] | E128 |
| 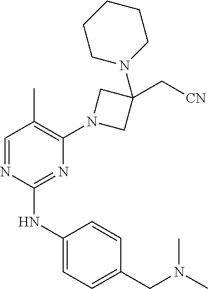 [SMILES: Cc1cnc(Nc2ccc(CN(C)C)cc2)nc1N1CC(CC#N)(N2CCCCC2)C1] | E129 |
| 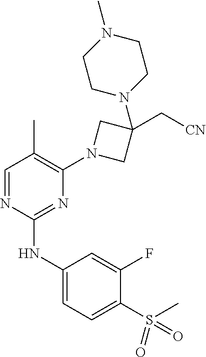 [SMILES: Cc1cnc(Nc2ccc(S(C)(=O)=O)c(F)c2)nc1N1CC(CC#N)(N2CCN(C)CC2)C1] | E130 |
| 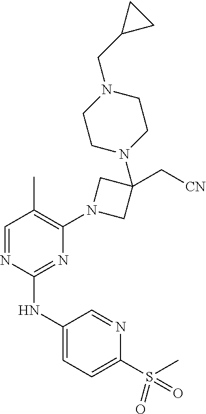 [SMILES: Cc1cnc(Nc2ccc(S(C)(=O)=O)nc2)nc1N1CC(CC#N)(N2CCN(CC3CC3)CC2)C1] | E131 |
| 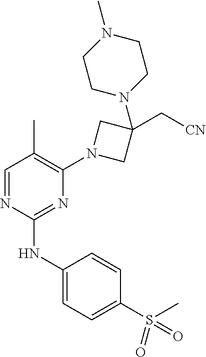 [SMILES: Cc1cnc(Nc2ccc(S(C)(=O)=O)cc2)nc1N1CC(CC#N)(N2CCN(C)CC2)C1] | E132 |
| 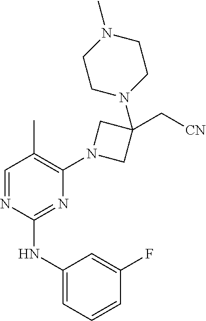 [SMILES: Cc1cnc(Nc2cccc(F)c2)nc1N1CC(CC#N)(N2CCN(C)CC2)C1] | E133 |
| 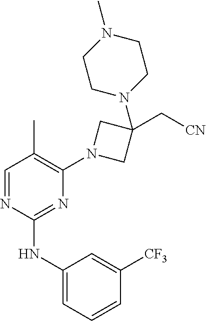 [SMILES: Cc1cnc(Nc2cccc(C(F)(F)F)c2)nc1N1CC(CC#N)(N2CCN(C)CC2)C1] | E134 |
| 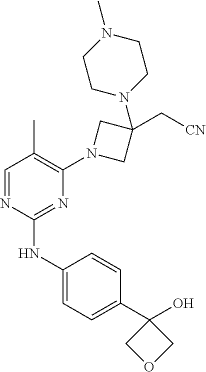 [SMILES: Cc1cnc(Nc2ccc(C3(O)COC3)cc2)nc1N1CC(CC#N)(N2CCN(C)CC2)C1] | E135 |
| 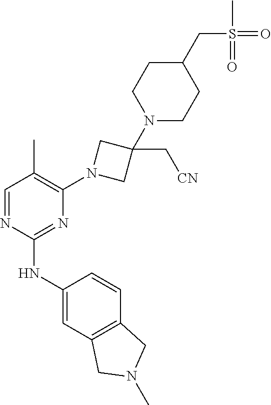 [SMILES: Cc1cnc(Nc2ccc3c(c2)CN(C)C3)nc1N1CC(CC#N)(N2CCC(CS(C)(=O)=O)CC2)C1] | E136 |
| 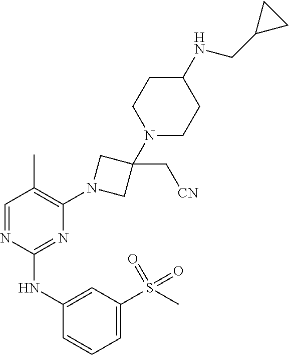 [SMILES: Cc1cnc(Nc2cccc(S(C)(=O)=O)c2)nc1N1CC(CC#N)(N2CCC(NCC3CC3)CC2)C1] | E137 |
| 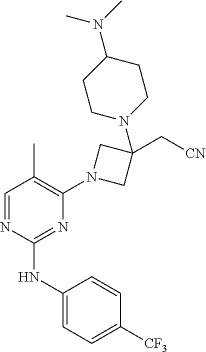 [SMILES: Cc1cnc(Nc2ccc(C(F)(F)F)cc2)nc1N1CC(CC#N)(N2CCC(N(C)C)CC2)C1] | E138 |
| 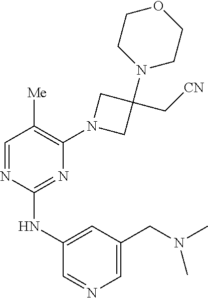 [SMILES: Cc1cnc(Nc2cncc(CN(C)C)c2)nc1N1CC(CC#N)(N2CCOCC2)C1] | E139 |
| 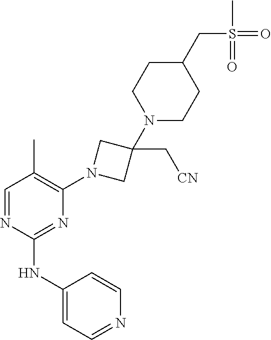 [SMILES: Cc1cnc(Nc2ccncc2)nc1N1CC(CC#N)(N2CCC(CS(C)(=O)=O)CC2)C1] | E140 |
| 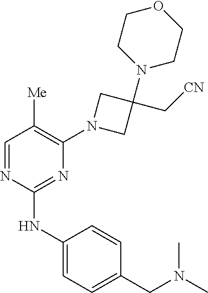 [SMILES: Cc1cnc(Nc2ccc(CN(C)C)cc2)nc1N1CC(CC#N)(N2CCOCC2)C1] | E141 |
| 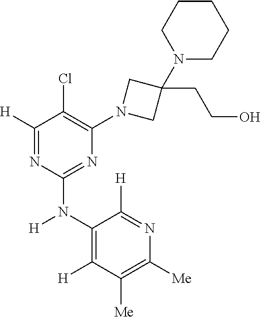 [SMILES: Cc1cc(Nc2ncc(Cl)c(N3CC(CCO)(N4CCCCC4)C3)n2)cnc1C] | E142 |
| 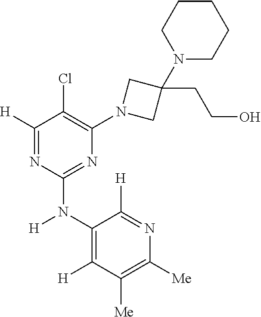 [SMILES: Cc1cc(Nc2ncc(Cl)c(N3CC(CCO)(N4CCCCC4)C3)n2)cnc1C] | E143 |
| 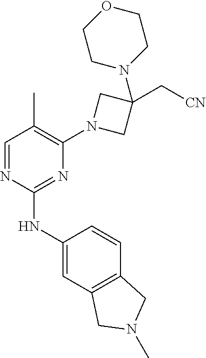 [SMILES: Cc1cnc(Nc2ccc3c(c2)CN(C)C3)nc1N1CC(CC#N)(N2CCOCC2)C1] | E144 |
| 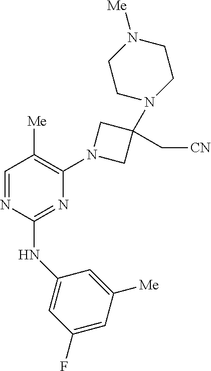 [SMILES: Cc1cc(F)cc(Nc2ncc(C)c(N3CC(CC#N)(N4CCN(C)CC4)C3)n2)c1] | E144A |

TABLE 13

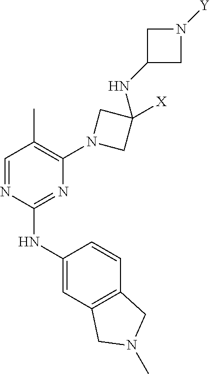

| Number | X | Y |
|---|---|---|
| S3-3-1 | methyl | methyl |
| S3-3-2 | ethyl | methyl |
| S3-3-3 | cyclobutyl | methyl |
| S3-3-4 | 3-fluorocyclobut-1-yl | methyl |
| S3-3-5 | cyclopentyl | methyl |
| S3-3-6 | tetrahydro-2H-pyran-4-yl | methyl |
| S3-3-7 | isopropyl | methyl |
| S3-3-8 | cyclohexyl | methyl |
| S3-3-9 | cycloheptyl | methyl |
| S3-3-10 | methylenecyclopropyl | methyl |
| S3-3-11 | 2-fluoroethyl | methyl |
| S3-3-12 | 2,2-difluoroethyl | methyl |
| S3-3-13 | 2-fluoroethyl | ethyl |
| S3-3-14 | —CH₂CN | isopropyl |

TABLE 14

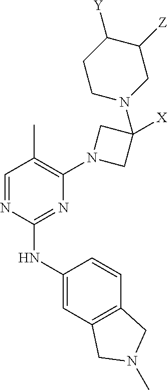

| Number | X | Y | Z |
|---|---|---|---|
| S3-4-1 | —CH₂CN | fluoro | methyl |
| S3-4-2 | —CH₂CH₂CN | fluoro | methyl |
| S3-4-3 | 2-fluoroethyl | fluoro | methyl |
| S3-4-4 | 3-fluorocyclobut-1-yl | fluoro | methyl |
| S3-4-5 | cyclopentyl | fluoro | methyl |
| S3-4-6 | tetrahydro-2H-pyran-4-yl | fluoro | methyl |
| S3-4-7 | isopropyl | fluoro | methyl |
| S3-4-8 | cyclohexyl | fluoro | methyl |
| S3-4-9 | cycloheptyl | fluoro | methyl |
| S3-4-10 | methylenecyclopropyl | fluoro | methyl |
| S3-4-11 | 2-fluoroethyl | fluoro | fluoro |
| S3-4-12 | 2,2-difluoroethyl | fluoro | methyl |
| S3-4-13 | 2-fluoroethyl | fluoro | ethyl |
| S3-4-14 | —CH₂CN | fluoro | isopropyl |
| S3-4-15 | —CH₂CN | chloro | methyl |

TABLE 15

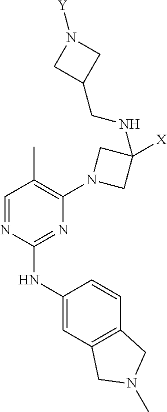

| Number | X | Y |
|---|---|---|
| S3-5-1 | methyl | methyl |
| S3-5-2 | ethyl | methyl |
| S3-5-3 | cyclobutyl | methyl |
| S3-5-4 | 3-fluorocyclobut-1-yl | methyl |
| S3-5-5 | cyclopentyl | methyl |
| S3-5-6 | tetrahydro-2H-pyran-4-yl | methyl |
| S3-5-7 | isopropyl | methyl |
| S3-5-8 | cyclohexyl | methyl |
| S3-5-9 | cycloheptyl | methyl |
| S3-5-10 | methylenecyclopropyl | methyl |
| S3-5-11 | 2-fluoroethyl | methyl |
| S3-5-12 | 2,2-difluoroethyl | methyl |
| S3-5-13 | 2-fluoroethyl | ethyl |
| S3-5-14 | —CH₂CN | isopropyl |
| S3-5-15 | —CH₂CN | methyl |

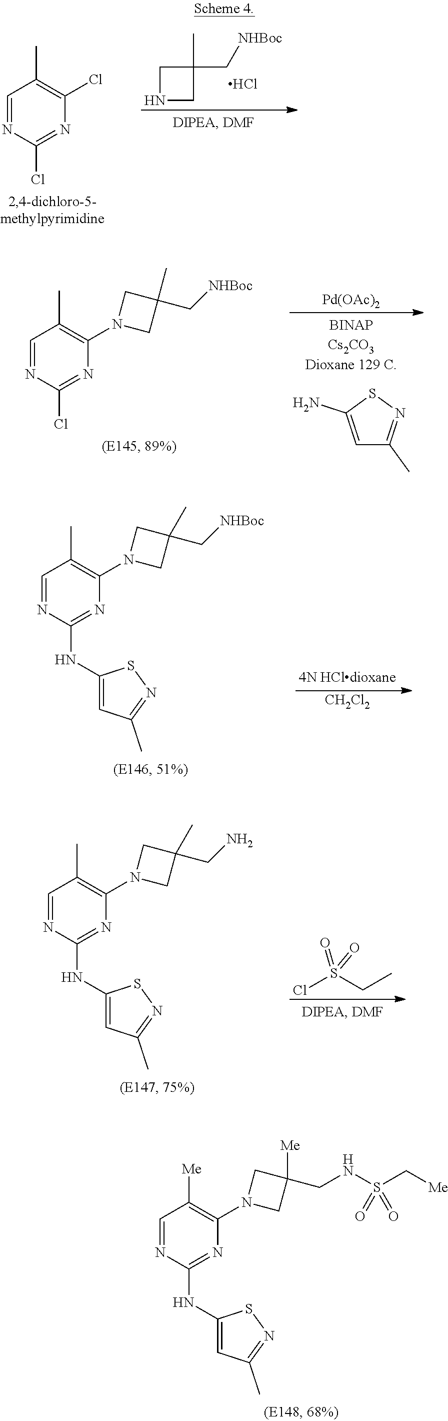

### Preparation of tert-butyl ((1-(2-chloro-5-methylpyrimidin-4-yl)-3-methylazetidin-3-yl)methyl)carbamate (E145)

To 2,4-dichloro-5-methylpyrimidine (196 mg, 1.2 mmol) in DMF (3.9 mL) was added tert-butyl ((3-methylazetidin-3-yl)methyl)carbamate hydrochloride (1.05 eq) and DIPEA (2 eq) and the solution was heated to 50° C. After 2 hours solution was cooled and extracted with EtOAc and NaHCO₃. The organics were dried (Na₂SO₄), filtered and evaporated. Column chromatography 0-100% EtOAc-Hexanes gave tert-butyl ((1-(2-chloro-5-methylpyrimidin-4-yl)-3-methylazetidin-3-yl)methyl)carbamate (E145, 89%).

### Preparation of tert-butyl ((3-methyl-1-(5-methyl-2-((3-methylisothiazol-5-yl)amino)pyrimidin-4-yl)azetidin-3-yl)methyl)carbamate (E146)

To tert-butyl ((1-(2-chloro-5-methylpyrimidin-4-yl)-3-methylazetidin-3-yl)methyl)carbamate (E145, 104 mg, 0.32 mmol) in dioxane (6.9 mL) in a pressure tube was added 3-methylisothiazol-5-amine (37.5 mg, 0.33 mmol), BINAP (39.8 mg, 0.06 mmol), Cs₂CO₃ (156 mg, 0.48 mmol), and Pd(OAc)₂ (7.1 mg, 0.03 mmol)) and the mixture was bubbled with N₂ for 1 min. The pressure tube was capped and heated to 129° C. for 1.5-2 h, cooled and the mixture was extracted with EtOAc and NaHCO₃(sat), dried (Na₂SO₄), filtered and evaporated. Column chromatography 0-5% MeOH—CH₂Cl₂ gave tert-butyl ((3-methyl-1-(5-methyl-2-((3-methylisothiazol-5-yl)amino)pyrimidin-4-yl)azetidin-3-yl)methyl)carbamate (E146, 51%)

### Preparation of N-(4-(3-(aminomethyl)-3-methylazetidin-1-yl)-5-methylpyrimidin-2-yl)-3-methylisothiazol-5-amine (E147)

To tert-butyl ((3-methyl-1-(5-methyl-2-((3-methylisothiazol-5-yl)amino)pyrimidin-4-yl)azetidin-3-yl)methyl)carbamate (E146, 67 mg, 0.17 mmol) in CH₂Cl₂ (0.9 mL) was added HCl (4N in dioxane, 0.33 mL, 1.33 mmol) and the solution was stirred for 3 h and evaporated. The compound was taken up in water and 2-methyl-THF. NaHCO₃(sat) and K₂CO₃ was added until pH 10 (pH paper) and the aqueous was extracted with 2-methyl THF. NaCl was added and the mixture was further extracted with 2-methyl THF. Column chromatography 0-90% (EtOH/10% 2N NH₃-MeOH)—CH₂Cl₂ gave N-(4-(3-(aminomethyl)-3-methylazetidin-1-yl)-5-methylpyrimidin-2-yl)-3-methylisothiazol-5-amine (E147, 39 mg, 75%).

### Preparation of N-((3-methyl-1-(5-methyl-2-((3-methylisothiazol-5-yl)amino)pyrimidin-4-yl)azetidin-3-yl)methyl)ethanesulfonamide (E148)

To N-(4-(3-(aminomethyl)-3-methylazetidin-1-yl)-5-methylpyrimidin-2-yl)-3-methylisothiazol-5-amine (E147, 17 mg, 0.06 mmol) in CH₂Cl₂ (4 mL) and DMF (0.2 mL) was added DIPEA (9.7 μL, 0.06 mmol) and the reaction was cooled to 0° C. Ethanesulfonyl chloride was added and the reaction was warmed to room temperature. The reaction was extracted with EtOAc, dried (Na₂SO₄), filtered and evaporated. Column chromatography 0-10% MeOH—CH₂Cl₂ gave N-((3-methyl-1-(5-methyl-2-((3-methylisothiazol-5-yl)amino)pyrimidin-4-yl)azetidin-3-yl)methyl)ethanesulfonamide (E148, 15.1 mg, 68%)

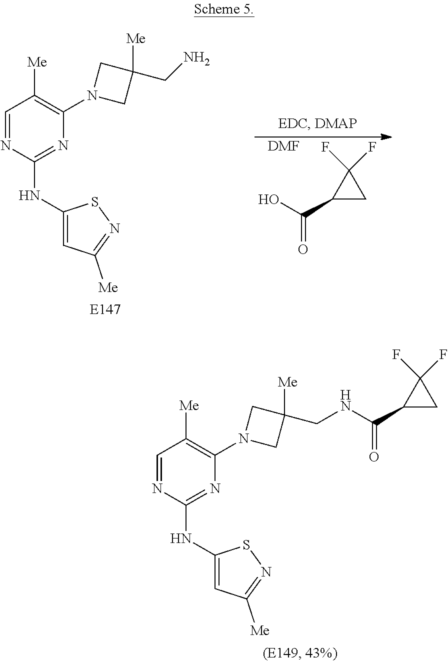

Preparation of (S)-2,2-difluoro-N-((3-methyl-1-(5-methyl-2-((3-methylisothiazol-5-yl)amino)pyrimidin-4-yl)azetidin-3-yl)methyl)cyclopropane-1-carboxamide (E149): To N-(4-(3-(aminomethyl)-3-methylazetidin-1-yl)-5-methylpyrimidin-2-yl)-3-methylisothiazol-5-amine (E147, 20.4 mg, 0.07 mmol) in DMF (0.35 mL) was added (S)-2,2-difluorocyclopropane-1-carboxylic acid (9.8 mg, 0.08 mmol), EDC (22 mg, 0.11 mmol), DMAP (1.6 mg, 0.01 mmol) and solution stirred overnight at room temperature. The reaction mixture was poured into EtOAc/NaHCO₃ (sat) and extracted with EtOAc, dried (Na₂SO₄), filtered and evaporated. Column chromatography (0-10% MeOH—CH₂Cl₂) gave pure (S)-2,2-difluoro-N-((3-methyl-1-(5-methyl-2-((3-methylisothiazol-5-yl)amino)pyrimidin-4-yl)azetidin-3-yl)methyl)cyclopropane-1-carboxamide (E149, 11.5 mg, 43%).

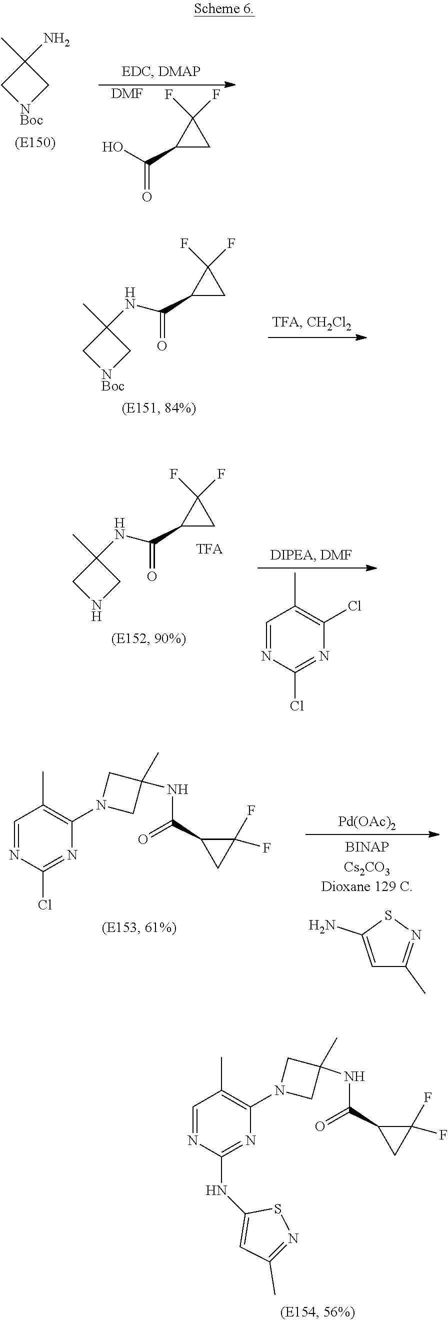

Preparation of tert-butyl (S)-3-(2,2-difluorocyclopropane-1-carboxamido)-3-methylazetidine-1-carboxylate (E151): To tert-butyl 3-amino-3-methylazetidine-1-carboxylate (E150, 400 mg, 2.1 mmol) in DMF (7 mL) was added (S)-2,2-difluorocyclopropane-1-carboxylic acid (313 mg, 2.6 mmol), EDC (659 mg, 3.4 mmol), and DMAP (52 mg, 0.4 mmol) and the solution stirred at room temperature overnight. The reaction mixture was poured into EtOAc and HCL (1N) and extracted with EtOAc, dried (Na₂SO₄) and filtered. Column chromatography 0-50% EtOAc-Hexanes gave pure tert-butyl (S)-3-(2,2-difluorocyclopropane-1-carboxamido)-3-methylazetidine-1-carboxylate (E151, 519 mg, 84%).

Preparation of (S)-2,2-difluoro-N-(3-methylazetidin-3-yl)cyclopropane-1-carboxamide trifluoroacetate (E152): To tert-butyl (S)-3-(2,2-difluorocyclopropane-1-carboxamido)-3-methylazetidine-1-carboxylate (E151, 519 mg, 1.79 mmol) in CH₂Cl₂ (13 mL) was added TFA (2 mL) and the solution stirred overnight. The solvents were evaporated and the residue azeotroped with toluene to give (S)-2,2-difluoro-N-(3-methylazetidin-3-yl)cyclopropane-1-carboxamide trifluoracetate (E152, 492 mg, 90%).

Preparation of N-(1-(2-chloro-5-methylpyrimidin-4-yl)-3-methylazetidin-3-yl)-2,2-difluorocyclopropane-1-carboxamide (E153): To (S)-2,2-difluoro-N-(3-methylazetidin-3-yl)cyclopropane-1-carboxamide trifluoracetate (E152, 492 mg, 1.6 mmol) in DMF (5.2 mL) was added DIPEA (0.57 mL, 3.2 mmol) and 2,4-dichloro-5-methylpyrimidine (264 mg, 1.6 mmol) and the solution was heated to 30° C. overnight. The reaction mixture was cooled and poured into EtOAc and NaHCO₃(sat) and extracted with EtOAc, dried (Na₂SO₄), filtered and evaporated. Automated column 0-80% EtOAc-Hexanes gave pure N-(1-(2-chloro-5-methylpyrimidin-4-yl)-3-methylazetidin-3-yl)-2,2-difluorocyclopropane-1-carboxamide (E153, 315 mg, 61%)

Preparation of 2,2-difluoro-N-(3-methyl-1-(5-methyl-2-((3-methylisothiazol-5-yl)amino)pyrimidin-4-yl)azetidin-3-yl)cyclopropane-1-carboxamide (E154): To 2,2-difluoro-N-(3-methyl-1-(5-methyl-2-((3-methylisothiazol-5-yl)amino)pyrimidin-4-yl)azetidin-3-yl)cyclopropane-1-carboxamide (E153, 73 mg, 0.23 mmol) in dioxane (4.9 mL) in a pressure tube was added 3-methylisothiazol-5-amine (27.4 mg, 0.24 mmol), BINAP (28.4 mg, 0.05 mmol), Cs₂CO₃ (111 mg, 0.34 mmol), and Pd(OAc)₂ (5.1 mg, 0.02 mmol)) and the mixture was bubbled with N₂ for 1 min. The pressure tube was capped and heated to 129° C. for 1.5-2 h, cooled and the mixture was extracted with EtOAc and NaHCO₃(sat), dried (Na₂SO₄), filtered and evaporated. Column chromatography 0-5% MeOH—CH₂Cl₂ gave 2,2-difluoro-N-(3-methyl-1-(5-methyl-2-((3-methylisothiazol-5-yl)amino)pyrimidin-4-yl)azetidin-3-yl)cyclopropane-1-carboxamide (E154, 50 mg, 56%).

Using the above schemes, S1-S6, with suitable modifications, the following compounds were synthesized E155-E182 (Table 16) and compounds E183-E207 (Table 17) could be synthesized.

TABLE 16

| | |
|---|---|
| 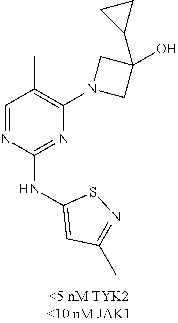 [SMILES: Cc1cc(Nc2ncc(C)c(N3CC(O)(C4CC4)C3)n2)sn1] | E155 |
| 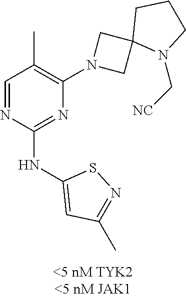 [SMILES: Cc1cc(Nc2ncc(C)c(N3CC4(CCCN4CC#N)C3)n2)sn1] | E156 |
| 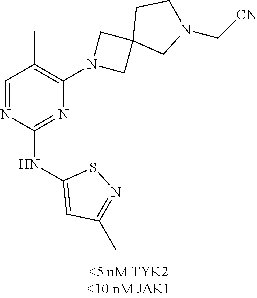 [SMILES: Cc1cc(Nc2ncc(C)c(N3CC4(CCN(CC#N)C4)C3)n2)sn1] | E157 |
| 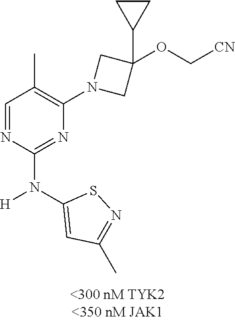 [SMILES: Cc1cc(Nc2ncc(C)c(N3CC(OCC#N)(C4CC4)C3)n2)sn1] | E158 |
| 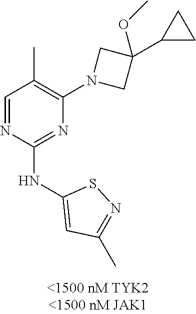 [SMILES: COC1(C2CC2)CN(c2nc(Nc3cc(C)ns3)ncc2C)C1] | E159 |
| 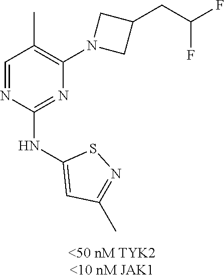 [SMILES: Cc1cc(Nc2ncc(C)c(N3CC(CC(F)F)C3)n2)sn1] | E160 |
| 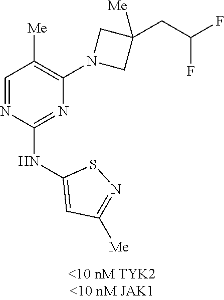 [SMILES: Cc1cc(Nc2ncc(C)c(N3CC(C)(CC(F)F)C3)n2)sn1] | E161 |
| 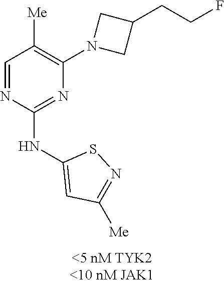 [SMILES: Cc1cc(Nc2ncc(C)c(N3CC(CCF)C3)n2)sn1] | E162 |
| 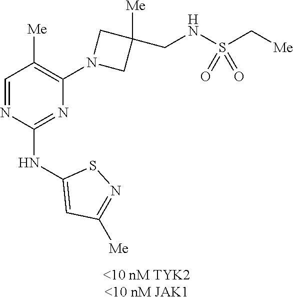 [SMILES: CCS(=O)(=O)NCC1(C)CN(c2nc(Nc3cc(C)ns3)ncc2C)C1] | E163 |
| 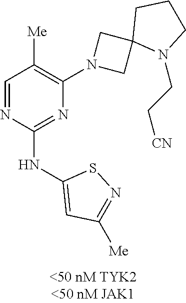 [SMILES: Cc1cc(Nc2ncc(C)c(N3CC4(CCCN4CCC#N)C3)n2)sn1] | E164 |
| 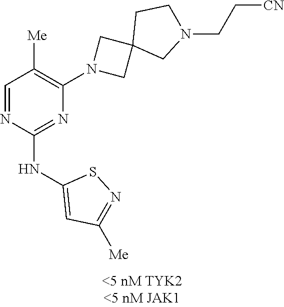 [SMILES: Cc1cc(Nc2ncc(C)c(N3CC4(CCN(CCC#N)C4)C3)n2)sn1] | E165 |
| 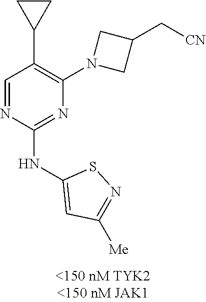 [SMILES: Cc1cc(Nc2ncc(C3CC3)c(N3CC(CC#N)C3)n2)sn1] | E166 |
| 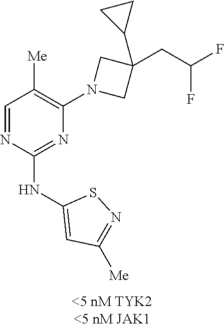 [SMILES: Cc1cc(Nc2ncc(C)c(N3CC(CC(F)F)(C4CC4)C3)n2)sn1] | E167 |
| 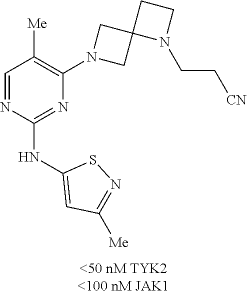 [SMILES: Cc1cc(Nc2ncc(C)c(N3CC4(CCN4CCC#N)C3)n2)sn1] | E168 |
| 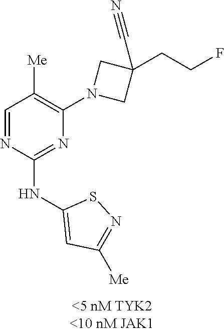 [SMILES: Cc1cc(Nc2ncc(C)c(N3CC(C#N)(CCF)C3)n2)sn1] | E169 |
| 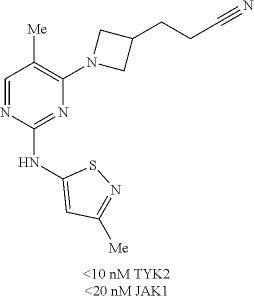 [SMILES: Cc1cc(Nc2ncc(C)c(N3CC(CCC#N)C3)n2)sn1] | E170 |
| 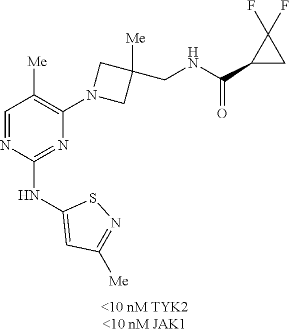 [SMILES: Cc1cc(Nc2ncc(C)c(N3CC(C)(CNC(=O)[C@@H]4CC4(F)F)C3)n2)sn1] | E171 |
| 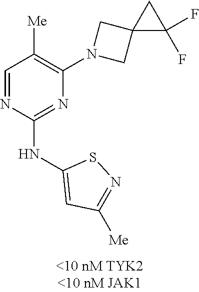 [SMILES: Cc1cc(Nc2ncc(C)c(N3CC4(C3)CC4(F)F)n2)sn1] | E172 |
| 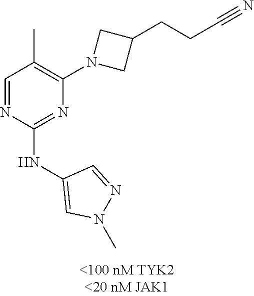 [SMILES: Cc1cnc(Nc2cnn(C)c2)nc1N1CC(CCC#N)C1] | E173 |
| 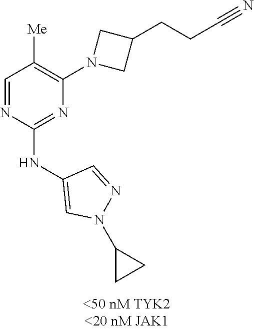 [SMILES: Cc1cnc(Nc2cnn(C3CC3)c2)nc1N1CC(CCC#N)C1] | E174 |
| 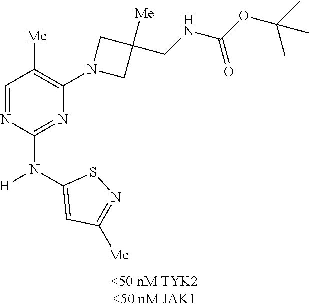 [SMILES: Cc1cc(Nc2ncc(C)c(N3CC(C)(CNC(=O)OC(C)(C)C)C3)n2)sn1] | E175 |
| 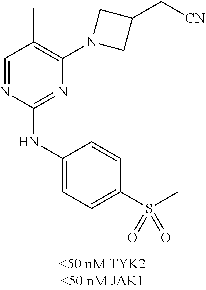 [SMILES: Cc1cnc(Nc2ccc(S(C)(=O)=O)cc2)nc1N1CC(CC#N)C1] | E176 |
| 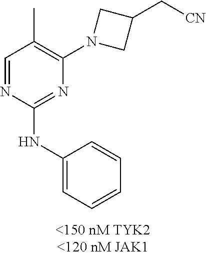 [SMILES: Cc1cnc(Nc2ccccc2)nc1N1CC(CC#N)C1] | E177 |
| 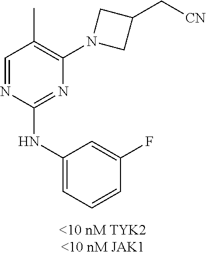 [SMILES: Cc1cnc(Nc2cccc(F)c2)nc1N1CC(CC#N)C1] | E178 |
| 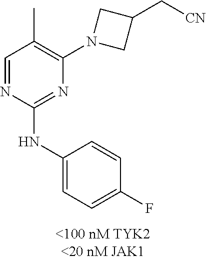 [SMILES: Cc1cnc(Nc2ccc(F)cc2)nc1N1CC(CC#N)C1] | E179 |
| 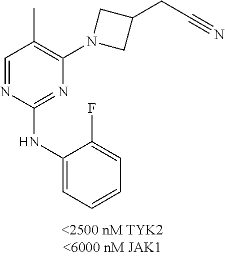 [SMILES: Cc1cnc(Nc2ccccc2F)nc1N1CC(CC#N)C1] | E180 |
| 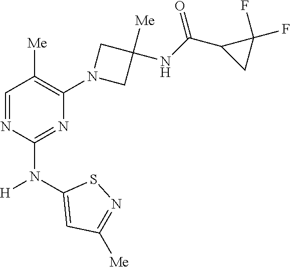 [SMILES: Cc1cc(Nc2ncc(C)c(N3CC(C)(NC(=O)C4CC4(F)F)C3)n2)sn1] | E181 |
| 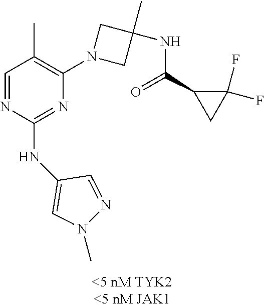 [SMILES: Cc1cnc(Nc2cnn(C)c2)nc1N1CC(C)(NC(=O)[C@@H]2CC2(F)F)C1] | E182 |

TABLE 17

| | |
|---|---|
| 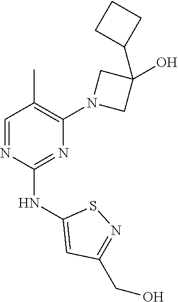 [SMILES: Cc1cnc(Nc2cc(CO)ns2)nc1N1CC(O)(C2CCC2)C1] | E183 |
| 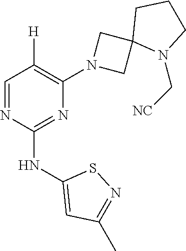 [SMILES: Cc1cc(Nc2nccc(N3CC4(CCCN4CC#N)C3)n2)sn1] | E184 |
| 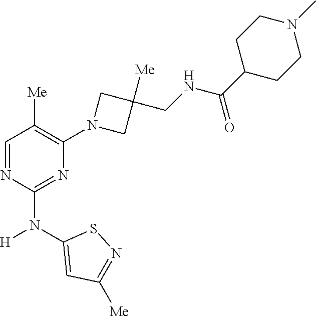 [SMILES: Cc1cc(Nc2ncc(C)c(N3CC(C)(CNC(=O)C4CCN(C)CC4)C3)n2)sn1] | E185 |
| 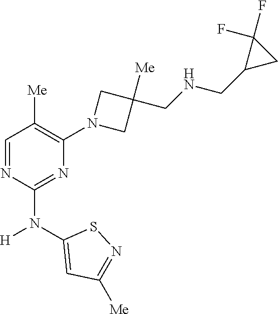 [SMILES: Cc1cc(Nc2ncc(C)c(N3CC(C)(CNCC4CC4(F)F)C3)n2)sn1] | E186 |
| 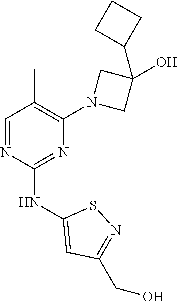 [SMILES: Cc1cnc(Nc2cc(CO)ns2)nc1N1CC(O)(C2CCC2)C1] | E187 |
| 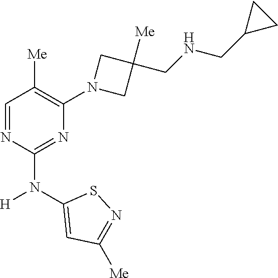 [SMILES: Cc1cc(Nc2ncc(C)c(N3CC(C)(CNCC4CC4)C3)n2)sn1] | E188 |
| 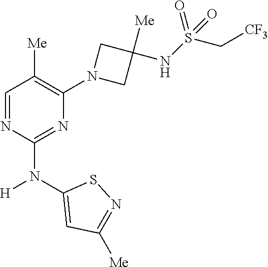 [SMILES: Cc1cc(Nc2ncc(C)c(N3CC(C)(NS(=O)(=O)CC(F)(F)F)C3)n2)sn1] | E189 |
| 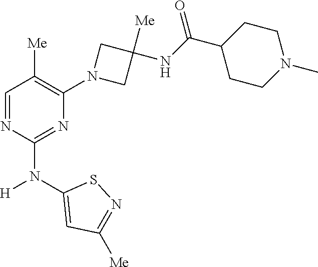 [SMILES: Cc1cc(Nc2ncc(C)c(N3CC(C)(NC(=O)C4CCN(C)CC4)C3)n2)sn1] | E190 |
| 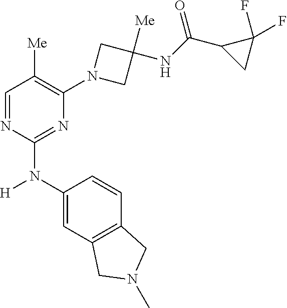 [SMILES: Cc1cnc(Nc2ccc3c(c2)CN(C)C3)nc1N1CC(C)(NC(=O)C2CC2(F)F)C1] | E191 |
| 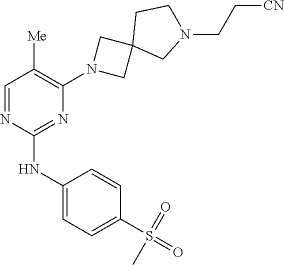 [SMILES: Cc1cnc(Nc2ccc(S(C)(=O)=O)cc2)nc1N1CC2(CCN(CCC#N)C2)C1] | E192 |
| 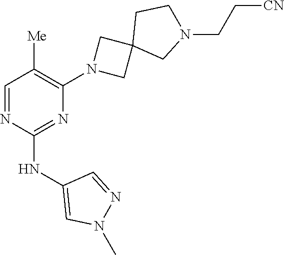 [SMILES: Cc1cnc(Nc2cnn(C)c2)nc1N1CC2(CCN(CCC#N)C2)C1] | E193 |
| 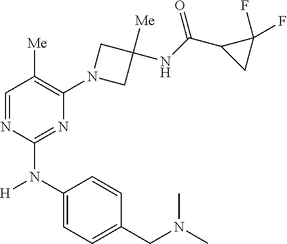 [SMILES: Cc1cnc(Nc2ccc(CN(C)C)cc2)nc1N1CC(C)(NC(=O)C2CC2(F)F)C1] | E194 |
| 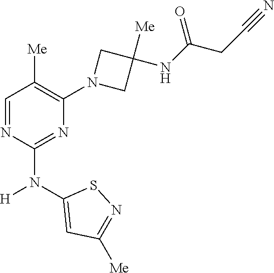 [SMILES: Cc1cc(Nc2ncc(C)c(N3CC(C)(NC(=O)CC#N)C3)n2)sn1] | E195 |
| 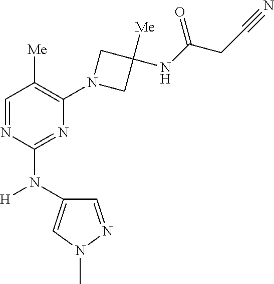 [SMILES: Cc1cnc(Nc2cnn(C)c2)nc1N1CC(C)(NC(=O)CC#N)C1] | E196 |
| 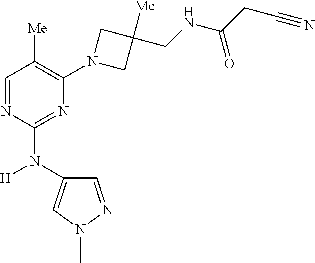 [SMILES: Cc1cnc(Nc2cnn(C)c2)nc1N1CC(C)(CNC(=O)CC#N)C1] | E197 |
| 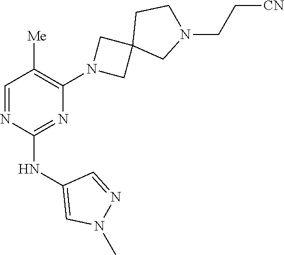 [SMILES: Cc1cnc(Nc2cnn(C)c2)nc1N1CC2(CCN(CCC#N)C2)C1] | E198 |
| 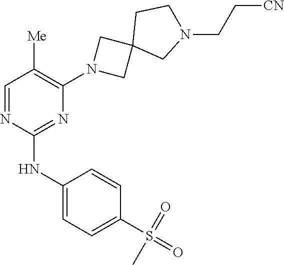 [SMILES: Cc1cnc(Nc2ccc(S(C)(=O)=O)cc2)nc1N1CC2(CCN(CCC#N)C2)C1] | E199 |
| 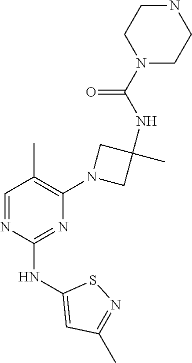 [SMILES: Cc1cc(Nc2ncc(C)c(N3CC(C)(NC(=O)N4CCNCC4)C3)n2)sn1] | E200 |
| 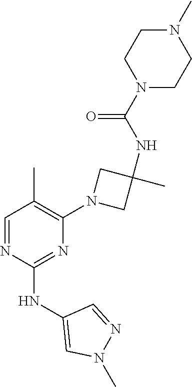 [SMILES: Cc1cnc(Nc2cnn(C)c2)nc1N1CC(C)(NC(=O)N2CCN(C)CC2)C1] | E201 |
| 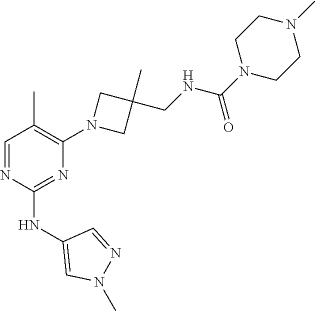 [SMILES: Cc1cnc(Nc2cnn(C)c2)nc1N1CC(C)(CNC(=O)N2CCN(C)CC2)C1] | E202 |
| 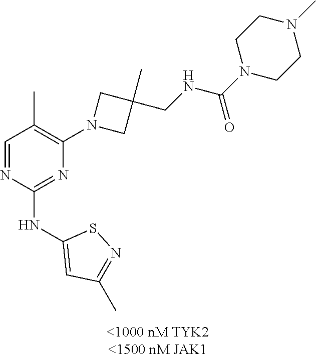 [SMILES: Cc1cc(Nc2ncc(C)c(N3CC(C)(CNC(=O)N4CCN(C)CC4)C3)n2)sn1] | E203 |
| 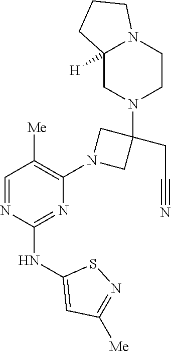 | E204 |
| 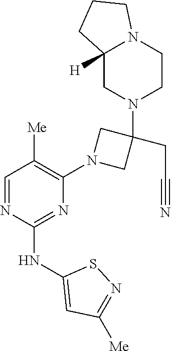 [SMILES: Cc1cc(Nc2ncc(C)c(N3CC(CC#N)(N4CCN5CCC[C@H]5C4)C3)n2)sn1] | E205 |
| 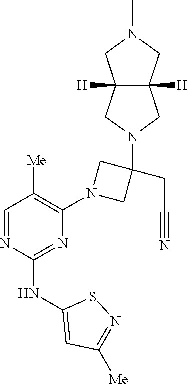 [SMILES: Cc1cc(Nc2ncc(C)c(N3CC(CC#N)(N4C[C@H]5CN(C)C[C@H]5C4)C3)n2)sn1] | E206 |
| 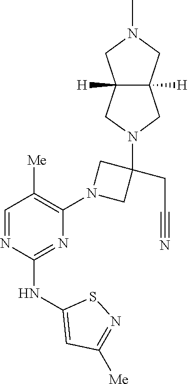 [SMILES: Cc1cc(Nc2ncc(C)c(N3CC(CC#N)(N4C[C@@H]5CN(C)C[C@H]5C4)C3)n2)sn1] | E207 |

Preparation of tert-butyl 3-(cyanomethyl)-3-(4-methylpiperazin-1-yl)azetidine-1-carboxylate (E2): To a solution of tert-butyl 3-(cyanomethylene)azetidine-1-carboxylate (E1, 200 mg, 1.030 mmol) in MeOH (1.3 mL) was added 1-methylpiperazine (172 μL, 1.545 mmol). The reaction mixture was stirred at room temperature until starting material was consumed by TLC (5%/MeOH/CH₂Cl₂, iodine stain). The reaction was poured over saturated NaHCO₃ and extracted with CH₂Cl₂. The organic layer was dried over MgSO₄, then concentrated under reduced pressure. Compound E2 was purified by column chromatography (0-5% MeOH:CH₂Cl₂) to give a white solid (E2, 94%).

Preparation of 2-(3-(4-methylpiperazin-1-yl)azetidin-3-yl)acetonitrile trifluoroacetate (E3): To a solution of tert-butyl 3-(cyanomethyl)-3-(4-methylpiperazin-1-yl)azetidine-1-carboxylate (E2, 285 mg, 0.97 mmol) in dichloromethane (9.7 mL) was added trifluoroacetic acid (0.97 mL). Reaction was stirred at room temperature until starting material was consumed based on TLC (5% MeOH/CH₂Cl₂, stained with KMnO₄). The solution was concentrated under reduced pressure and the clear, colorless, crude oil (E3) was carried on to the next step.

Preparation of 2-(1-(2-chloro-5-methylpyrimidin-4-yl)-3-(4-methylpiperazin-1-yl)azetidin-3-yl) acetonitrile (E4): To a solution of E3 (188 mg, 0.97 mmol) in DMF (3 mL) at 0 deg C. was added 2,4-dichloro-5-methylpyrimidine (150 mg, 0.92 mmol). Reaction was stirred at room temperature overnight. The reaction was poured over saturated NaHCO₃ and extracted with EtOAc. The organic layer was dried over MgSO₄, then concentrated under reduced pressure. Compound E4 was purified by column chromatography (0-5% MeOH:CH₂Cl₂) to give a white solid in quantitative yield.

Preparation of 2-(1-(5-methyl-2-((3-methylisothiazol-5-yl)amino)pyrimidin-4-yl)-3-(4-methyl-piperazin-1-yl)azetidin-3-yl)acetonitrile (E5): To a microwave vial was added E4 (100 mg, 0.312 mmol), 3-methylisothiazol-5-amine (39 mg, 0.328 mmol), Pd(OAc)₂ (3.5 mg, 5 mol %), BINAP (29 mg, 15 mol %), K₂CO₃ (173 mg, 1.25 mmol) and dioxane:tert-butyl alcohol (1:1, 3.1 mL). The reaction mixture was heated overnight at 110 deg C. The reaction mixture was filtered through celite into 1M HCl_{aq} in a separatory funnel. The celite was washed with ethyl acetate. Aqueous layer was washed with ethyl acetate to remove significant yellow color. Then, sat. aq. NaHCO₃ was added to the aqueous layer until basic and a precipitate has formed. Aqueous layer was then extracted with ethyl acetate. The organic layer was dried over Na₂SO₄, then concentrated under reduced pressure. Compound E5 was purified by column chromatography (0-5% MeOH:CH₂Cl₂) to give a white solid (37%).

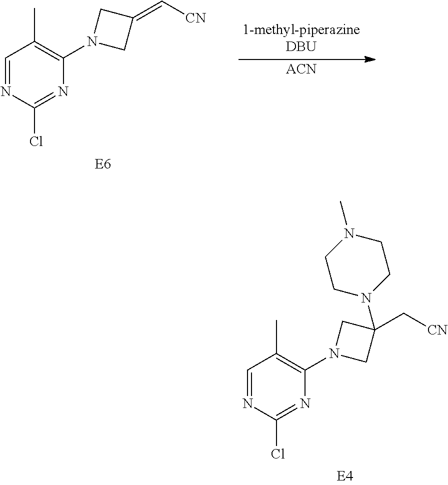 [SMILES: Cc1cnc(Cl)nc1N1CC(=CC#N)C1 ; "ACN" ; Cc1cnc(Cl)nc1N1CC(CC#N)(N2CCN(C)CC2)C1]

Alternate preparation of 2-(1-(2-chloro-5-methylpyrimidin-4-yl)-3-(4-methylpiperazin-1-yl)azetidin-3-yl)acetonitrile (E4): To a dry vial was charged intermediate (100 mg; 0.45 mmol; 1.0 eq) and DBU (671 μL; 2.25 mmol; 10.0 eq) in dry ACN (0.6 mL). To the solution was added 1-methylpiperazine (76 μL; 0.68 mmol; 1.5 eq). The solution turned red. After 30 minutes, the reaction was complete by TLC and LC. The reaction mixture was poured over saturated sodium bicarbonate and sodium chloride and extracted with CH₂Cl₂:IPA (3:1). The combined organics were dried over sodium sulfate, filtered, then evaporated. The crude residue was purified by column chromatography (CH₂Cl₂:EtOH) to afford intermediate (E4) in 70% yield.

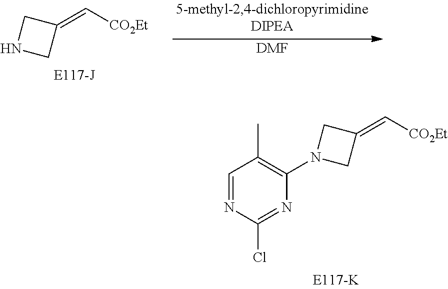

To 2,4-dichloro-5-methyl-pyrimidine (58 mL, 1.75 mmol, 1.0 eq) and E117-J (300 mg, 1.83 mmol, 1.05 eq) in 7 mL dry DMF was charged DIPEA (N,N-diisopropylethylamine, or Hünig's base) (367 mL, 2.10 mmol, 1.2 eq) under nitrogen. The reaction mixture was stirred overnight. The reaction mixture was poured into water and extracted with ethyl acetate. The combined organics were dried over sodium sulfate, filtered, then evaporated. The crude residue was purified by column chromatography (Hex:EtOAc) to afford E117-K in 67% yield (299 mg, 1.18 mmol).

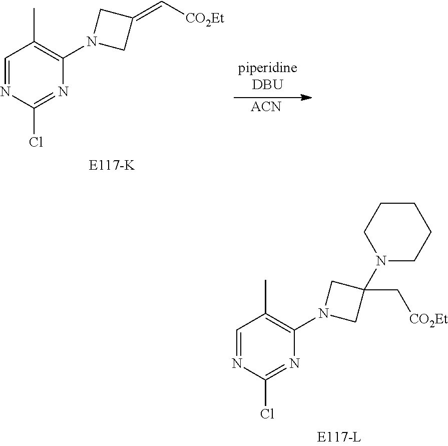 [SMILES: CCOC(=O)C=C1CN(c2nc(Cl)ncc2C)C1 ; "ACN" ; CCOC(=O)CC1(N2CCCCC2)CN(c2nc(Cl)ncc2C)C1]

To E117-K (100 mg, 0.39 mmol, 1.0 eq) under nitrogen was charged 1 mL dry acetonitrile and DBU (58 mL, 0.39 mmol, 1 eq). To this solution as added piperidine (41 mL, 0.41 mmol, 1.05 eq). The reaction mixture was stirred overnight. The reaction mixture was poured into water and extracted with CH₂Cl₂. The combined organics were dried over sodium sulfate, filtered, then evaporated. The crude residue was purified by column chromatography (Hex:EtOAc) to afford E117-L in 62% yield (82 mg, 0.24 mmol).

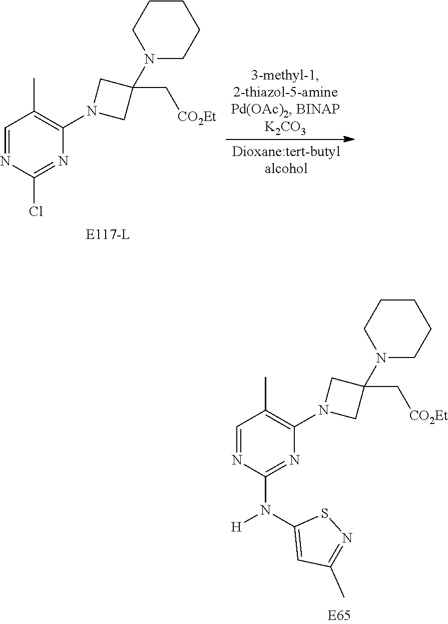 [SMILES: CCOC(=O)CC1(N2CCCCC2)CN(c2nc(Cl)ncc2C)C1 ; CCOC(=O)CC1(N2CCCCC2)CN(c2nc(Nc3cc(C)ns3)ncc2C)C1]

To E117-L (82 mg, 0.24 mmol, 1.0 eq) under nitrogen was 2-methylisoindolin-5-amine hydrochloride (29 mg, 0.25 mmol, 1.05 eq), palladium (II) acetate (2.2 mg, 0.01 mmol, 5 mol %), BINAP (22 mg, 0.04 mmol, 15 mol %), and potassium carbonate (133 mg, 0.96 mmol, 4.0 eq) followed by dioxane and tert-butyl alcohol (4 mL, 1:1). The reaction mixture was heated overnight at 120 deg C. The reaction mixture was poured into saturated sodium bicarbonate/saturated sodium chloride and CH₂Cl₂. The organic layer was separated, and the aqueous layer was extracted with CH₂Cl₂. The combined organic layers were dried over sodium sulfate, then filtered and evaporated. The crude residue was purified by column chromatography (Hex:EtOAc then CH₂Cl₂:MeOH) to afford E65 in 80% yield (80 mg, 0.19 mmol).

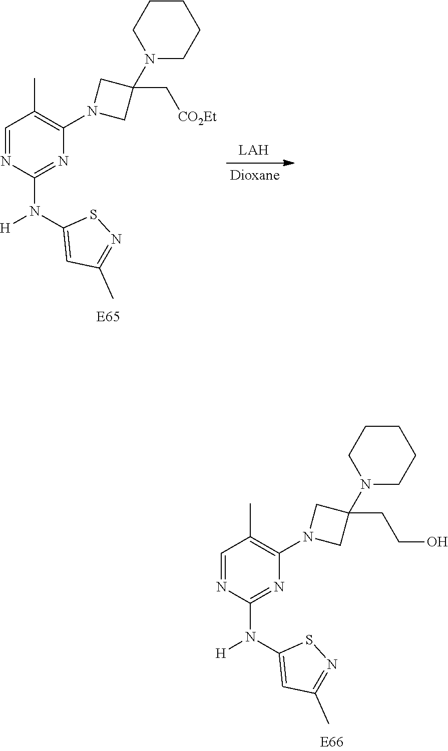 [SMILES: CCOC(=O)CC1(N2CCCCC2)CN(c2nc(Nc3cc(C)ns3)ncc2C)C1 ; Cc1cc(Nc2ncc(C)c(N3CC(CCO)(N4CCCCC4)C3)n2)sn1]

To E65 (70 mg, 0.17 mmol, 1.0 eq) in 2 mL dry dioxane at 0 deg C. was charged lithium aluminum hydride (1 M in THF, 0.21 mL) dropwise. The reaction mixture was stirred at 0 deg C. for 0.5 h, then at room temperature for 1 h. The reaction mixture was quenched with 1:1 EtOAc:H₂O dropwise, then filtered through Celite. The filtrate was evaporated and purified by column chromatography (CH₂C₂:MeOH) to afford E66 in 78% yield (51.5 mg, 0.13 mmol).

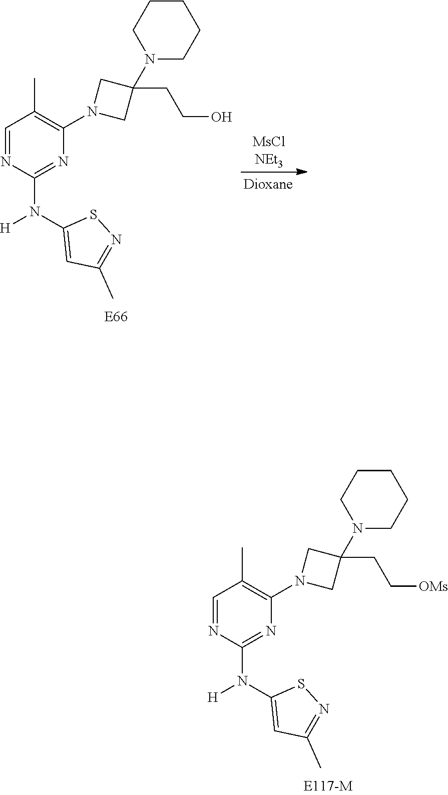 [SMILES: Cc1cc(Nc2ncc(C)c(N3CC(CCO)(N4CCCCC4)C3)n2)sn1 ; Cc1cc(Nc2ncc(C)c(N3CC(CCOS(C)(=O)=O)(N4CCCCC4)C3)n2)sn1]

To a solution of E66 (50 mg, 0.13 mmol, 1.0 eq) and triethylamine (30 mL, 0.39 mmol, 3 eq) in 4 mL dry dioxane was added methanesulfonyl chloride (30 mL, 0.19 mmol, 1.5 eq) dropwise. The reaction mixture was stirred at rt for two hours. The reaction mixture was evaporated, and crude E117-M was carried forward in the next step without further purification.

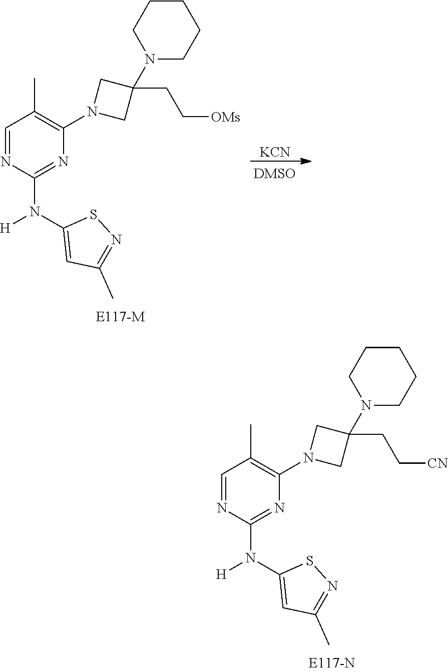

To a solution of E117-M (35 mg, 75 mmol, 1.0 eq) in 1 mL dry DMSO was charged KCN (15 mg, 0.23 mmol, 3.0 eq). The reaction mixture was heated to 80 deg C. and allowed to stir overnight. The reaction mixture was poured into water and extracted with ethyl acetate. The combined organics were dried over sodium sulfate, filtered, then evaporated. The crude residue as purified via column chromatography (CH₂Cl₂:MeOH) to afford E117-N (7 mg, 18 mmol) in 25% yield.

TABLE 18

| Com- | | |
|---|---|---|
| pound | | |
| Num- | | |
| ber | Structure | IC₅₀ |
| E55a | 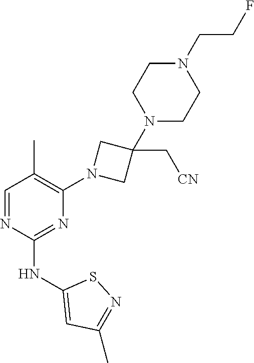 [SMILES: Cc1cc(Nc2ncc(C)c(N3CC(CC#N)(N4CCN(CCF)CC4)C3)n2)sn1] | <10 nM TYK2 <10 nM JAK1 |
| E61a | 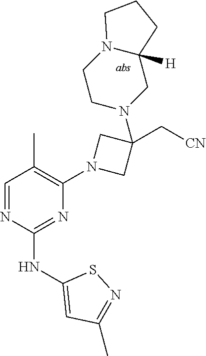 [SMILES: Cc1cc(Nc2ncc(C)c(N3CC(CC#N)(N4CCN5CCC[C@@H]5C4)C3)n2)sn1] | <50 nM TYK2 <50 nM JAK1 |
| E62a | 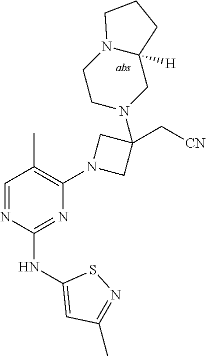 [SMILES: Cc1cc(Nc2ncc(C)c(N3CC(CC#N)(N4CCN5CCC[C@H]5C4)C3)n2)sn1] | <50 nM TYK2 <50 nM JAK1 |
| E63a | 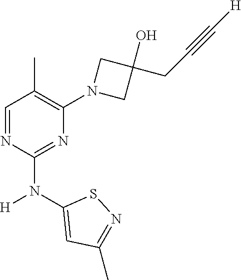 [SMILES: C#CCC1(O)CN(c2nc(Nc3cc(C)ns3)ncc2C)C1] | <50 nM TYK2 <100 nM JAK1 |
| E64a | 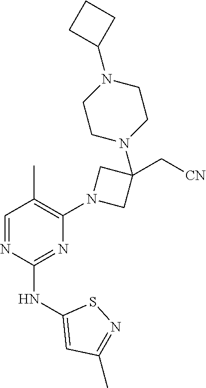 [SMILES: Cc1cc(Nc2ncc(C)c(N3CC(CC#N)(N4CCN(C5CCC5)CC4)C3)n2)sn1] | <10 nM TYK2 <10 nM JAK1 |
| E65a | 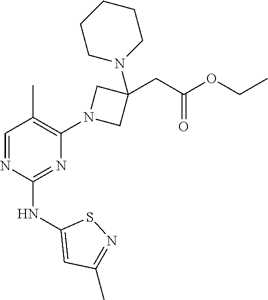 [SMILES: CCOC(=O)CC1(N2CCCCC2)CN(c2nc(Nc3cc(C)ns3)ncc2C)C1] | <50 nM TYK2 <1 μM JAK1 |
| E66a | 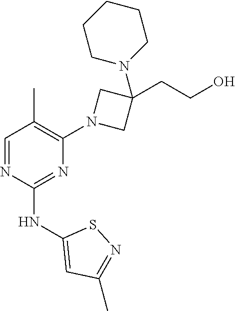 [SMILES: Cc1cc(Nc2ncc(C)c(N3CC(CCO)(N4CCCCC4)C3)n2)sn1] | <10 nM TYK2 <50 nM JAK1 |
| E67a | 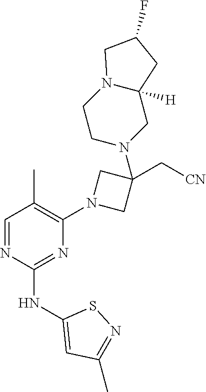 [SMILES: Cc1cc(Nc2ncc(C)c(N3CC(CC#N)(N4CCN5C[C@H](F)C[C@H]5C4)C3)n2)sn1] | <10 nM TYK2 <10 nM JAK1 |
| E68a | 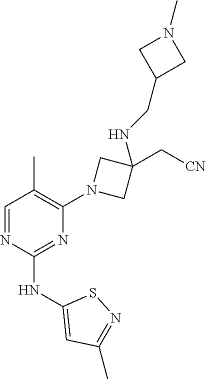 [SMILES: Cc1cc(Nc2ncc(C)c(N3CC(CC#N)(NCC4CN(C)C4)C3)n2)sn1] | <500 nM TYK2 <500 nM JAK1 |
| E69a | 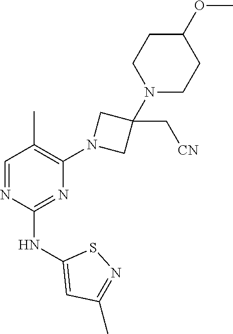 [SMILES: COC1CCN(C2(CC#N)CN(c3nc(Nc4cc(C)ns4)ncc3C)C2)CC1] | <10 nM TYK2 <10 nM JAK1 |
| E70a | 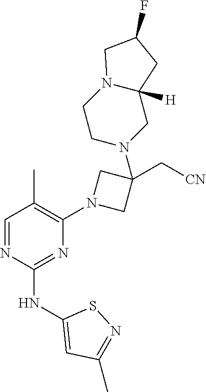 [SMILES: Cc1cc(Nc2ncc(C)c(N3CC(CC#N)(N4CCN5C[C@@H](F)C[C@@H]5C4)C3)n2)sn1] | <10 nM TYK2 <20 nM JAK1 |
| E71a | 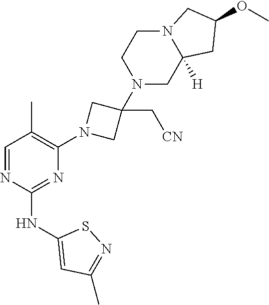 [SMILES: CO[C@H]1C[C@H]2CN(C3(CC#N)CN(c4nc(Nc5cc(C)ns5)ncc4C)C3)CCN2C1] | <10 nM TYK2 <10 nM JAK1 |
| E72a | 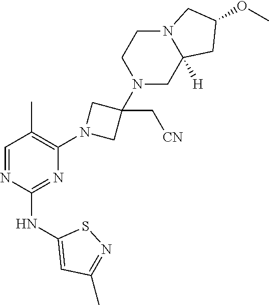 [SMILES: CO[C@@H]1C[C@H]2CN(C3(CC#N)CN(c4nc(Nc5cc(C)ns5)ncc4C)C3)CCN2C1] | <10 nM TYK2 <10 nM JAK1 |
| E73a | 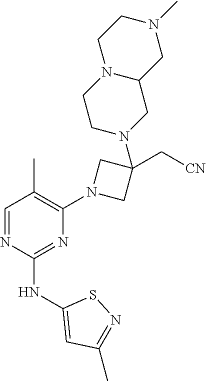 [SMILES: Cc1cc(Nc2ncc(C)c(N3CC(CC#N)(N4CCN5CCN(C)CC5C4)C3)n2)sn1] | <100 nM TYK2 <100 nM JAK1 |
| E74a | 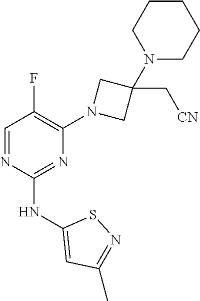 [SMILES: Cc1cc(Nc2ncc(F)c(N3CC(CC#N)(N4CCCCC4)C3)n2)sn1] | <20 nM TYK2 <500 nM JAK1 |
| E75a | 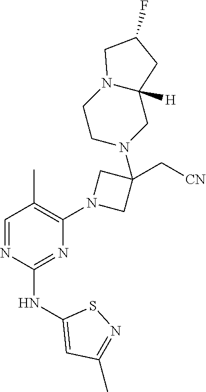 [SMILES: Cc1cc(Nc2ncc(C)c(N3CC(CC#N)(N4CCN5C[C@H](F)C[C@@H]5C4)C3)n2)sn1] | <10 nM TYK2 <50 nM JAK1 |
| E76a | 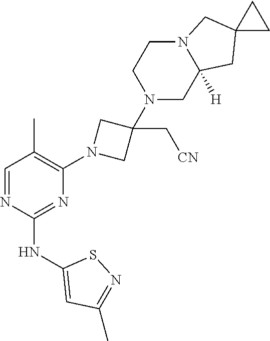 [SMILES: Cc1cc(Nc2ncc(C)c(N3CC(CC#N)(N4CCN5CC6(CC6)C[C@H]5C4)C3)n2)sn1] | <10 nM TYK2 <10 nM JAK1 |
| E77a | 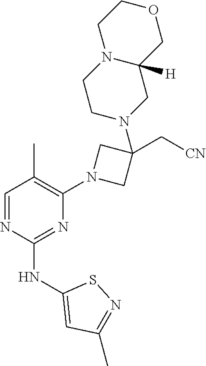 [SMILES: Cc1cc(Nc2ncc(C)c(N3CC(CC#N)(N4CCN5CCOC[C@@H]5C4)C3)n2)sn1] | <10 nM TYK2 <50 nM JAK1 |
| E78a | 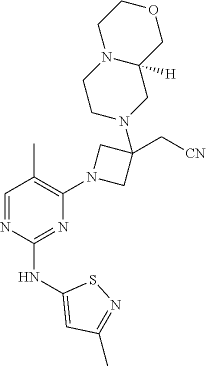 [SMILES: Cc1cc(Nc2ncc(C)c(N3CC(CC#N)(N4CCN5CCOC[C@H]5C4)C3)n2)sn1] | <10 nM TYK2 <10 nM JAK1 |
| E79a | 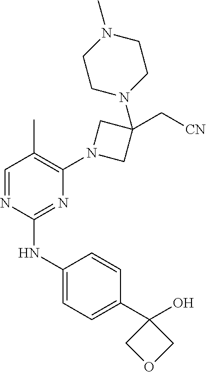 [SMILES: Cc1cnc(Nc2ccc(C3(O)COC3)cc2)nc1N1CC(CC#N)(N2CCN(C)CC2)C1] | <10 nM TYK2 <50 nM JAK1 |
| E80a | 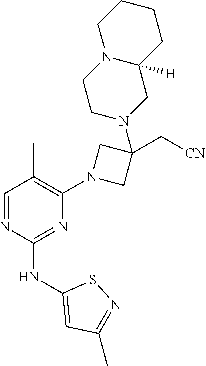 [SMILES: Cc1cc(Nc2ncc(C)c(N3CC(CC#N)(N4CCN5CCCC[C@H]5C4)C3)n2)sn1] | <10 nM TYK2 <10 nM JAK1 |
| E81a | 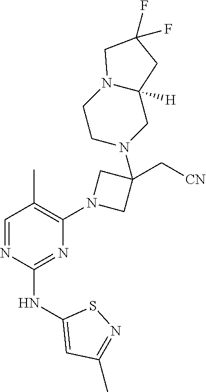 [SMILES: Cc1cc(Nc2ncc(C)c(N3CC(CC#N)(N4CCN5CC(F)(F)C[C@H]5C4)C3)n2)sn1] | <20 nM TYK2 <20 nM JAK1 |
| E82a | 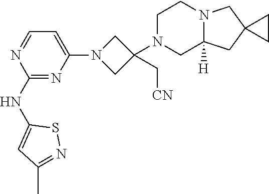 [SMILES: Cc1cc(Nc2nccc(N3CC(CC#N)(N4CCN5CC6(CC6)C[C@H]5C4)C3)n2)sn1] | <20 nM TYK2 <50 nM JAK1 |
| E83a | 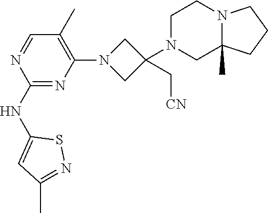 [SMILES: Cc1cc(Nc2ncc(C)c(N3CC(CC#N)(N4CCN5CCC[C@]5(C)C4)C3)n2)sn1] | <50 nM TYK2 <100 nM JAK1 |
| E84a | 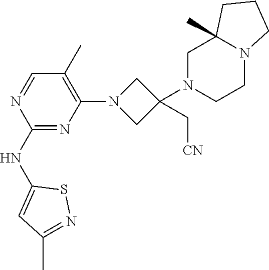 [SMILES: Cc1cc(Nc2ncc(C)c(N3CC(CC#N)(N4CCN5CCC[C@@]5(C)C4)C3)n2)sn1] | <10 nM TYK2 <10 nM JAK1 |
| E85a | 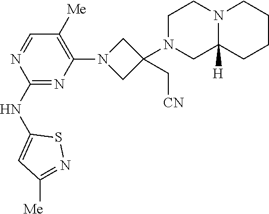 [SMILES: Cc1cc(Nc2ncc(C)c(N3CC(CC#N)(N4CCN5CCCC[C@@H]5C4)C3)n2)sn1] | <10 nM TYK2 <50 nM JAK1 |
| E86a | 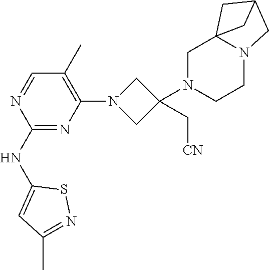 [SMILES: Cc1cc(Nc2ncc(C)c(N3CC(CC#N)(N4CCN5CC6CC5(C6)C4)C3)n2)sn1] | <20 nM TYK2 <100 nM JAK1 |
| E87a | 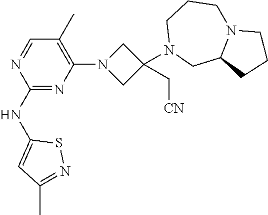 [SMILES: Cc1cc(Nc2ncc(C)c(N3CC(CC#N)(N4CCCN5CCC[C@H]5C4)C3)n2)sn1] | <10 nM TYK2 <10 nM JAK1 |
| E88a | 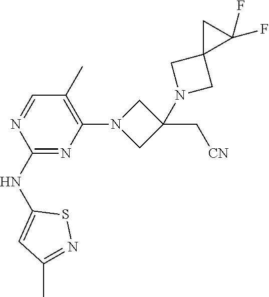 [SMILES: Cc1cc(Nc2ncc(C)c(N3CC(CC#N)(N4CC5(C4)CC5(F)F)C3)n2)sn1] | <10 nM TYK2 <10 nM JAK1 |
| E89a | 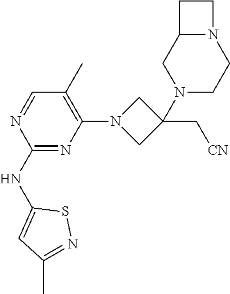 [SMILES: Cc1cc(Nc2ncc(C)c(N3CC(CC#N)(N4CCN5CCC5C4)C3)n2)sn1] | <50 nM TYK2 <100 nM JAK1 |
| E90a | 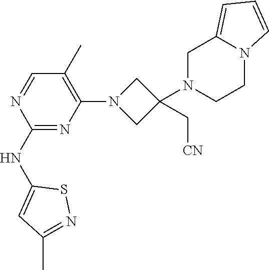 [SMILES: Cc1cc(Nc2ncc(C)c(N3CC(CC#N)(N4CCn5cccc5C4)C3)n2)sn1] | <10 nM TYK2 <10 nM JAK1 |
| E91a | 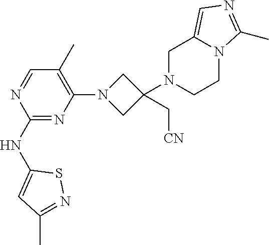 [SMILES: Cc1cc(Nc2ncc(C)c(N3CC(CC#N)(N4CCn5c(cnc5C)C4)C3)n2)sn1] | <10 nM TYK2 <10 nM JAK1 |
| E92a | 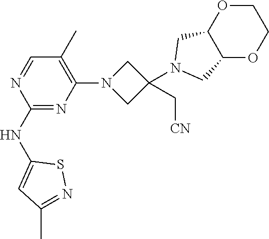 [SMILES: Cc1cc(Nc2ncc(C)c(N3CC(CC#N)(N4C[C@@H]5OCCO[C@@H]5C4)C3)n2)sn1] | <10 nM TYK2 <10 nM JAK1 |
| E93a | 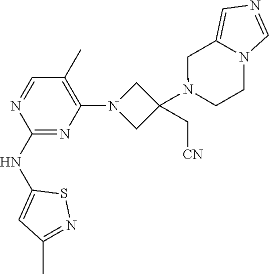 [SMILES: Cc1cc(Nc2ncc(C)c(N3CC(CC#N)(N4CCn5cncc5C4)C3)n2)sn1] | <10 nM TYK2 <10 nM JAK1 |
| E94a | 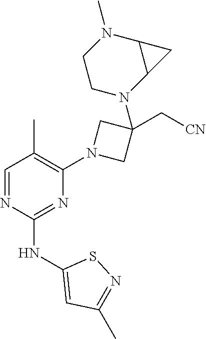 [SMILES: Cc1cc(Nc2ncc(C)c(N3CC(CC#N)(N4CCN(C)C5CC54)C3)n2)sn1] | <10 nM TYK2 <10 nM JAK1 |
| E95a | 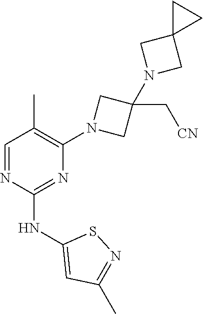 [SMILES: Cc1cc(Nc2ncc(C)c(N3CC(CC#N)(N4CC5(CC5)C4)C3)n2)sn1] | <10 nM TYK2 <50 nM JAK1 |
| E96a | 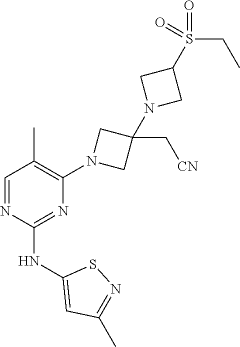 [SMILES: CCS(=O)(=O)C1CN(C2(CC#N)CN(c3nc(Nc4cc(C)ns4)ncc3C)C2)C1] | <50 nM TYK2 <100 nM JAK1 |
| E97a | 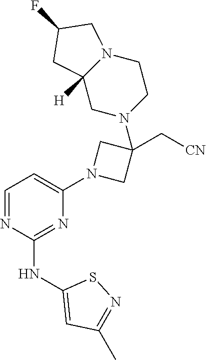 [SMILES: Cc1cc(Nc2nccc(N3CC(CC#N)(N4CCN5C[C@H](F)C[C@H]5C4)C3)n2)sn1] | <10 nM TYK2 <50 nM JAK1 |
| E98a | 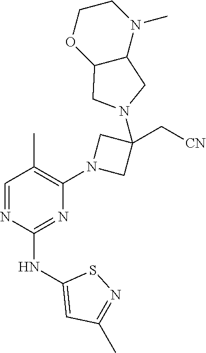 [SMILES: Cc1cc(Nc2ncc(C)c(N3CC(CC#N)(N4CC5OCCN(C)C5C4)C3)n2)sn1] | <10 nM TYK2 <50 nM JAK1 |
| E99a | 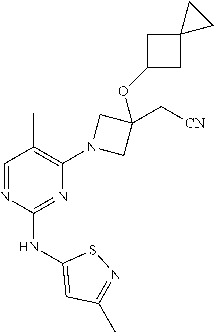 [SMILES: Cc1cc(Nc2ncc(C)c(N3CC(CC#N)(OC4CC5(CC5)C4)C3)n2)sn1] | <10 nM TYK2 <10 nM JAK1 |
| E100a | 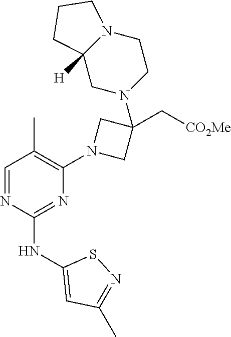 [SMILES: COC(=O)CC1(N2CCN3CCC[C@H]3C2)CN(c2nc(Nc3cc(C)ns3)ncc2C)C1] | <10 nM TYK2 <10 nM JAK1 |
| E101a | 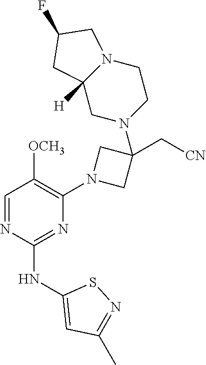 [SMILES: COc1cnc(Nc2cc(C)ns2)nc1N1CC(CC#N)(N2CCN3C[C@H](F)C[C@H]3C2)C1] | <10 nM TYK2 <10 nM JAK1 |
| E102a | 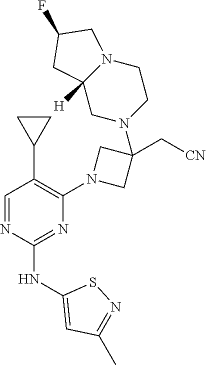 [SMILES: Cc1cc(Nc2ncc(C3CC3)c(N3CC(CC#N)(N4CCN5C[C@H](F)C[C@H]5C4)C3)n2)sn1] | <10 nM TYK2 <10 nM JAK1 |
| E103a | 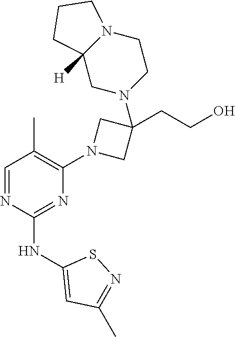 [SMILES: Cc1cc(Nc2ncc(C)c(N3CC(CCO)(N4CCN5CCC[C@H]5C4)C3)n2)sn1] | <10 nM TYK2 <10 nM JAK1 |
| E104a | 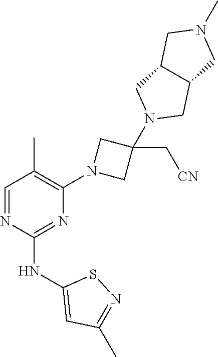 [SMILES: Cc1cc(Nc2ncc(C)c(N3CC(CC#N)(N4C[C@H]5CN(C)C[C@H]5C4)C3)n2)sn1] | <100 nM TYK2 <100 nM JAK1 |
| E105a | 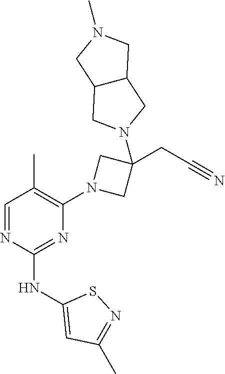 [SMILES: Cc1cc(Nc2ncc(C)c(N3CC(CC#N)(N4CC5CN(C)CC5C4)C3)n2)sn1] | <50 nM TYK2 <100 nM JAK1 |
| E106a | 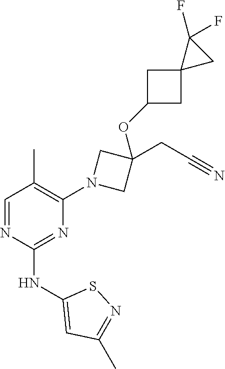 [SMILES: Cc1cc(Nc2ncc(C)c(N3CC(CC#N)(OC4CC5(C4)CC5(F)F)C3)n2)sn1] | <10 nM TYK2 <10 nM JAK1 |
| E107a | 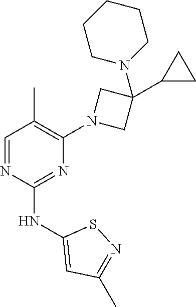 [SMILES: Cc1cc(Nc2ncc(C)c(N3CC(C4CC4)(N4CCCCC4)C3)n2)sn1] | <50 nM TYK2 <100 nM JAK1 |
| E108a | 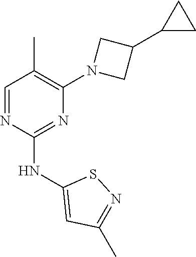 [SMILES: Cc1cc(Nc2ncc(C)c(N3CC(C4CC4)C3)n2)sn1] | <10 nM TYK2 <50 nM JAK1 |
| E109a | 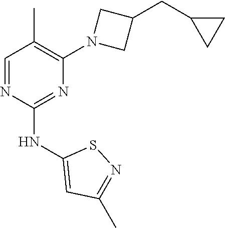 [SMILES: Cc1cc(Nc2ncc(C)c(N3CC(CC4CC4)C3)n2)sn1] | <10 nM TYK2 <50 nM JAK1 |
| E110a | 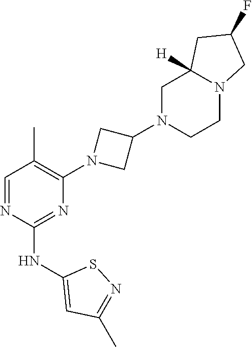 [SMILES: Cc1cc(Nc2ncc(C)c(N3CC(N4CCN5C[C@H](F)C[C@H]5C4)C3)n2)sn1] | <10 nM TYK2 <50 nM JAK1 |
| E111a | 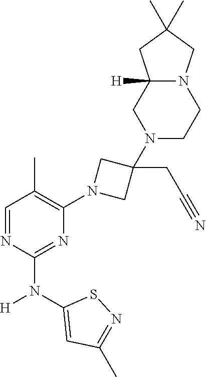 [SMILES: Cc1cc(Nc2ncc(C)c(N3CC(CC#N)(N4CCN5CC(C)(C)C[C@H]5C4)C3)n2)sn1] | <10 nM TYK2 <10 nM JAK1 |
| E112a | 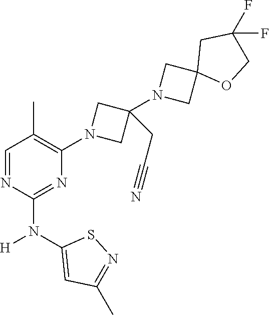 [SMILES: Cc1cc(Nc2ncc(C)c(N3CC(CC#N)(N4CC5(C4)CC(F)(F)CO5)C3)n2)sn1] | <50 nM TYK2 <50 nM JAK1 |
| E113a | 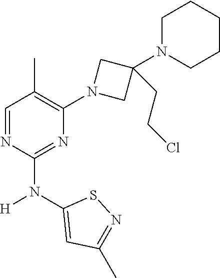 [SMILES: Cc1cc(Nc2ncc(C)c(N3CC(CCCl)(N4CCCCC4)C3)n2)sn1] | <10 nM TYK2 <10 nM JAK1 |
| E114a | 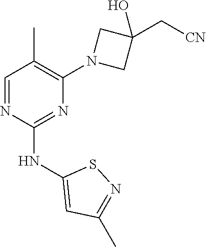 [SMILES: Cc1cc(Nc2ncc(C)c(N3CC(O)(CC#N)C3)n2)sn1] | <10 nM TYK2 <20 nM JAK1 |
| E115a | 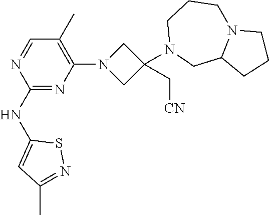 [SMILES: Cc1cc(Nc2ncc(C)c(N3CC(CC#N)(N4CCCN5CCCC5C4)C3)n2)sn1] | <50 nM TYK2 <200 nM JAK1 |
| E116a | 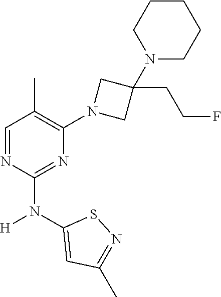 [SMILES: Cc1cc(Nc2ncc(C)c(N3CC(CCF)(N4CCCCC4)C3)n2)sn1] | <10 nM TYK2 <50 nM JAK1 |
| E117a | 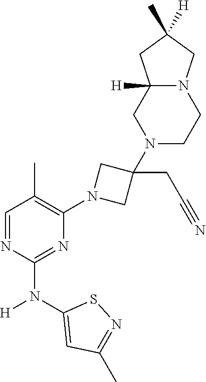 [SMILES: Cc1cc(Nc2ncc(C)c(N3CC(CC#N)(N4CCN5C[C@H](C)C[C@H]5C4)C3)n2)sn1] | <10 nM TYK2 <50 nM JAK1 |
| E117-A | 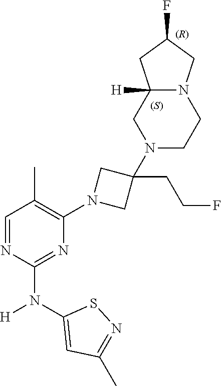 | <10 nM TYK2 <50 nM JAK1 |
| E117-B | 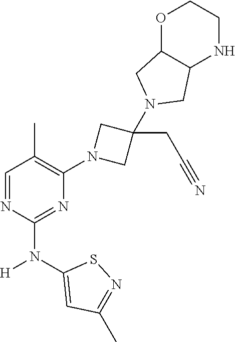 [SMILES: Cc1cc(Nc2ncc(C)c(N3CC(CC#N)(N4CC5NCCOC5C4)C3)n2)sn1] | <100 nM TYK2 <50 nM JAK1 |
| E117-C | 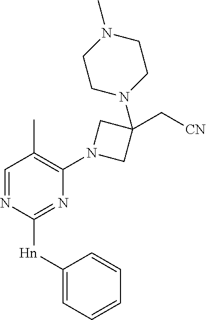 | <50 nM TYK2 <100 nM JAK1 |
| E117-E | 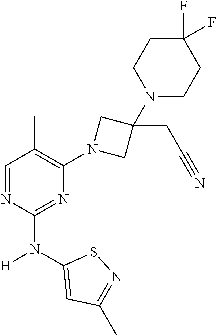 [SMILES: Cc1cc(Nc2ncc(C)c(N3CC(CC#N)(N4CCC(F)(F)CC4)C3)n2)sn1] | <5 nM TYK2 <5 nM JAK1 |
| E117-G | 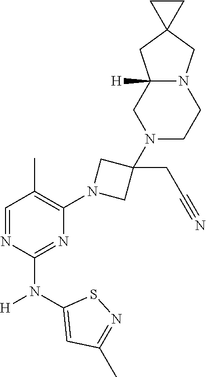 [SMILES: Cc1cc(Nc2ncc(C)c(N3CC(CC#N)(N4CCN5CC6(CC6)C[C@H]5C4)C3)n2)sn1] | <5 nM TYK2 <5 nM JAK1 |
| E117-J | 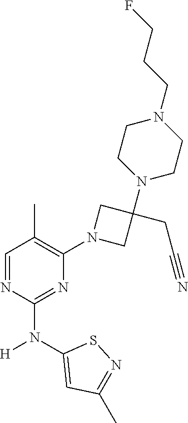 [SMILES: Cc1cc(Nc2ncc(C)c(N3CC(CC#N)(N4CCN(CCCF)CC4)C3)n2)sn1] | <5 nM TYK2 <5 nM JAK1 |
| E117-N | 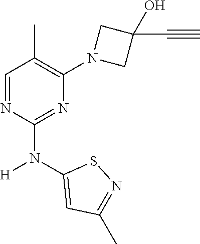 [SMILES: C#CC1(O)CN(c2nc(Nc3cc(C)ns3)ncc2C)C1] | <5 nM TYK2 <10 nM JAK1 |
| E117-P | 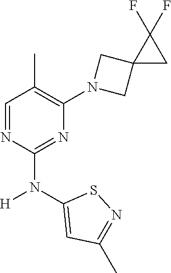 [SMILES: Cc1cc(Nc2ncc(C)c(N3CC4(C3)CC4(F)F)n2)sn1] | <5 nM TYK2 <10 nM JAK1 |
| E117-R | 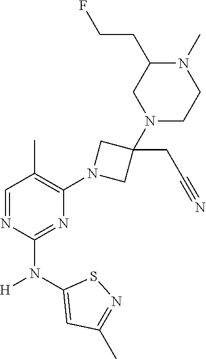 [SMILES: Cc1cc(Nc2ncc(C)c(N3CC(CC#N)(N4CCN(C)C(CCF)C4)C3)n2)sn1] | <5 nM TYK2 <10 nM JAK1 |
| E117-S | 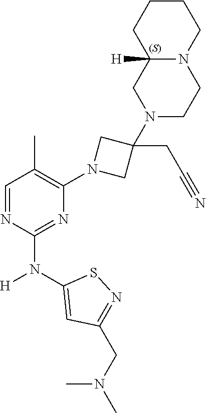 | <10 nM TYK2 <25 nM JAK1 |
| E117-T | 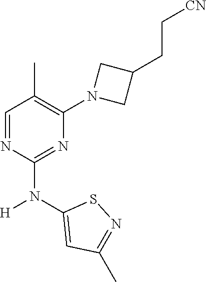 [SMILES: Cc1cc(Nc2ncc(C)c(N3CC(CCC#N)C3)n2)sn1] | <10 nM TYK2 <25 nM JAK1 |
| E117-U | 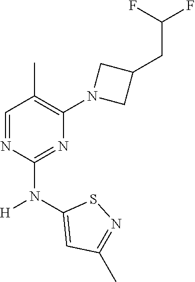 [SMILES: Cc1cc(Nc2ncc(C)c(N3CC(CC(F)F)C3)n2)sn1] | <10 nM TYK2 <25 nM JAK1 |
| E117-V | 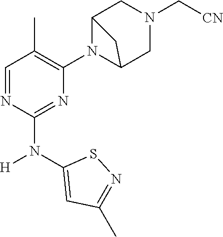 [SMILES: Cc1cc(Nc2ncc(C)c(N3C4CC3CN(CC#N)C4)n2)sn1] | <50 nM TYK2 <50 nM JAK1 |
| E117-X | 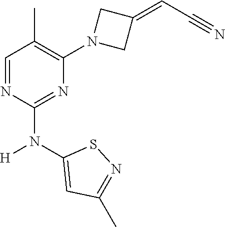 [SMILES: Cc1cc(Nc2ncc(C)c(N3CC(=CC#N)C3)n2)sn1] | <75 nM TYK2 <75 nM JAK1 |
| E117-Y | 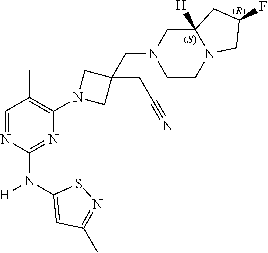 | <50 nM TYK2 <100 nM JAK1 |
| E117-AA | 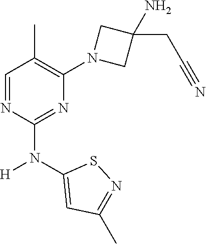 [SMILES: Cc1cc(Nc2ncc(C)c(N3CC(N)(CC#N)C3)n2)sn1] | <50 nM TYK2 <100 nM JAK1 |
| E117-AD | 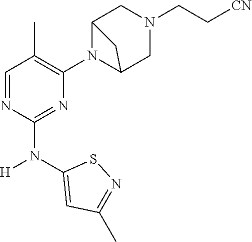 [SMILES: Cc1cc(Nc2ncc(C)c(N3C4CC3CN(CCC#N)C4)n2)sn1] | <1 μM TYK2 <1 μM JAK1 |

Using substantially the same procedures as described above, with appropriate modifications, compounds E118a-E150a can be prepared.

TABLE 19

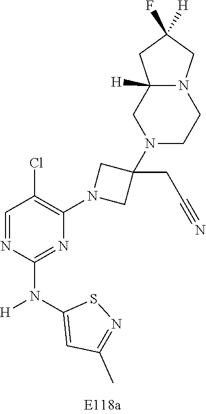 [SMILES: Cc1cc(Nc2ncc(Cl)c(N3CC(CC#N)(N4CCN5C[C@H](F)C[C@H]5C4)C3)n2)sn1]

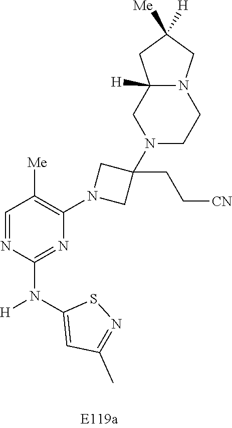 [SMILES: Cc1cc(Nc2ncc(C)c(N3CC(CCC#N)(N4CCN5C[C@H](C)C[C@H]5C4)C3)n2)sn1]

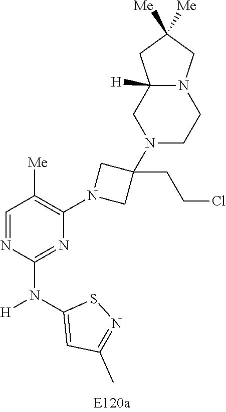

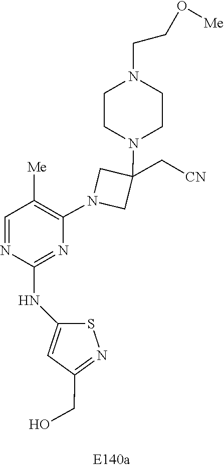 [SMILES: COCCN1CCN(C2(CC#N)CN(c3nc(Nc4cc(CO)ns4)ncc3C)C2)CC1]

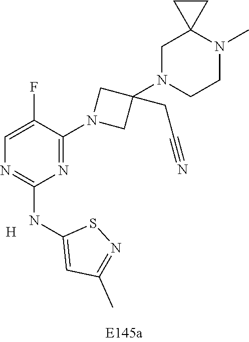

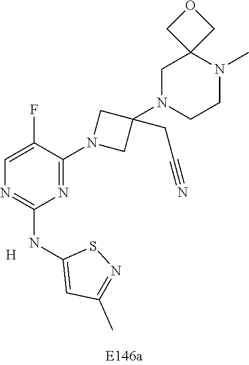

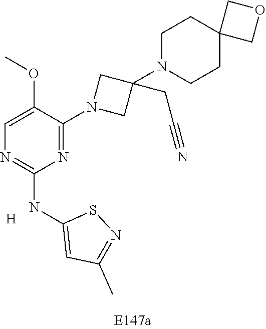

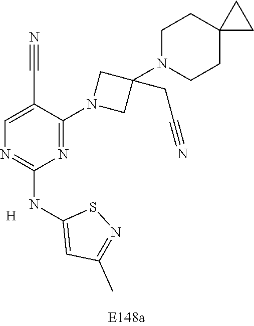

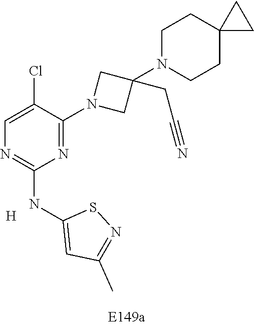

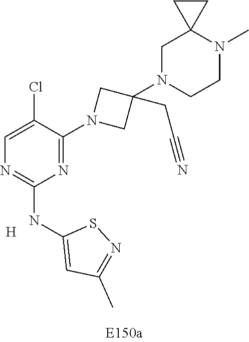

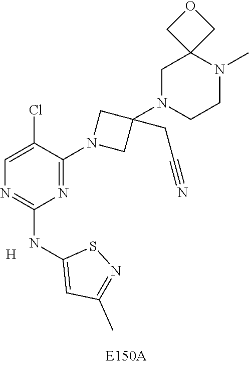

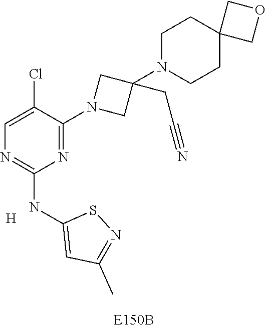

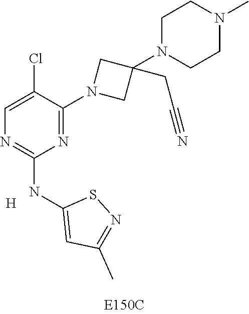

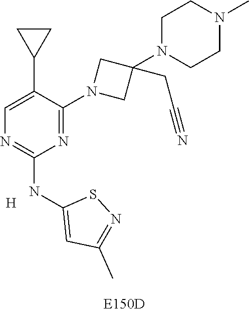

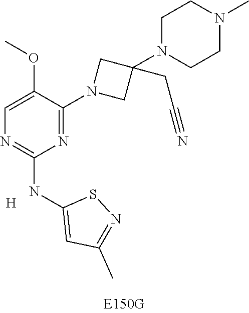

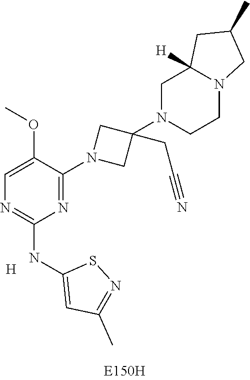

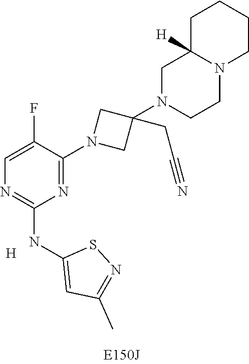

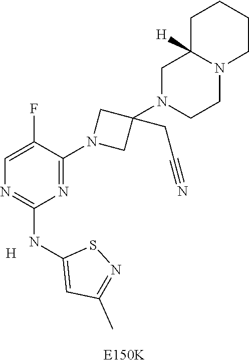

E150L
E150M
E150Q
E150R
E150S
E150T
E150U
E150V

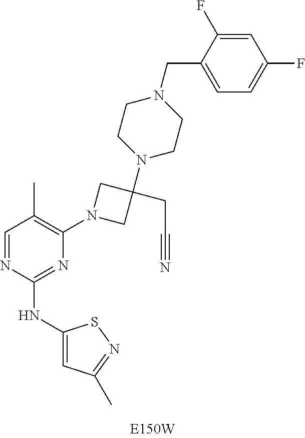 [SMILES: Cc1cc(Nc2ncc(C)c(N3CC(CC#N)(N4CCN(Cc5ccc(F)cc5F)CC4)C3)n2)sn1]

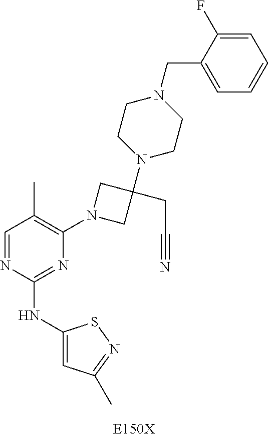 [SMILES: Cc1cc(Nc2ncc(C)c(N3CC(CC#N)(N4CCN(Cc5ccccc5F)CC4)C3)n2)sn1]

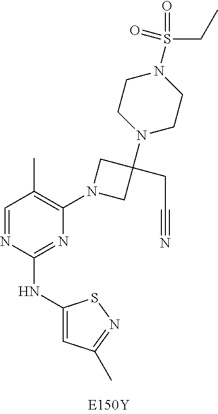 [SMILES: CCS(=O)(=O)N1CCN(C2(CC#N)CN(c3nc(Nc4cc(C)ns4)ncc3C)C2)CC1]

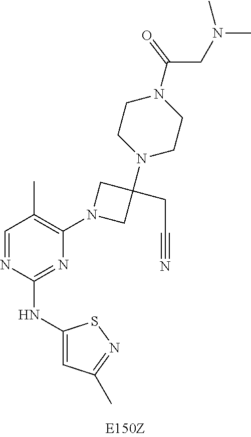 [SMILES: Cc1cc(Nc2ncc(C)c(N3CC(CC#N)(N4CCN(C(=O)CN(C)C)CC4)C3)n2)sn1]

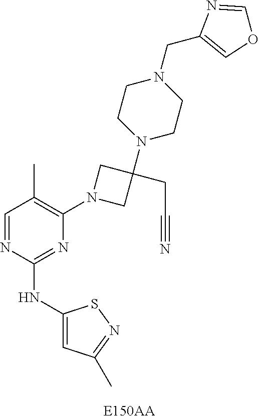 [SMILES: Cc1cc(Nc2ncc(C)c(N3CC(CC#N)(N4CCN(Cc5cocn5)CC4)C3)n2)sn1]

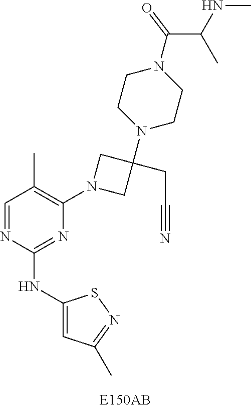 [SMILES: CNC(C)C(=O)N1CCN(C2(CC#N)CN(c3nc(Nc4cc(C)ns4)ncc3C)C2)CC1]

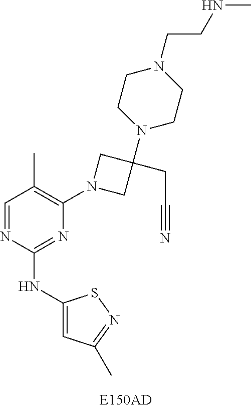 [SMILES: CNCCN1CCN(C2(CC#N)CN(c3nc(Nc4cc(C)ns4)ncc3C)C2)CC1]

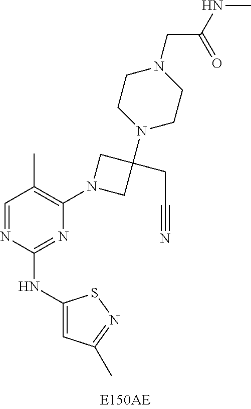 [SMILES: CNC(=O)CN1CCN(C2(CC#N)CN(c3nc(Nc4cc(C)ns4)ncc3C)C2)CC1]

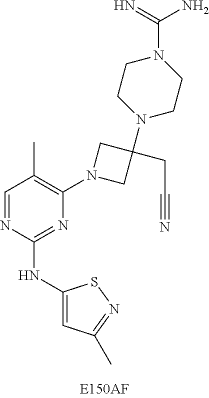 [SMILES: Cc1cc(Nc2ncc(C)c(N3CC(CC#N)(N4CCN(C(=N)N)CC4)C3)n2)sn1]

E150AG
E150AH
E150AJ
E150AK

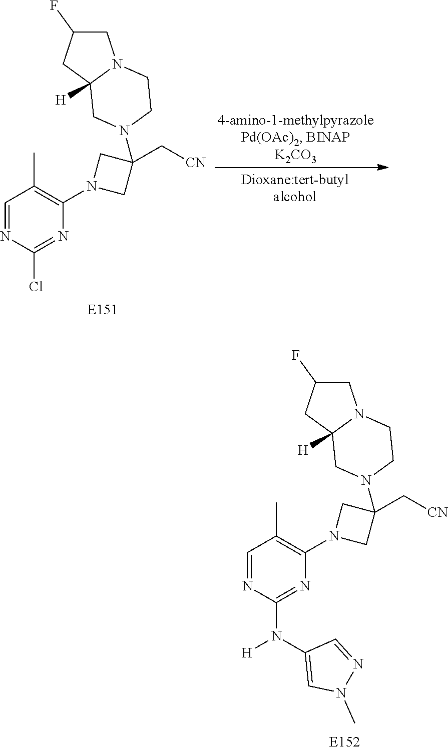 [SMILES: Cc1cnc(Cl)nc1N1CC(CC#N)(N2CCN3CC(F)C[C@H]3C2)C1 ; Cc1cnc(Nc2cnn(C)c2)nc1N1CC(CC#N)(N2CCN3CC(F)C[C@H]3C2)C1]

Preparation of 2-(3-((7R,8aS)-7-fluorohexahydropyrrolo[1,2-a]pyrazin-2(1H)-yl)-1-(5-methyl-2-((1-methyl-1H-pyrazol-4-yl)amino)pyrimidin-4-yl)azetidin-3-yl)acetonitrile (E152): To 2-(1-(2-chloro-5-methylpyrimidin-4-yl)-3-((7R,8aS)-7-fluorohexahydropyrrolo[1,2-a]pyrazin-2(1H)-yl)azetidin-3-yl)acetonitrile (160 mg, 0.44 mmol, 1.0 eq) under nitrogen was 4-amino-1-methylpyrazole hydrochloride (79 mg, 0.46 mmol, 1.05 eq), palladium (II) acetate (4.9 mg, 0.012 mmol, 5 mol %), BINAP (41 mg, 0.07 mmol, 15 mol %), and potassium carbonate (243 mg, 1.76 mmol, 4.0 eq) followed by dioxane and tert-butyl alcohol (4 mL, 1:1). The reaction mixture was heated overnight at 120 deg C. The reaction mixture was poured into saturated sodium bicarbonate/saturated sodium chloride and CH₂Cl₂. The organic layer was separated, and the aqueous layer was extracted with CH₂Cl₂. The combined organic layers were dried over sodium sulfate, then filtered and evaporated. The crude residue was purified by column chromatography (Hex:EtOAc then CH₂Cl₂:MeOH) to afford methyl 2-(1-(5-methyl-2-((3-methylisothiazol-5-yl)amino)pyrimidin-4-yl)-3-(piperidin-1-yl)azetidin-3-yl)acetate in 33% yield (61 mg, 0.14 mmol).

Using the same general technique, the following compounds of Table 20 were synthesized.

TABLE 20
Pyrazoles

| | | |
|---|---|---|
| E152a | 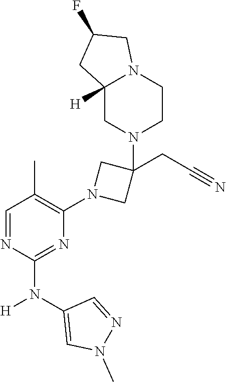 [SMILES: Cc1cnc(Nc2cnn(C)c2)nc1N1CC(CC#N)(N2CCN3C[C@H](F)C[C@H]3C2)C1] | <10 nM TYK2 <10 nM JAK1 |
| E153a | 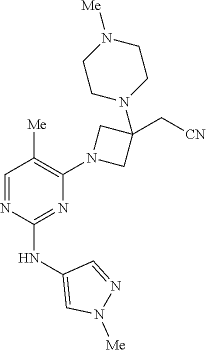 [SMILES: Cc1cnc(Nc2cnn(C)c2)nc1N1CC(CC#N)(N2CCN(C)CC2)C1] | <20 nM TYK2 <20 nM JAK1 |
| E156a | 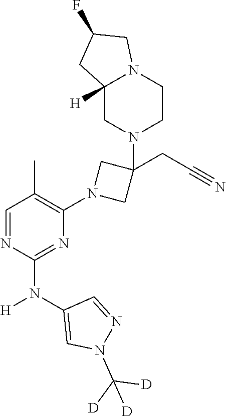 [SMILES: [2H]C([2H])([2H])n1cc(Nc2ncc(C)c(N3CC(CC#N)(N4CCN5C[C@H](F)C[C@H]5C4)C3)n2)cn1] | <10 nM TYK2 <10 nM JAK1 |
| E157a | 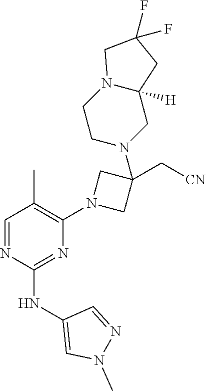 [SMILES: Cc1cnc(Nc2cnn(C)c2)nc1N1CC(CC#N)(N2CCN3CC(F)(F)C[C@H]3C2)C1] | <10 nM TYK2 <10 nM JAK1 |
| E158a | 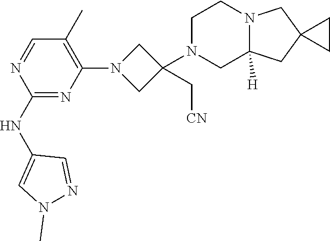 | <20 nM TYK2 <10 nM JAK1 |
| E159a | 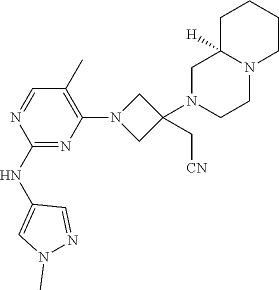 [SMILES: Cc1cnc(Nc2cnn(C)c2)nc1N1CC(CC#N)(N2CCN3CCCC[C@@H]3C2)C1] | <100 nM TYK2 <120 nM JAK1 |
| E160a | 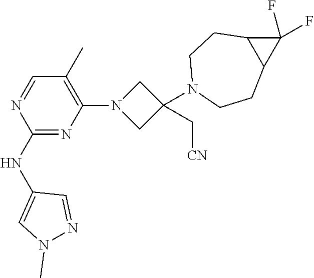 [SMILES: Cc1cnc(Nc2cnn(C)c2)nc1N1CC(CC#N)(N2CCC3C(CC2)C3(F)F)C1] | <10 nM TYK2 <10 nM JAK1 |
| E161a | 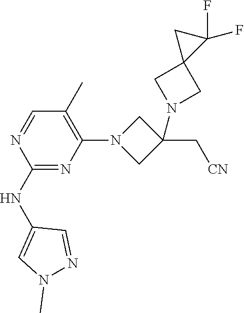 [SMILES: Cc1cnc(Nc2cnn(C)c2)nc1N1CC(CC#N)(N2CC3(C2)CC3(F)F)C1] | <20 nM TYK2 <20 nM JAK1 |
| E162a | 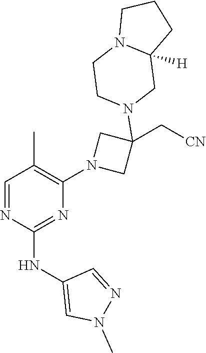 [SMILES: Cc1cnc(Nc2cnn(C)c2)nc1N1CC(CC#N)(N2CCN3CCC[C@H]3C2)C1] | <50 nM TYK2 <50 nM JAK1 |
| E163a | 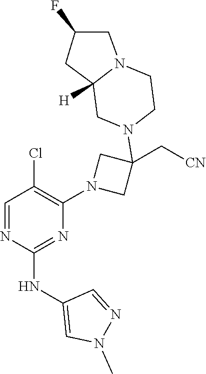 [SMILES: Cn1cc(Nc2ncc(Cl)c(N3CC(CC#N)(N4CCN5C[C@H](F)C[C@H]5C4)C3)n2)cn1] | <10 nM TYK2 <10 nM JAK1 |
| E164a | 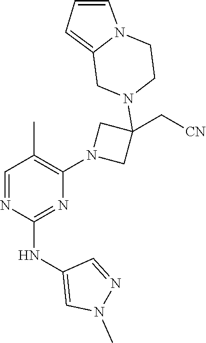 [SMILES: Cc1cnc(Nc2cnn(C)c2)nc1N1CC(CC#N)(N2CCn3cccc3C2)C1] | <10 nM TYK2 <10 nM JAK1 |
| E165a | 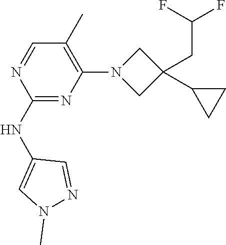 [SMILES: Cc1cnc(Nc2cnn(C)c2)nc1N1CC(CC(F)F)(C2CC2)C1] | <10 nM TYK2 <10 nM JAK1 |
| E166a | 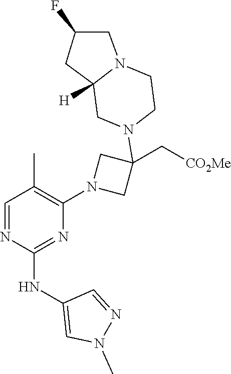 [SMILES: COC(=O)CC1(N2CCN3C[C@H](F)C[C@H]3C2)CN(c2nc(Nc3cnn(C)c3)ncc2C)C1] | <200 nM TYK2 <1 μM JAK1 |
| E167a | 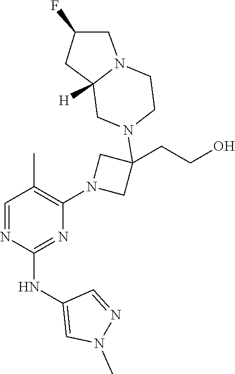 [SMILES: Cc1cnc(Nc2cnn(C)c2)nc1N1CC(CCO)(N2CCN3C[C@H](F)C[C@H]3C2)C1] | <50 nM TYK2 <50 nM JAK1 |
| E168a | 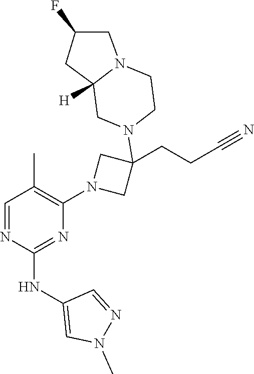 [SMILES: Cc1cnc(Nc2cnn(C)c2)nc1N1CC(CCC#N)(N2CCN3C[C@H](F)C[C@H]3C2)C1] | <20 nM TYK2 <20 nM JAK1 |
| E169a | 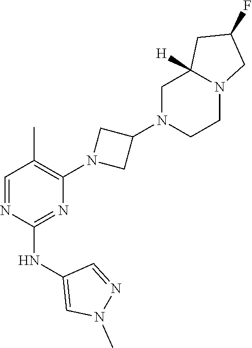 [SMILES: Cc1cnc(Nc2cnn(C)c2)nc1N1CC(N2CCN3C[C@H](F)C[C@H]3C2)C1] | <120 nM TYK2 <250 nM JAK1 |
| E170a | 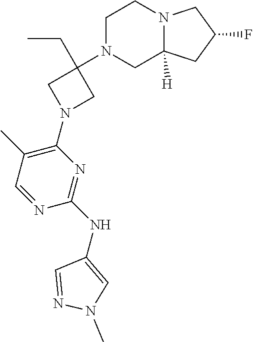 [SMILES: CCC1(N2CCN3C[C@H](F)C[C@H]3C2)CN(c2nc(Nc3cnn(C)c3)ncc2C)C1] | <100 nM TYK2 <50 nM JAK1 |
| E171a | 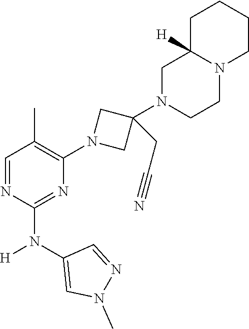 [SMILES: Cc1cnc(Nc2cnn(C)c2)nc1N1CC(CC#N)(N2CCN3CCCC[C@H]3C2)C1] | <10 nM TYK2 <10 nM JAK1 |
| E172a | 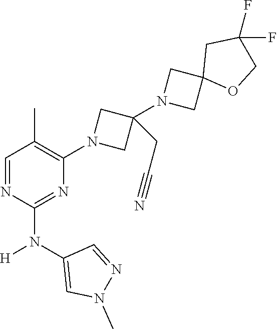 [SMILES: Cc1cnc(Nc2cnn(C)c2)nc1N1CC(CC#N)(N2CC3(C2)CC(F)(F)CO3)C1] | <100 nM TYK2 <100 nM JAK1 |
| E173a | 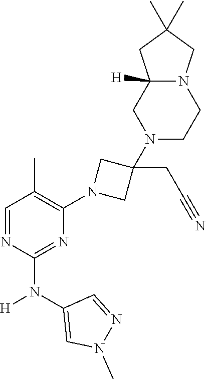 [SMILES: Cc1cnc(Nc2cnn(C)c2)nc1N1CC(CC#N)(N2CCN3CC(C)(C)C[C@H]3C2)C1] | <10 nM TYK2 <10 nM JAK1 |
| E174a | 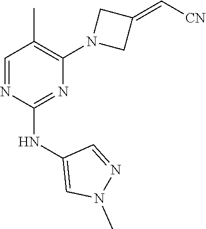 [SMILES: Cc1cnc(Nc2cnn(C)c2)nc1N1CC(=CC#N)C1] | <200 nM TYK2 <100 nM JAK1 |
| E175a | 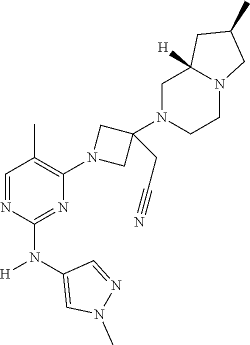 [SMILES: Cc1cnc(Nc2cnn(C)c2)nc1N1CC(CC#N)(N2CCN3C[C@H](C)C[C@H]3C2)C1] | <50 nM TYK2 <50 nM JAK1 |
| E175- A | 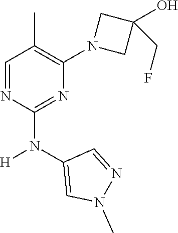 [SMILES: Cc1cnc(Nc2cnn(C)c2)nc1N1CC(O)(CF)C1] | <120 nM TYK2 <100 nM JAK1 |
| E175- B | 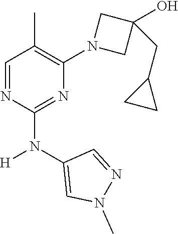 [SMILES: Cc1cnc(Nc2cnn(C)c2)nc1N1CC(O)(CC2CC2)C1] | <200 nM TYK2 <200 nM JAK1 |
| E175- C | 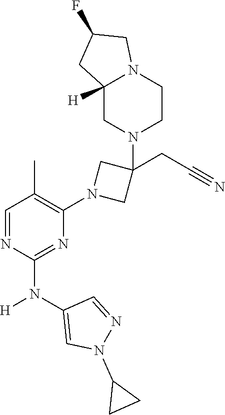 [SMILES: Cc1cnc(Nc2cnn(C3CC3)c2)nc1N1CC(CC#N)(N2CCN3C[C@H](F)C[C@H]3C2)C1] | <10 nM TYK2 <10 nM JAK1 |
| E175- D | 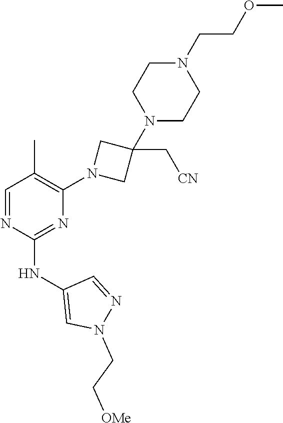 [SMILES: COCCN1CCN(C2(CC#N)CN(c3nc(Nc4cnn(CCOC)c4)ncc3C)C2)CC1] | <120 nM TYK2 <120 nM JAK1 |
| E175- E | 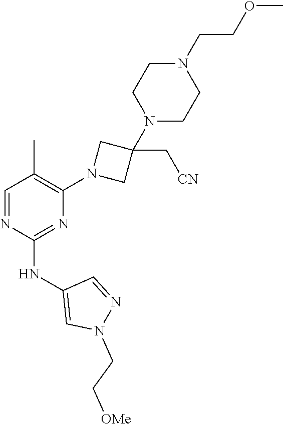 [SMILES: COCCN1CCN(C2(CC#N)CN(c3nc(Nc4cnn(CCOC)c4)ncc3C)C2)CC1] | <200 nM TYK2 <200 nM JAK1 |
| E175- | | <100 nM |
| H | | TYK2 |
| | | <50 nM |
| | | JAK1 |
| E175- | | |
| I | | |
| E175- | | |
| J | | |

TABLE 21

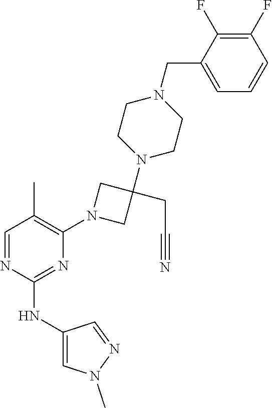 [SMILES: Cc1cnc(Nc2cnn(C)c2)nc1N1CC(CC#N)(N2CCN(Cc3cccc(F)c3F)CC2)C1]

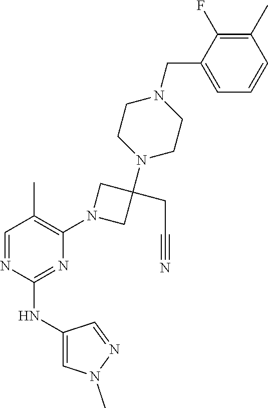 [SMILES: Cc1cnc(Nc2cnn(C)c2)nc1N1CC(CC#N)(N2CCN(Cc3cccc(C)c3F)CC2)C1]

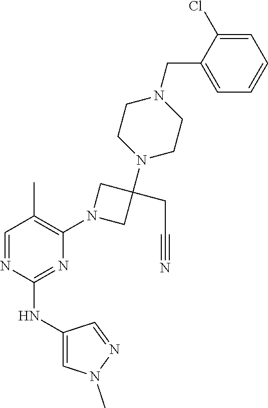 [SMILES: Cc1cnc(Nc2cnn(C)c2)nc1N1CC(CC#N)(N2CCN(Cc3ccccc3Cl)CC2)C1]

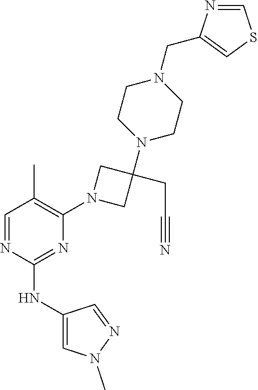 [SMILES: Cc1cnc(Nc2cnn(C)c2)nc1N1CC(CC#N)(N2CCN(Cc3cscn3)CC2)C1]

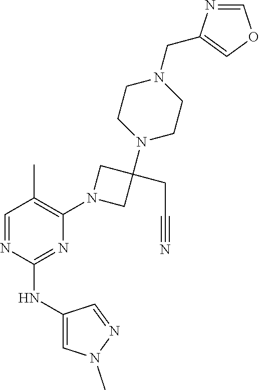 [SMILES: Cc1cnc(Nc2cnn(C)c2)nc1N1CC(CC#N)(N2CCN(Cc3cocn3)CC2)C1]

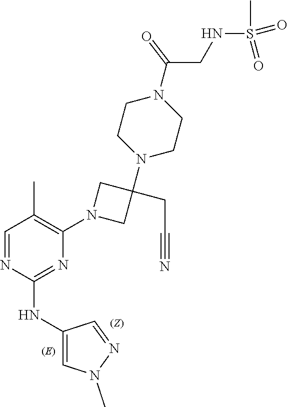 [SMILES: Cc1cnc(Nc2cnn(C)c2)nc1N1CC(CC#N)(N2CCN(C(=O)CNS(C)(=O)=O)CC2)C1]

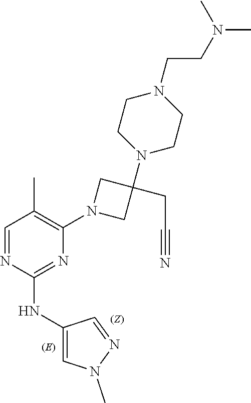 [SMILES: Cc1cnc(Nc2cnn(C)c2)nc1N1CC(CC#N)(N2CCN(CCN(C)C)CC2)C1]

### General Scheme 2 Preparation of Aryl & Heteroaryl Compounds.

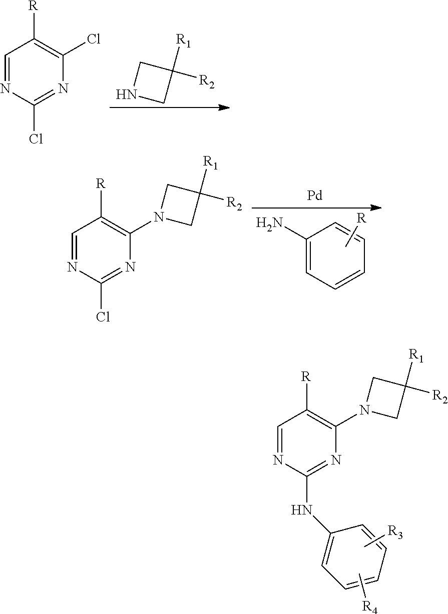

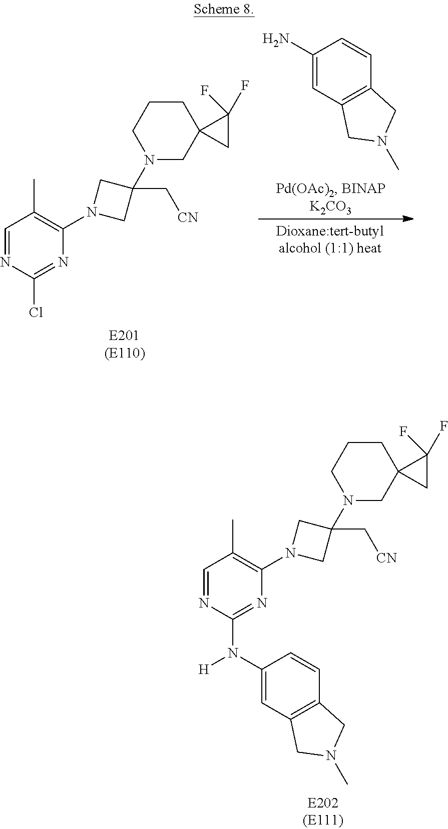 [SMILES: Cc1cnc(Cl)nc1N1CC(CC#N)(N2CCCC3(C2)CC3(F)F)C1 ; CN1Cc2ccc(N)cc2C1 ; Cc1cnc(Nc2ccc3c(c2)CN(C)C3)nc1N1CC(CC#N)(N2CCCC3(C2)CC3(F)F)C1]

Preparation of 2-(3-(1,1-difluoro-5-azaspiro[2.5]octan-5-yl)-1-(5-methyl-2-((2-methylisoindolin-5-yl)amino)pyrimidin-4-yl)azetidin-3-yl)acetonitrile (E202). To 2-(1-(2-chloro-5-methylpyrimidin-4-yl)-3-(1,1-difluoro-5-azaspiro[2.5]octan-5-yl)azetidin-3-yl)acetonitrile (E201), 58 mg, 0.16 mmol, 1.0 eq) under nitrogen was charged 2-methyl-2,3-dihydro-1H-isoindol-5-amine dihydrochloride (37 mg, 0.17 mmol, 1.05 eq), palladium (II) acetate (2.2 mg, 0.01 mmol, 5 mol %), BINAP (15 mg, 0.02 mmol, 15 mol %), and potassium carbonate (231 mg, 0.63 mmol, 4.0 eq) followed by dioxane and tert-butyl alcohol (1.6 mL, 1:1). The reaction mixture was heated overnight at 100 deg C. The reaction mixture was poured into saturated sodium bicarbonate and ethyl acetate. The organic layer was separated, and the aqueous layer was extracted with 3:1 CH₂Cl₂/Isopropyl alcohol (IPA). The combined organic layers were dried over sodium sulfate, then filtered and evaporated. The crude residue was purified by column chromatography (Hexanes:EtOAc, then CH₂Cl₂:EtOH) to afford 2-(3-(1,1-difluoro-5-azaspiro[2.5]octan-5-yl)-1-(5-methyl-2-((2-methylisoindolin-5-yl)amino)pyrimidin-4-yl)azetidin-3-yl)acetonitrile in 33% yield (E202, 25 mg, 0.05 mmol).

Using procedures to those set forth above for Schemes 1-3 and substituting the appropriate starting materials, the compounds E203-E218 (Table 22) were synthesized and E219-E268 (Table 23) could be synthesized.

TABLE 22

| | | |
|---|---|---|
| E213 | 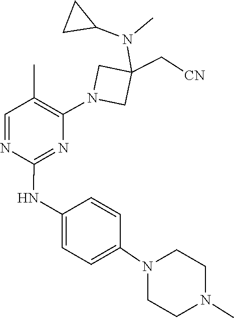 [SMILES: Cc1cnc(Nc2ccc(N3CCN(C)CC3)cc2)nc1N1CC(CC#N)(N(C)C2CC2)C1] | <10 nM TYK2 <50 nM JAK1 |
| E216 | 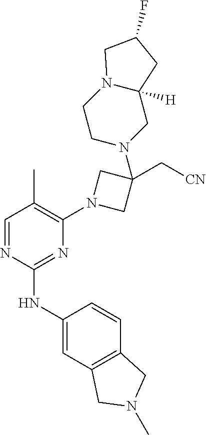 [SMILES: Cc1cnc(Nc2ccc3c(c2)CN(C)C3)nc1N1CC(CC#N)(N2CCN3C[C@H](F)C[C@H]3C2)C1] | <10 nM TYK2 <10 nM JAK1 |
| E216-A | 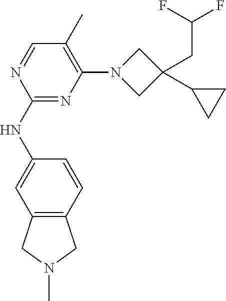 [SMILES: Cc1cnc(Nc2ccc3c(c2)CN(C)C3)nc1N1CC(CC(F)F)(C2CC2)C1] | <10 nM TYK2 <10 nM JAK1 |
| E216-B | 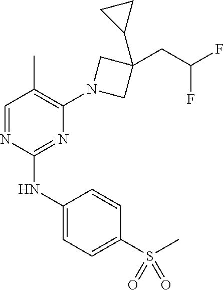 [SMILES: Cc1cnc(Nc2ccc(S(C)(=O)=O)cc2)nc1N1CC(CC(F)F)(C2CC2)C1] | <10 nM TYK2 <10 nM JAK1 |
| E216-C | 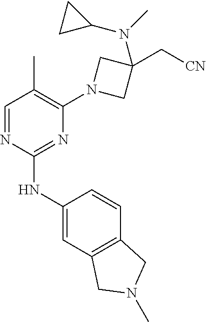 [SMILES: Cc1cnc(Nc2ccc3c(c2)CN(C)C3)nc1N1CC(CC#N)(N(C)C2CC2)C1] | <10 nM TYK2 <10 nM JAK1 |
| E216-E | 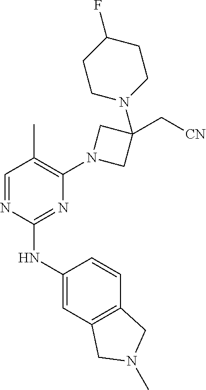 [SMILES: Cc1cnc(Nc2ccc3c(c2)CN(C)C3)nc1N1CC(CC#N)(N2CCC(F)CC2)C1] | <10 nM TYK2 <10 nM JAK1 |
| E216-F | 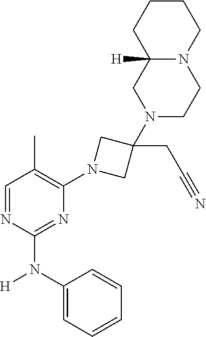 [SMILES: Cc1cnc(Nc2ccccc2)nc1N1CC(CC#N)(N2CCN3CCCC[C@H]3C2)C1] | <10 nM TYK2 <20 nM JAK1 |
| E216-G | 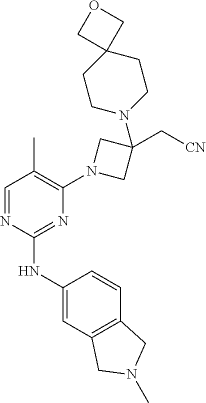 [SMILES: Cc1cnc(Nc2ccc3c(c2)CN(C)C3)nc1N1CC(CC#N)(N2CCC3(CC2)COC3)C1] | <10 nM TYK2 <10 nM JAK1 |
| E216-K | 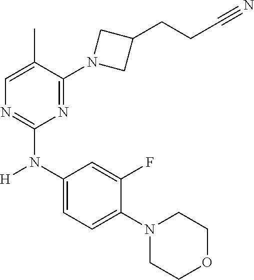 [SMILES: Cc1cnc(Nc2ccc(N3CCOCC3)c(F)c2)nc1N1CC(CCC#N)C1] | <10 nM TYK2 <50 nM JAK1 |
| E216-L | 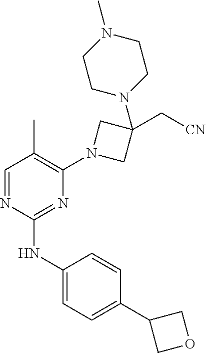 [SMILES: Cc1cnc(Nc2ccc(C3COC3)cc2)nc1N1CC(CC#N)(N2CCN(C)CC2)C1] | <20 nM TYK2 <50 nM JAK1 |
| E135 | 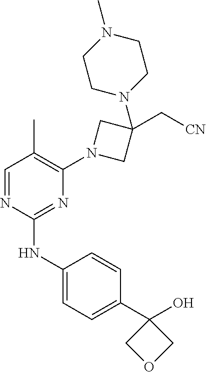 [SMILES: Cc1cnc(Nc2ccc(C3(O)COC3)cc2)nc1N1CC(CC#N)(N2CCN(C)CC2)C1] | <10 nM TYK2 <50 nM JAK1 |
| E216-N | 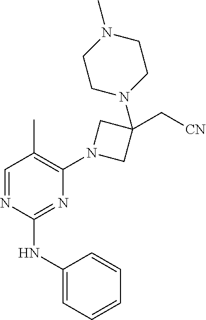 [SMILES: Cc1cnc(Nc2ccccc2)nc1N1CC(CC#N)(N2CCN(C)CC2)C1] | <30 nM TYK2 <70 nM JAK1 |
| E216-O | 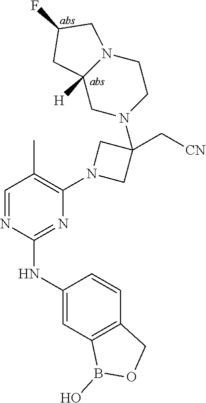 [SMILES: Cc1cnc(Nc2ccc3c(c2)B(O)OC3)nc1N1CC(CC#N)(N2CCN3C[C@H](F)C[C@H]3C2)C1] | <30 nM TYK2 <120 nM JAK1 |
| E216-S | 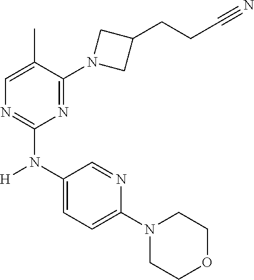 [SMILES: Cc1cnc(Nc2ccc(N3CCOCC3)nc2)nc1N1CC(CCC#N)C1] | <100 nM TYK2 <100 nM JAK1 |
| E216-T | 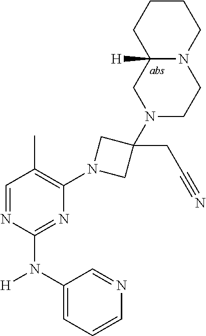 [SMILES: Cc1cnc(Nc2cccnc2)nc1N1CC(CC#N)(N2CCN3CCCC[C@H]3C2)C1] | <50 nM TYK2 <50 nM JAK1 |
| E135 | 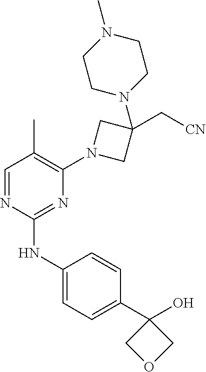 [SMILES: Cc1cnc(Nc2ccc(C3(O)COC3)cc2)nc1N1CC(CC#N)(N2CCN(C)CC2)C1] | <10 nM TYK2 <50 nM JAK1 |
| E216-V | 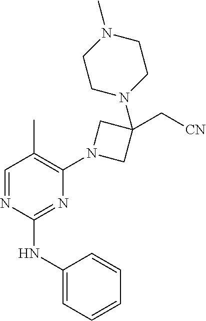 [SMILES: Cc1cnc(Nc2ccccc2)nc1N1CC(CC#N)(N2CCN(C)CC2)C1] | <50 nM TYK2 <100 nM JAK1 |

TABLE 23

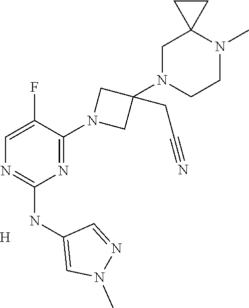

E231

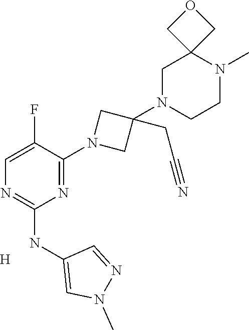

E232

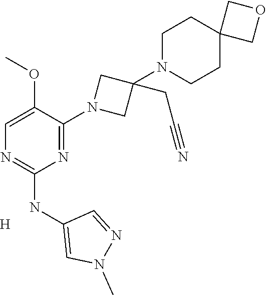

E233

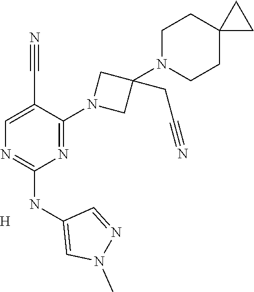

E234

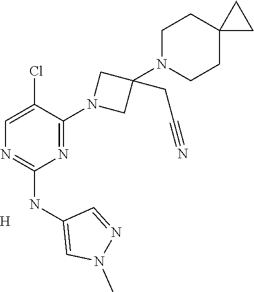

E235

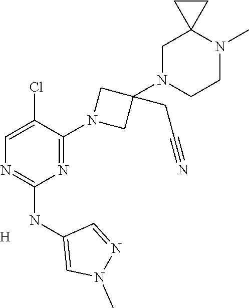

E236

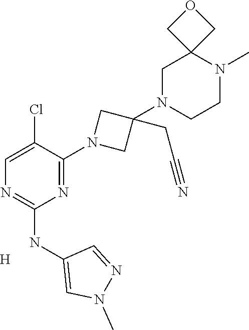

E237

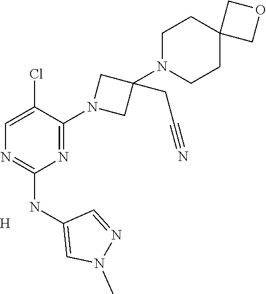

E238

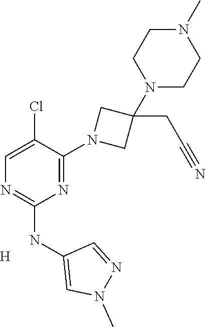

E239

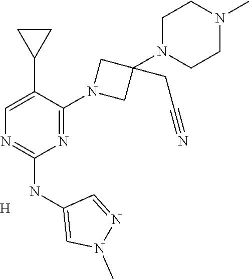

E240

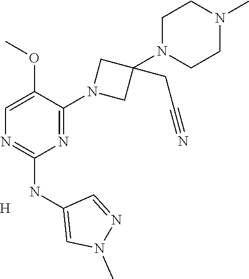

E242

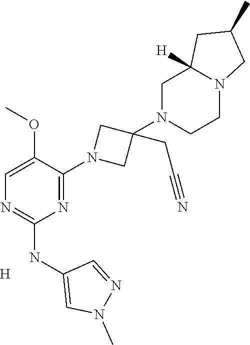

E243

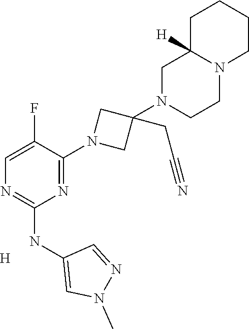

E244

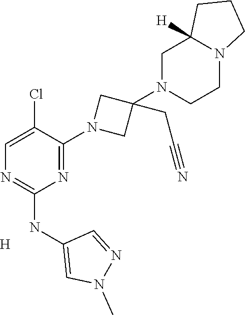

E246

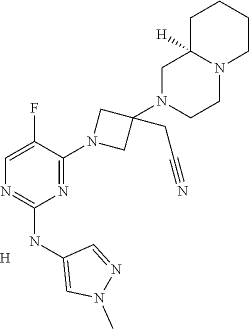

E247

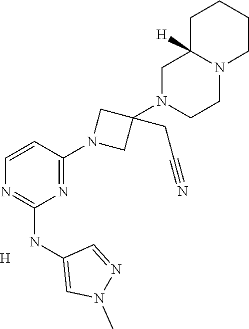

E250

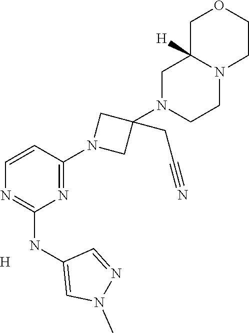

E251

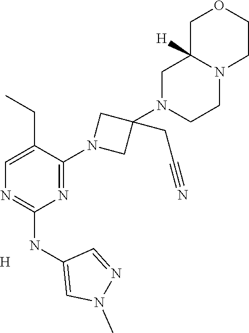

E252

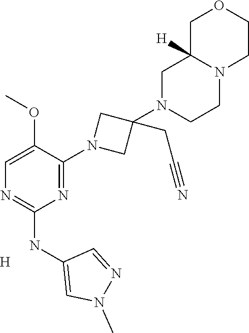

E253

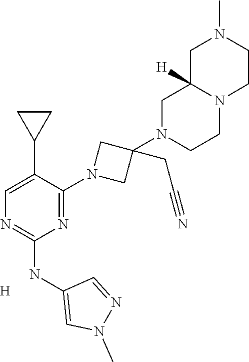

E254

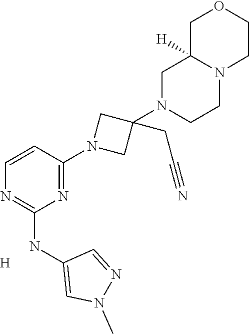

E255

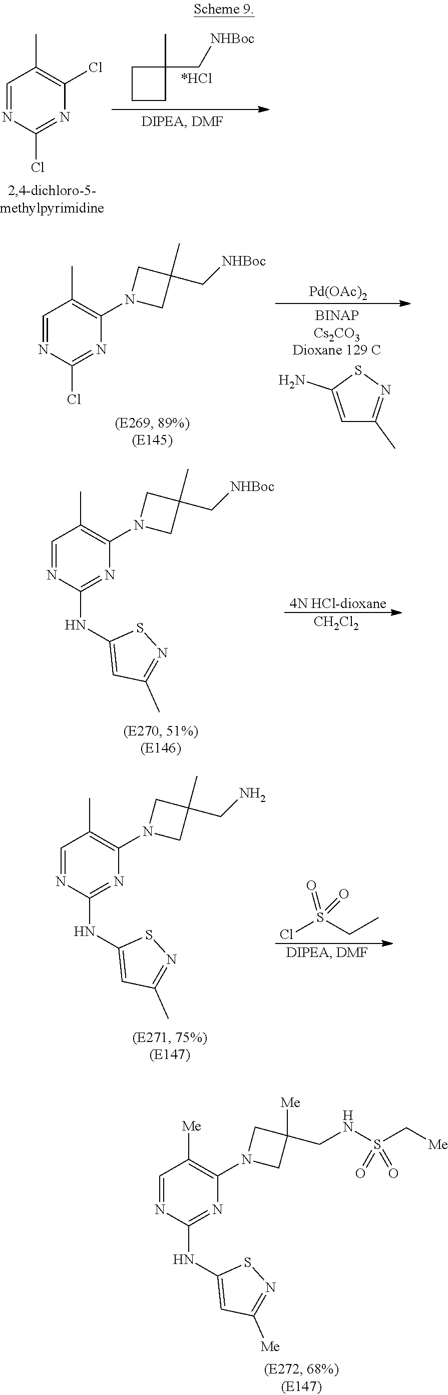

Preparation of tert-butyl ((1-(2-chloro-5-methylpyrimidin-4-yl)-3-methylazetidin-3-yl)methyl)carbamate (E269). To 2,4-dichloro-5-methylpyrimidine (196 mg, 1.2 mmol) in DMF (3.9 mL) was added tert-butyl ((3-methylazetidin-3-yl)methyl)carbamate hydrochloride (1.05 eq) and DIPEA (2 eq) and the solution was heated to 50° C. After 2 hours solution was cooled and extracted with EtOAc and NaHCO₃. The organics were dried (Na₂SO₄), filtered and evaporated. Column chromatography 0-100% EtOAc-Hexanes gave pure tert-butyl ((1-(2-chloro-5-methylpyrimidin-4-yl)-3-methylazetidin-3-yl)methyl)carbamate (E269, 89%).

Preparation of tert-butyl ((3-methyl-1-(5-methyl-2-((3-methylisothiazol-5-yl)amino)pyrimidin-4-yl)azetidin-3-yl)methyl)carbamate (E270). To tert-butyl ((1-(2-chloro-5-methylpyrimidin-4-yl)-3-methylazetidin-3-yl)methyl)carbamate (E269, 104 mg, 0.32 mmol) in dioxane (6.9 mL) in a pressure tube was added 3-methylisothiazol-5-amine (37.5 mg, 0.33 mmol), BINAP (39.8 mg, 0.06 mmol), Cs₂CO₃ (156 mg, 0.48 mmol), and Pd(OAc)₂ (7.1 mg, 0.03 mmol)) and the mixture was bubbled with N₂ for 1 min. The pressure tube was capped and heated to 129° C. for 1.5-2 h, cooled and the mixture was extracted with EtOAc and NaHCO₃(sat), dried (Na₂SO₄), filtered and evaporated. Column chromatography 0-5% MeOH—CH₂Cl₂ gave pure tert-butyl ((3-methyl-1-(5-methyl-2-((3-methylisothiazol-5-yl)amino)pyrimidin-4-yl)azetidin-3-yl)methyl)carbamate (E270, 51%.

Preparation of N-(4-(3-(aminomethyl)-3-methylazetidin-1-yl)-5-methylpyrimidin-2-yl)-3-methylisothiazol-5-amine (E271). To tert-butyl ((3-methyl-1-(5-methyl-2-((3-methylisothiazol-5-yl)amino)pyrimidin-4-yl)azetidin-3-yl)methyl)carbamate (270 67 mg, 0.17 mmol) in CH₂Cl₂ (0.9 mL) was added HCl (4N in dioxane, 0.33 mL, 1.33 mmol) and the solution was stirred for 3 h and evaporated. The compound was taken up in water and 2-methyl-THF. NaHCO₃(sat) and K₂CO₃ was added until pH 10 (pH paper) and the aqueous was extracted with 2-methyl THF. NaCl was added and the mixture was further extracted with 2-methyl THF. Column chromatography 0-90% (EtOH/10% 2N NH₃-MeOH)—CH₂Cl₂ gave pure N-(4-(3-(aminomethyl)-3-methylazetidin-1-yl)-5-methylpyrimidin-2-yl)-3-methylisothiazol-5-amine (E271, 39 mg, 75%).

Preparation of N-((3-methyl-1-(5-methyl-2-((3-methylisothiazol-5-yl)amino)pyrimidin-4-yl)azetidin-3-yl)methyl)ethanesulfonamide (E272). To N-(4-(3-(aminomethyl)-3-methylazetidin-1-yl)-5-methylpyrimidin-2-yl)-3-methylisothiazol-5-amine (E271, 17 mg, 0.06 mmol) in CH₂Cl₂ (4 mL) and DMF (0.2 mL) was added DIPEA (9.7 μL, 0.06 mmol) and the reaction was cooled to 0° C. Ethanesulfonyl chloride was added and the reaction was warmed to room temperature. The reaction was extracted with EtOAc, dried (Na₂SO₄), filtered and evaporated. Column chromatography 0-10% MeOH—CH₂Cl₂ gave pure N-((3-methyl-1-(5-methyl-2-((3-methylisothiazol-5-yl)amino)pyrimidin-4-yl)azetidin-3-yl)methyl)ethanesulfonamide (272 15.1 mg, 68%)

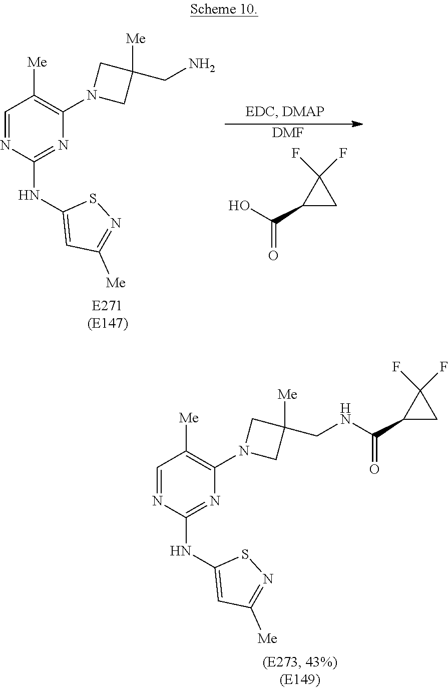

Preparation of (S)-2,2-difluoro-N-((3-methyl-1-(5-methyl-2-((3-methylisothiazol-5-yl)amino)pyrimidin-4-yl)azetidin-3-yl)methyl)cyclopropane-1-carboxamide (E273). To N-(4-(3-(aminomethyl)-3-methylazetidin-1-yl)-5-methylpyrimidin-2-yl)-3-methylisothiazol-5-amine (E271, 20.4 mg, 0.07 mmol) in DMF (0.35 mL) was added (S)-2,2-difluorocyclopropane-1-carboxylic acid (9.8 mg, 0.08 mmol), EDC (22 mg, 0.11 mmol), DMAP (1.6 mg, 0.01 mmol) and solution stirred overnight at room temperature. The reaction mixture was poured into EtOAc/NaHCO₃(sat) and extracted with EtOAc, dried (Na₂SO₄), filtered and evaporated. Column chromatography (0-10% MeOH—CH₂Cl₂) gave pure (S)-2,2-difluoro-N-((3-methyl-1-(5-methyl-2-((3-methylisothiazol-5-yl)amino)pyrimidin-4-yl)azetidin-3-yl)methyl)cyclopropane-1-carboxamide (E273, 11.5 mg, 43%).

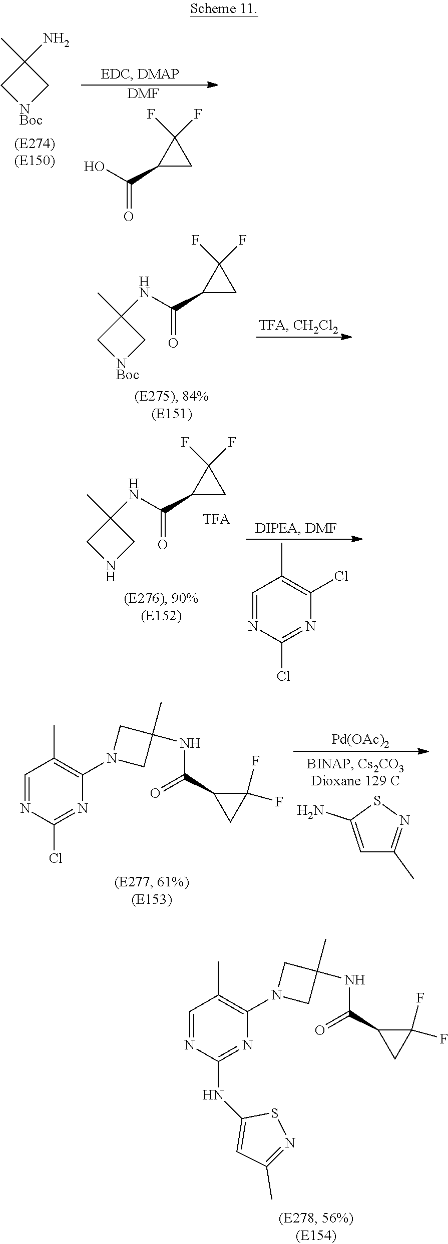

Preparation of tert-butyl (S)-3-(2,2-difluorocyclopropane-1-carboxamido)-3-methylazetidine-1-carboxylate (E275). To tert-butyl 3-amino-3-methylazetidine-1-carboxylate (E274, 400 mg, 2.1 mmol) in DMF (7 mL) was added (S)-2,2-difluorocyclopropane-1-carboxylic acid (313 mg, 2.6 mmol), EDC (659 mg, 3.4 mmol), and DMAP (52 mg, 0.4 mmol) and the solution stirred at room temperature overnight. The reaction mixture was poured into EtOAc and HCL (1N) and extracted with EtOAc, dried (Na₂SO₄) and filtered. Column chromatography 0-50% EtOAc-Hexanes gave pure tert-butyl (S)-3-(2,2-difluorocyclopropane-1-carboxamido)-3-methylazetidine-1-carboxylate (E275, 519 mg, 84%).

Preparation of (S)-2,2-difluoro-N-(3-methylazetidin-3-yl)cyclopropane-1-carboxamide trifluoroacetate (E276). To tert-butyl (S)-3-(2,2-difluorocyclopropane-1-carboxamido)-3-methylazetidine-1-carboxylate (E275, 519 mg, 1.79 mmol) in CH₂Cl₂ (13 mL) was added TFA (2 mL) and the solution stirred overnight. The solvents were evaporated and the residue azeotroped with toluene to give (S)-2,2-difluoro-N-(3-methylazetidin-3-yl)cyclopropane-1-carboxamide trifluoracetate (E276, 492 mg, 90%).

Preparation of N-(1-(2-chloro-5-methylpyrimidin-4-yl)-3-methylazetidin-3-yl)-2,2-difluorocyclopropane-1-carboxamide (E277). To (S)-2,2-difluoro-N-(3-methylazetidin-3-yl)cyclopropane-1-carboxamide trifluoracetate (E276, 492 mg, 1.6 mmol) in DMF (5.2 mL) was added DIPEA (0.57 mL, 3.2 mmol) and 2,4-dichloro-5-methylpyrimidine (264 mg, 1.6 mmol) and the solution was heated to 30° C. overnight. The reaction mixture was cooled and poured into EtOAc and NaHCO₃(sat) and extracted with EtOAc, dried (Na₂SO₄), filtered and evaporated. Automated column 0-80% EtOAc-Hexanes gave pure N-(1-(2-chloro-5-methylpyrimidin-4-yl)-3-methylazetidin-3-yl)-2,2-difluorocyclopropane-1-carboxamide (E277, 315 mg, 61%)

Preparation of 2,2-difluoro-N-(3-methyl-1-(5-methyl-2-((3-methylisothiazol-5-yl)amino)pyrimidin-4-yl)azetidin-3-yl)cyclopropane-1-carboxamide (E278). To 2,2-difluoro-N-(3-methyl-1-(5-methyl-2-((3-methylisothiazol-5-yl)amino)pyrimidin-4-yl)azetidin-3-yl)cyclopropane-1-carboxamide (E277, 73 mg, 0.23 mmol) in dioxane (4.9 mL) in a pressure tube was added 3-methylisothiazol-5-amine (27.4 mg, 0.24 mmol), BINAP (28.4 mg, 0.05 mmol), Cs₂CO₃ (111 mg, 0.34 mmol), and Pd(OAc)₂ (5.1 mg, 0.02 mmol)) and the mixture was bubbled with N₂ for 1 min. The pressure tube was capped and heated to 129° C. for 1.5-2 h, cooled and the mixture was extracted with EtOAc and NaHCO₃(sat), dried (Na₂SO₄), filtered and evaporated. Column chromatography 0-5% MeOH—CH₂Cl₂ gave pure 2,2-difluoro-N-(3-methyl-1-(5-methyl-2-((3-methylisothiazol-5-yl)amino)pyrimidin-4-yl)azetidin-3-yl)cyclopropane-1-carboxamide (E278, 50 mg, 56%)

Using the above schemes, S1-S4, with suitable modifications, the following compounds were synthesized.

```
TABLE 24

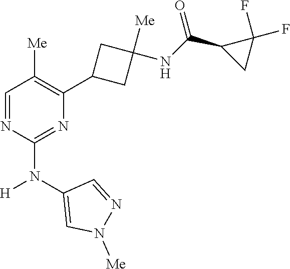 [SMILES: Cc1cnc(Nc2cnn(C)c2)nc1C1CC(C)(NC(=O)[C@@H]2CC2(F)F)C1]

  <10 nM TYK2
  <5 nM JAK1
E279

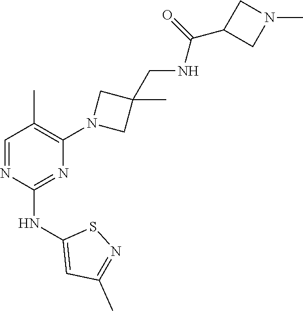 [SMILES: Cc1cc(Nc2ncc(C)c(N3CC(C)(CNC(=O)C4CN(C)C4)C3)n2)sn1]

<400 nM TYK2
<400 nM JAK1
E280

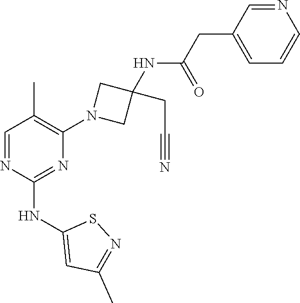 [SMILES: Cc1cc(Nc2ncc(C)c(N3CC(CC#N)(NC(=O)Cc4cccnc4)C3)n2)sn1]

  <50 nM TYK2
<100 nM JAK1
E281

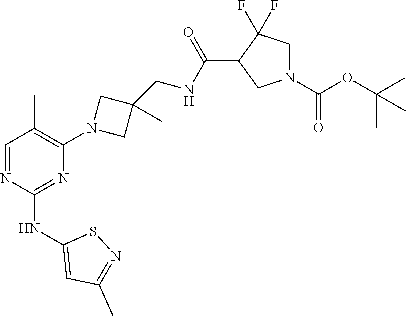 [SMILES: Cc1cc(Nc2ncc(C)c(N3CC(C)(CNC(=O)C4CN(C(=O)OC(C)(C)C)CC4(F)F)C3)n2)sn1]

<100 nM TYK2
<100 nM JAK1
E283

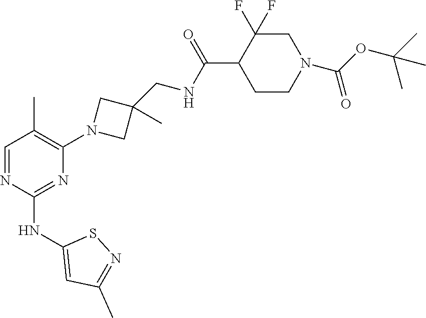 [SMILES: Cc1cc(Nc2ncc(C)c(N3CC(C)(CNC(=O)C4CCN(C(=O)OC(C)(C)C)CC4(F)F)C3)n2)sn1]

<200 nM TYK2
<100 nM JAK1
E284

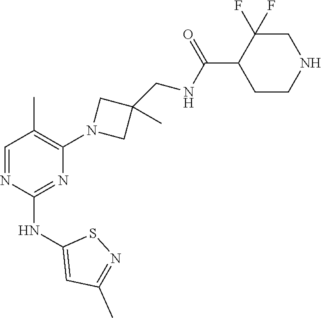 [SMILES: Cc1cc(Nc2ncc(C)c(N3CC(C)(CNC(=O)C4CCNCC4(F)F)C3)n2)sn1]

<150 nM TYK2
<100 nM JAK1
E285

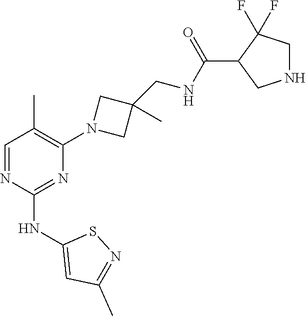 [SMILES: Cc1cc(Nc2ncc(C)c(N3CC(C)(CNC(=O)C4CNCC4(F)F)C3)n2)sn1]

  <50 nM TYK2
  <50 nM JAK1
E286
```

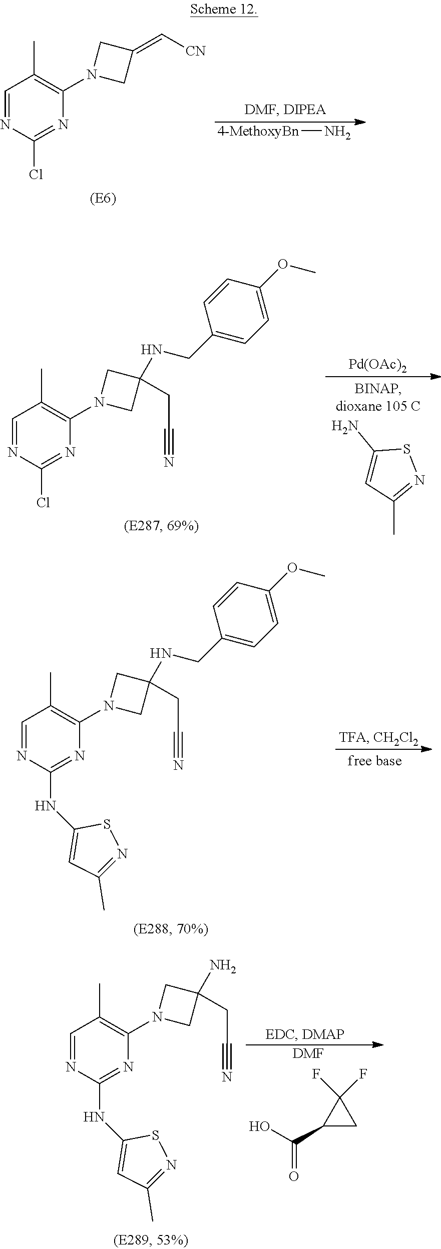

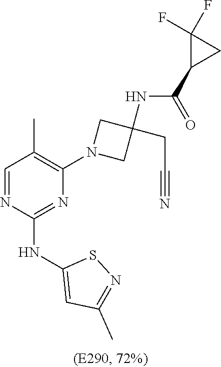 [SMILES: Cc1cc(Nc2ncc(C)c(N3CC(CC#N)(NC(=O)[C@@H]4CC4(F)F)C3)n2)sn1]

Preparation of 2-(1-(2-chloro-5-methylpyrimidin-4-yl)-3-((4-methoxybenzyl)amino)azetidin-3-yl)acetonitrile (E287). To 2-(1-(2-chloro-5-methylpyrimidin-4-yl)azetidin-3-ylidene)acetonitrile (E6, 250 mg, 1.1 mmol) in DMF (3.7 mL) was added 4-methoxy benzylamine (163 μL, 1.25 mmol) and DIPEA (177 μL, 1.0 mmol) and the solution was stirred at room temperature overnight. Additional 4-methoxy benzylamine (146 μL, 1.1 mmol) and DIPEA (78 μL, 0.46 mmol) in two portions were added and stirring continued for 30 hours. The reaction was extracted with EtOAc and NaHCO₃ (sat), then washed with NaCl (sat), dried (Na₂SO₄) filtered and evaporated. Column chromatography (Teledyne, 12 g gold cartridge, 0-5% MeOH—CH₂Cl₂) gave pure 2-(1-(2-chloro-5-methylpyrimidin-4-yl)-3-((4-methoxybenzyl)amino)azetidin-3-yl)acetonitrile (E287, 69%)

Preparation of 2-(3-((4-methoxybenzyl)amino)-1-(5-methyl-2-((3-methylisothiazol-5-yl)amino)pyrimidin-4-yl)azetidin-3-yl)acetonitrile (E288). To 2-(1-(2-chloro-5-methylpyrimidin-4-yl)-3-((4-methoxybenzyl)amino)azetidin-3-yl)acetonitrile (E287, 279 mg, 0.78), 3-methylisothiazol-5-amine (120 mg, 1.1 mmol), Cs₂CO₃ (381 mg, 1.17 mmol), BINAP (97 mg, 0.16 mmol) and Pd(OAc)₂ (17.5 mg, 0.08 mmol) in a pressure tube was added 1,4 dioxane (10.4 mL) and the sealed tube was heated to 105° C. for 4 hours. The reaction was cooled and extracted with EtOAc and water. Then the organics were extracted with NaCl (sat), dried (Na₂SO₄), filtered and evaporated. Column chromatography (Teledyne, 12 g Gold cartridge, 0-8% MeOH—CH₂Cl₂) gave pure 2-(3-((4-methoxybenzyl)amino)-1-(5-methyl-2-((3-methylisothiazol-5-yl)amino)pyrimidin-4-yl)azetidin-3-yl)acetonitrile (E288, 239 mg, 70%).

Preparation of 2-(3-amino-1-(5-methyl-2-((3-methylisothiazol-5-yl)amino)pyrimidin-4-yl)azetidin-3-yl)acetonitrile. (E289). To 2-(3-((4-methoxybenzyl)amino)-1-(5-methyl-2-((3-methylisothiazol-5-yl)amino)pyrimidin-4-yl)azetidin-3-yl)acetonitrile (E288, 239 mg, 0.56 mmol) in CH₂Cl₂ (3.4 mL) was added TFA (2.7 mL) and the solution was heated to 50° C. overnight. The solvents were evaporated and azeotroped with toluene. The residue was extracted with 2-methyl THF and NaHCO₃(sat), dried (Na₂SO₄), filtered and evaporated. Column chromatography (Teledyne, 4 g cartridge, 0-20% MeOH—CH₂Cl₂) gave pure 2-(3-amino-1-(5-methyl-2-((3-methylisothiazol-5-yl)amino)pyrimidin-4-yl)azetidin-3-yl)acetonitrile. (E289, 92 mg, 53%).

Preparation of (S)—N-(3-(cyanomethyl)-1-(5-methyl-2-((3-methylisothiazol-5-yl)amino)pyrimidin-4-yl)azetidin-3-yl)-2,2-difluorocyclopropane-1-carboxamide (E290). To 2-(3-amino-1-(5-methyl-2-((3-methylisothiazol-5-yl)amino)pyrimidin-4-yl)azetidin-3-yl)acetonitrile (E289, 26 mg, 0.08 mmol) in DMF (0.35 mL) was added EDC*HCl (26 mg, 0.13 mmol), DMAP (2 mg, 0.016 mmol) and (S)-2,2-difluorocyclopropane-1-carboxylic acid (12 mg, 0.099 mmol) and the solution was stirred overnight at room temperature. The reaction mixture was extracted with NaHCO₃(sat) and EtOAc, dried (Na₂SO₄), filtered and evaporated. Column chromatography (Teledyne, 4 g cartridge, 0-10% MeOH—CH₂Cl₂) gave pure (S)—N-(3-(cyanomethyl)-1-(5-methyl-2-((3-methylisothiazol-5-yl)amino)pyrimidin-4-yl)azetidin-3-yl)-2,2-difluorocyclopropane-1-carboxamide (E290, 25.1 mg, 72%).

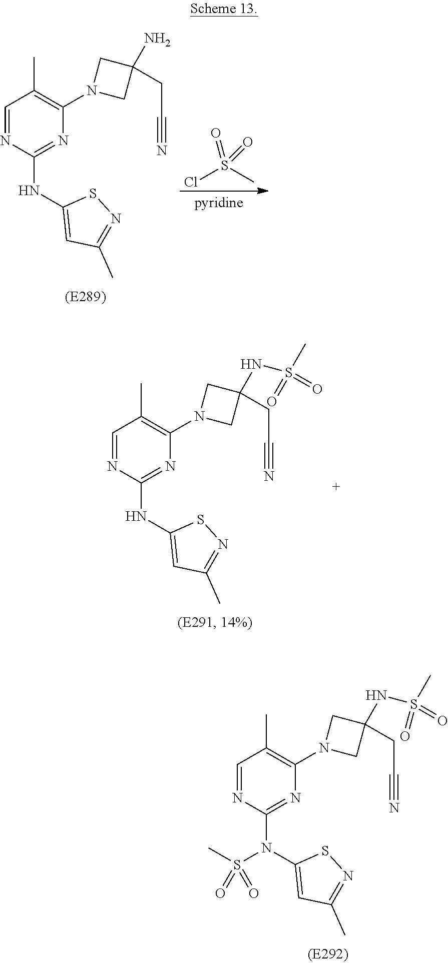

Preparation of N-(3-(cyanomethyl)-1-(5-methyl-2-((3-methylisothiazol-5-yl)amino)pyrimidin-4-yl)azetidin-3-yl)methanesulfonamide (E291). To 2-(3-amino-1-(5-methyl-2-((3-methylisothiazol-5-yl)amino)pyrimidin-4-yl)azetidin-3-yl)acetonitrile. (E289, 27 mg, 0.08 mmol) in pyridine (0.5 mL) was added methanesulfonyl chloride (7.1 μμL, 0.092 mmol) and the solution stirred at room temperature. Additional methanesulfonyl chloride was added (6.5 μL) and the reaction mixture stirred at room temperature. The reaction was extracted with EtOAc and NaHCO₃(sat), dried (Na₂SO₄), filtered and evaporated. Column chromatography (Teledyne, 4 g gold cartridge, 0-10% MeOH—CH₂Cl₂ gave pure N-(3-(cyanomethyl)-1-(5-methyl-2-((3-methylisothiazol-5-yl)amino)pyrimidin-4-yl)azetidin-3-yl)methanesulfonamide (E291, 4.6 mg, 14%) and byproduct E292.

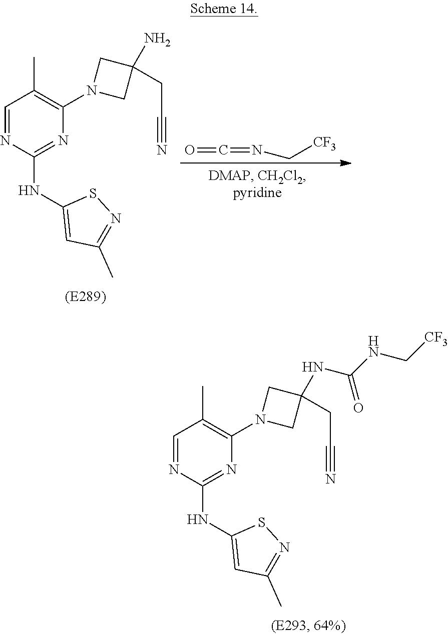

Preparation of 1-(3-(cyanomethyl)-1-(5-methyl-2-((3-methylisothiazol-5-yl)amino)pyrimidin-4-yl)azetidin-3-yl)-3-(2,2,2-trifluoroethyl)urea (E293). To 2-(3-amino-1-(5-methyl-2-((3-methylisothiazol-5-yl)amino)pyrimidin-4-yl)azetidin-3-yl)acetonitrile (E289, 25 mg, 0.08 mmol) in CH₂Cl₂ (0.3 mL) and pyridine (0.2 mL) was added DMAP (1-2 mg, 0.006 mmol) and 1,1,1-trifluoro-2-isocyanatoethane (11.9 mg, 0.095 mmol) and the reaction stirred at room temperature. After 2.5 hours additional 1,1,1-trifluoro-2-isocyanatoethane (30.6 mg in portions) was added and the solution was stirred at room temperature. The reaction was extracted with EtOAc and NaHCO₃ (sat), dried (Na₂SO₄), filtered and evaporated. Column chromatography (Teledyne, 4 g, 0-10% MeOH—CH₂Cl₂) provided 1-(3-(cyanomethyl)-1-(5-methyl-2-((3-methylisothiazol-5-yl)amino)pyrimidin-4-yl)azetidin-3-yl)-3-(2,2,2-trifluoroethyl)urea (E293, 22.4 mg, 64%).

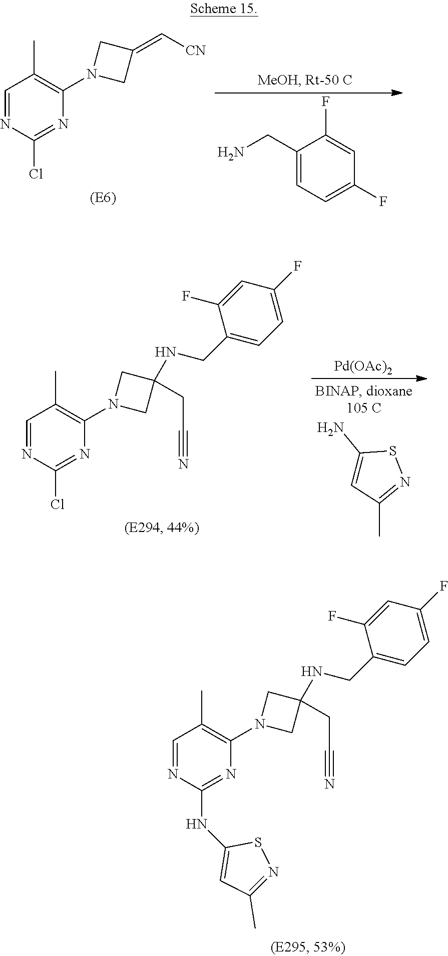

Preparation of 2-(1-(2-chloro-5-methylpyrimidin-4-yl)-3-((2,4-difluorobenzyl)amino)azetidin-3-yl)acetonitrile (E294). To 2-(1-(2-chloro-5-methylpyrimidin-4-yl)azetidin-3-ylidene)acetonitrile (E6, 131 mg, 0.59 mmol) in MeOH (1.8 mL) and DMF (0.5 mL) was added (2,4-difluorophenyl)methanamine (180 mg, 1.25 mmol) and DIPEA (113 μL, 0.65 mmol) and the solution was stirred at 50° C. overnight. The reaction was cooled to room temperature and extracted with EtOAc and NaHCO₃ (sat), dried (Na₂SO₄) filtered and evaporated. Column chromatography (Teledyne, 12 g gold cartridge, 0-5% MeOH—CH₂Cl₂) gave 2-(1-(2-chloro-5-methylpyrimidin-4-yl)-3-((2,4-difluorobenzyl)amino)azetidin-3-yl)acetonitrile (E294, 94.3 mg, 44%)

Preparation of 2-(3-((2,4-difluorobenzyl)amino)-1-(5-methyl-2-((3-methylisothiazol-5-yl)amino)pyrimidin-4-yl)azetidin-3-yl)acetonitrile (E295). To 2-(1-(2-chloro-5-methylpyrimidin-4-yl)-3-((2,4-difluorobenzyl)amino)azetidin-3-yl)acetonitrile (E294, 68 mg, 0.19 mmol), 3-methylisothiazol-5-amine (32 mg, 0.28 mmol), Cs₂CO₃ (91 mg, 0.28 mmol), BINAP (23 mg, 0.04 mmol) and Pd(OAc)₂ (4.2 mg, 0.02 mmol) in a pressure tube was added 1,4 dioxane (2.5 mL) and the solution was bubbled with N₂ (1 min) then sealed and heated to 105° C. for 4 hours. The reaction was cooled and extracted with EtOAc and water. Then the organics were extracted with NaCl (sat), dried (Na₂SO₄), filtered and evaporated. Column chromatography (Teledyne, 12 g Gold cartridge, 0-100% EtOAc-Hexanes) gave pure 2-(3-((2,4-difluorobenzyl)amino)-1-(5-methyl-2-((3-methylisothiazol-5-yl)amino)pyrimidin-4-yl)azetidin-3-yl)acetonitrile (E295, 44 mg, 53%).

Using the techniques outlined in the above schemes, with suitable modifications, the compounds of Table 25 and Table 26 were or can be made.

TABLE 25

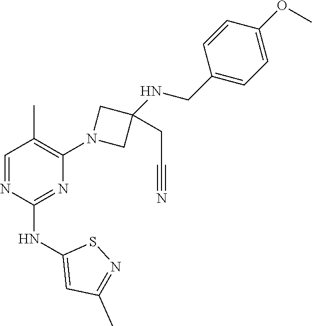 [SMILES: COc1ccc(CNC2(CC#N)CN(c3nc(Nc4cc(C)ns4)ncc3C)C2)cc1]

<50 nM TYK2
<50 nM JAK1
E288

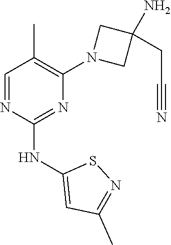 [SMILES: Cc1cc(Nc2ncc(C)c(N3CC(N)(CC#N)C3)n2)sn1]

<100 nM TYK2
<150 nM JAK1
E289

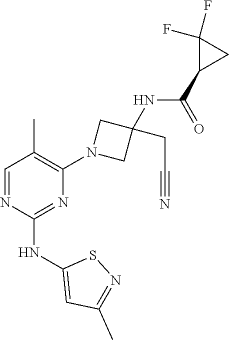 [SMILES: Cc1cc(Nc2ncc(C)c(N3CC(CC#N)(NC(=O)[C@@H]4CC4(F)F)C3)n2)sn1]

<10 nM TYK2
<10 nM JAK1
E290

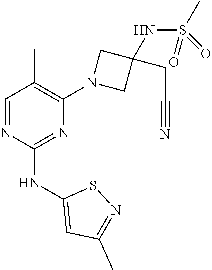 [SMILES: Cc1cc(Nc2ncc(C)c(N3CC(CC#N)(NS(C)(=O)=O)C3)n2)sn1]

<10 nM TYK2
<20 nM JAK1
E291

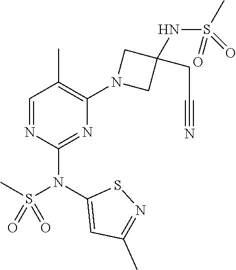 [SMILES: Cc1cc(N(c2ncc(C)c(N3CC(CC#N)(NS(C)(=O)=O)C3)n2)S(C)(=O)=O)sn1]

<1000 nM TYK2
<5000 nM JAK1
E292

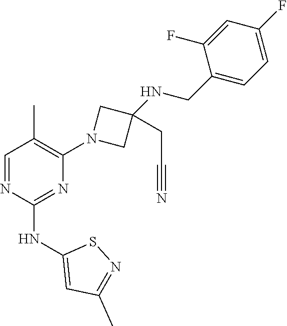 [SMILES: Cc1cc(Nc2ncc(C)c(N3CC(CC#N)(NCc4ccc(F)cc4F)C3)n2)sn1]

<10 nM TYK2
<20 nM JAK1
E295

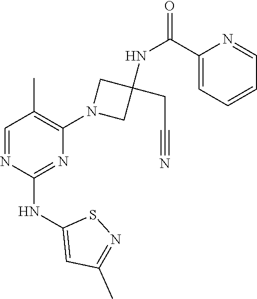 [SMILES: Cc1cc(Nc2ncc(C)c(N3CC(CC#N)(NC(=O)c4ccccn4)C3)n2)sn1]

<20 nM TYK2
<20 nM JAK1
E296

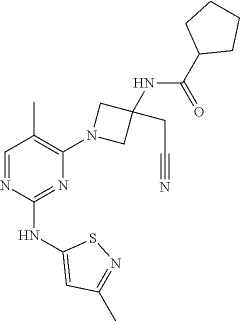 [SMILES: Cc1cc(Nc2ncc(C)c(N3CC(CC#N)(NC(=O)C4CCCC4)C3)n2)sn1]

<20 nM TYK2
<50 nM JAK1
E297

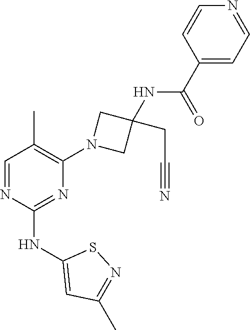 [SMILES: Cc1cc(Nc2ncc(C)c(N3CC(CC#N)(NC(=O)c4ccncc4)C3)n2)sn1]

<50 nM TYK2
<50 nM JAK1
E298

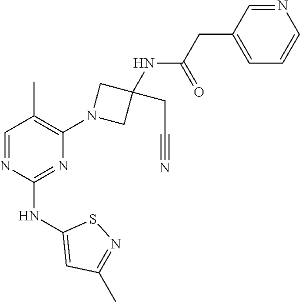 [SMILES: Cc1cc(Nc2ncc(C)c(N3CC(CC#N)(NC(=O)Cc4cccnc4)C3)n2)sn1]

<50 nM TYK2
<100 nM JAK1
E299

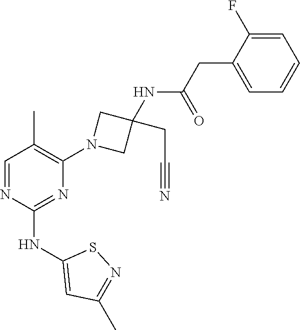 [SMILES: Cc1cc(Nc2ncc(C)c(N3CC(CC#N)(NC(=O)Cc4ccccc4F)C3)n2)sn1]

<20 nM TYK2
<20 nM JAK1
E300

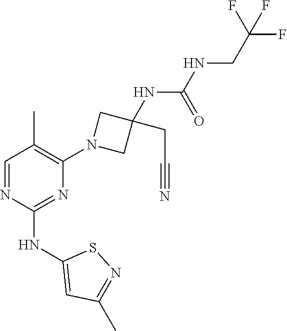 [SMILES: Cc1cc(Nc2ncc(C)c(N3CC(CC#N)(NC(=O)NCC(F)(F)F)C3)n2)sn1]

<20 nM TYK2
<20 nM JAK1
E293

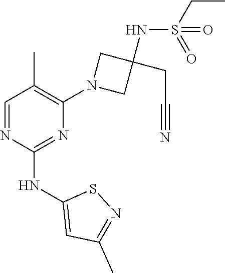 [SMILES: CCS(=O)(=O)NC1(CC#N)CN(c2nc(Nc3cc(C)ns3)ncc2C)C1]

<20 nM TYK2
<50 nM JAK1
E301

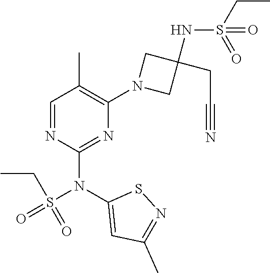 [SMILES: CCS(=O)(=O)NC1(CC#N)CN(c2nc(N(c3cc(C)ns3)S(=O)(=O)CC)ncc2C)C1]

<1000 nM TYK2
<5000 nM JAK1
E302

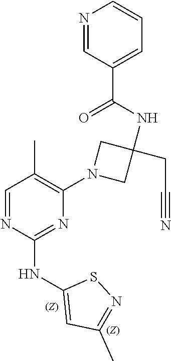 [SMILES: Cc1cc(Nc2ncc(C)c(N3CC(CC#N)(NC(=O)c4cccnc4)C3)n2)sn1]

<30 nM TYK2
<20 nM JAK1
E303

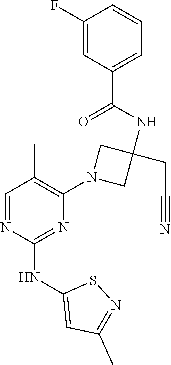 [SMILES: Cc1cc(Nc2ncc(C)c(N3CC(CC#N)(NC(=O)c4cccc(F)c4)C3)n2)sn1]

<10 nM TYK2
<10 nM JAK1
E304

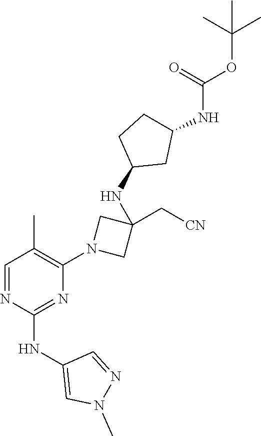 [SMILES: Cc1cnc(Nc2cnn(C)c2)nc1N1CC(CC#N)(N[C@H]2CC[C@H](NC(=O)OC(C)(C)C)C2)C1]

>2000 nM TYK2
>1500 nM JAK1
E305

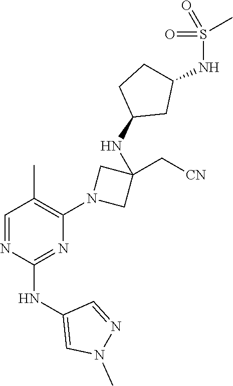 [SMILES: Cc1cnc(Nc2cnn(C)c2)nc1N1CC(CC#N)(N[C@H]2CC[C@H](NS(C)(=O)=O)C2)C1]

<60 nM TYK2
<120 nM JAK1
E306

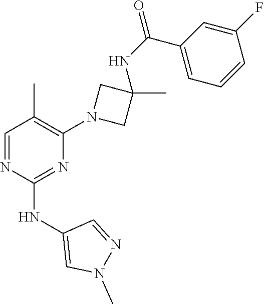 [SMILES: Cc1cnc(Nc2cnn(C)c2)nc1N1CC(C)(NC(=O)c2cccc(F)c2)C1]

<100 nM TYK2
<20 nM JAK1
E307

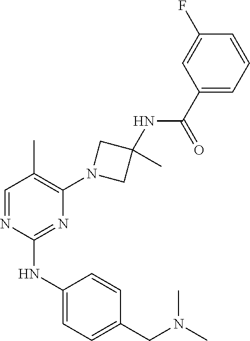 [SMILES: Cc1cnc(Nc2ccc(CN(C)C)cc2)nc1N1CC(C)(NC(=O)c2cccc(F)c2)C1]

<50 nM TYK2
<100 nM JAK1
E308

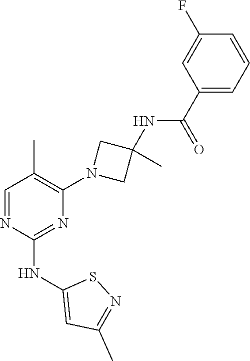 [SMILES: Cc1cc(Nc2ncc(C)c(N3CC(C)(NC(=O)c4cccc(F)c4)C3)n2)sn1]

<20 nM TYK2
<10 nM JAK1
E309

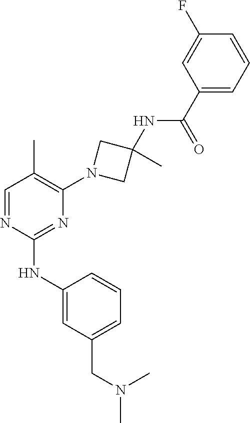 [SMILES: Cc1cnc(Nc2cccc(CN(C)C)c2)nc1N1CC(C)(NC(=O)c2cccc(F)c2)C1]

<50 nM TYK2
<20 nM JAK1
E310

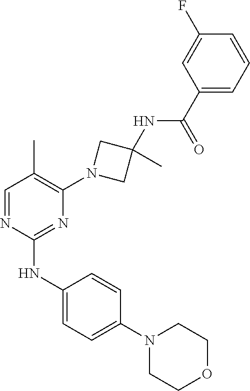 [SMILES: Cc1cnc(Nc2ccc(N3CCOCC3)cc2)nc1N1CC(C)(NC(=O)c2cccc(F)c2)C1]

<50 nM TYK2
<50 nM JAK1
E311

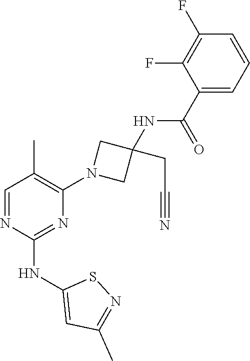 [SMILES: Cc1cc(Nc2ncc(C)c(N3CC(CC#N)(NC(=O)c4cccc(F)c4F)C3)n2)sn1]

<10 nM TYK2
<10 nM JAK1
E312

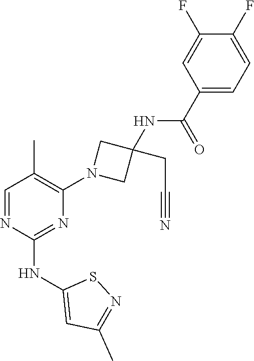 [SMILES: Cc1cc(Nc2ncc(C)c(N3CC(CC#N)(NC(=O)c4ccc(F)c(F)c4)C3)n2)sn1]

<50 nM TYK2
<20 nM JAK1
E313

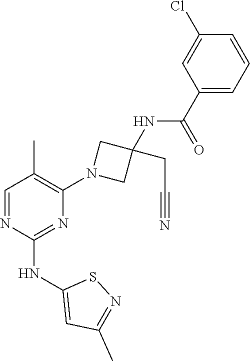 [SMILES: Cc1cc(Nc2ncc(C)c(N3CC(CC#N)(NC(=O)c4cccc(Cl)c4)C3)n2)sn1]

<10 nM TYK2
<10 nM JAK1
E314

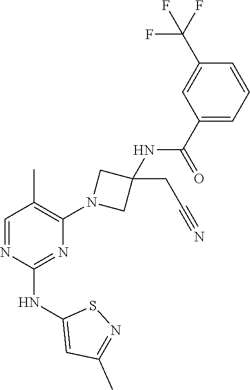 [SMILES: Cc1cc(Nc2ncc(C)c(N3CC(CC#N)(NC(=O)c4cccc(C(F)(F)F)c4)C3)n2)sn1]

<20 nM TYK2
<10 nM JAK1
E315
<10 nM TYK2
<10 nM JAK1
E316

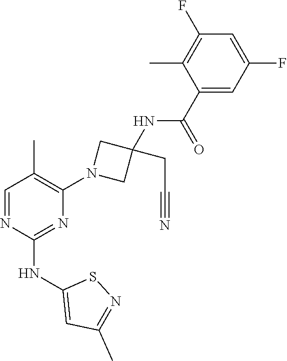 [SMILES: Cc1cc(Nc2ncc(C)c(N3CC(CC#N)(NC(=O)c4cc(F)cc(F)c4C)C3)n2)sn1]

<20 nM TYK2
<30 nM JAK1
E317

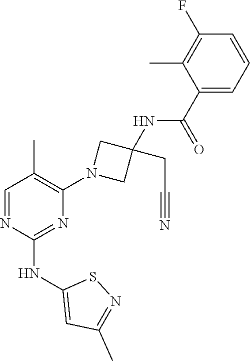 [SMILES: Cc1cc(Nc2ncc(C)c(N3CC(CC#N)(NC(=O)c4cccc(F)c4C)C3)n2)sn1]

<30 nM TYK2
<50 nM JAK1
E318
<30 nM TYK2
<20 nM JAK1
E318A

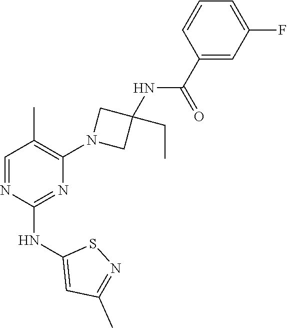 [SMILES: CCC1(NC(=O)c2cccc(F)c2)CN(c2nc(Nc3cc(C)ns3)ncc2C)C1]

<10 nM TYK2
<10 nM JAK1
E318B

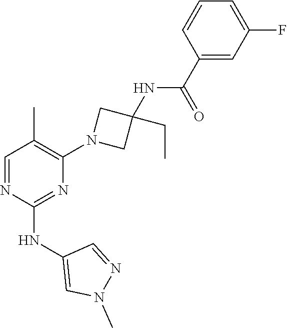 [SMILES: CCC1(NC(=O)c2cccc(F)c2)CN(c2nc(Nc3cnn(C)c3)ncc2C)C1]

<50 nM TYK2
<10 nM JAK1
E318C

TABLE 26

| | |
|---|---|
| 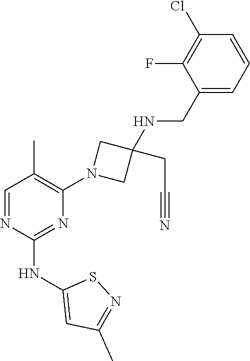 [SMILES: Cc1cc(Nc2ncc(C)c(N3CC(CC#N)(NCc4cccc(Cl)c4F)C3)n2)sn1] | E319 |
| 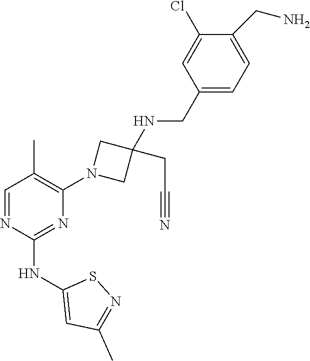 [SMILES: Cc1cc(Nc2ncc(C)c(N3CC(CC#N)(NCc4ccc(CN)c(Cl)c4)C3)n2)sn1] | E320 |
| 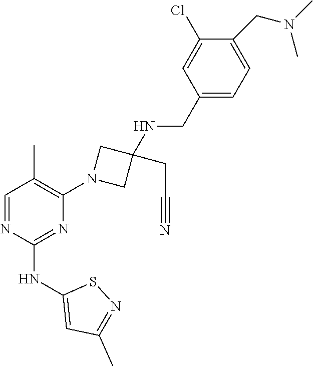 [SMILES: Cc1cc(Nc2ncc(C)c(N3CC(CC#N)(NCc4ccc(CN(C)C)c(Cl)c4)C3)n2)sn1] | E321 |
| 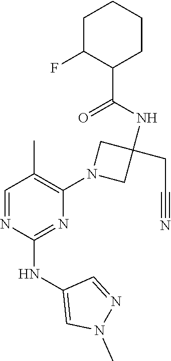 [SMILES: Cc1cnc(Nc2cnn(C)c2)nc1N1CC(CC#N)(NC(=O)C2CCCCC2F)C1] | E322 |
| 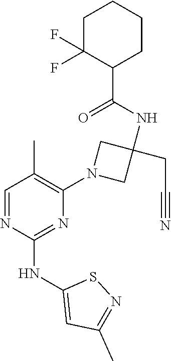 [SMILES: Cc1cc(Nc2ncc(C)c(N3CC(CC#N)(NC(=O)C4CCCCC4(F)F)C3)n2)sn1] | E323 |
| 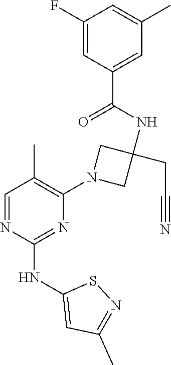 [SMILES: Cc1cc(F)cc(C(=O)NC2(CC#N)CN(c3nc(Nc4cc(C)ns4)ncc3C)C2)c1] | E324 |
| 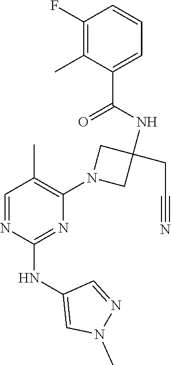 [SMILES: Cc1cnc(Nc2cnn(C)c2)nc1N1CC(CC#N)(NC(=O)c2cccc(F)c2C)C1] | E325 |
| 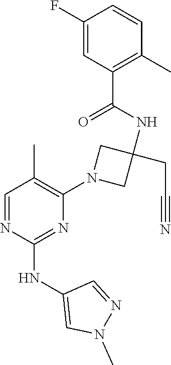 [SMILES: Cc1ccc(F)cc1C(=O)NC1(CC#N)CN(c2nc(Nc3cnn(C)c3)ncc2C)C1] | E326 |
| 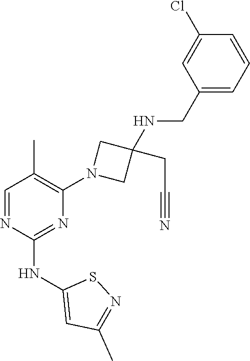 [SMILES: Cc1cc(Nc2ncc(C)c(N3CC(CC#N)(NCc4cccc(Cl)c4)C3)n2)sn1] | E327 |
| 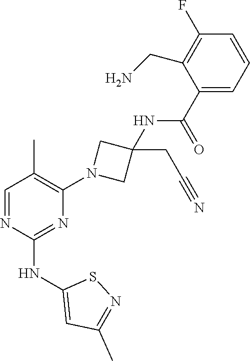 [SMILES: Cc1cc(Nc2ncc(C)c(N3CC(CC#N)(NC(=O)c4cccc(F)c4CN)C3)n2)sn1] | E328 |
| 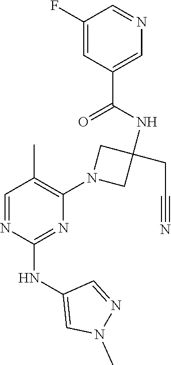 [SMILES: Cc1cnc(Nc2cnn(C)c2)nc1N1CC(CC#N)(NC(=O)c2cncc(F)c2)C1] | E329 |
| 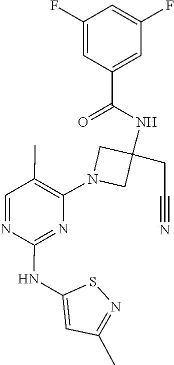 [SMILES: Cc1cc(Nc2ncc(C)c(N3CC(CC#N)(NC(=O)c4cc(F)cc(F)c4)C3)n2)sn1] | E330 |
| 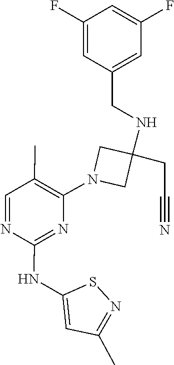 [SMILES: Cc1cc(Nc2ncc(C)c(N3CC(CC#N)(NCc4cc(F)cc(F)c4)C3)n2)sn1] | E331 |
| 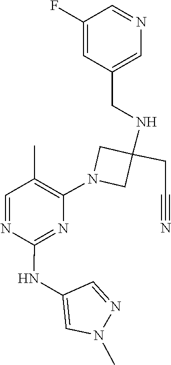 [SMILES: Cc1cnc(Nc2cnn(C)c2)nc1N1CC(CC#N)(NCc2cncc(F)c2)C1] | E332 |
| 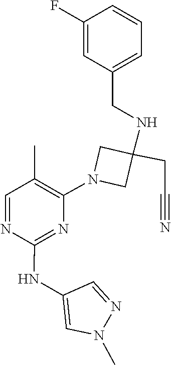 [SMILES: Cc1cnc(Nc2cnn(C)c2)nc1N1CC(CC#N)(NCc2cccc(F)c2)C1] | E333 |
| 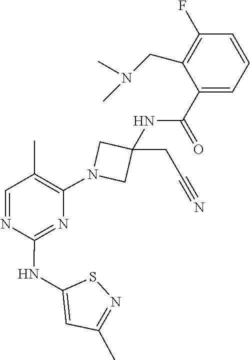 [SMILES: Cc1cc(Nc2ncc(C)c(N3CC(CC#N)(NC(=O)c4cccc(F)c4CN(C)C)C3)n2)sn1] | E334 |
| 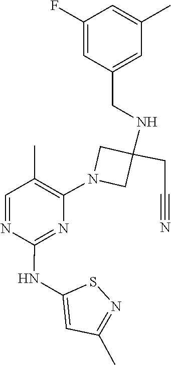 [SMILES: Cc1cc(F)cc(CNC2(CC#N)CN(c3nc(Nc4cc(C)ns4)ncc3C)C2)c1] | E335 |
| 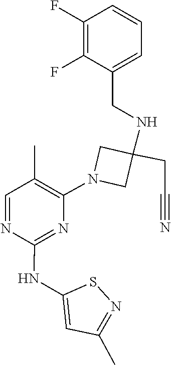 [SMILES: Cc1cc(Nc2ncc(C)c(N3CC(CC#N)(NCc4cccc(F)c4F)C3)n2)sn1] | E336 |
| 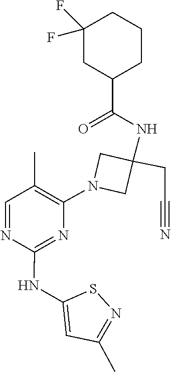 [SMILES: Cc1cc(Nc2ncc(C)c(N3CC(CC#N)(NC(=O)C4CCCC(F)(F)C4)C3)n2)sn1] | E337 |
| 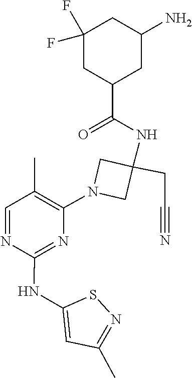 [SMILES: Cc1cc(Nc2ncc(C)c(N3CC(CC#N)(NC(=O)C4CC(N)CC(F)(F)C4)C3)n2)sn1] | E338 |
| 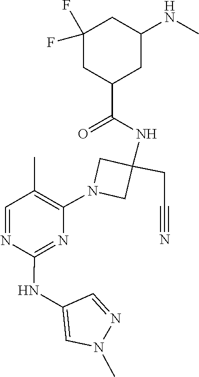 [SMILES: CNC1CC(C(=O)NC2(CC#N)CN(c3nc(Nc4cnn(C)c4)ncc3C)C2)CC(F)(F)C1] | E339 |
| | E340 |
| 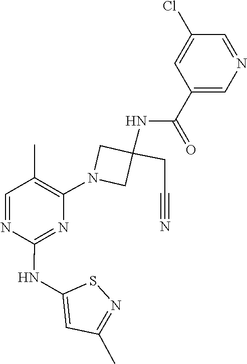 [SMILES: Cc1cc(Nc2ncc(C)c(N3CC(CC#N)(NC(=O)c4cncc(Cl)c4)C3)n2)sn1] | E341 |
| 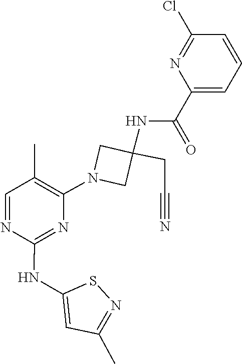 [SMILES: Cc1cc(Nc2ncc(C)c(N3CC(CC#N)(NC(=O)c4cccc(Cl)n4)C3)n2)sn1] | E342 |
| 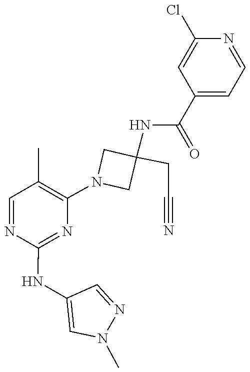 [SMILES: Cc1cnc(Nc2cnn(C)c2)nc1N1CC(CC#N)(NC(=O)c2ccnc(Cl)c2)C1] | E343 |
| 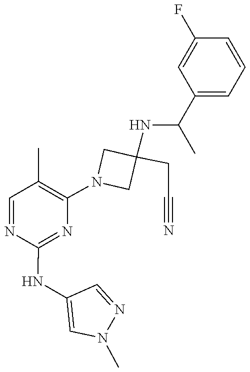 [SMILES: Cc1cnc(Nc2cnn(C)c2)nc1N1CC(CC#N)(NC(C)c2cccc(F)c2)C1] | E344 |
| 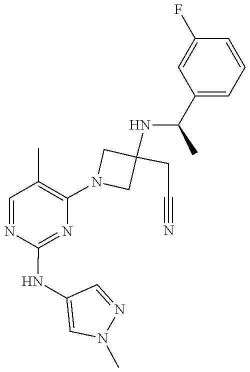 [SMILES: Cc1cnc(Nc2cnn(C)c2)nc1N1CC(CC#N)(N[C@H](C)c2cccc(F)c2)C1] | E345 |
| 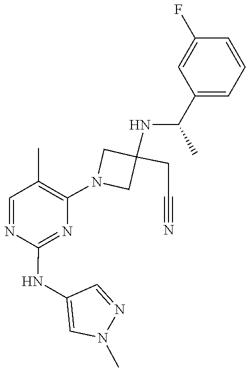 [SMILES: Cc1cnc(Nc2cnn(C)c2)nc1N1CC(CC#N)(N[C@@H](C)c2cccc(F)c2)C1] | E346 |
| 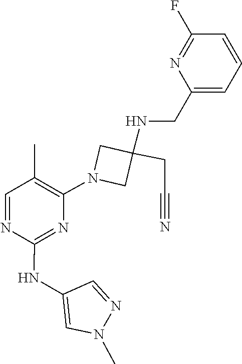 [SMILES: Cc1cnc(Nc2cnn(C)c2)nc1N1CC(CC#N)(NCc2cccc(F)n2)C1] | E347 |
| 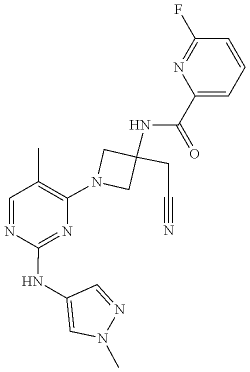 [SMILES: Cc1cnc(Nc2cnn(C)c2)nc1N1CC(CC#N)(NC(=O)c2cccc(F)n2)C1] | E348 |
| 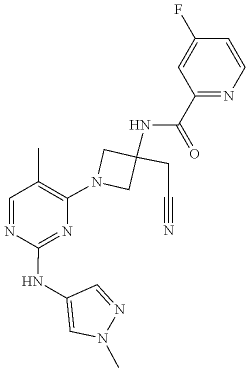 [SMILES: Cc1cnc(Nc2cnn(C)c2)nc1N1CC(CC#N)(NC(=O)c2cc(F)ccn2)C1] | E349 |

Additional compounds prepared according to the procedures described herein include compounds of Table 27, or pharmaceutically acceptable salts thereof.

TABLE 27

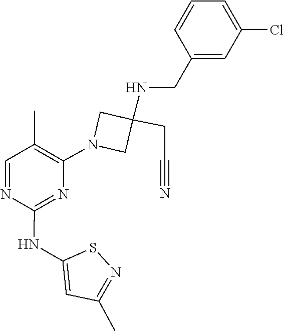 [SMILES: Cc1cc(Nc2ncc(C)c(N3CC(CC#N)(NCc4cccc(Cl)c4)C3)n2)sn1]

E350
<10 nM TYK2
<10 nM JAK1

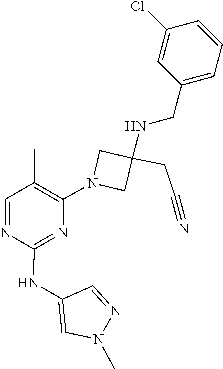 [SMILES: Cc1cnc(Nc2cnn(C)c2)nc1N1CC(CC#N)(NCc2cccc(Cl)c2)C1]

E351
<50 nM TYK2
<20 nM JAK1

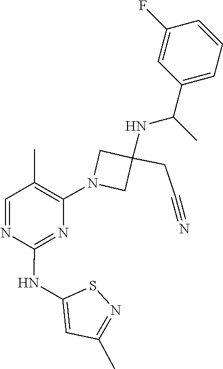 [SMILES: Cc1cc(Nc2ncc(C)c(N3CC(CC#N)(NC(C)c4cccc(F)c4)C3)n2)sn1]

E352
<50 nM TYK2
<100 nM JAK1

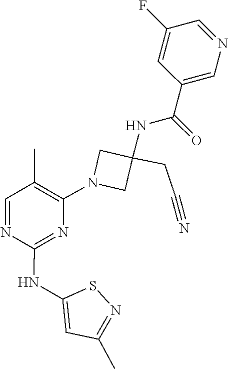 [SMILES: Cc1cc(Nc2ncc(C)c(N3CC(CC#N)(NC(=O)c4cncc(F)c4)C3)n2)sn1]

E353
<50 nM TYK2
<20 nM JAK1

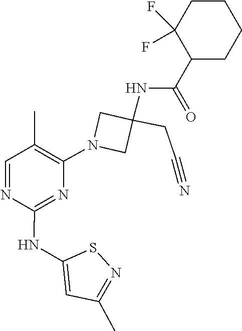 [SMILES: Cc1cc(Nc2ncc(C)c(N3CC(CC#N)(NC(=O)C4CCCCC4(F)F)C3)n2)sn1]

E354
<50 nM TYK2
<50 nM JAK1
E355
<10 nM TYK2
<10 nM JAK1

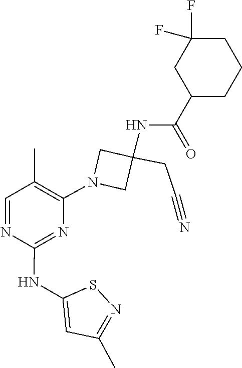 [SMILES: Cc1cc(Nc2ncc(C)c(N3CC(CC#N)(NC(=O)C4CCCC(F)(F)C4)C3)n2)sn1]

E356
<10 nM TYK2
<10 nM JAK1

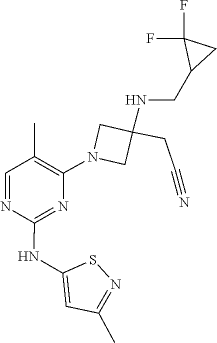 [SMILES: Cc1cc(Nc2ncc(C)c(N3CC(CC#N)(NCC4CC4(F)F)C3)n2)sn1]

E357
<10 nM TYK2
<20 nM JAK1

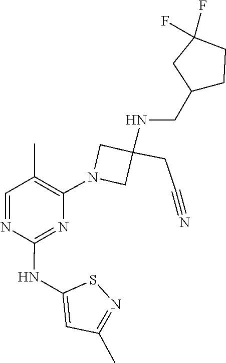 [SMILES: Cc1cc(Nc2ncc(C)c(N3CC(CC#N)(NCC4CCC(F)(F)C4)C3)n2)sn1]

E358
<10 nM TYK2
<10 nM JAK1

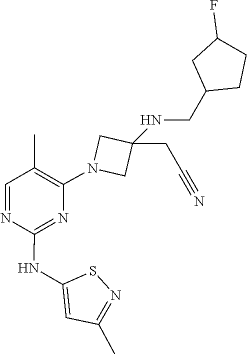 [SMILES: Cc1cc(Nc2ncc(C)c(N3CC(CC#N)(NCC4CCC(F)C4)C3)n2)sn1]

E359
<10 nM TYK2
<10 nM JAK1

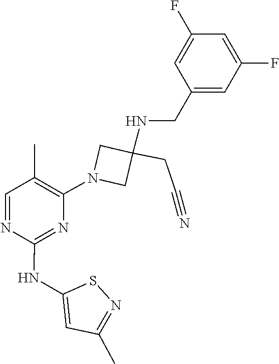 [SMILES: Cc1cc(Nc2ncc(C)c(N3CC(CC#N)(NCc4cc(F)cc(F)c4)C3)n2)sn1]

E360

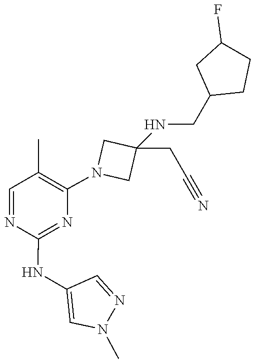 [SMILES: Cc1cnc(Nc2cnn(C)c2)nc1N1CC(CC#N)(NCC2CCC(F)C2)C1]

E361

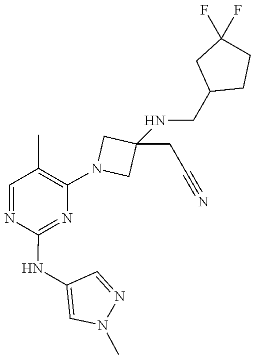 [SMILES: Cc1cnc(Nc2cnn(C)c2)nc1N1CC(CC#N)(NCC2CCC(F)(F)C2)C1]

E362

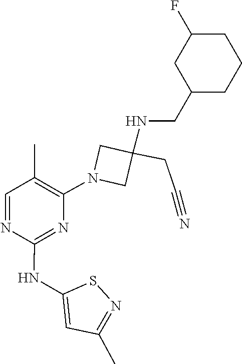 [SMILES: Cc1cc(Nc2ncc(C)c(N3CC(CC#N)(NCC4CCCC(F)C4)C3)n2)sn1]

E363

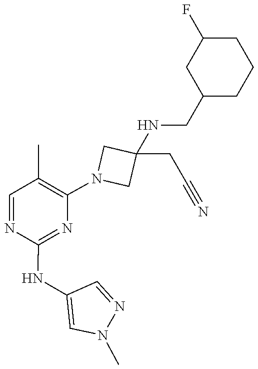 [SMILES: Cc1cnc(Nc2cnn(C)c2)nc1N1CC(CC#N)(NCC2CCCC(F)C2)C1]

E364

Additional compounds that may be prepared according to the procedures described herein include compounds of Table 28, or pharmaceutically acceptable salts thereof.

TABLE 28

| | |
|---|---|
| | E365 |
| | E366 |
| | E367 |
| | E368 |
| 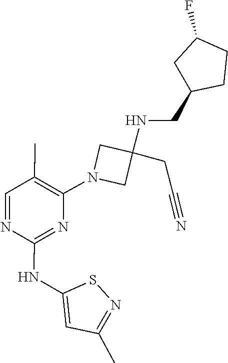 [SMILES: Cc1cc(Nc2ncc(C)c(N3CC(CC#N)(NC[C@@H]4CC[C@@H](F)C4)C3)n2)sn1] | E369 |
| 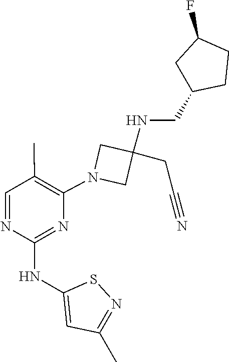 [SMILES: Cc1cc(Nc2ncc(C)c(N3CC(CC#N)(NC[C@H]4CC[C@H](F)C4)C3)n2)sn1] | E370 |
| 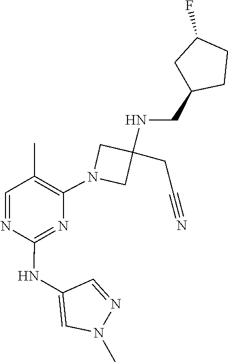 [SMILES: Cc1cnc(Nc2cnn(C)c2)nc1N1CC(CC#N)(NC[C@@H]2CC[C@@H](F)C2)C1] | E371 |
| 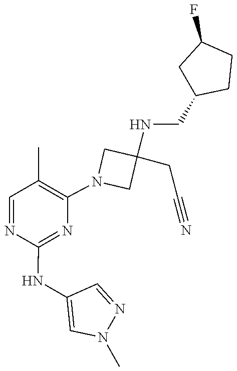 [SMILES: Cc1cnc(Nc2cnn(C)c2)nc1N1CC(CC#N)(NC[C@H]2CC[C@H](F)C2)C1] | E372 |
| 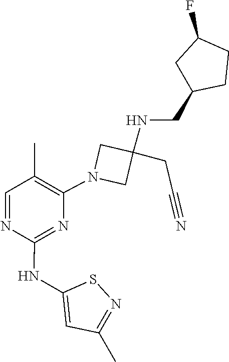 [SMILES: Cc1cc(Nc2ncc(C)c(N3CC(CC#N)(NC[C@@H]4CC[C@H](F)C4)C3)n2)sn1] | E373 |
| 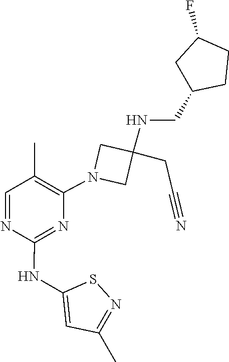 [SMILES: Cc1cc(Nc2ncc(C)c(N3CC(CC#N)(NC[C@H]4CC[C@@H](F)C4)C3)n2)sn1] | E374 |
| 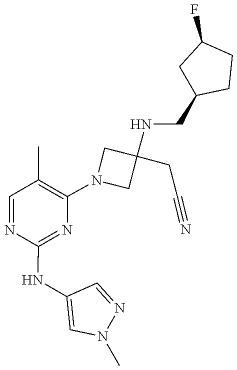 [SMILES: Cc1cnc(Nc2cnn(C)c2)nc1N1CC(CC#N)(NC[C@@H]2CC[C@H](F)C2)C1] | E375 |
| 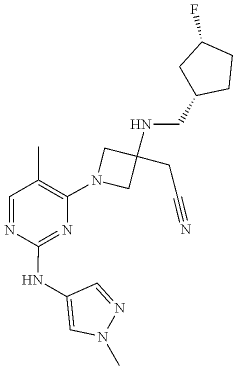 [SMILES: Cc1cnc(Nc2cnn(C)c2)nc1N1CC(CC#N)(NC[C@H]2CC[C@@H](F)C2)C1] | E376 |
| 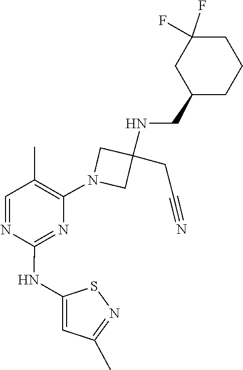 [SMILES: Cc1cc(Nc2ncc(C)c(N3CC(CC#N)(NC[C@@H]4CCCC(F)(F)C4)C3)n2)sn1] | E377 |
| 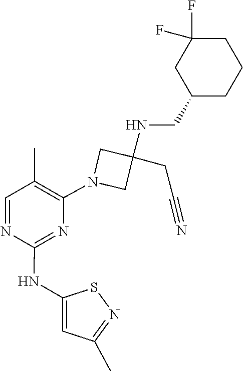 [SMILES: Cc1cc(Nc2ncc(C)c(N3CC(CC#N)(NC[C@H]4CCCC(F)(F)C4)C3)n2)sn1] | E378 |
| 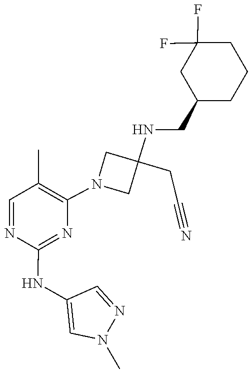 [SMILES: Cc1cnc(Nc2cnn(C)c2)nc1N1CC(CC#N)(NC[C@@H]2CCCC(F)(F)C2)C1] | E379 |
| 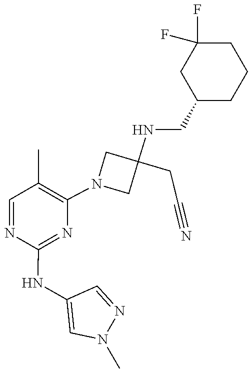 [SMILES: Cc1cnc(Nc2cnn(C)c2)nc1N1CC(CC#N)(NC[C@H]2CCCC(F)(F)C2)C1] | E380 |
| 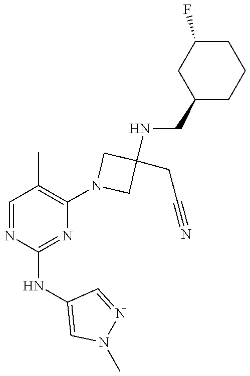 [SMILES: Cc1cnc(Nc2cnn(C)c2)nc1N1CC(CC#N)(NC[C@@H]2CCC[C@@H](F)C2)C1] | E381 |
| 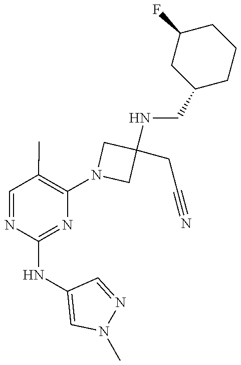 [SMILES: Cc1cnc(Nc2cnn(C)c2)nc1N1CC(CC#N)(NC[C@H]2CCC[C@H](F)C2)C1] | E382 |
| 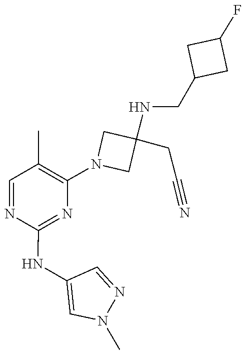 [SMILES: Cc1cnc(Nc2cnn(C)c2)nc1N1CC(CC#N)(NCC2CC(F)C2)C1] | E383 |
| 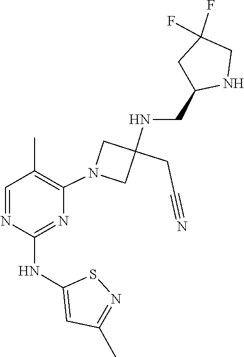 [SMILES: Cc1cc(Nc2ncc(C)c(N3CC(CC#N)(NC[C@H]4CC(F)(F)CN4)C3)n2)sn1] | E384 |
| 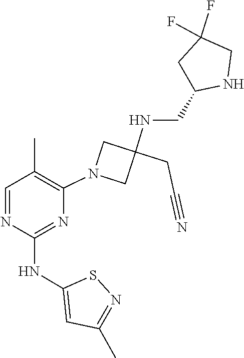 [SMILES: Cc1cc(Nc2ncc(C)c(N3CC(CC#N)(NC[C@@H]4CC(F)(F)CN4)C3)n2)sn1] | E385 |
| 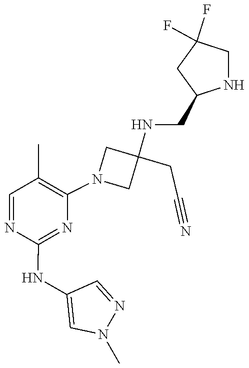 [SMILES: Cc1cnc(Nc2cnn(C)c2)nc1N1CC(CC#N)(NC[C@H]2CC(F)(F)CN2)C1] | E386 |
| 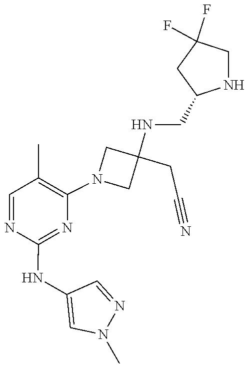 [SMILES: Cc1cnc(Nc2cnn(C)c2)nc1N1CC(CC#N)(NC[C@@H]2CC(F)(F)CN2)C1] | E387 |
| 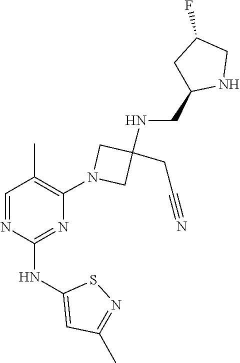 [SMILES: Cc1cc(Nc2ncc(C)c(N3CC(CC#N)(NC[C@H]4C[C@H](F)CN4)C3)n2)sn1] | E388 |
| 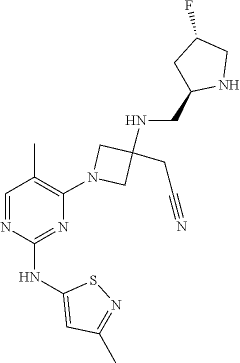 [SMILES: Cc1cc(Nc2ncc(C)c(N3CC(CC#N)(NC[C@H]4C[C@H](F)CN4)C3)n2)sn1] | E389 |
| 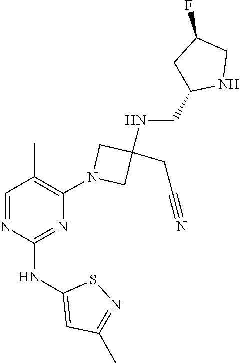 [SMILES: Cc1cc(Nc2ncc(C)c(N3CC(CC#N)(NC[C@@H]4C[C@@H](F)CN4)C3)n2)sn1] | E390 |
| 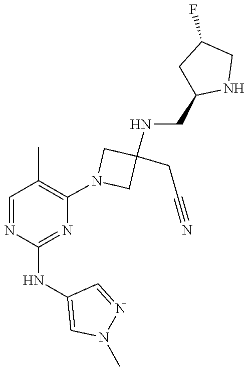 [SMILES: Cc1cnc(Nc2cnn(C)c2)nc1N1CC(CC#N)(NC[C@H]2C[C@H](F)CN2)C1] | E391 |
| 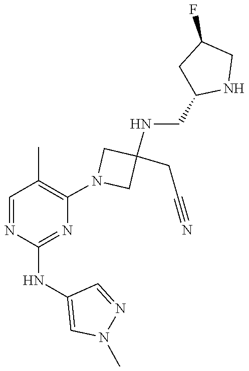 [SMILES: Cc1cnc(Nc2cnn(C)c2)nc1N1CC(CC#N)(NC[C@@H]2C[C@@H](F)CN2)C1] | E392 |
| 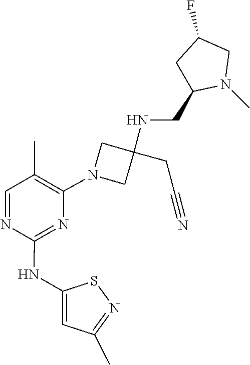 [SMILES: Cc1cc(Nc2ncc(C)c(N3CC(CC#N)(NC[C@H]4C[C@H](F)CN4C)C3)n2)sn1] | E393 |
| 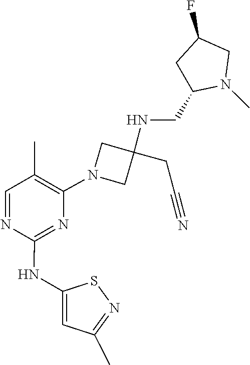 [SMILES: Cc1cc(Nc2ncc(C)c(N3CC(CC#N)(NC[C@@H]4C[C@@H](F)CN4C)C3)n2)sn1] | E394 |
| 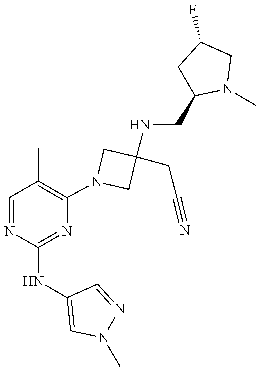 [SMILES: Cc1cnc(Nc2cnn(C)c2)nc1N1CC(CC#N)(NC[C@H]2C[C@H](F)CN2C)C1] | E395 |
| 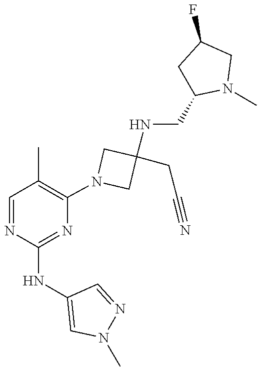 [SMILES: Cc1cnc(Nc2cnn(C)c2)nc1N1CC(CC#N)(NC[C@@H]2C[C@@H](F)CN2C)C1] | E396 |

Additional compounds that may be prepared according to the procedures described herein include compounds of Table 29, or pharmaceutically acceptable salts thereof.

TABLE 29

| | |
|---|---|
| 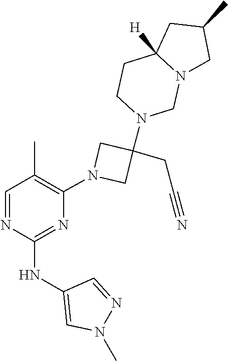 [SMILES: Cc1cnc(Nc2cnn(C)c2)nc1N1CC(CC#N)(N2CC[C@@H]3C[C@@H](C)CN3C2)C1] | E397 |
| 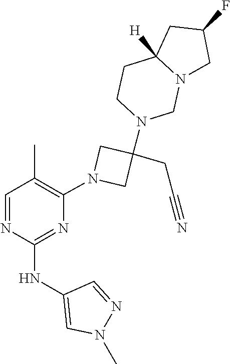 [SMILES: Cc1cnc(Nc2cnn(C)c2)nc1N1CC(CC#N)(N2CC[C@@H]3C[C@@H](F)CN3C2)C1] | E398 |
| 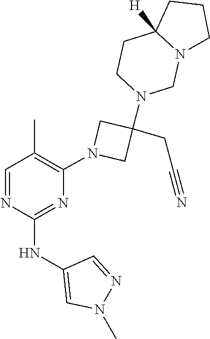 [SMILES: Cc1cnc(Nc2cnn(C)c2)nc1N1CC(CC#N)(N2CC[C@@H]3CCCN3C2)C1] | E399 |
| 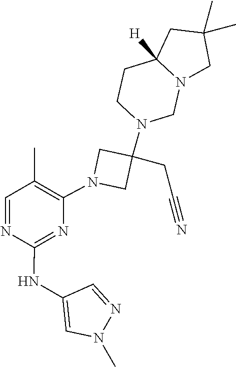 [SMILES: Cc1cnc(Nc2cnn(C)c2)nc1N1CC(CC#N)(N2CC[C@@H]3CC(C)(C)CN3C2)C1] | E400 |
| 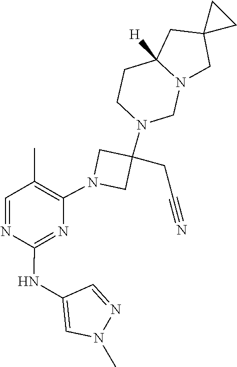 [SMILES: Cc1cnc(Nc2cnn(C)c2)nc1N1CC(CC#N)(N2CC[C@@H]3CC4(CC4)CN3C2)C1] | E401 |

### Isomers

Compounds described herein may exist in one or more particular geometric, optical, enantiomeric, diastereomeric, epimeric, atropic, stereoisomer, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

In one embodiment, a compound described herein may be an enantiomerically enriched isomer of a stereoisomer described herein. For example, the compound may have an enantiomeric excess of at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99%. Enantiomer, when used herein, refers to either of a pair of chemical compounds whose molecular structures have a mirror-image relationship to each other.

In one embodiment, a preparation of a compound disclosed herein is enriched for an isomer of the compound having a selected stereochemistry, e.g., R or S, corresponding to a selected stereocenter. For example, the compound has a purity corresponding to a compound having a selected stereochemistry of a selected stereocenter of at least about 60%, 65%, 70%, 75%, 80%, 85%, 90%, 95%, 96%, 97%, 98%, or 99%.

In one embodiment, a composition described herein includes a preparation of a compound disclosed herein that is enriched for a structure or structures having a selected stereochemistry, e.g., R or S, at a selected stereocenter. Exemplary R/S configurations can be those provided in an example described herein.

An “enriched preparation,” as used herein, is enriched for a selected stereoconfiguration of one, two, three or more selected stereocenters within the subject compound. Exemplary selected stereocenters and exemplary stereoconfigurations thereof can be selected from those provided herein, e.g., in an example described herein. By enriched is meant at least 60%, e.g., of the molecules of compound in the preparation have a selected stereochemistry of a selected stereocenter. In an embodiment it is at least 65%, 70%, 75%, 80%, 85%, 90%, 95%, 96%, 97%, 98%, or 99%. Enriched refers to the level of a subject molecule(s) and does not connote a process limitation unless specified.

Compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral chromatography column. The enantiomers also may be obtained from kinetic resolution of the racemate of corresponding esters using lipase enzymes.

Except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₃-alkyl or propyl includes n-propyl and iso-propyl; C₄-alkyl or butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hydroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Thus, Compounds described herein also include isotopically-labeled compounds wherein one or more atoms is replaced by an atom having the same atomic number, but an atomic mass or mass number different from the atomic mass or mass number predominantly found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In some embodiments, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

### Salts

A compound described herein can be in the form of a salt, e.g., a pharmaceutically acceptable salt. The term “pharmaceutically acceptable salt” includes salts of the active compounds that are prepared with relatively nontoxic acids or bases, depending on the particular substituents found on the compounds described herein. Neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure. Examples of pharmaceutically acceptable salts are discussed in Berge et al, 1977, “Pharmaceutically Acceptable Salts.” J. Pharm. Sci. Vol. 66, pp. 1-19. In an embodiment, the compound is present in mono-salt form. In embodiments, the compound is present in di-salt form.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations. Examples of suitable organic cations include, but are not limited to, ammonium ion (i.e., NH₄+) and substituted ammonium ions (e.g., NH₃R₁⁺, NH₂R₂⁺, NHR₃⁺, NR₄⁺). Examples of some suitable substituted ammonium ions are those derived from: ethylamine, diethylamine, triethylamine, butylamine, ethylenediamine, ethanolamine, diethanolamine, piperazine, benzylamine, phenylbenzylamine, choline, meglumine, and tromethamine, as well as dibasic amino acids, such as lysine and arginine.

If the compound is cationic or has a functional group that may be cationic (e.g., —NH₂ may be —NH₃⁺), then a salt may be formed with a suitable anion. Examples of suitable inorganic anions include, but are not limited to, those derived from the following inorganic acids: boric, hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, p-toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Chemically Protected Forms

It may be convenient or desirable to prepare, purify, and/or handle an active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999). Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

A hydroxyl group may be protected as an ether (—OR) or an ester (—OC(O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(O)CH₃, —OAc).

An aldehyde or ketone group may be protected as an acetal (RCH(OR)₂) or ketal (R₂C(OR)₂), respectively, in which the carbonyl group (R₂C=O) is converted to a diether (R₂C(OR)₂), by reaction with, for example, a primary alcohol. The aldehyde or ketone group is readily regenerated by hydrolysis using a large excess of water in the presence of acid.

An amine group may be protected, for example, as an amide (—NRC(O)R) or a urethane (—NRC(O)OR), for example, as: a methyl amide (—NHC(O)CH₃); a benzyloxy amide (—NHC(O)OCH₂C₆H₅, —NH-Cbz); as a tert-butoxy amide (—NHC(O)OC(CH₃)₃, —NH-Boc); a 2-biphenyl-2-propoxy amide (—NHCO(O)C(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases (e.g., cyclic amines), as a nitroxide radical (>N—O«).

A carboxylic acid group may be protected as an ester, for example, as: an alkyl ester (e.g., a methyl ester; a t-butyl ester); a haloalkyl ester (e.g., a haloalkyl ester); a trialkylsilylalkyl ester; or an arylalkyl ester (e.g., a benzyl ester; a nitrobenzyl ester); or as an amide, for example, as a methyl amide.

A thiol group may be protected as a thioether (—SR), for example, as: a benzyl thioether; an acetamidomethyl ether (—S—CH₂NHC(O)CH₃)

### Prodrugs and Other Modifications

In addition to salt forms, the present disclosure may also provide compounds that are in a prodrug form. Prodrugs of the compounds described herein are those compounds that readily undergo chemical changes under physiological conditions to provide the compounds described herein. Prodrugs can be converted to the compounds of the present disclosure by chemical or biochemical methods in an ex vivo environment. For example, prodrugs can be slowly converted to the compounds of the present disclosure when placed in a transdermal patch reservoir with or without a suitable enzyme or chemical reagent.

A compound described herein can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom substitution in aromatic rings.

### Methods of Use and Activity

The compounds as disclosed herein and compositions including them have kinase inhibitory activity and are thus useful in modulating the action of kinases, and in treatment and/or prevention of diseases or conditions influenced by kinases. The above compounds and compositions may be used to modulate (e.g., influence or inhibit) the action of kinases either in a cell in vitro or in a cell in a living body in vivo. Specifically, in one embodiment, a method is provided of modulating the action of a kinase comprising applying to a medium such as an assay medium or contacting with a cell either in a cell in vitro or in a cell in a living body in vivo an effective inhibitory amount of a compound as disclosed herein. In another embodiment, a method is provided of inhibiting the action of a kinase comprising applying to a medium such as an assay medium or contacting with a cell either in a cell in vitro or in a cell in a living body in vivo an effective inhibitory amount of a compound as disclosed herein. In one embodiment the kinase inhibited is a JAK (e.g., JAK2) kinase.

JAK inhibitors are useful in treating various JAK-associated diseases or conditions. Examples of JAK-associated diseases include diseases and conditions involving the immune system including, for example, organ transplant rejection (e.g., allograft rejection and graft versus host disease). Further examples of JAK-associated diseases or conditions include autoimmune diseases such as alopecia areata, alopecia universalis, polycythemia vera, vitiligo, multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, myocarditis, autoimmune thyroid conditions, chronic obstructive pulmonary disease (COPD), and the like. In some embodiments, the autoimmune disease is arthritis.

Further examples of JAK-associated diseases or conditions include allergic conditions such as asthma, food allergies, eczematous dermatitis, contact dermatitis, atopic dermatitis (atropic eczema), and rhinitis. Further examples of JAK-associated diseases or conditions include viral diseases such as Epstein Barr Virus (EBV), Hepatitis B, Hepatitis C, HIV, HTLV 1, Varicella-Zoster Virus (VZV) and Human Papilloma Virus (HPV).

Further examples of JAK-associated diseases or conditions include those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides. Other examples of JAK-associated diseases or conditions include pulmonary arterial hypertension.

Other examples of JAK-associated diseases or conditions include inflammation-associated cancers. In some embodiments, the cancer is associated with inflammatory bowel disease. In some embodiments, the inflammatory bowel disease is ulcerative colitis. In some embodiments, the inflammatory bowel disease is Crohn's disease. In some embodiments, the inflammation-associated cancer is colitis-associated cancer. In some embodiments, the inflammation-associated cancer is colon cancer or colorectal cancer. In some embodiments, the cancer is gastric cancer, gastrointestinal carcinoid tumor, gastrointestinal stromal tumor (GIST), adenocarcinoma, small intestine cancer, hematological cancers, or rectal cancer.

The compounds of the present disclosure have applications in methods of inhibiting kinases in a cell, a tissue, or a subject such as a human comprising contacting the cell with an amount of one or more of the compounds of the present disclosure effective to modulate, and in particular inhibit the activity of the kinase. In one embodiment, the compounds are administered in a pharmaceutically acceptable composition, such as in or with a pharmaceutically acceptable carrier.

In another embodiment, the compounds of the present disclosure are used in methods for modulating the action of a kinase in a cell comprising contacting the cell with amount of one or more compounds of the present disclosure effective to modulate the action of a kinase in a cell. In one embodiment, the compounds of the present disclosure are administered in a pharmaceutically acceptable composition, such as in or with a pharmaceutically acceptable carrier.

Treatment or prevention of ocular diseases or conditions for which the compounds of the present disclosure may be useful includes any of the diseases or conditions associated with kinase activity or diseases or conditions affected by kinases. Examples of these types of diseases or conditions include inflammatory diseases, autoimmune diseases, ocular inflammatory conditions such as non-infectious uveitis, chorioretinitis, iritis, sterile conjunctivitis, keratitis, episcleritis, dry eye diseases, meibomian gland dysfunction, allergic conjunctivitis, MGD, injury-related ocular inflammation or dry eye syndrome, Primary and Secondary Sjögren's syndrome, redness, blepharitis, keratoconjunctivitis sicca, ocular hyperemia, macular degeneration (wet and dry), Diabetic retinopathy, DME, RVO, Posterior uveitis, retinal inflammation, inflammation due to gene therapy vectors (e.g. viral vectors), graft versus host disease, Thygeson superficial punctate keratitis (TSPK), or a combination of thereof, neurodegenerative diseases or conditions such as Alzheimer's; ocular diseases, such as diabetic eye diseases, wet age-related macular degeneration, or dry age-related macular degeneration, inflammatory eye diseases, retinal degradation, and glaucoma; cardiovascular diseases; and cancer. Additional examples include bone condition, obesity, hepatic disease, renal disease, pancreatitis, gastric disturbance, hypertension, fertility control, conditions of hair growth, nasal congestion, neurogenic bladder condition, gastrointestinal condition, dermatological condition, and respiratory indications.

In some embodiments, the compounds of the present disclosure will be administered in conjunction with one or more additional therapeutic agents. Suitable classes of additional therapeutic agents include, but are not limited to, corticosteroids, other JAK inhibitors, IKK inhibitors, ROCK inhibitors, beta blockers, alpha-agonists, carbonic anhydrase inhibitors, prostaglandin-like compounds, miotic or cholinergic agents, epinephrine compounds, or neuroprotective or other anti-inflammatory compounds. The administration of a compound of the present disclosure in conjunction with one or more additional therapeutic agents can be concomitant administration, as a mixture in a single pharmaceutical composition, or a compound of the present disclosure and one or more additional therapeutic agents can be chemically conjugated directly to each other or through a linker such that upon exposure to physiological conditions, the conjugation of the two therapeutic agents is cleaved.

Beta blockers. These compounds are thought to lower intraocular pressure (IOP) by reducing the production of aqueous humor. Examples include levobunolol (BETAGAN™), timolol (BETIMOL™, TIMOPTIC™), betaxolol (BETOPTIC™) and metipranolol (OPTIPRANOLOL™).

Alpha-agonists. These compounds are thought to lower IOP by reducing the production of aqueous humor and increasing drainage. Examples include apraclonidine (IOPIDINE™) and brimonidine (ALPHAGAN™).

Carbonic anhydrase inhibitors. These compounds are thought to lower IOP by also reducing the production of aqueous humor. Examples include dorzolamide (TRUSOPT™) and brinzolamide (AZOPT™).

Prostaglandin-like compounds. These compounds are thought to lower IOP principally by increasing the outflow of aqueous humor by the uveoscleral pathway. Examples include AR-102, latanoprost (XALATAN™), bimatoprost (LUMIGAN™), tafluprost (ZIOPTAN™), and travoprost (TRAVATAN™).

Miotic or cholinergic agents. These agents are thought to function by causing the pupil to constrict. Examples include pilocarpine (ISOPTO CARPINE™, PILOPINE™) and carbachol (ISOPTO CARBACHOL™).

Epinephrine and nor-epinephrin compounds. These compounds, such as dipivefrin (PROPINE™), are thought to function by both decreasing the outflow of aqueous humor, as well as increasing fluid drainage.

Neuroprotective or anti-inflammatory compounds. These compounds, such as Aflibercept (EYLEA™) are treatments for conditions of the retina such as Macular Degeneration and are designed as anti-VEGF treatments or have similar types of anti-growth or anti-inflammatory activity.

Corticosteroids. These compounds, such as dexamethasone, are thought to function by decreasing inflammatory mediators via nuclear transcription.

Thus, provided herein are methods of treating an ocular condition in a subject in need thereof, comprising administering to the subject a therapeutically effective amount of a compound, a composition, or a pharmaceutical composition provided herein.

Also provided herein are methods of reducing ocular inflammation in a subject in need thereof, comprising administering to the subject a therapeutically effective amount of compound, a composition, or a pharmaceutical composition provided herein.

In one aspect, provided herein are methods of treating an ocular condition in a subject in need thereof, comprising administering to the subject a therapeutically effective amount of a compound, or a pharmaceutically acceptable salt thereof, provided herein.

In some embodiments, the ocular disease or condition is dry eye.

In some embodiments, the ocular disease or condition is meibomian gland dysfunction (MGD).

In some embodiments, the ocular disease or condition is uveitis.

In some embodiments, the ocular disease or condition is blepharitis.

In another aspect, provided herein are methods of reducing inflammation in a subject in need thereof, comprising administering to the subject a therapeutically effective amount of a compound, or a pharmaceutically acceptable salt thereof, provided herein.

In some embodiments of these aspects, a compound or pharmaceutical composition is administered topically to an eye of the subject.

In some embodiments, provided herein are methods of treating an ocular disease or condition in a subject in need thereof, comprising administering to the subject a therapeutically effective amount of a compound of one of the Formulae provided herein, or a pharmaceutically acceptable salt thereof.

In some embodiments, provided herein are methods of treating an ocular disease or condition in a subject in need thereof, comprising administering to the subject a therapeutically effective amount of a compound provided in any one of the Tables herein, or a pharmaceutically acceptable salt thereof.

In some embodiments, provided herein are methods of reducing the signs or symptoms of dry eye disease (DED) or meibomian gland dysfunction (MGD) in a subject in need thereof, comprising administering to the subject a therapeutically effective amount of a compound of one of the Formulae provided herein, or a pharmaceutically acceptable salt thereof.

In some embodiments, provided herein are methods of reducing intraocular pressure in a subject in need thereof, comprising administering to the subject a therapeutically effective amount of a compound provided in the various Tables, or a pharmaceutically acceptable salt thereof.

Some embodiments of a method of the present disclosure further comprise administering a therapeutically effective amount of one or more additional therapeutic agents. In some embodiments, the one or more additional therapeutic agents is a beta blocker, an alpha-agonist, a carbonic anhydrase inhibitor, a prostaglandin, or a prostaglandin-like compound, a miotic or cholinergic agent, an epinephrine compound, or a neuroprotective or anti-inflammatory compound. In some embodiments, the one or more additional therapeutic agents is a prostaglandin or a prostaglandin-like compound. In some embodiment, the prostaglandin-like compound is AR-102, latanoprost, bimatoprost, tafluprost, or travoprost. In another embodiment, the additional therapeutic agents are other JAK inhibitors or corticosteroids. As explained above, the administration of a compound of the present disclosure can be concomitantly, as a mixture in a single pharmaceutical composition, or chemically conjugated directly to each other or through a linker.

Also provided herein are methods of treating an autoimmune disease or condition in a subject in need thereof, comprising administering to the subject a therapeutically effective amount of compound, a composition, or a pharmaceutical composition provided herein.

In some embodiments, provided herein are methods of treating an autoimmune disease or condition in a subject in need thereof, comprising administering to the subject a therapeutically effective amount of a compound of one of the Formulae provided herein, or a pharmaceutically acceptable salt thereof.

In some embodiments, provided herein are methods of treating an autoimmune disease or condition in a subject in need thereof, comprising administering to the subject a therapeutically effective amount of a compound provided in any one of the Tables herein, or a pharmaceutically acceptable salt thereof.

In some embodiments, the autoimmune disease or condition is multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, myocarditis, autoimmune thyroid conditions, or chronic obstructive pulmonary disease.

### Compositions and Administration

The additional therapeutic agent or agents can be administered simultaneously or sequentially with the compounds of the present disclosure. Sequential administration includes administration before or after the compounds of the present disclosure. In some embodiments, the additional therapeutic agent or agents can be administered in the same composition as the compounds of the present disclosure. In other embodiments, there can be an interval of time between administration of the additional therapeutic agent and the compounds of the present disclosure.

In some embodiments, the administration of an additional therapeutic agent with a compound of the present disclosure will enable lower doses of the other therapeutic agents to be administered for a longer period of time.

Also provided herein are compositions comprising a compound provided herein, or a pharmaceutically acceptable salt thereof. In one embodiment, the compositions provided herein are pharmaceutical compositions comprising a pharmaceutically acceptable carrier.

Pharmaceutical compositions for use in accordance with the present disclosure may be formulated in a conventional manner using one or more physiologically acceptable carriers or excipients. Thus, the compounds and their physiologically acceptable salts and solvates may be formulated for administration by, for example, solid dosing, eyedrop, in a topical oil-based formulation, injection (including injection of a drug-eluting device either into the body as a whole, or into specific tissues of the eye), inhalation (either through the mouth or the nose), implants, or oral, buccal, parenteral or rectal administration. Techniques and formulations may generally be found in “Remington's Pharmaceutical Sciences,” (Meade Publishing Co., Easton, PA).

The route by which the compounds of the present disclosure (component A) will be administered, and the form of the composition will dictate the type of carrier (component B) to be used. The composition may be in a variety of forms, suitable, for example, for systemic administration (e.g., oral, rectal, nasal, sublingual, buccal, implants, or parenteral), or by ocular injection into one of the chambers of the eye, such as subconjunctival, subretinal, retrobulbar, intravitreal injection, intracameral injection, or injection into the aqueous humour, or by topical administration e.g., local application on the skin including the eyelid, or into the eye, using solutions, suspensions, nanosuspensions, ocular liposomal delivery systems, or iontophoresis.

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, combinations thereof, and others. All carriers are optional in the systemic compositions.

Ingredient a) is a diluent. Suitable diluents for solid dosage forms include sugars such as glucose, lactose, dextrose, and sucrose; diols such as propylene glycol; calcium carbonate; sodium carbonate; sugar alcohols, such as glycerin; mannitol; and sorbitol. The amount of ingredient a) in the systemic or topical composition is typically about 50 to about 90%.

Ingredient b) is a lubricant. Suitable lubricants for solid dosage forms are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma. The amount of ingredient b) in the systemic or topical composition is typically about 5 to about 10%.

Ingredient c) is a binder. Suitable binders for solid dosage forms include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of ingredient c) in the systemic composition is typically about 5 to about 50%, and in ocular solid dosing forms up to 99%.

Ingredient d) is a disintegrant. Suitable disintegrants for solid dosage forms include agar, alginic acid and the sodium salt thereof, effervescent mixtures, croscarmelose, crospovidone, sodium carboxymethyl starch, sodium starch glycolate, clays, and ion exchange resins. The amount of ingredient d) in the systemic or topical composition is typically about 0.1 to about 10%.

Ingredient e) for solid dosage forms is a colorant such as an FD&C dye. When used, the amount of ingredient e) in the systemic or topical composition is typically about 0.005 to about 0.1%.

Ingredient f) for solid dosage forms is a flavor such as menthol, peppermint, and fruit flavors. The amount of ingredient f), when used, in the systemic or topical composition is typically about 0.1 to about 1.0%.

Ingredient g) for solid dosage forms is a sweetener such as aspartame and saccharin. The amount of ingredient g) in the systemic or topical composition is typically about 0.001 to about 1%.

Ingredient h) is an antioxidant such as butylated hydroxyanisole (“BHA”), butylated hydroxytoluene (“BHT”), and vitamin E. The amount of ingredient h) in the systemic or topical composition is typically about 0.1 to about 5%.

Ingredient j) is a preservative such as benzalkonium chloride, methyl paraben and sodium benzoate. The amount of ingredient j) in the systemic or topical composition is typically about 0.01 to about 5%.

Ingredient k) for solid dosage forms is a glidant such as silicon dioxide. The amount of ingredient k) in the systemic or topical composition is typically about 1 to about 5%.

Ingredient m) is a solvent, such as water, isotonic saline, ethyl oleate, glycerine, hydroxylated castor oils, alcohols such as ethanol, and phosphate buffer solutions. The amount of ingredient m) in the systemic or topical composition is typically from about 0 to about 100%.

Ingredient n) is a suspending agent. Suitable suspending agents include Avicel® RC-591 (from FMC Corporation of Philadelphia, PA) and sodium alginate. The amount of ingredient n) in the systemic or topical composition is typically about 1 to about 8%.

Ingredient o) is a surfactant or a permeation enhancer, such as lecithin, Polysorbate 80, and sodium lauryl sulfate, and the TWEENS® from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of ingredient o) in the systemic or topical composition is typically about 0.1% to about 5%.

Although the amounts of components A and B in the systemic compositions will vary depending on the type of systemic composition prepared, the specific derivative selected for component A and the ingredients of component B, in general, system compositions comprise 0.01% to 50% of component A and 50 to 99.99% of component B.

Compositions for parenteral administration typically comprise A) 0.1 to 10% of the compounds of the present disclosure and B) 90 to 99.9% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and effective amount, usually at least about 5%, and more particularly from about 25% to about 50% of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A, and component B a carrier comprising ingredients selected from the group consisting of a) diluents, b) lubricants, c) binders, d) disintegrants, e) colorants, f) flavors, g) sweeteners, k) glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain g) sweeteners such as aspartame and saccharin, or f) flavors such as menthol, peppermint, fruit flavors, or a combination thereof.

Capsules (including implants, time release and sustained release formulations) typically comprise component A, and a carrier comprising one or more a) diluents disclosed above in a capsule comprising gelatin. Granules typically comprise component A, and preferably further comprise k) glidants such as silicon dioxide to improve flow characteristics. Implants can be of the biodegradable or the non-biodegradable type. Implants may be prepared using any known biocompatible formulation.

The selection of ingredients in the carrier for oral compositions depends on secondary considerations like taste, cost, and shelf stability, which are not critical for the purposes of this disclosure. One skilled in the art would know how to select appropriate ingredients without undue experimentation.

The solid compositions may also be coated by conventional methods, typically with pH or time-dependent coatings, such that component A is released in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise component A and component B, namely, a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject compounds include injection, sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants.

In one embodiment of the disclosure, the compounds of the present disclosure are topically administered. Topical compositions that can be applied locally to the eye may be in any form known in the art, non-limiting Examples of which include solubilized into an eyedrop, a suspension or nanosuspension, an emulsion or nanoemulsion, dosed as a solid, gelable drops, sprays, ointments, or a sustained or non-sustained release unit placed in the conjunctival cul-du-sac of the eye or another appropriate location.

Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions comprise: component A, the compounds described above, and component B, a carrier. The carrier of the topical composition preferably aids penetration of the compounds into the eye. Component B may further comprise one or more optional components.

An effective amount of a compound according to the present disclosure will vary with the particular condition being treated, the age and physical condition of the patient being treated, the severity of the condition, the duration of treatment, the nature of concurrent therapy, the route of administration, the particular pharmaceutically-acceptable carrier utilized, and like factors within the knowledge and expertise of the attending physician. For example, an effective amount of the compounds of the present disclosure for systemic administration is from about 0.01 to about 1000 μg/kg body weight, preferably from about 0.1 to about 100 μg/kg per body weight, most preferably form about 1 to about 50 μg/kg body weight per day. The transdermal dosages will be designed to attain similar serum or plasma levels, based upon techniques known to those skilled in the art of pharmacokinetics and transdermal formulations. Plasma levels for systemic administration are expected to be in the range of 0.01 to 100 ng/mL, more preferably from 0.05 to 50 ng/mL and most preferably from 0.1 to 10 ng/mL. While these dosages are based upon a daily administration rate, the compounds of the present disclosure may also be administered at other intervals, such as twice per day, twice weekly, once weekly, or once a month. One of ordinary skill in the art would be able to calculate suitable effective amounts for other intervals of administration.

The compounds of the present disclosure are useful in a method of reducing or decreasing ocular inflammation. The compounds of the present disclosure may be administered to a subject in need of treatment in an amount effective to reduce irritation or inflammation. Thus, these compounds are useful in the treatment of meibomian gland dysfunction (MGD). The preferred route of administration for treating inflammation is topically.

The exact amounts of each component in the topical composition depend on various factors. The amount of component A added to the topical composition is dependent on the IC₅₀ of component A, typically expressed in nanomolar (nM) units. For example, if the IC₅₀ of the medicament is 1 nM, the amount of component A will be from about 0.001 to about 0.3%. If the IC₅₀ of the medicament is 10 nM, the amount of component A) will be from about 0.01 to about 1%. If the IC₅₀ of the medicament is 100 nM, the amount of component A will be from about 0.1 to about 10%. If the amount of component A is outside the ranges specified above (i.e., lower), efficacy of the treatment may be reduced. One skilled in the art understands how to calculate and understand an IC₅₀. The remainder of the composition, up to 100%, is component B.

The amount of the carrier employed in conjunction with component A is sufficient to provide a practical quantity of composition for administration per unit dose of the medicament. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

Component B may comprise a single ingredient or a combination of two or more ingredients. In the topical compositions, component B comprises a topical carrier. Suitable topical carriers comprise one or more ingredients selected from the group consisting of phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols and symmetrical alcohols.

The carrier of the topical composition may further comprise one or more ingredients selected from the group consisting of q) emollients, r) propellants, s) solvents, t) humectants, u) thickeners, v) powders, w) fragrances, x) pigments, and y) preservatives.

Ingredient q) is an emollient. The amount of ingredient q) in a skin-based topical composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane.

Ingredient r) is a propellant. The amount of ingredient r) in the topical composition is typically about 0 to about 95%. Suitable propellants include propane, butane, isobutane, dimethyl ether, carbon dioxide, nitrous oxide, and combinations thereof.

Ingredient s) is a solvent. The amount of ingredient s) in the topical composition is typically about 0 to about 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols.

Ingredient t) is a humectant. The amount of ingredient t) in the topical composition is typically 0 to 95%. Suitable humectants include glycerin, sorbitol, sodium 2-pyrrolidone-5-carboxylate, soluble collagen, dibutyl phthalate, gelatin, and combinations thereof. Specific humectants include glycerin.

Ingredient u) is a thickener. The amount of ingredient u) in the topical composition is typically about 0 to about 95%.

Ingredient v) is a powder. The amount of ingredient v) in the topical composition is typically 0 to 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. For ocular applications, specific powders include beta-cyclodextrins, such as betadex sulfobutyl ether sodium, hydroxypropyl cyclodextrins, such as (2-hydroxypropyl)-beta-cyclodextrin and sodium polyacrylate. For gel dosing ocular formulations, sodium polyacrylate may be used. The powders may serve as permeation enhancers or solubilizing or stabilizing agents.

Ingredient w) is a fragrance. The amount of ingredient w) in the topical composition is typically about 0 to about 1.5%, particularly, about 0.001 to about 0.1%. For ocular applications a fragrance is not typically used.

Ingredient x) is a pigment. Suitable pigments for skin applications include inorganic pigments, organic lake pigments, pearlescent pigments, and mixtures thereof. Inorganic pigments useful in this disclosure include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The organic pigments and lakes useful in this disclosure include those selected from the group consisting of D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430), the dye or lakes based on Cochineal Carmine (CI 75,570) and mixtures thereof.

The pearlescent pigments useful in this disclosure include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof. The amount of pigment in the topical composition is typically about 0 to about 10%. For ocular applications a pigment is generally not used.

In a particularly preferred embodiment of the disclosure, topical pharmaceutical compositions for ocular administration are prepared typically comprising component A and B (a carrier), such as purified water, and one or more ingredients selected from the group consisting of y) sugars or sugar alcohols such as dextrans, particularly mannitol and dextran 70, z) cellulose or a derivative thereof, aa) a salt, bb) disodium EDTA (Edetate disodium), and cc) a pH adjusting additive.

Examples of z) cellulose derivatives suitable for use in the topical pharmaceutical composition for ocular administration include sodium carboxymethylcellulose, ethylcellulose, methylcellulose, and hydroxypropyl-methylcellulose, particularly, hydroxypropyl-methylcellulose.

Examples of aa) salts suitable for use in the topical pharmaceutical composition for ocular administration include borate salts, mono-, di- and trisodium phosphate, sodium chloride, potassium chloride, and combinations thereof.

Examples of cc) pH adjusting additives include HCl or NaOH in amounts sufficient to adjust the pH of the topical pharmaceutical composition for ocular administration to the range of 4.5-7.5 pH units.

Component A may be included in kits comprising a compound as described herein, a systemic or topical composition described above, or both; and information, instructions, or both that use of the kit will provide treatment for cosmetic and medical conditions in mammals (particularly humans). The information and instructions may be in the form of words, pictures, or both, and the like. In addition, or in the alternative, the kit may comprise the medicament, a composition, or both; and information, instructions, or both, regarding methods of application of medicament, or of composition, preferably with the benefit of treating or preventing cosmetic and medical conditions in mammals (e.g., humans).

The disclosure will be further explained by the following illustrative Examples that are to be considered non-limiting.

### EXAMPLES

### Example 1: ROCK and JAK Assays

### ROCK Kinase Inhibition Assays.

All compounds were initially prepared as 10 mM stocks in anhydrous dimethylsulfoxide (DMSO). A 20 μL aliquot of the 10 mM solutions was transferred to individual wells in column 1 of a 96-well polypropylene microtiter plate (Corning #3363) and diluted with DMSO to give a final compound concentration of 4 mM. Test compounds were then serially diluted 1:5 in DMSO for an 11-point concentration response and further diluted in the assay buffer bringing all compound concentrations to a final range of 100 μM to 10 μM in 2.5% DMSO. The assay was performed in white 96-well, flat-bottom, half-area, non-binding assay plate (Corning #3642) in assay buffer consisting of 20 mM HEPES (pH 7.5), 10 mM MgCl₂*6H₂O, 100 μM sodium orthovanadate, 0.05% CHAPS and 0.1% bovine serum albumin. A 10 μL aliquot of compound from each well of the intermediate dilution plate and 20 μL of a 2× substrate/enzyme solution containing acceptor substrate (800 nM RSK2 peptide KRRRLSSLRA (SEQ ID NO: 1)), ROCK2 enzyme (10 nM), or ROCK1 enzyme, and 1,4-Dithiothreitol (DTT, 2 uM) were added to all wells. The reaction was initiated by the addition of 10 μL of 4× stock solution ATP (2 μM). Reactions were thoroughly mixed manually, covered, and allowed to incubate at room temperature for 75 min. Protein kinase activity was quantitated using Promega's KINASE-GLO™ luminescent Kinase Assay Kit according to the manufacturer's directions. ATP concentrations remaining in Test wells following the termination of the enzymatic reaction were compared against control wells containing equivalent amounts of DMSO containing no inhibitor (CTRL). ATP concentrations in both Test wells and CTRL wells were normalized against background (BKG) ATP concentrations in wells containing concentrations of inhibitor that completely inhibited the protein kinase under investigation (i.e. a concentration that prevented any consumption of ATP over the course of the incubation). Percent of Control (POC) values were determined for each concentration of compound tested according to the equation:

$$POC = ( ( Test well value - BKG ) / ( CTRL - BKG ) ) * 100$$

IC₅₀ values were calculated using the following 4-parameter logistic curve-fitting algorithm:

$$f ( x ) = ( A + ( ( B - A ) / ( 1 + ( ( x / C ) \hat{} D ) ) ) )$$

IC₅₀ values were converted to K; values using the following Cheng-Prusoff Equation:

$${K}_{i} = {IC}_{50} / ( 1 + ( [ ATP ] / Km ATP ] ) ) .$$

### JAK Kinase Assays.

Compounds were prepared in the exact same manner as described in the ROCK Kinase Assay with the exception to the substrate and enzyme. The JAK 2× substrate/enzyme solution consisted of acceptor substrate (800 nM Abl peptide EAIYAAPFAKKK (SEQ ID NO:2)), JAK1, TYK2, JAK2 or JAK3 enzyme (10 nM) and DTT (2 uM). All other steps and solutions remain identical to the ROCK Kinase Assay above. Results are shown above throughout the tables.

### Example 2. PTM-HTM Assay

Porcine Trabecular Meshwork cells (PTM) were isolated from freshly obtained enucleated porcine eyes. Immortalized Human Trabecular Meshwork cells (TM-1) were obtained through a kind gift from Donna Peters in the Department of Ophthalmology and Visual Sciences at the University of Wisconsin. Cells were plated onto fibronectin coated glass-bottom 96-well plates and allowed to attach overnight. Media was removed and replaced with test compound in media with 1% fetal bovine serum and incubated for various times. After incubation, cells were formaldehyde fixed, Triton solubilized, and stained. PTM cells were stained with Alexa Fluor® 488 phalloidin (F-actin) and Hoechst 33342 (nuclei). TM-1 cells were stained with anti-paxillin followed by Alexa Fluor® 488 goat-anti-mouse IgG (focal adhesions) and Hoechst 33342 (nuclei). All staining reagents were obtained through Invitrogen. Images were collected on an INCeII 2200 imager with a 20× objective. The actin fiber length and total area of focal adhesions were analyzed using custom algorithms developed in the INCeII Developer Toolbox, v 1.9.3. Data collected were converted to percent of control (untreated cells). Curves were fit to data in GraphPad Prizm using sigmoidal dose-response and constraining top and bottom to 100% and 0%, respectively.

### Example 3

Topical ocular pharmaceutical compositions for treating inflammation are prepared by conventional methods and may be formulated as shown in Table 29.

TABLE 29

| Ingredient | Amount (wt %) |
|---|---|
| azetidine | 0.50 |
| Dextran 70 | 0.1 |
| Hydroxypropyl methylcellulose | 0.3 |
| Sodium Chloride | 0.77 |
| Potassium chloride | 0.12 |
| Disodium EDTA | 0.05 |
| Benzalkonium chloride | 0.01 |
| HCl and/or NaOH | pH 4.5-6.5 |
| Purified water | q.s. to 100% |

A compound provided herein is used as the azetidine. When the composition is topically administered to the eyes once daily, the above composition decreases ocular inflammation in a subject suffering from MGD or DED.

### Example 4

### Reference Example One. Pharmacological Activity for Glaucoma Assay

Pharmacological activity for glaucoma can also be demonstrated using assays designed to test the ability of the subject compounds to decrease intraocular pressure. Examples of such assays are described in the following reference, incorporated herein by reference: C. Liljebris, G. Selen, B. Resul, J. Stjernschantz, and U. Hacksell, “Derivatives of 17-phenyl-18, 19, 20-trinorprostaglandin F_{2α} Isopropyl Ester: Potential Anti-glaucoma Agents”, Journal of Medicinal Chemistry 1995, 38 (2): 289-304.

While the disclosure has been described in detail and with reference to specific embodiments thereof, it will be apparent to one skilled in the art that various changes and modifications can be made without departing from the spirit and scope of the disclosure.

Groupings of alternative elements or embodiments disclosed herein may be referred to and claimed individually or in any combination with other members of the group or other elements found herein.

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

Those skilled in the art will recognize, or be able to ascertain, using no more than routine experimentation, many equivalents of the specific embodiments of the subject matter described herein. Such equivalents are intended to be encompassed by the following claims.

1. A compound having a formula: 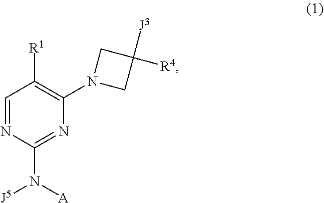
or a pharmaceutically acceptable salt thereof;
wherein
J⁵ is H or S(O)₂—(C₁₋₆ alkyl);
R¹ is H, CN, halogen, C₁₋₆ alkyl, O—(C₁₋₆ alkyl), or C₃₋₇ cycloalkyl;
A is C₆₋₁₆ aryl or C₂₋₁₅ heterocyclyl, each of which may be substituted with 1 or 2 groups selected, independently, from OH, halogen, C₁₋₆ alkyl, (C₁₋₆ alkylene)-OH, O—(C₁₋₆ alkyl), (C₁₋₆ alkylene)-O—(C₁₋₆ alkyl), S(O)₂—(C₁₋₆ alkyl), C₃₋₇ cycloalkyl, (C₁₋₁₆ alkylene)-C(O)OH, (C₁₋₁₆ alkylene)-C(O)N(H)—(C₁₋₆ alkyl), (C₁₋₁₆ alkylene)-C(O)N(H)—OH, or (C₁₋₆ alkylene)-N(C₁₋₆ alkyl)-(C₁₋₆ alkyl);
J³ is CN, OH, NH₂, C₁₋₆ alkyl, C₁₋₆ alkynyl, C₃₋₇ cycloalkyl, (C₁₋₃ alkylene)-CN, (C₁₋₃ alkylene)-NH₂, (C₁₋₃ alkylene)-N(H)C(O)—(C₃₋₇ cycloalkyl), (C₁₋₃ alkylene)-N(H)C(O)—(C₂₋₈ heterocycloalkyl), O—(C₃₋₇ cycloalkyl), O—(C₁₋₃ alkylene)-CN, N(H)C(O)—(C₃₋₇ cycloalkyl), N(H)C(O)—(C₂₋₈ heterocycloalkyl), N(H)C(O)—(C₂₋₅ heteroaryl), N(H)C(O)—(C₁₋₃ alkylene)-(C₂₋₅ heteroaryl), N(H)C(O)—(C₆₋₁₀ aryl), N(H)C(O)—(C₁₋₃ alkylene)-(C₆₋₁₀ aryl), N(H)C(O)NH₂, N(H)(C₁₋₆ alkyl), N(H)(C₃₋₇ cycloalkyl), N(C₁₋₆ alkyl)(C₃₋₇ cycloalkyl), N(H)(C₁₋₃ alkylene)-(C₂₋₈ heterocycloalkyl), N(H)(C₁₋₃ alkylene)-(C₂₋₅ heteroaryl), N(H)(C₁₋₃ alkylene)-(C₆₋₁₀ aryl), or C₂₋₁₅ heterocyclyl, each of which may be substituted with 1, 2, 3, or 4 groups selected, independently, from halogen, O, OH, O—(C₁₋₆ alkyl), S(O)₂—(C₁₋₆ alkyl), S(O)₂—(C₁₋₆ haloalkyl), C(NH₂)(NH), C₁₋₆ alkyl, C₁₋₆ haloalkyl, (C₁₋₆ alkylene)-OH, (C₁₋₆ alkylene)-O—(C₁₋₆ alkyl), (C₁₋₆ alkylene)-O—(C₁₋₆ haloalkyl), (C₁₋₆ alkylene)-S(O)₂—(C₁₋₆ alkyl), (C₁₋₆ alkylene)-NH₂, (C₁₋₆ alkylene)-N(H)(C₁₋₆ alkyl), (C₁₋₆ alkylene)-N(C₁₋₆ alkyl)(C₁₋₆ alkyl), (C₁₋₆ alkylene)-(C₃₋₇ cycloalkyl), (C₁₋₆ alkylene)-(C₃₋₇ halocycloalkyl), (C₁₋₆ alkylene)-(C₂₋₅ heteroaryl), (C₁₋₆ alkylene)-(C₆₋₁₀ haloaryl), NH₂, N(C₁₋₆ alkyl)(C₁₋₆ alkyl), N(H)—(C₁₋₃ alkylene)-(C₃₋₇ cycloalkyl), N(C₁₋₆ alkyl)-(C₁₋₃ alkylene)-(C₃₋₇ cycloalkyl), N(H)C(O)O—(C₁₋₆ alkyl), N(H)S(O)₂—(C₁₋₆ alkyl), C(O)—(C₁₋₆ alkyl), C(O)—(C₁₋₆ alkylene)-CN, C(O)—(C₁₋₆ alkylene)-NH₂, C(O)—(C₁₋₆ alkylene)-N(H)(C₁₋₆ alkyl), C(O)—(C₁₋₆ alkylene)-N(C₁₋₆ alkyl)(C₁₋₆ alkyl), C(O)—(C₁₋₆ alkylene)-N(H)[S(O)₂—C₁₋₆ alkyl], C(O)—(C₁₋₆ alkylene)-N(C₁₋₆ alkyl)[S(O)₂—C₁₋₆ alkyl], or C(O)O—(C₁₋₆ alkyl); and
R⁴ is H, OH, C₁₋₆ alkyl, (C₁₋₆ alkylene)-OH, C₁₋₆ haloalkyl, C₃₋₇ cycloalkyl, C₃₋₇ halocycloalkyl, (C₁₋₃ alkylene)-(C₃₋₇ cycloalkyl), (C₁₋₆ alkylene)CN, (C₁₋₆ alkylene)-C(O)O—(C₁₋₆ alkyl), (C₁₋₆ alkylene)-OC(O)—(C₁₋₆ alkyl), or C₂₋₈ heterocycloalkyl;
or J³ and R⁴, together with the atoms to which they are attached, combine to form C₃-7 cycloalkyl, C₂₋₈ heterocycloalkyl, CC(H)—(C₀₋₆ alkylene)CN, C₃₋₇ cycloalkyl substituted with 1 or 2 groups selected, independently, from halogen or (C₁₋₆ alkylene)CN, or C₂₋₈ heterocycloalkyl substituted with 1 or 2 groups selected, independently, from halogen or (C₁₋₆ alkylene)CN.

2. The compound of claim 1, having a formula: 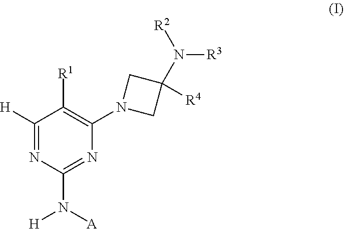
or a pharmaceutically acceptable salt thereof,
wherein
R¹ is H, F, Cl, Br, I, CN, NH₂, OH, OCF₃, OCH₂CF₃, C₁₋₆ alkyl, C₃₋₆ cycloalkyl, or C₁₋₆ haloalkyl,
R² and R³ are C₁₋₆ alkyl, C₁₋₆ heteroalkyl or C₁₋₆ haloalkyl, or R² and R³ combine to form a heteroalkyl or polycyclic ring of at least 4 member atoms and at most 12 member atoms,
R⁴ is C₁₋₆ alkyl, C₁₋₆ heteroalkyl or C₁₋₆ haloalkyl, —CH₂CN, —CH₂CH₂CN, —CH₂CH₂CH₂CN, alkylenecycloalkyl, alkylenecyclopropyl, or hydroxyl, and
A is phenyl, or a monocyclic C₃₋₅ heteroaryl having 1-3 heteroatoms, wherein each heteroatom is, independently, N, O, or S.

3. The compound of claim 2, wherein A is a monocyclic C₃₋₅ heteroaryl having 1-3 heteroatoms, wherein each heteroatom is, independently, N, O, or S.

4. A compound of having a formula selected from: 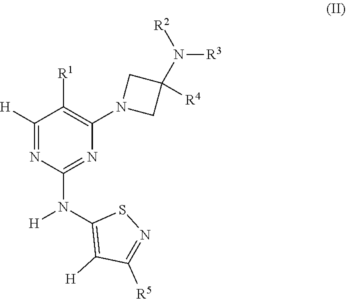
or a pharmaceutically acceptable salt thereof,
wherein
R² and R³ are C₁₋₆ alkyl, C₁₋₆ heteroalkyl or C₁₋₆ haloalkyl, or R² and R³ combine to form a heteroalkyl or polycyclic ring of at least 4 member atoms and at most 10 member atoms, and
R⁵ is C₁₋₁₂ alkyl, C₁₋₁₂ heteroalkyl or C₁₋₁₂ haloalkyl, CH₂OH, CH₂CH₂OH, CH₂CH₂CH₂OH, alkylenecycloalkyl, alkylenecyclopropyl, or methoxy; or 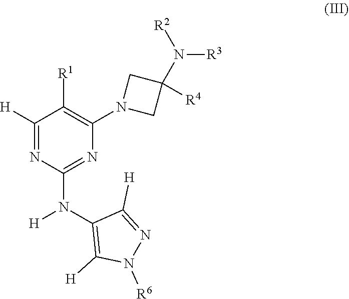
or a pharmaceutically acceptable salt thereof,
wherein
R² and R³ are C₁₋₆ alkyl, C₁₋₆ heteroalkyl or C₁₋₆ haloalkyl, or R² and R³ combine to form a heteroalkyl or polycyclic ring of at least 4 member atoms and at most 8 member atoms,
R⁶ is C₁₋₁₂ alkyl, C₁₋₁₂ heteroalkyl or C₁₋₁₂ haloalkyl, —CH₂OH, —CH₂CH₂OH, —CH₂CH₂CH₂OH, —(CH₂)n-COOR¹⁰ alkylenecycloalkyl, alkylenecyclopropyl, or methoxy,
n is an integer between one and ten, and
R¹⁰ is hydrogen, alkyl, cycloalkyl, heteroalkyl; or 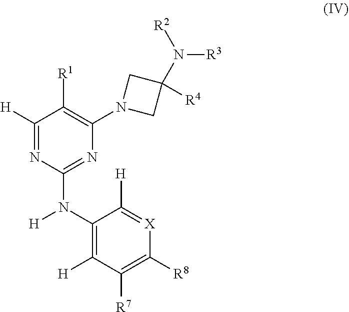
or a pharmaceutically acceptable salt thereof,
wherein
R² and R³ are C₁₋₆ alkyl, C₁₋₆ heteroalkyl or C₁₋₆ haloalkyl, or R² and R³ combine to form a heteroalkyl ring of at least 4 member atoms and at most 8 member atoms,
X is CH or N, and
R⁷ and R⁸ are independently H, C₁₋₁₂ alkyl, C₁₋₁₂ heteroalkyl, or C₁₋₁₂ haloalkyl, CH₂OH, CH₂CH₂OH, CH₂CH₂CH₂OH, alkylenecycloalkyl, alkylenecyclopropyl, or methoxy, or R⁷ and R⁸ combine to form a heteroalkyl ring of at least 4 member atoms and at most 8 member atoms.

5-6. (canceled)

7. The compound of claim 4, wherein the compound is of the formula: 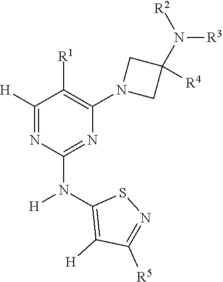
or a pharmaceutically acceptable salt thereof,
wherein
R¹ is H, Cl, C₁₋₆ alkyl, C₃₋₆ cycloalkyl, or C₁₋₃ haloalkyl,
R² and R³ are C₁₋₆ alkyl, C₁₋₆ heteroalkyl or C₁₋₆ haloalkyl, or R² and R³ combine to form a heteroalkyl ring of at least 4 member atoms and at most 8 member atoms, and
R⁵ is C₁₋₆ alkyl, C₃₋₅ cycloalkyl, CH₂OH, CH₂CH₂OH, CH₂CH₂CH₂OH, alkylenecycloalkyl, alkylenecyclopropyl, or methoxy.

8. The compound of claim 4, wherein the compound is of the formula: 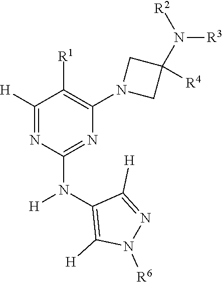
or a pharmaceutically acceptable salt thereof,
wherein
R¹ is H, F, Cl, OCF₃, OCH₂CF₃, C₁₋₆ alkyl, C₃₋₆ cycloalkyl, or C₁₋₆ haloalkyl,
R² and R³ are C₁₋₆ alkyl, C₁₋₆ heteroalkyl or C₁₋₆ haloalkyl, or R² and R³ combine to form a heteroalkyl ring of at least 4 member atoms and at most 8 member atoms,
R⁶ is C₁₋₆ alkyl, C₁₋₆ heteroalkyl or C₁₋₆ haloalkyl, —CH₂OH, —CH₂CH₂OH, —CH₂CH₂CH₂OH, —(CH₂)n-COOR¹⁰, alkylenecyclopropyl, or methoxy,
n is an integer between one and ten, and
R¹⁰ is hydrogen, alkyl, cycloalkyl, heteroalkyl.

9. The compound of claim 1, which is selected from: 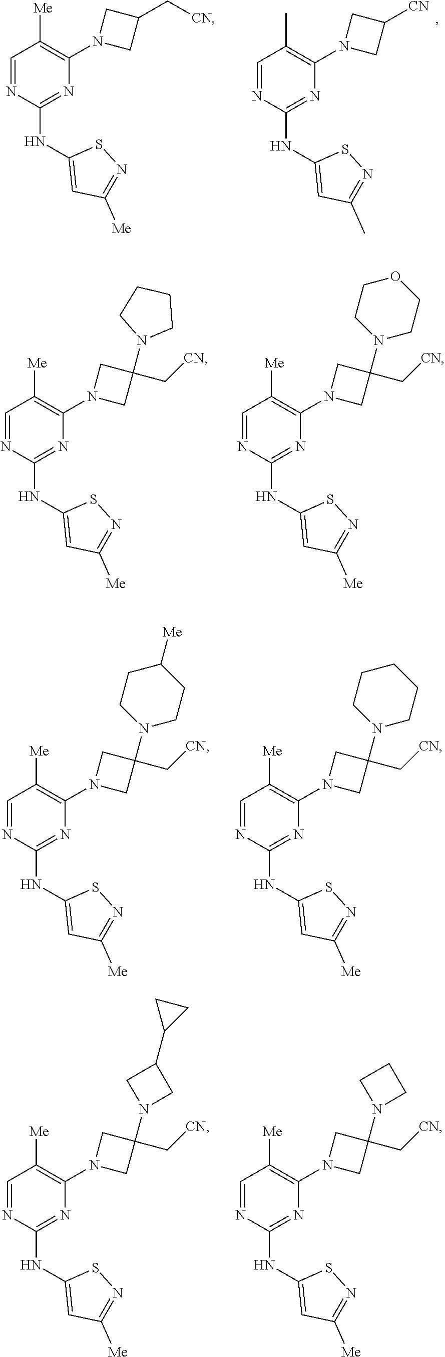 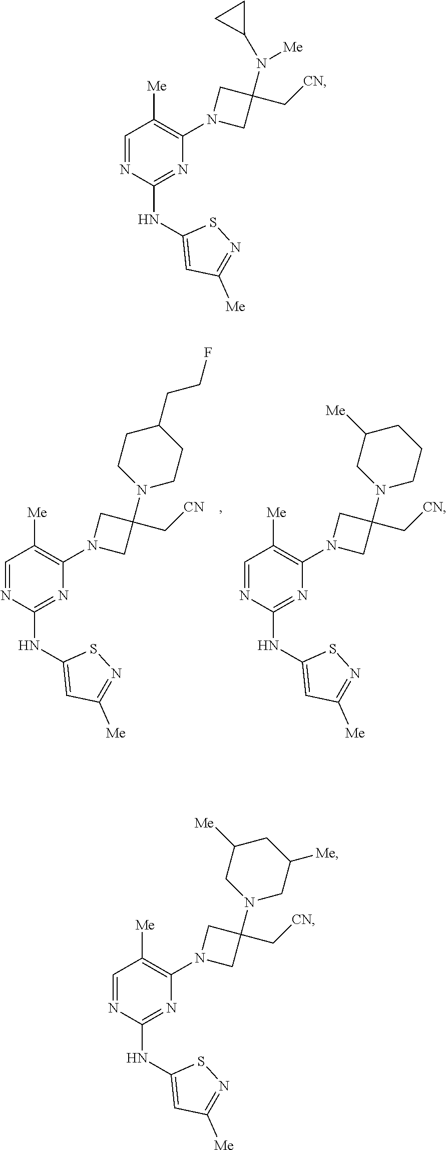 [SMILES: Cc1cc(Nc2ncc(C)c(N3CC(CC#N)(N(C)C4CC4)C3)n2)sn1 ; Cc1cc(Nc2ncc(C)c(N3CC(CC#N)(N4CCC(CCF)CC4)C3)n2)sn1 ; Cc1cc(Nc2ncc(C)c(N3CC(CC#N)(N4CCCC(C)C4)C3)n2)sn1 ; Cc1cc(Nc2ncc(C)c(N3CC(CC#N)(N4CC(C)CC(C)C4)C3)n2)sn1] 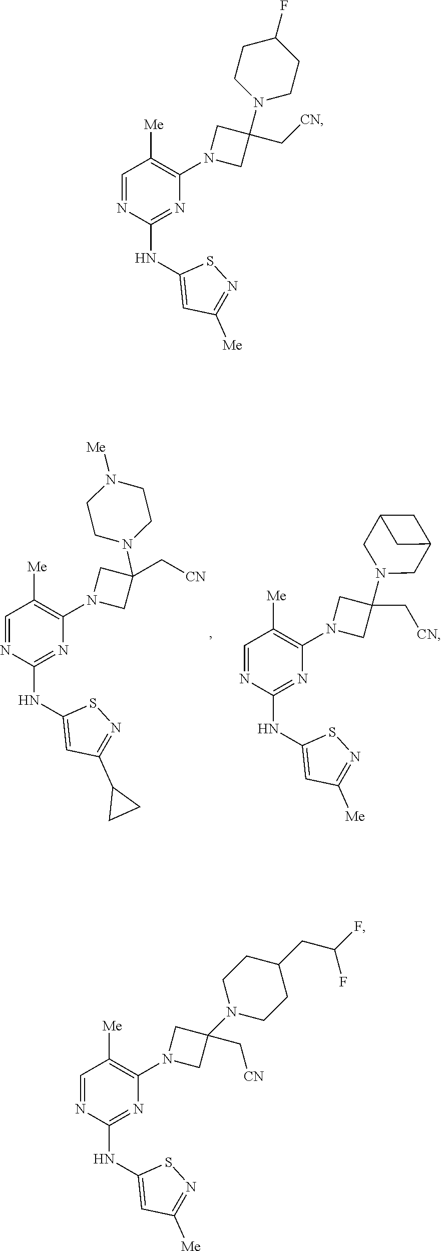 [SMILES: Cc1cc(Nc2ncc(C)c(N3CC(CC#N)(N4CCC(F)CC4)C3)n2)sn1 ; Cc1cnc(Nc2cc(C3CC3)ns2)nc1N1CC(CC#N)(N2CCN(C)CC2)C1 ; Cc1cc(Nc2ncc(C)c(N3CC(CC#N)(N4CC5CC(C5)C4)C3)n2)sn1 ; Cc1cc(Nc2ncc(C)c(N3CC(CC#N)(N4CCC(CC(F)F)CC4)C3)n2)sn1] 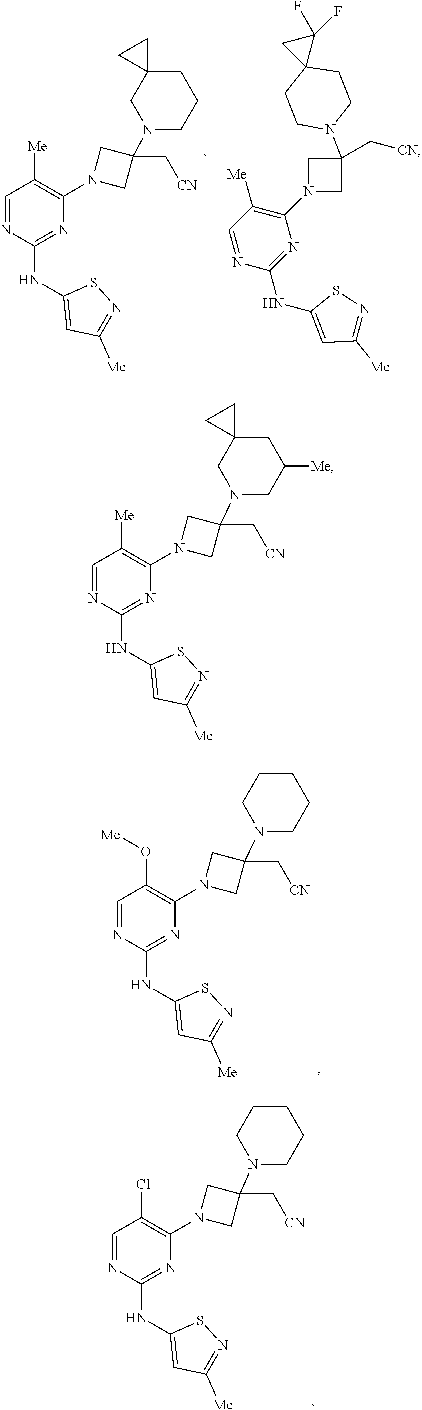 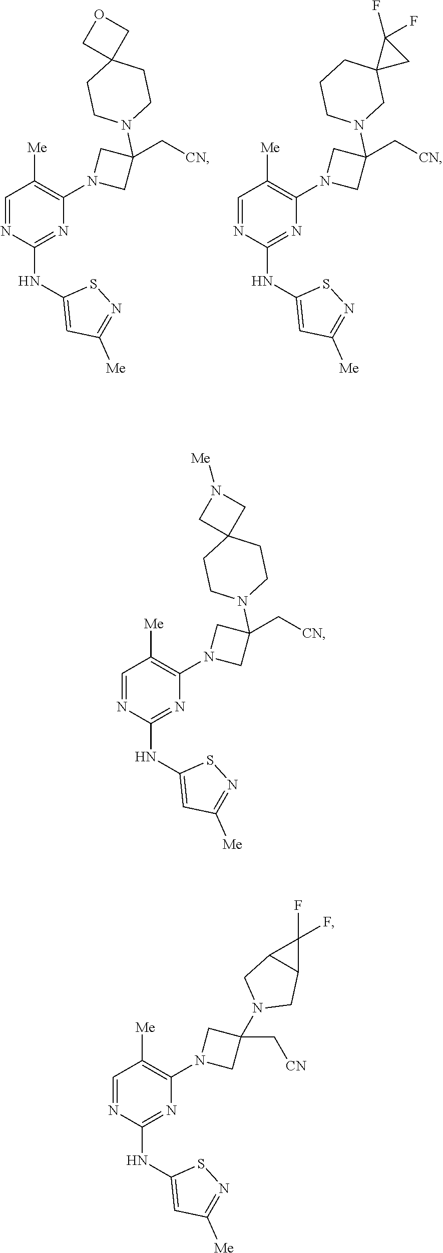 [SMILES: Cc1cc(Nc2ncc(C)c(N3CC(CC#N)(N4CCC5(CC4)COC5)C3)n2)sn1 ; Cc1cc(Nc2ncc(C)c(N3CC(CC#N)(N4CCCC5(C4)CC5(F)F)C3)n2)sn1 ; Cc1cc(Nc2ncc(C)c(N3CC(CC#N)(N4CCC5(CC4)CN(C)C5)C3)n2)sn1 ; Cc1cc(Nc2ncc(C)c(N3CC(CC#N)(N4CC5C(C4)C5(F)F)C3)n2)sn1] 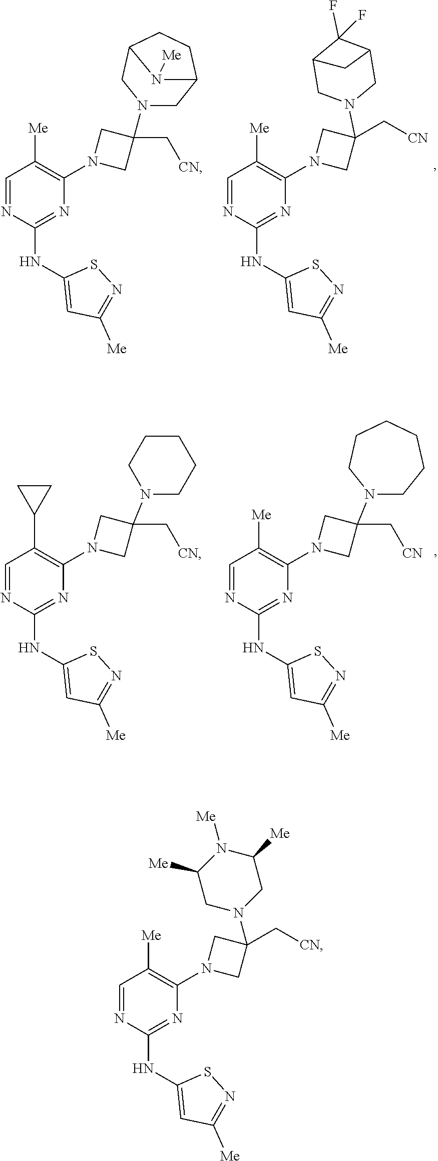 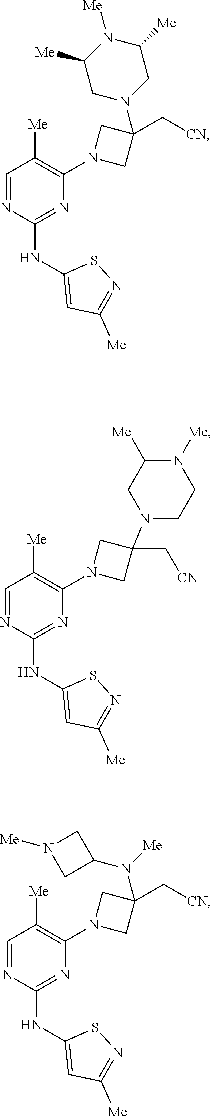 [SMILES: Cc1cc(Nc2ncc(C)c(N3CC(CC#N)(N4C[C@@H](C)N(C)[C@H](C)C4)C3)n2)sn1 ; Cc1cc(Nc2ncc(C)c(N3CC(CC#N)(N4CCN(C)C(C)C4)C3)n2)sn1 ; Cc1cc(Nc2ncc(C)c(N3CC(CC#N)(N(C)C4CN(C)C4)C3)n2)sn1] 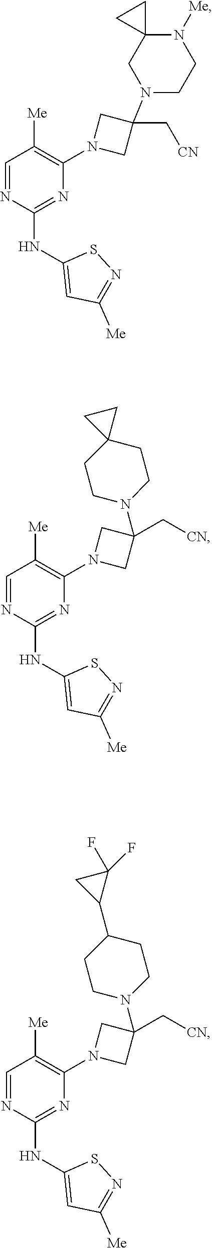 [SMILES: Cc1cc(Nc2ncc(C)c(N3CC(CC#N)(N4CCN(C)C5(CC5)C4)C3)n2)sn1 ; Cc1cc(Nc2ncc(C)c(N3CC(CC#N)(N4CCC5(CC4)CC5)C3)n2)sn1 ; Cc1cc(Nc2ncc(C)c(N3CC(CC#N)(N4CCC(C5CC5(F)F)CC4)C3)n2)sn1] 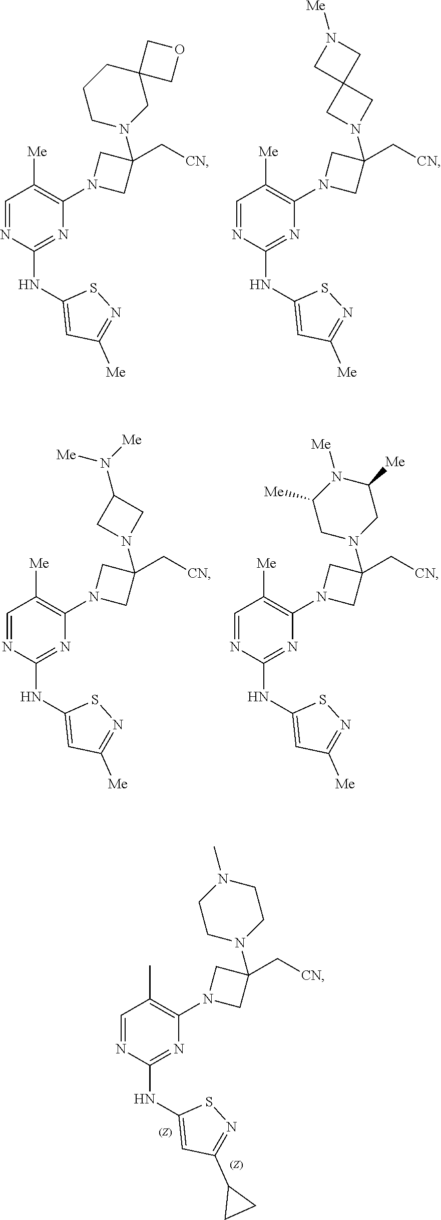 [SMILES: Cc1cc(Nc2ncc(C)c(N3CC(CC#N)(N4CCCC5(COC5)C4)C3)n2)sn1 ; Cc1cc(Nc2ncc(C)c(N3CC(CC#N)(N4CC5(CN(C)C5)C4)C3)n2)sn1 ; Cc1cc(Nc2ncc(C)c(N3CC(CC#N)(N4CC(N(C)C)C4)C3)n2)sn1 ; Cc1cc(Nc2ncc(C)c(N3CC(CC#N)(N4C[C@H](C)N(C)[C@@H](C)C4)C3)n2)sn1 ; Cc1cnc(Nc2cc(C3CC3)ns2)nc1N1CC(CC#N)(N2CCN(C)CC2)C1] 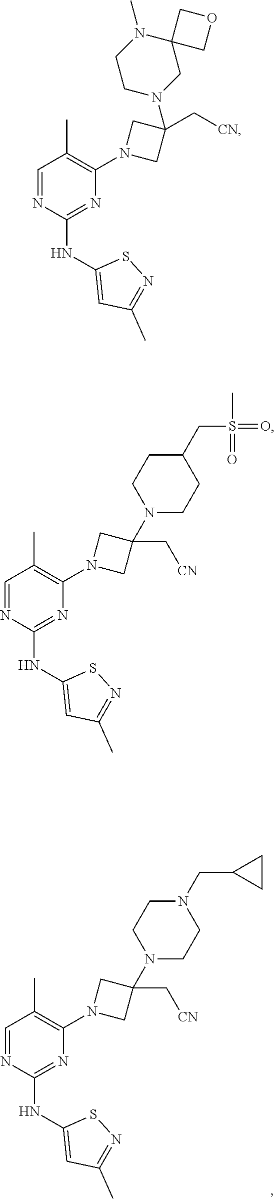 [SMILES: Cc1cc(Nc2ncc(C)c(N3CC(CC#N)(N4CCN(C)C5(COC5)C4)C3)n2)sn1 ; Cc1cc(Nc2ncc(C)c(N3CC(CC#N)(N4CCC(CS(C)(=O)=O)CC4)C3)n2)sn1 ; Cc1cc(Nc2ncc(C)c(N3CC(CC#N)(N4CCN(CC5CC5)CC4)C3)n2)sn1] 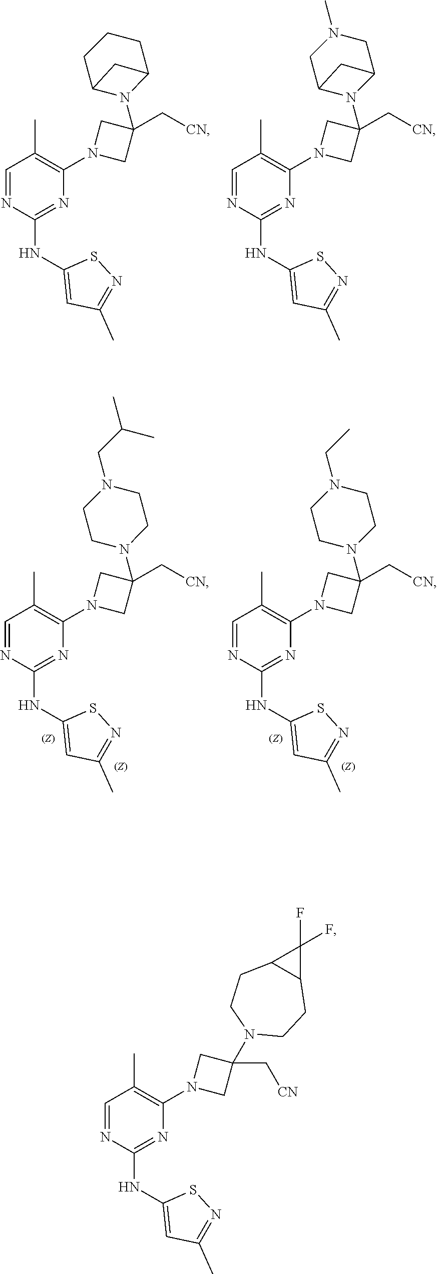 [SMILES: Cc1cc(Nc2ncc(C)c(N3CC(CC#N)(N4C5CCCC4C5)C3)n2)sn1 ; Cc1cc(Nc2ncc(C)c(N3CC(CC#N)(N4C5CC4CN(C)C5)C3)n2)sn1 ; Cc1cc(Nc2ncc(C)c(N3CC(CC#N)(N4CCN(CC(C)C)CC4)C3)n2)sn1 ; CCN1CCN(C2(CC#N)CN(c3nc(Nc4cc(C)ns4)ncc3C)C2)CC1 ; Cc1cc(Nc2ncc(C)c(N3CC(CC#N)(N4CCC5C(CC4)C5(F)F)C3)n2)sn1] 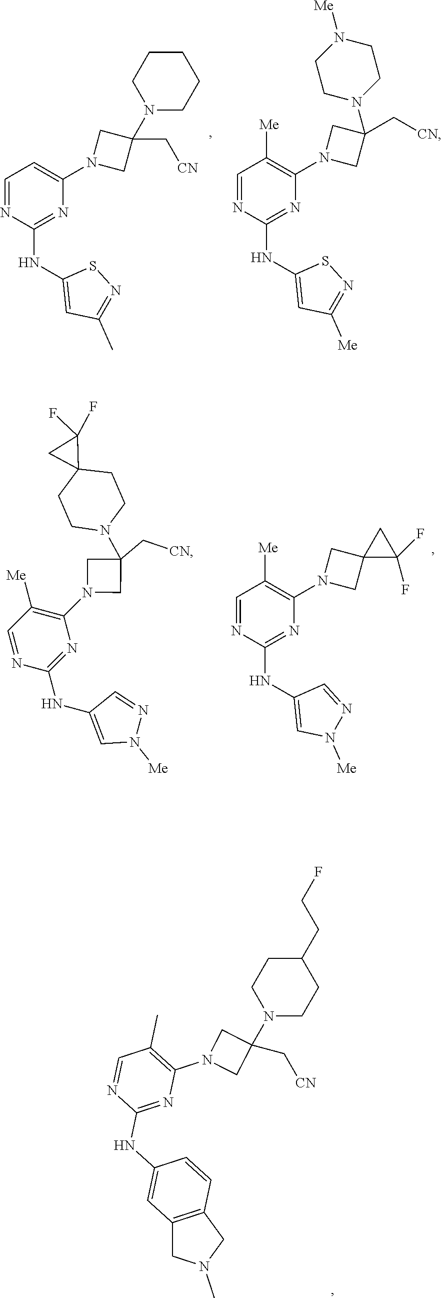 [SMILES: Cc1cc(Nc2nccc(N3CC(CC#N)(N4CCCCC4)C3)n2)sn1 ; Cc1cc(Nc2ncc(C)c(N3CC(CC#N)(N4CCN(C)CC4)C3)n2)sn1 ; Cc1cnc(Nc2cnn(C)c2)nc1N1CC(CC#N)(N2CCC3(CC2)CC3(F)F)C1 ; Cc1cnc(Nc2cnn(C)c2)nc1N1CC2(C1)CC2(F)F ; Cc1cnc(Nc2ccc3c(c2)CN(C)C3)nc1N1CC(CC#N)(N2CCC(CCF)CC2)C1] 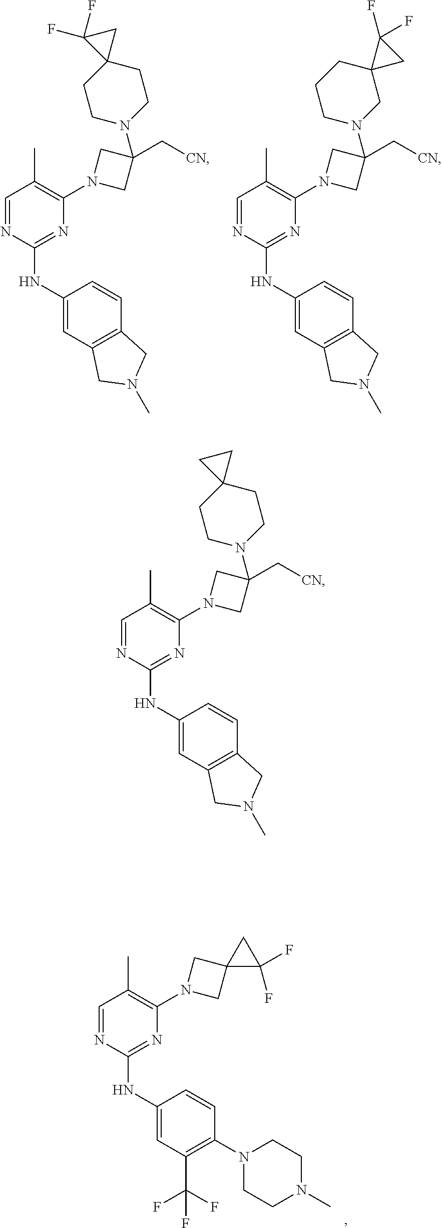 [SMILES: Cc1cnc(Nc2ccc3c(c2)CN(C)C3)nc1N1CC(CC#N)(N2CCC3(CC2)CC3(F)F)C1 ; Cc1cnc(Nc2ccc3c(c2)CN(C)C3)nc1N1CC(CC#N)(N2CCCC3(C2)CC3(F)F)C1 ; Cc1cnc(Nc2ccc3c(c2)CN(C)C3)nc1N1CC(CC#N)(N2CCC3(CC2)CC3)C1 ; Cc1cnc(Nc2ccc(N3CCN(C)CC3)c(C(F)(F)F)c2)nc1N1CC2(C1)CC2(F)F] 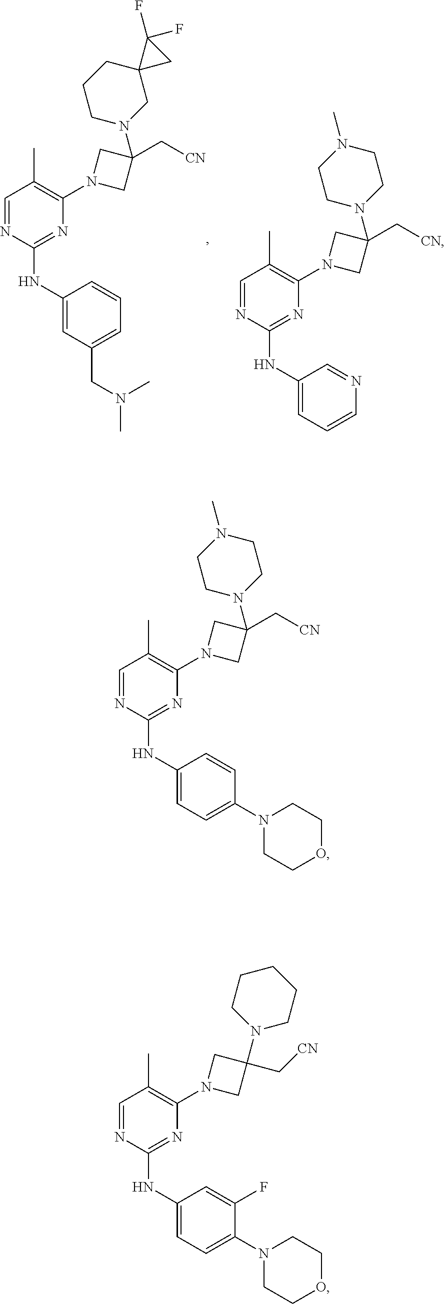 [SMILES: Cc1cnc(Nc2cccc(CN(C)C)c2)nc1N1CC(CC#N)(N2CCCC3(C2)CC3(F)F)C1 ; Cc1cnc(Nc2cccnc2)nc1N1CC(CC#N)(N2CCN(C)CC2)C1 ; Cc1cnc(Nc2ccc(N3CCOCC3)cc2)nc1N1CC(CC#N)(N2CCN(C)CC2)C1 ; Cc1cnc(Nc2ccc(N3CCOCC3)c(F)c2)nc1N1CC(CC#N)(N2CCCCC2)C1] 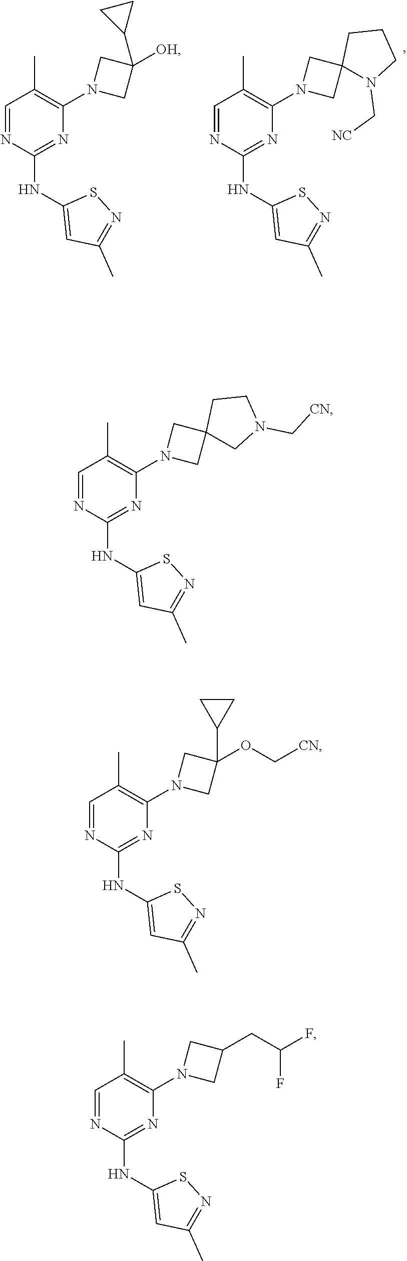 [SMILES: Cc1cc(Nc2ncc(C)c(N3CC(O)(C4CC4)C3)n2)sn1 ; Cc1cc(Nc2ncc(C)c(N3CC4(CCCN4CC#N)C3)n2)sn1 ; Cc1cc(Nc2ncc(C)c(N3CC4(CCN(CC#N)C4)C3)n2)sn1 ; Cc1cc(Nc2ncc(C)c(N3CC(OCC#N)(C4CC4)C3)n2)sn1 ; Cc1cc(Nc2ncc(C)c(N3CC(CC(F)F)C3)n2)sn1] 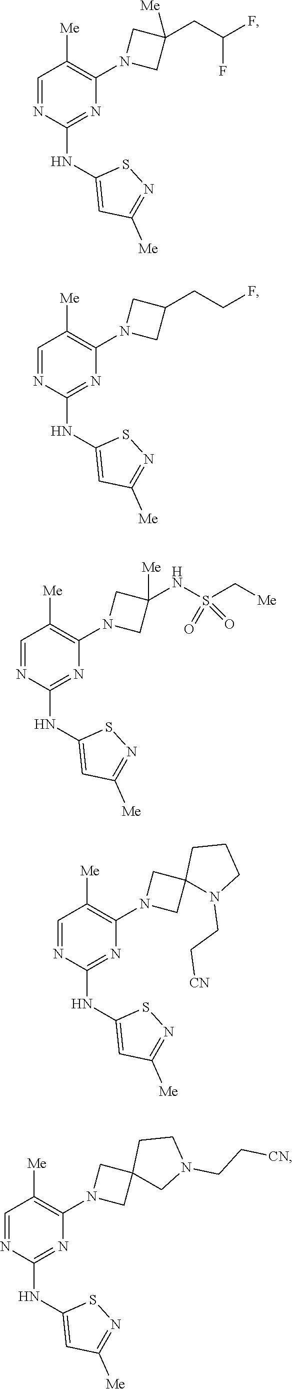 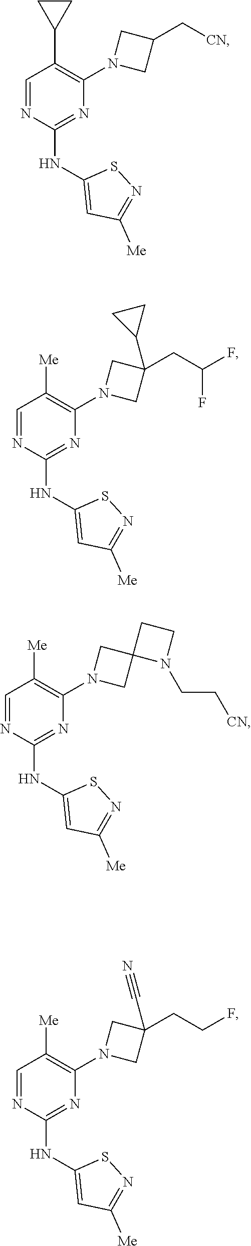 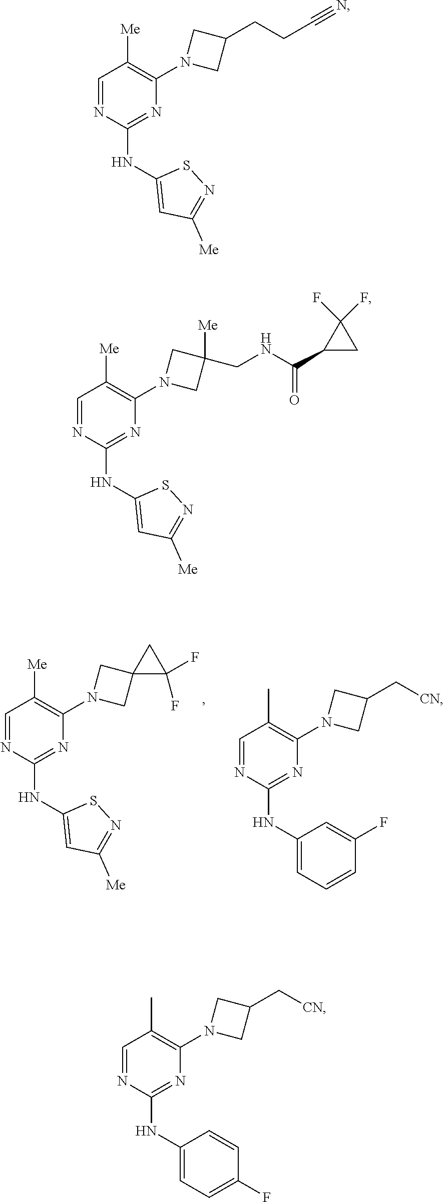 [SMILES: Cc1cc(Nc2ncc(C)c(N3CC(CCC#N)C3)n2)sn1 ; Cc1cc(Nc2ncc(C)c(N3CC(C)(CNC(=O)[C@@H]4CC4(F)F)C3)n2)sn1 ; Cc1cc(Nc2ncc(C)c(N3CC4(C3)CC4(F)F)n2)sn1 ; Cc1cnc(Nc2cccc(F)c2)nc1N1CC(CC#N)C1 ; Cc1cnc(Nc2ccc(F)cc2)nc1N1CC(CC#N)C1] 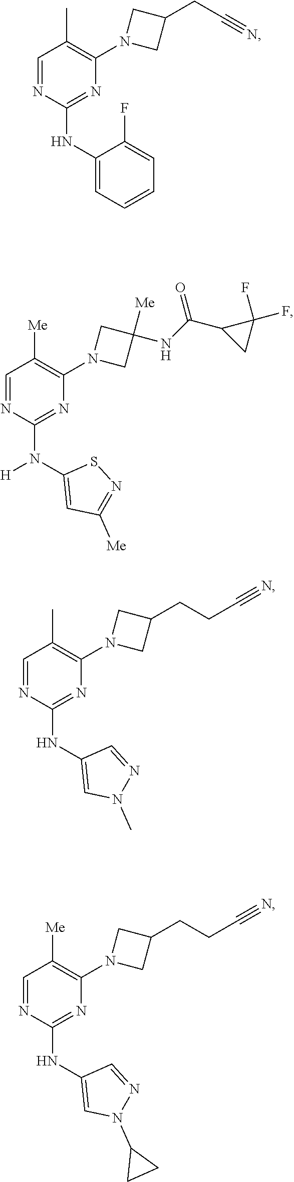 [SMILES: Cc1cnc(Nc2ccccc2F)nc1N1CC(CC#N)C1 ; Cc1cc(Nc2ncc(C)c(N3CC(C)(NC(=O)C4CC4(F)F)C3)n2)sn1 ; Cc1cnc(Nc2cnn(C)c2)nc1N1CC(CCC#N)C1 ; Cc1cnc(Nc2cnn(C3CC3)c2)nc1N1CC(CCC#N)C1] 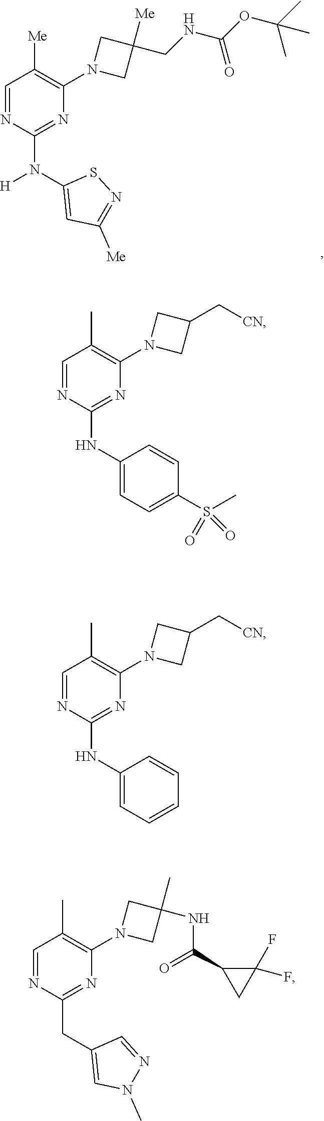 [SMILES: Cc1cc(Nc2ncc(C)c(N3CC(C)(CNC(=O)OC(C)(C)C)C3)n2)sn1 ; Cc1cnc(Nc2ccc(S(C)(=O)=O)cc2)nc1N1CC(CC#N)C1 ; Cc1cnc(Nc2ccccc2)nc1N1CC(CC#N)C1 ; Cc1cnc(Cc2cnn(C)c2)nc1N1CC(C)(NC(=O)[C@@H]2CC2(F)F)C1]
or a pharmaceutically acceptable salt thereof.

10. The compound of claim 1, having the formula: 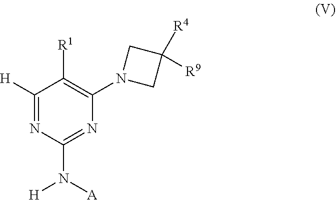
or a pharmaceutically acceptable salt thereof,
R¹ is H, F, Cl, Br, I, CN, OCF₃, OCH₂CF₃, C₁₋₃ alkyl, C₃₋₆ cycloalkyl, or C₁₋₆ haloalkyl,
R⁴ is C₁₋₆ alkyl, C₁₋₆ heteroalkyl or C₁₋₆ haloalkyl, CH₂CN, CH₂CH₂CN, CH₂CH₂CH₂CN, alkylenecycloalkyl, alkylenecyclopropyl, or hydroxyl,
R⁹ is C₁₋₆ alkyl, C₁₋₆ heteroalkyl or C₁₋₆ haloalkyl, or R⁴ and R⁹ combine to form a carbocyclic, polycyclic, or heteroalkyl ring of at least 4 member atoms and at most 8 member atoms, and
A is phenyl, or a monocyclic C₃₋₅ heteroaryl having 1-3 heteroatoms, wherein each heteroatom is, independently, N, O, or S.

11. The compound of claim 1, which is selected from: 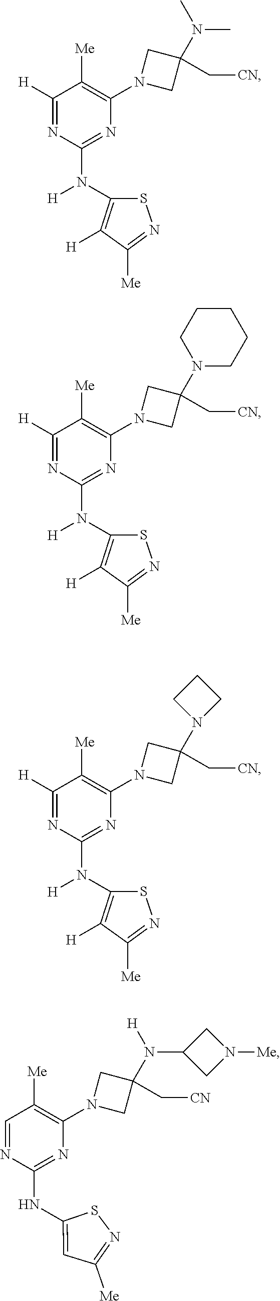 [SMILES: Cc1cc(Nc2ncc(C)c(N3CC(CC#N)(N(C)C)C3)n2)sn1 ; Cc1cc(Nc2ncc(C)c(N3CC(CC#N)(N4CCCCC4)C3)n2)sn1 ; Cc1cc(Nc2ncc(C)c(N3CC(CC#N)(N4CCC4)C3)n2)sn1 ; Cc1cc(Nc2ncc(C)c(N3CC(CC#N)(NC4CN(C)C4)C3)n2)sn1] 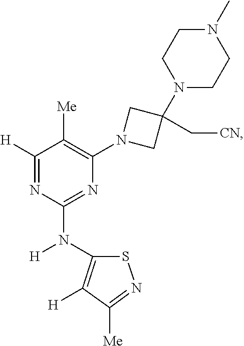 [SMILES: Cc1cc(Nc2ncc(C)c(N3CC(CC#N)(N4CCN(C)CC4)C3)n2)sn1]
or a pharmaceutically acceptable salt thereof.

12. The compound of claim 1, having a formula: 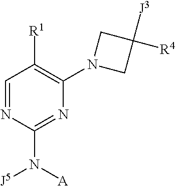
or a pharmaceutically acceptable salt thereof;
wherein
J⁵ is H, S(O)₂CH₃, or S(O)₂CH₂CH₃;
R¹ is H, CH₃, CH₂CH₃, cyclopropyl, OCH₃, F, Cl, or CN;
A is 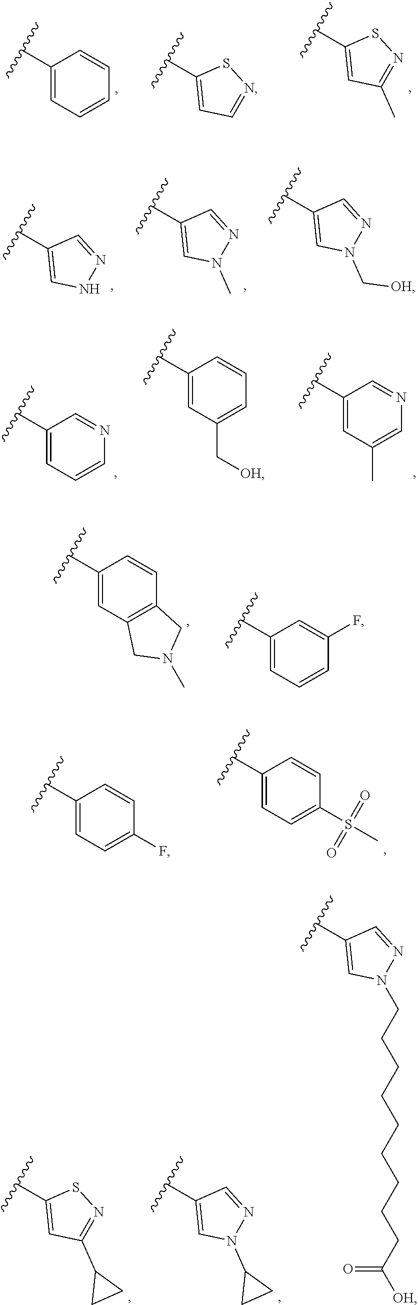 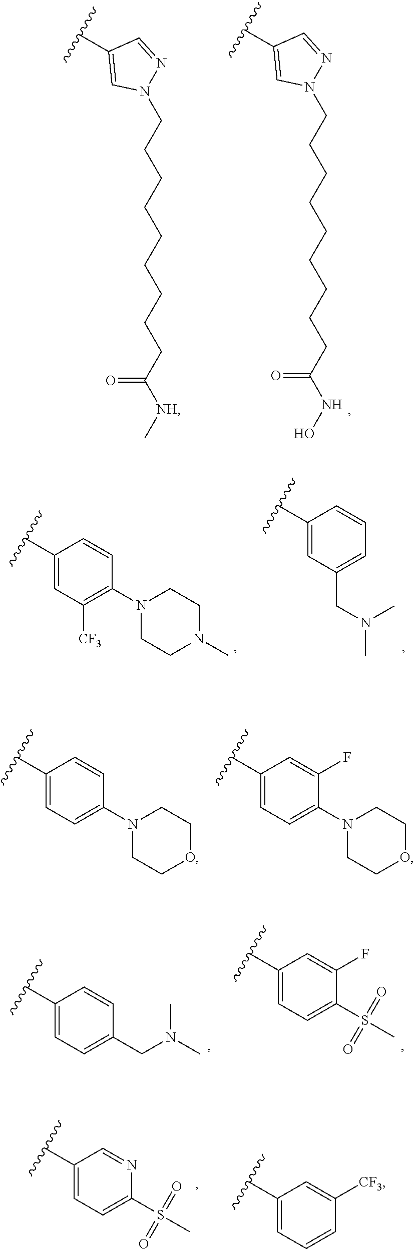 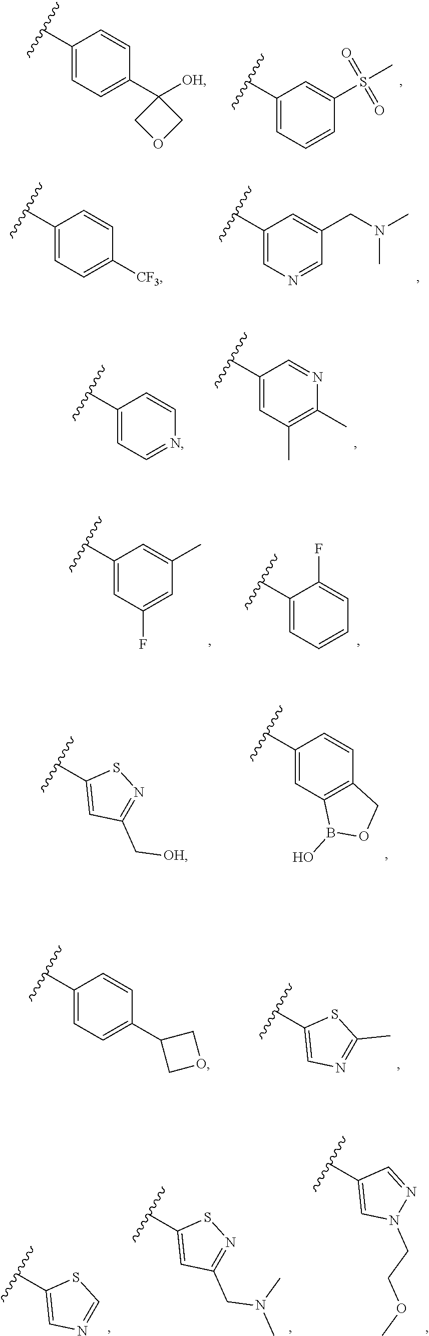 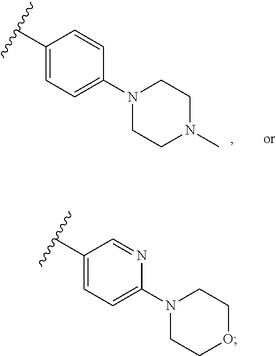 [SMILES: *c1ccc(N2CCN(C)CC2)cc1 ; *c1ccc(N2CCOCC2)nc1]
J³ is CN, OH, NH₂, 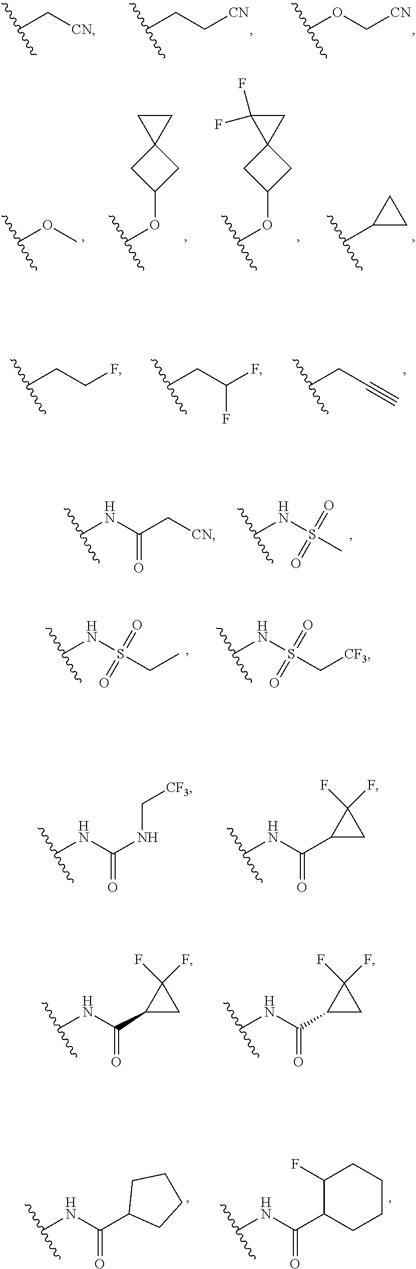 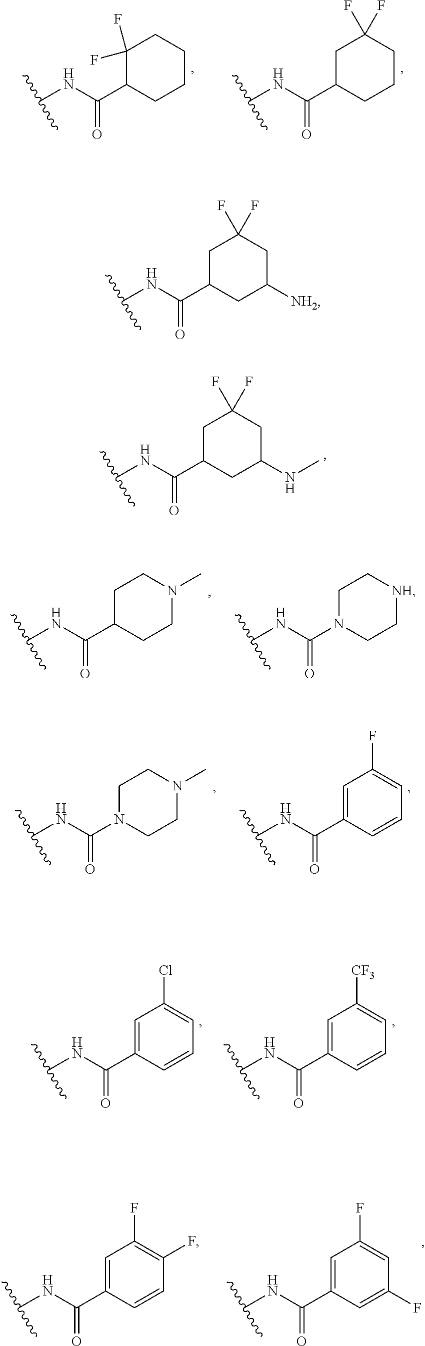 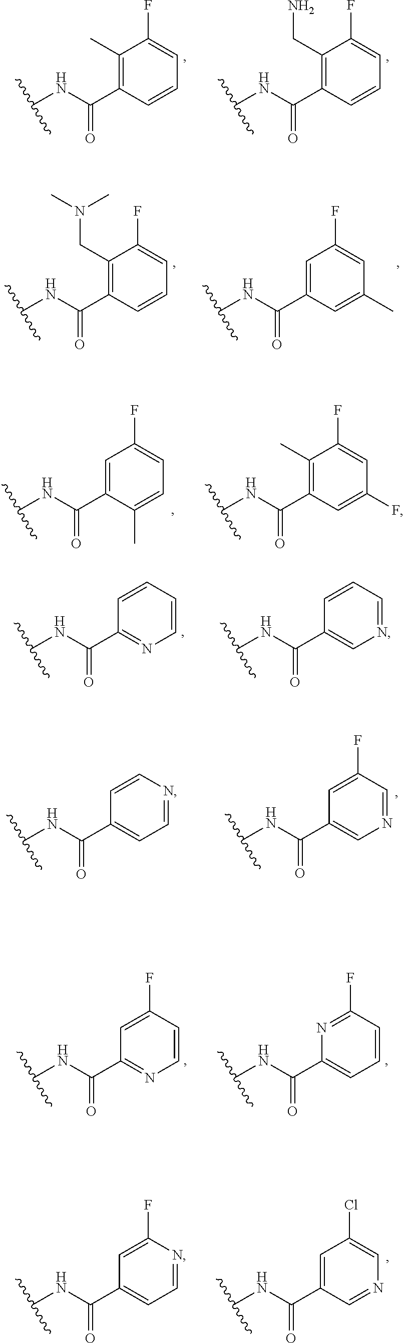 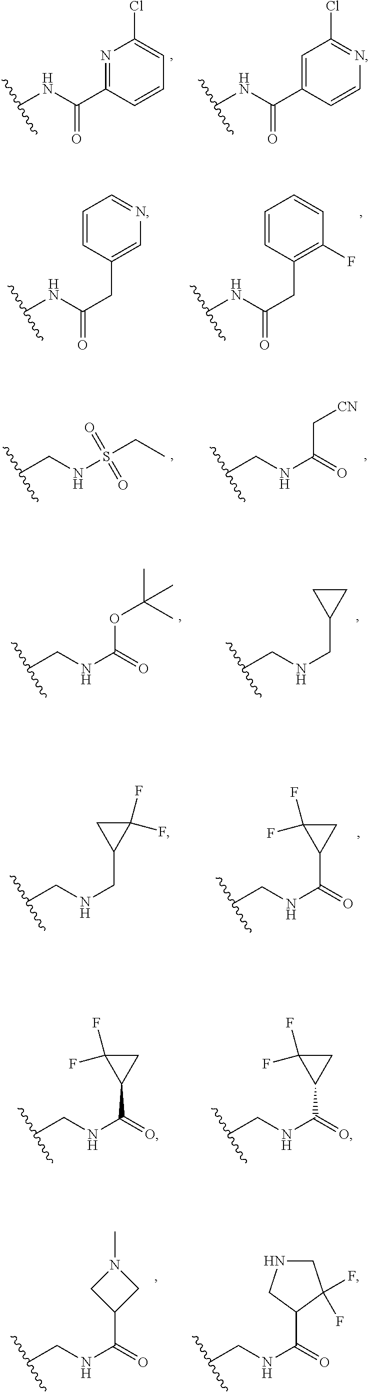 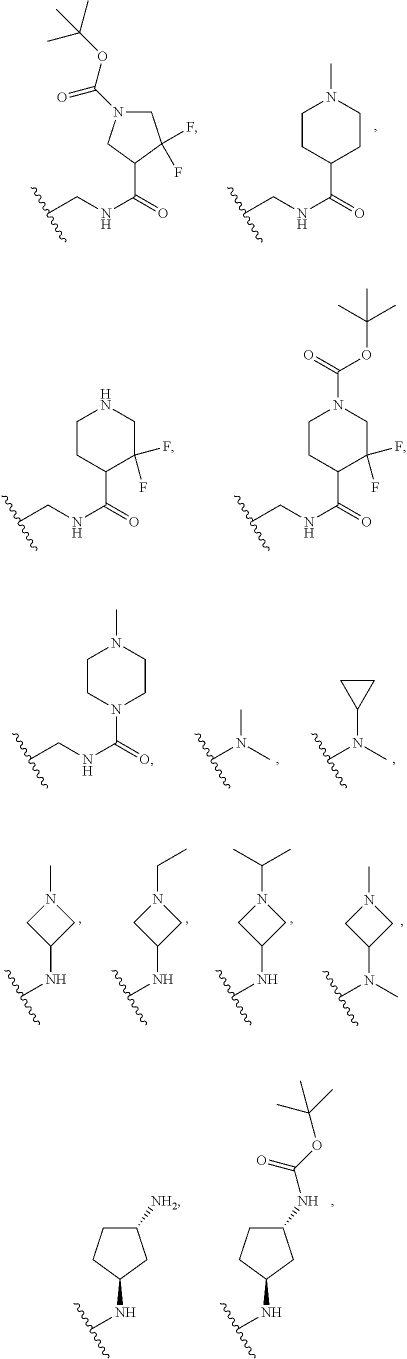 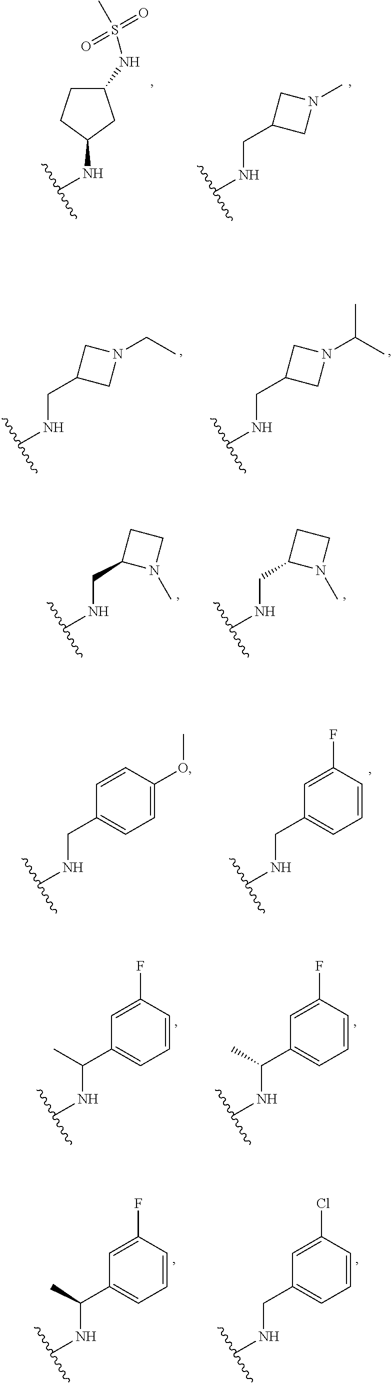 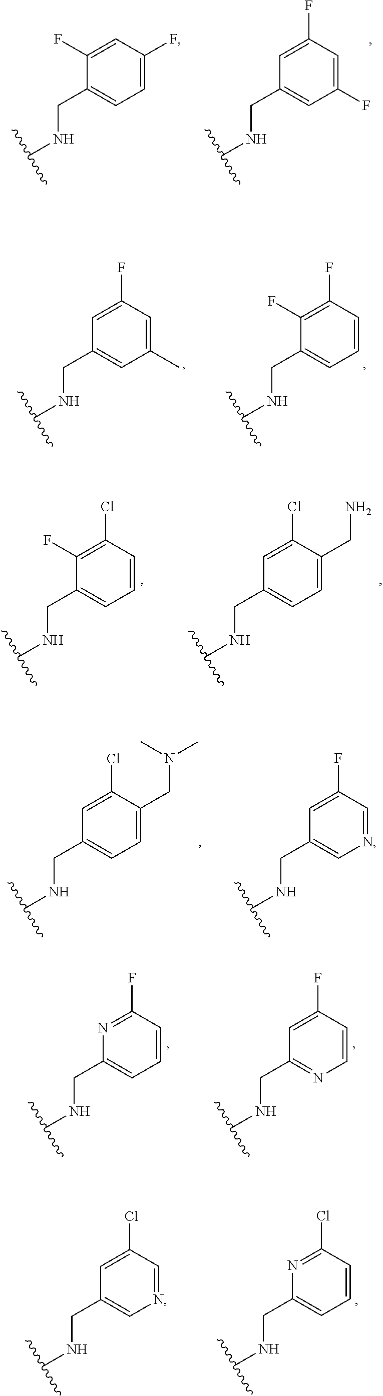 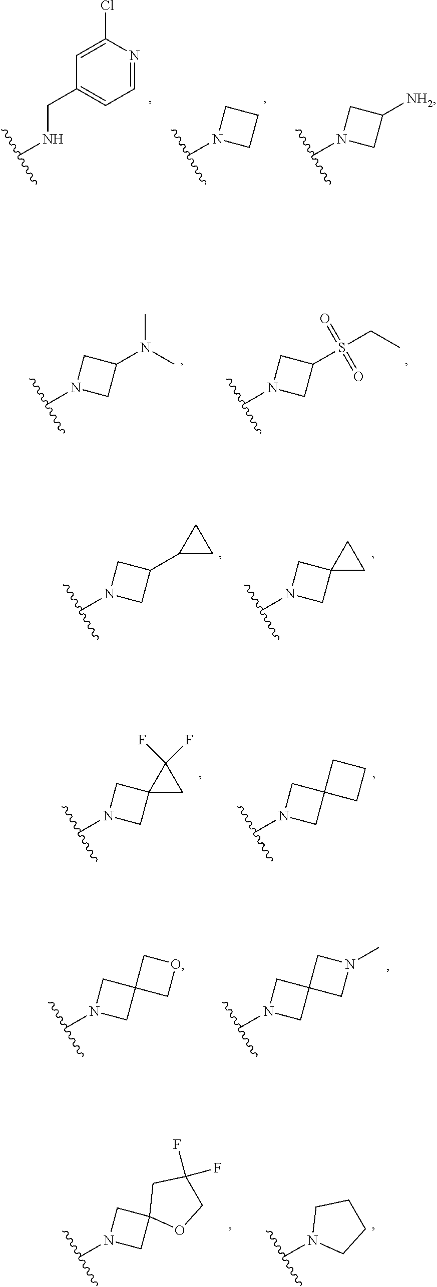 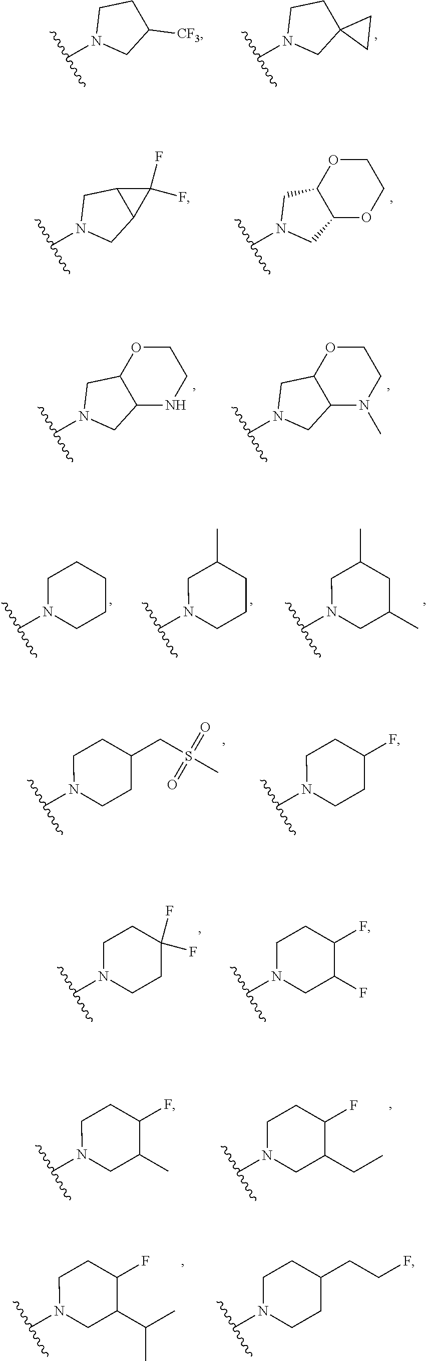 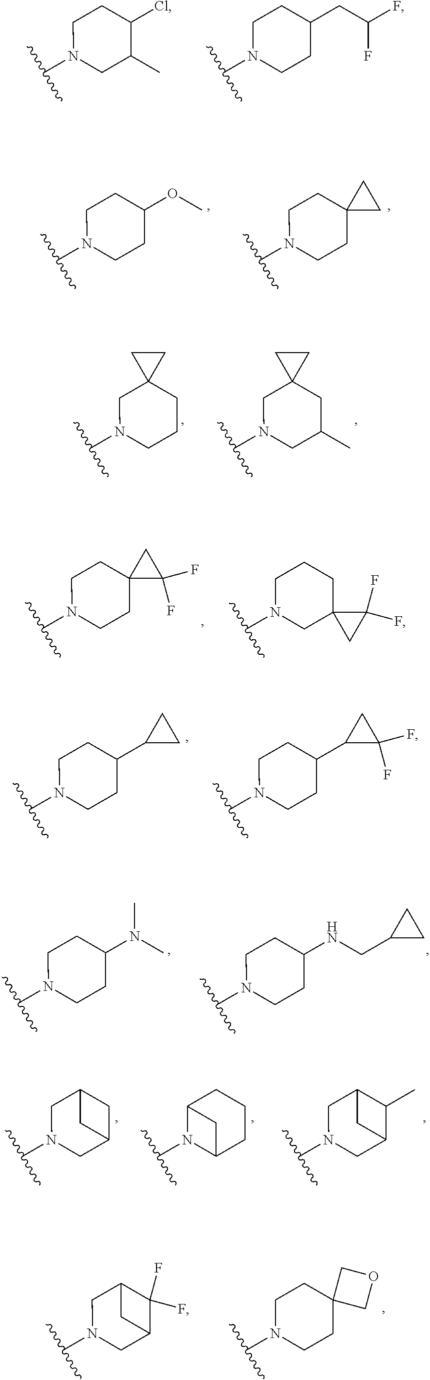 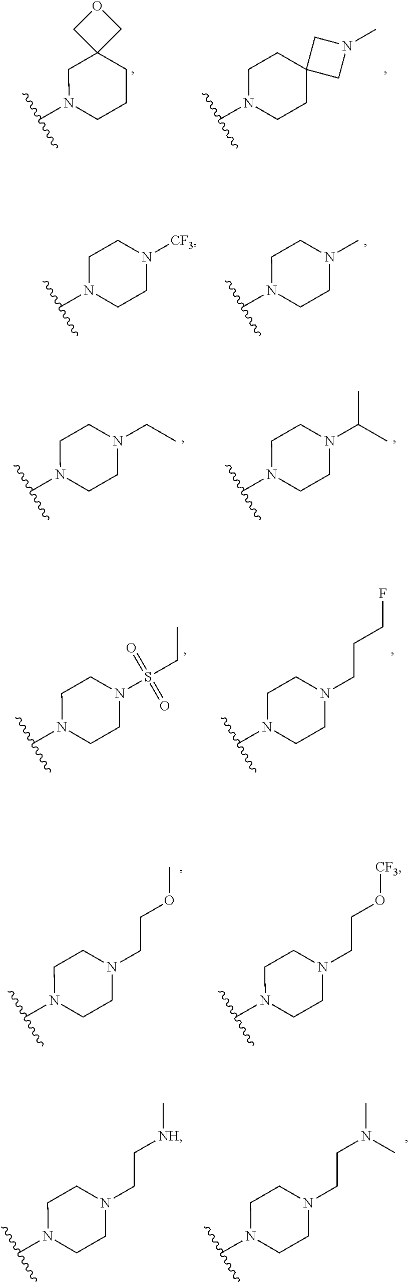 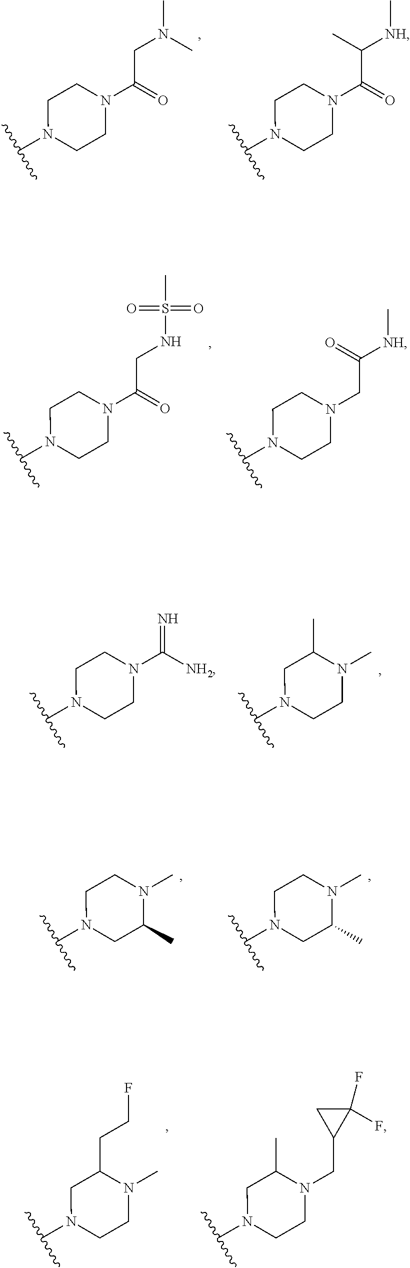 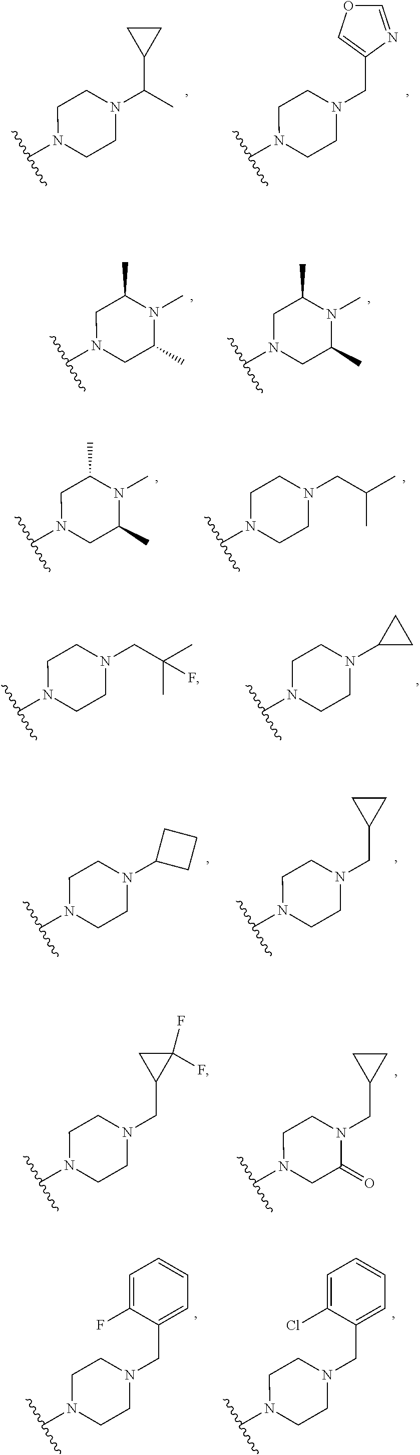 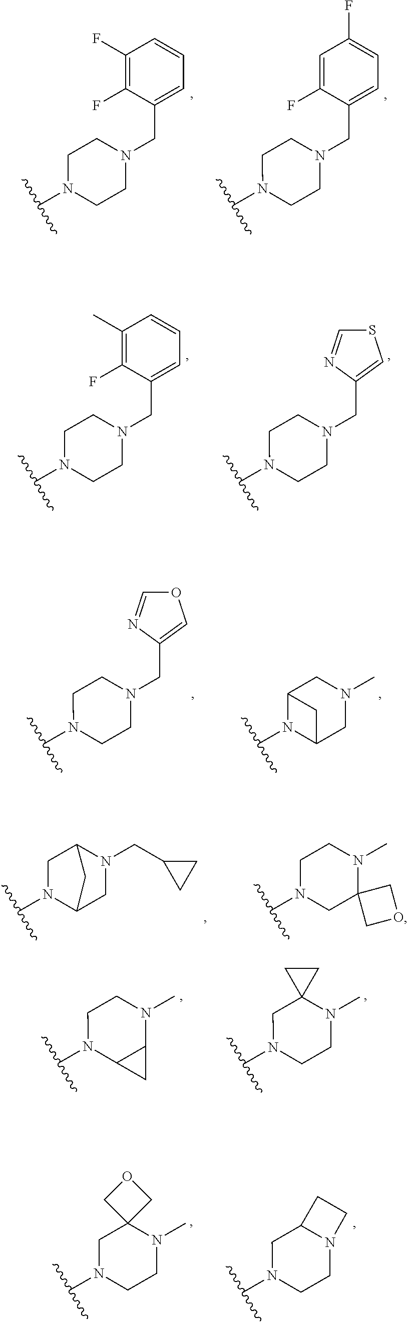 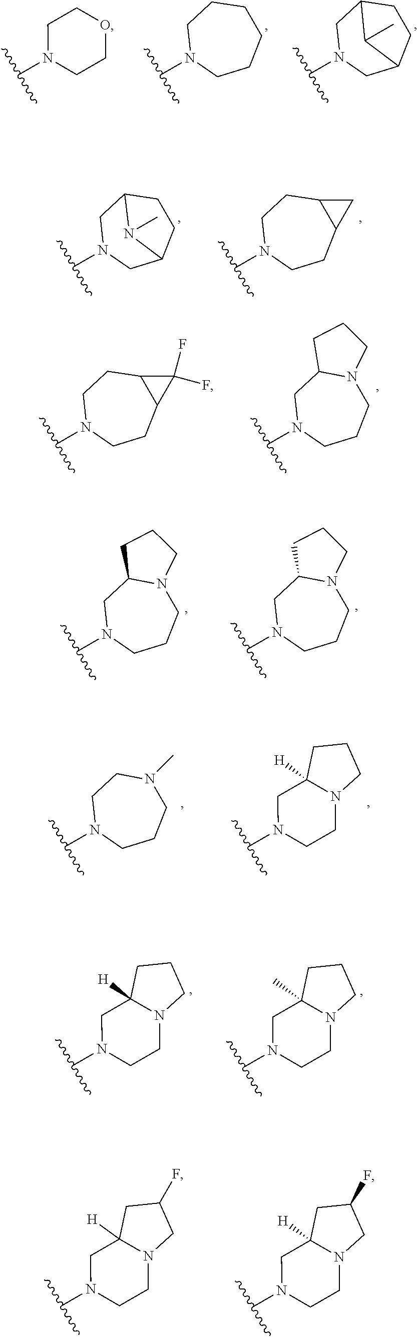 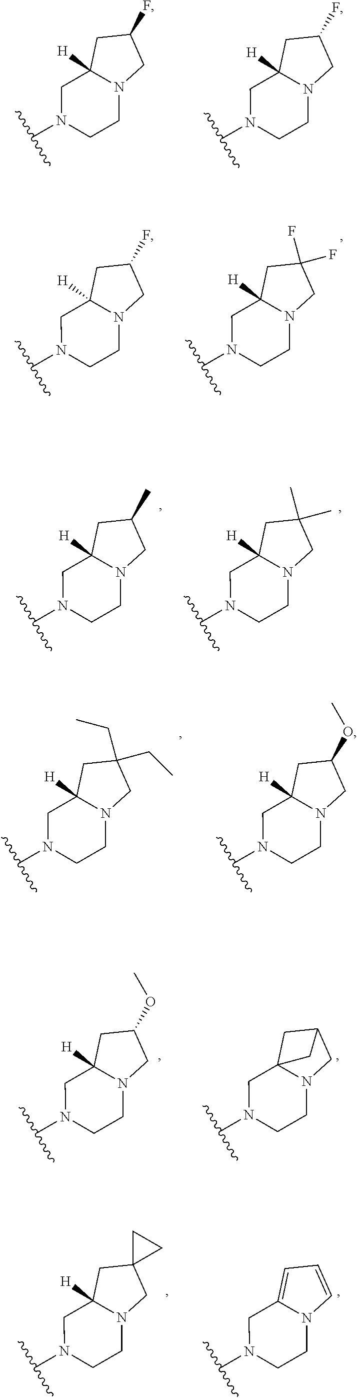 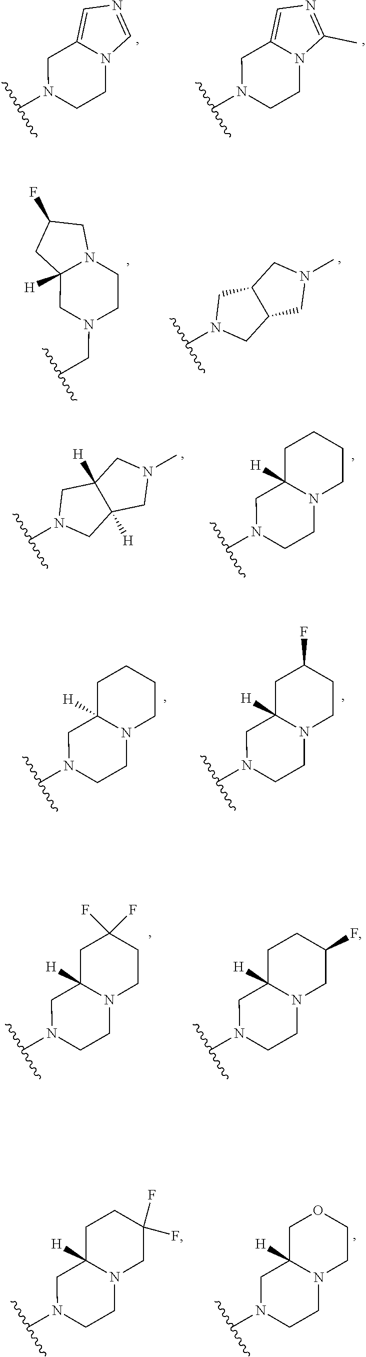 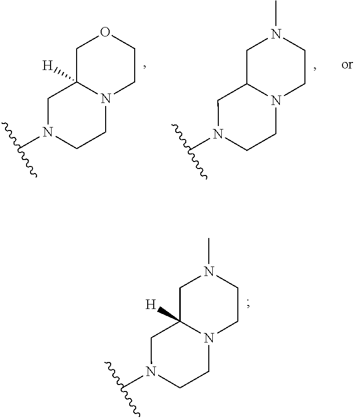 [SMILES: *N1CCN2CCOC[C@@H]2C1 ; *N1CCN2CCN(C)CC2C1 ; *N1CCN2CCN(C)C[C@H]2C1]
and
R⁴ is H, CH₃, CH₂CH₃, isopropyl, cyclopropyl, cyclobutyl, cyclopentyl, cyclohexyl, cycloheptyl, OH, CH₂OH, CH₂CH₂OH, CH₂F, CH₂CHF₂, CH₂CH₂F, CH₂CH₂Cl, CH₂-cyclopropyl, CH₂CN, CH₂CH₂CN, 3-fluorocyclobut-1-yl, tetrahydro-2H-pyran-4-yl, CH₂C(O)OCH₃, or CH₂OC(O)CH₃;
or J³ and R⁴, together with the atoms to which they are attached, combine to form

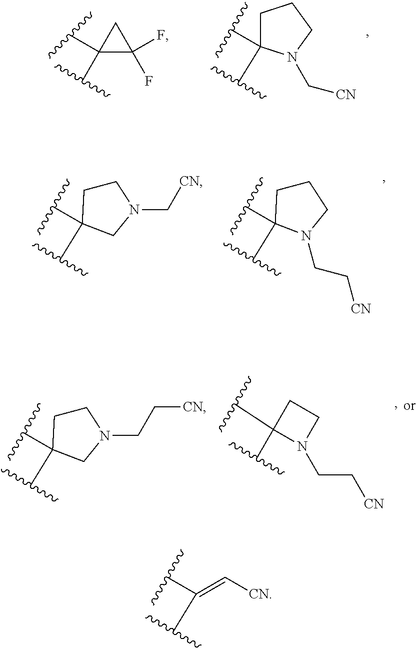

13. The compound of claim 1, which is selected from: 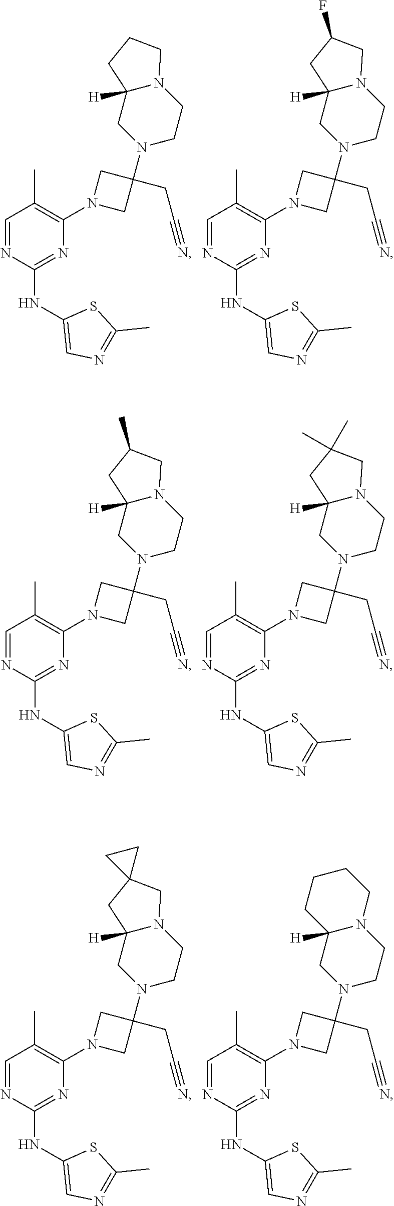 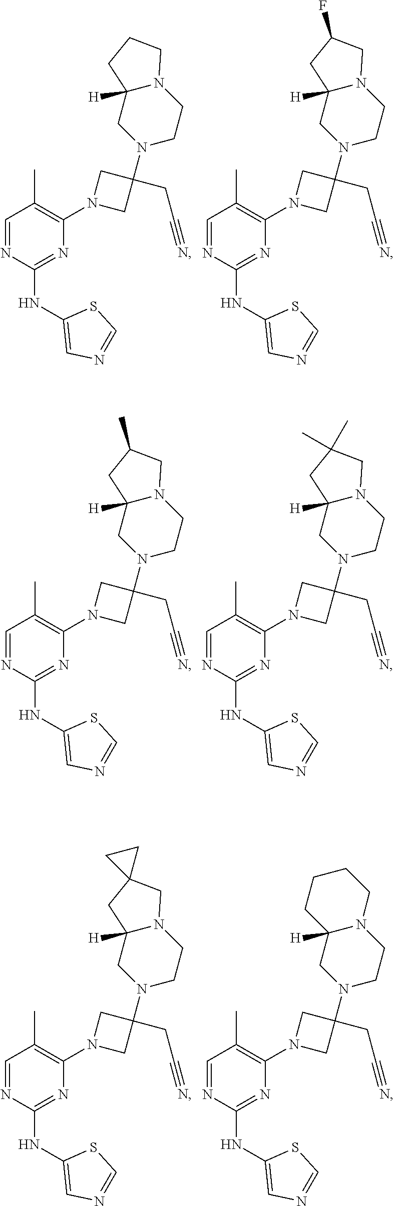 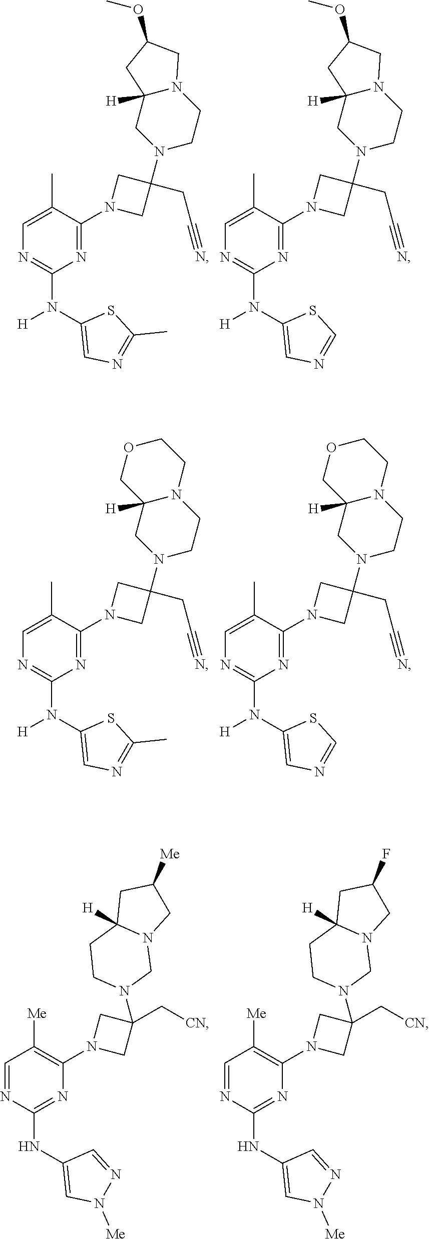 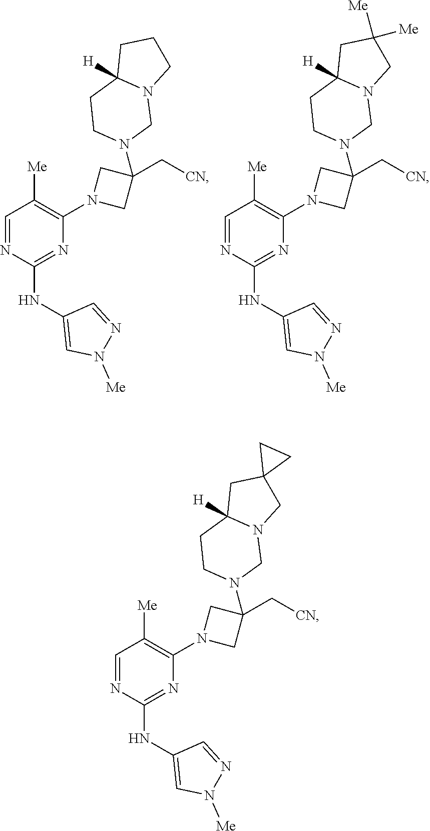 [SMILES: Cc1cnc(Nc2cnn(C)c2)nc1N1CC(CC#N)(N2CC[C@@H]3CCCN3C2)C1 ; Cc1cnc(Nc2cnn(C)c2)nc1N1CC(CC#N)(N2CC[C@@H]3CC(C)(C)CN3C2)C1 ; Cc1cnc(Nc2cnn(C)c2)nc1N1CC(CC#N)(N2CC[C@@H]3CC4(CC4)CN3C2)C1]
or a pharmaceutically acceptable salt thereof.

14-27. (canceled)

28. The compound of claim 1, which is selected from: 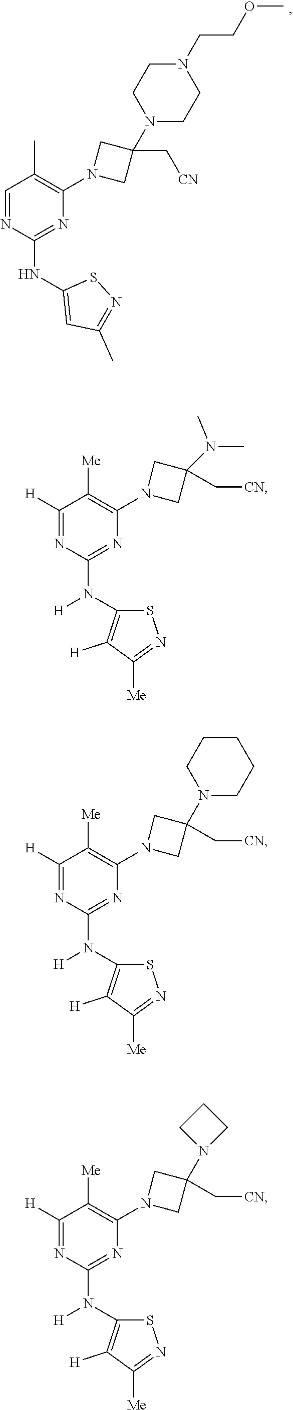 [SMILES: COCCN1CCN(C2(CC#N)CN(c3nc(Nc4cc(C)ns4)ncc3C)C2)CC1 ; Cc1cc(Nc2ncc(C)c(N3CC(CC#N)(N(C)C)C3)n2)sn1 ; Cc1cc(Nc2ncc(C)c(N3CC(CC#N)(N4CCCCC4)C3)n2)sn1 ; Cc1cc(Nc2ncc(C)c(N3CC(CC#N)(N4CCC4)C3)n2)sn1] 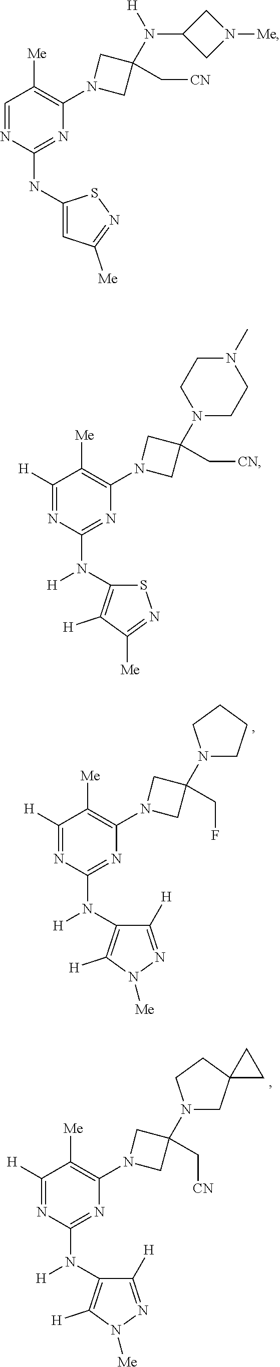 [SMILES: Cc1cc(Nc2ncc(C)c(N3CC(CC#N)(NC4CN(C)C4)C3)n2)sn1 ; Cc1cc(Nc2ncc(C)c(N3CC(CC#N)(N4CCN(C)CC4)C3)n2)sn1 ; Cc1cnc(Nc2cnn(C)c2)nc1N1CC(CF)(N2CCCC2)C1 ; Cc1cnc(Nc2cnn(C)c2)nc1N1CC(CC#N)(N2CCC3(CC3)C2)C1] 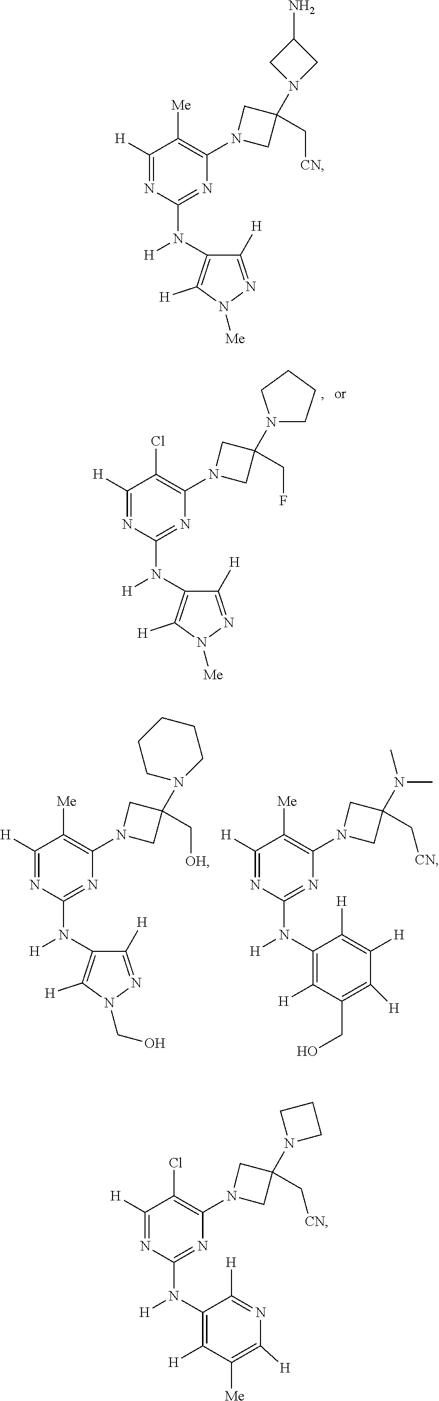 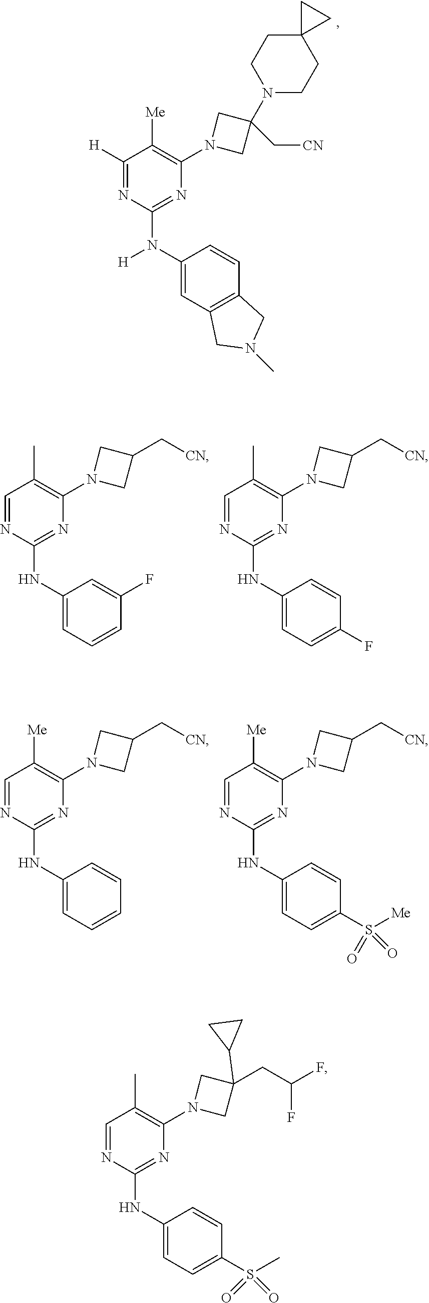 [SMILES: Cc1cnc(Nc2ccc3c(c2)CN(C)C3)nc1N1CC(CC#N)(N2CCC3(CC2)CC3)C1 ; Cc1cnc(Nc2cccc(F)c2)nc1N1CC(CC#N)C1 ; Cc1cnc(Nc2ccc(F)cc2)nc1N1CC(CC#N)C1 ; Cc1cnc(Nc2ccccc2)nc1N1CC(CC#N)C1 ; Cc1cnc(Nc2ccc(S(C)(=O)=O)cc2)nc1N1CC(CC#N)C1 ; Cc1cnc(Nc2ccc(S(C)(=O)=O)cc2)nc1N1CC(CC(F)F)(C2CC2)C1] 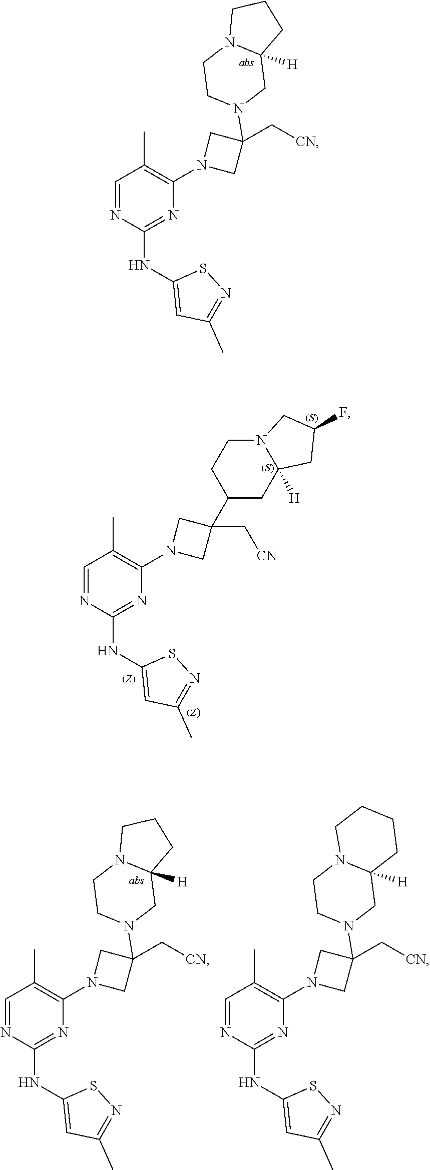 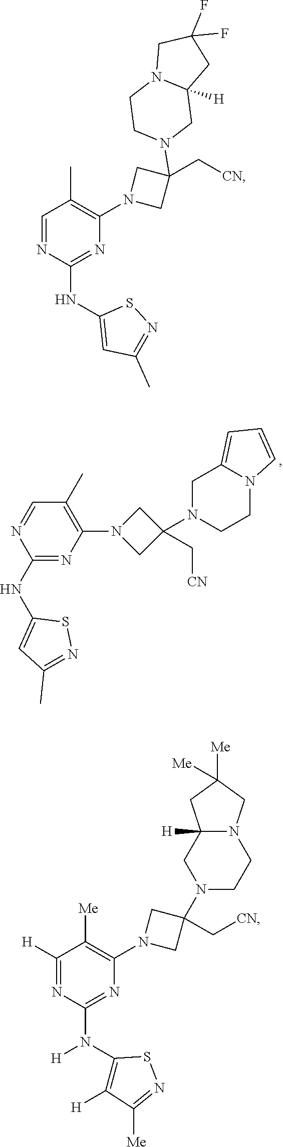 [SMILES: Cc1cc(Nc2ncc(C)c(N3CC(CC#N)(N4CCN5CC(F)(F)C[C@H]5C4)C3)n2)sn1 ; Cc1cc(Nc2ncc(C)c(N3CC(CC#N)(N4CCn5cccc5C4)C3)n2)sn1 ; Cc1cc(Nc2ncc(C)c(N3CC(CC#N)(N4CCN5CC(C)(C)C[C@H]5C4)C3)n2)sn1] 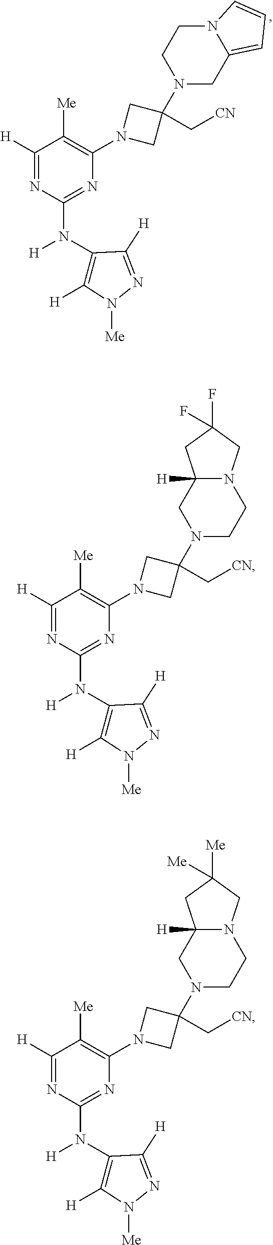 [SMILES: Cc1cnc(Nc2cnn(C)c2)nc1N1CC(CC#N)(N2CCn3cccc3C2)C1 ; Cc1cnc(Nc2cnn(C)c2)nc1N1CC(CC#N)(N2CCN3CC(F)(F)C[C@H]3C2)C1 ; Cc1cnc(Nc2cnn(C)c2)nc1N1CC(CC#N)(N2CCN3CC(C)(C)C[C@H]3C2)C1] 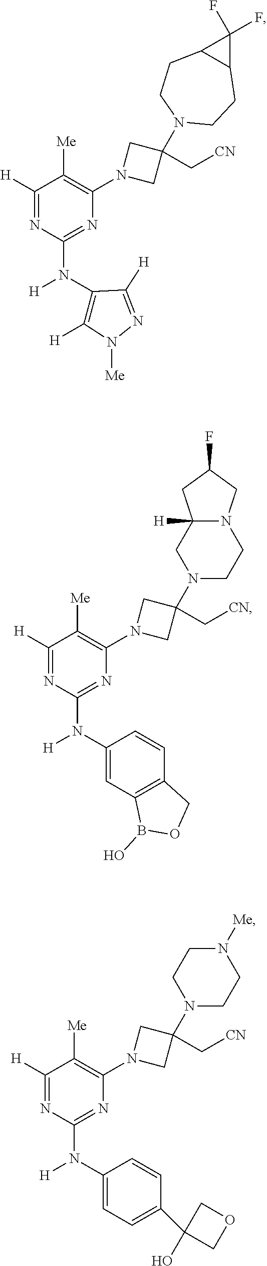 [SMILES: Cc1cnc(Nc2cnn(C)c2)nc1N1CC(CC#N)(N2CCC3C(CC2)C3(F)F)C1 ; Cc1cnc(Nc2ccc3c(c2)B(O)OC3)nc1N1CC(CC#N)(N2CCN3C[C@H](F)C[C@H]3C2)C1 ; Cc1cnc(Nc2ccc(C3(O)COC3)cc2)nc1N1CC(CC#N)(N2CCN(C)CC2)C1] 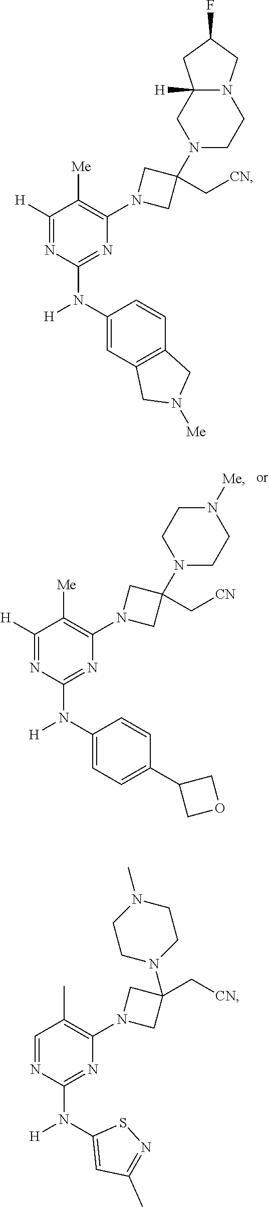 [SMILES: Cc1cnc(Nc2ccc3c(c2)CN(C)C3)nc1N1CC(CC#N)(N2CCN3C[C@H](F)C[C@H]3C2)C1 ; Cc1cnc(Nc2ccc(C3COC3)cc2)nc1N1CC(CC#N)(N2CCN(C)CC2)C1 ; "or" ; Cc1cc(Nc2ncc(C)c(N3CC(CC#N)(N4CCN(C)CC4)C3)n2)sn1]
or a pharmaceutically acceptable salt thereof.

29. The compound of claim 1, which is selected from: 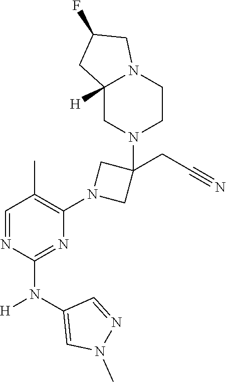 [SMILES: Cc1cnc(Nc2cnn(C)c2)nc1N1CC(CC#N)(N2CCN3C[C@H](F)C[C@H]3C2)C1]
or a pharmaceutically acceptable salt thereof.

30. The compound, which is selected from: 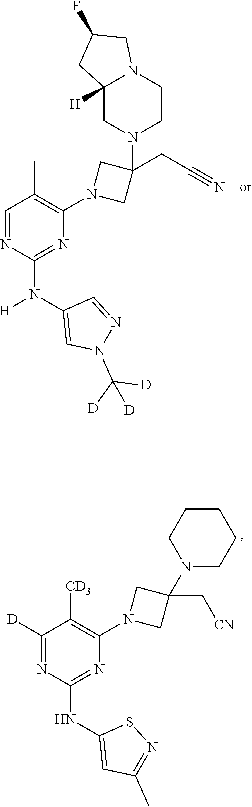 [SMILES: [2H]C([2H])([2H])n1cc(Nc2ncc(C)c(N3CC(CC#N)(N4CCN5C[C@H](F)C[C@H]5C4)C3)n2)cn1 ; [2H]c1nc(Nc2cc(C)ns2)nc(N2CC(CC#N)(N3CCCCC3)C2)c1C([2H])([2H])[2H]]
or a pharmaceutically acceptable salt thereof.

31. The compound of claim 1, which is selected from: 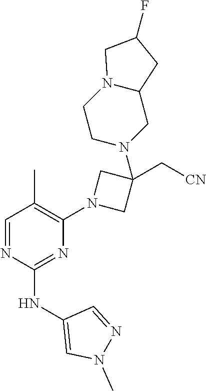 [SMILES: Cc1cnc(Nc2cnn(C)c2)nc1N1CC(CC#N)(N2CCN3CC(F)CC3C2)C1]
or a pharmaceutically acceptable salt thereof.

32. The compound of claim 1, which is selected from: 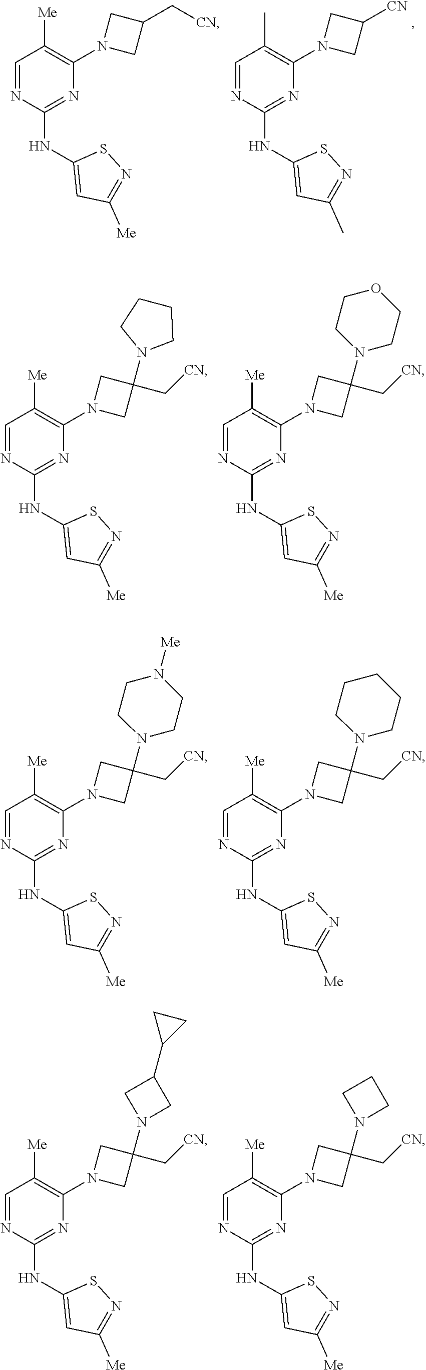 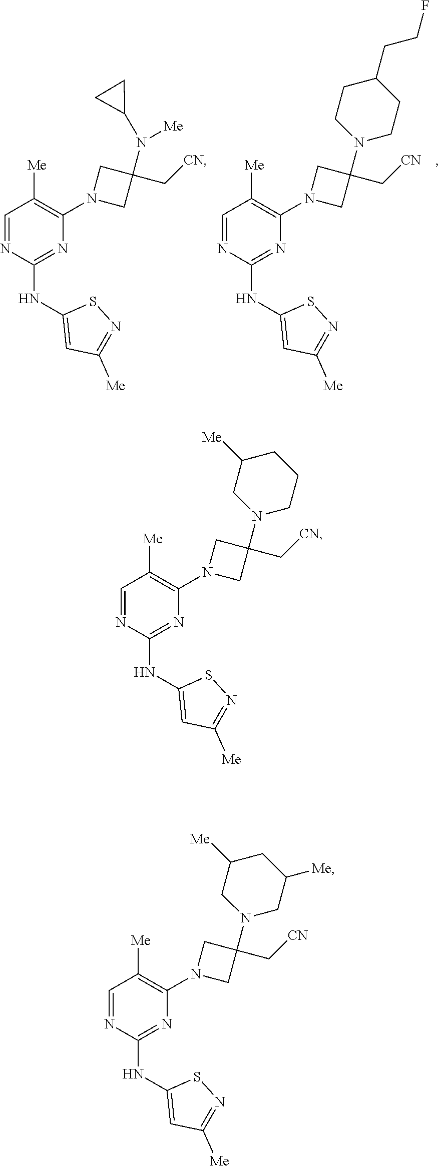 [SMILES: Cc1cc(Nc2ncc(C)c(N3CC(CC#N)(N(C)C4CC4)C3)n2)sn1 ; Cc1cc(Nc2ncc(C)c(N3CC(CC#N)(N4CCC(CCF)CC4)C3)n2)sn1 ; Cc1cc(Nc2ncc(C)c(N3CC(CC#N)(N4CCCC(C)C4)C3)n2)sn1 ; Cc1cc(Nc2ncc(C)c(N3CC(CC#N)(N4CC(C)CC(C)C4)C3)n2)sn1] 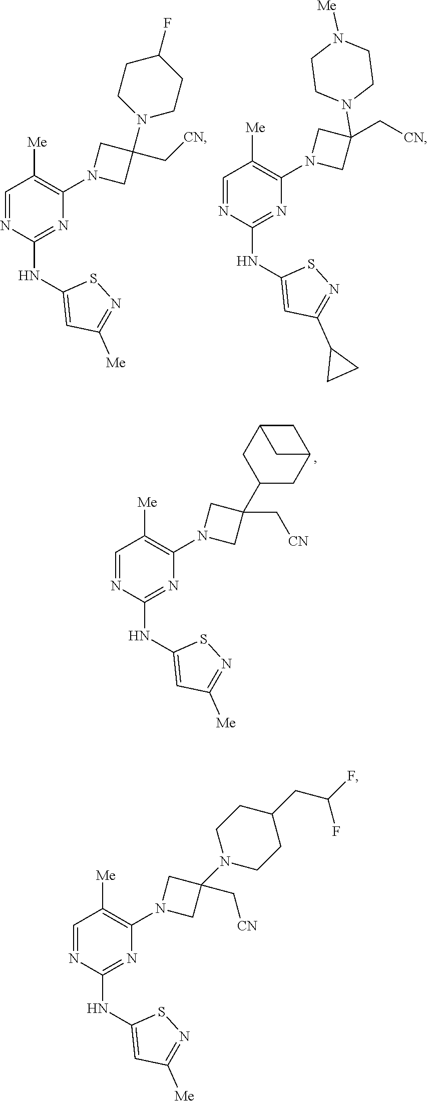 [SMILES: Cc1cc(Nc2ncc(C)c(N3CC(CC#N)(N4CCC(F)CC4)C3)n2)sn1 ; Cc1cnc(Nc2cc(C3CC3)ns2)nc1N1CC(CC#N)(N2CCN(C)CC2)C1 ; Cc1cc(Nc2ncc(C)c(N3CC(CC#N)(C4CC5CC(C5)C4)C3)n2)sn1 ; Cc1cc(Nc2ncc(C)c(N3CC(CC#N)(N4CCC(CC(F)F)CC4)C3)n2)sn1] 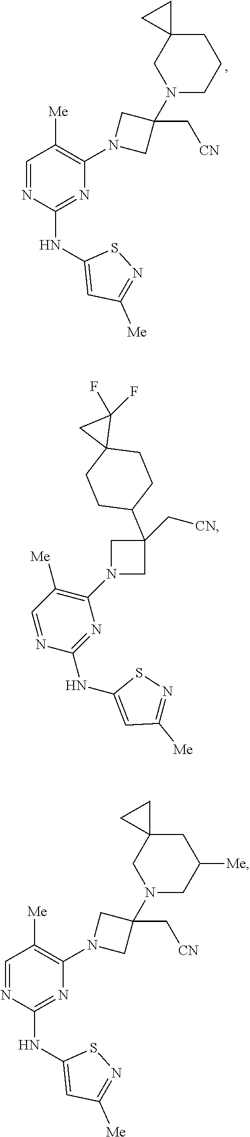 [SMILES: Cc1cc(Nc2ncc(C)c(N3CC(CC#N)(N4CCCC5(CC5)C4)C3)n2)sn1 ; Cc1cc(Nc2ncc(C)c(N3CC(CC#N)(C4CCC5(CC4)CC5(F)F)C3)n2)sn1 ; Cc1cc(Nc2ncc(C)c(N3CC(CC#N)(N4CC(C)CC5(CC5)C4)C3)n2)sn1] 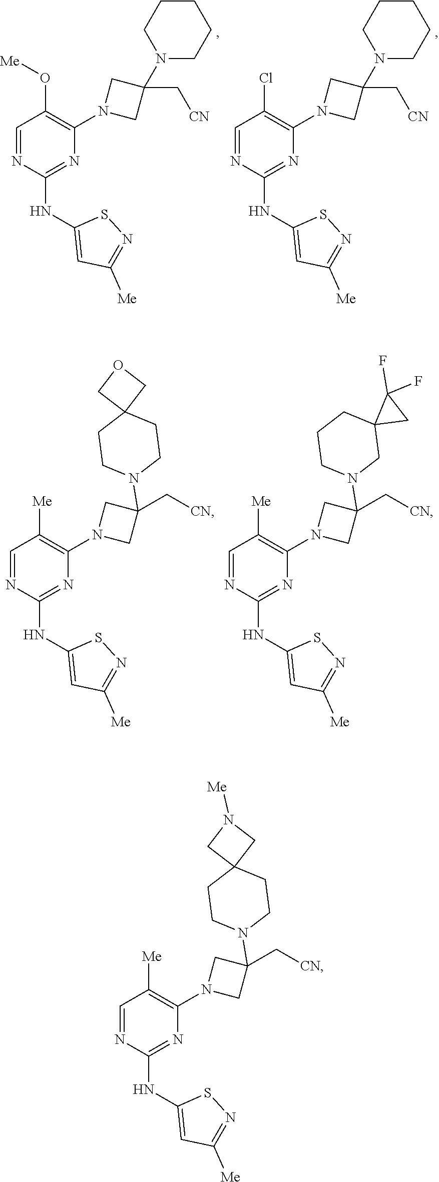 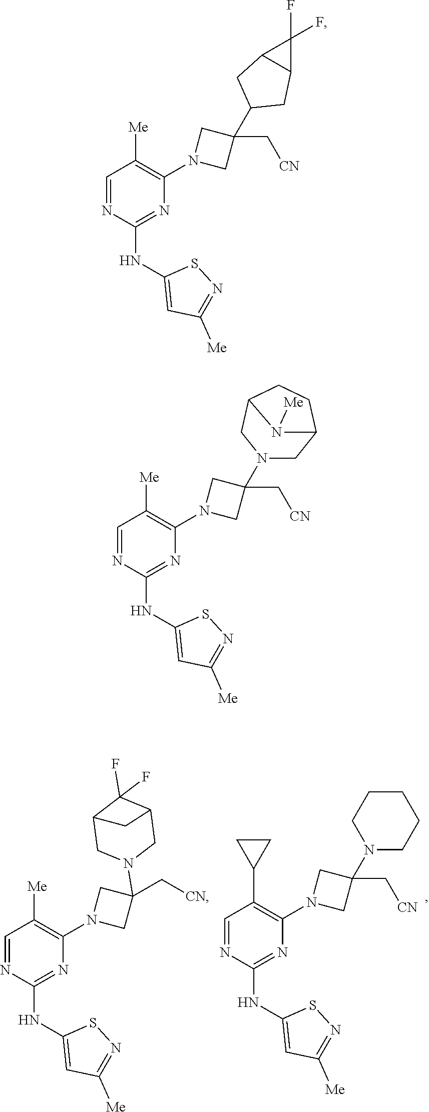 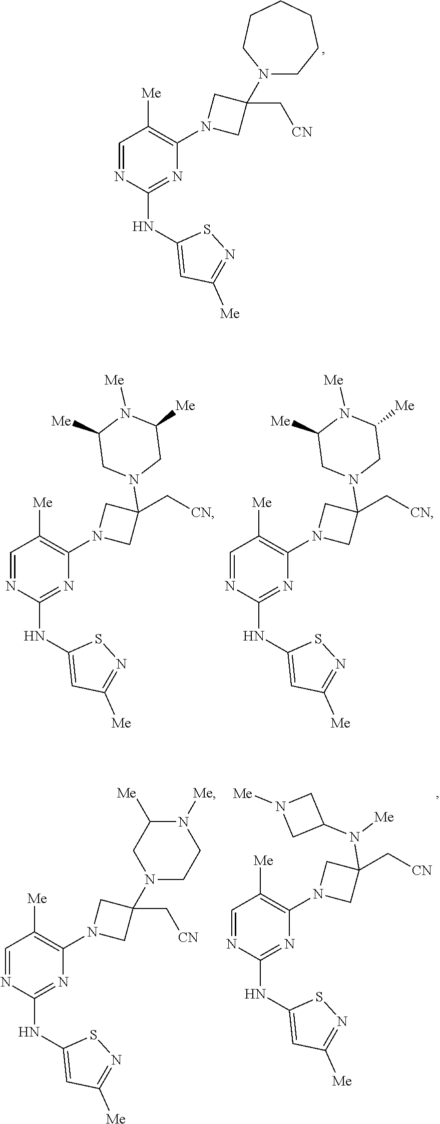 [SMILES: Cc1cc(Nc2ncc(C)c(N3CC(CC#N)(N4CCCCCC4)C3)n2)sn1 ; Cc1cc(Nc2ncc(C)c(N3CC(CC#N)(N4C[C@@H](C)N(C)[C@@H](C)C4)C3)n2)sn1 ; Cc1cc(Nc2ncc(C)c(N3CC(CC#N)(N4C[C@@H](C)N(C)[C@H](C)C4)C3)n2)sn1 ; Cc1cc(Nc2ncc(C)c(N3CC(CC#N)(N4CCN(C)C(C)C4)C3)n2)sn1 ; Cc1cc(Nc2ncc(C)c(N3CC(CC#N)(N(C)C4CN(C)C4)C3)n2)sn1] 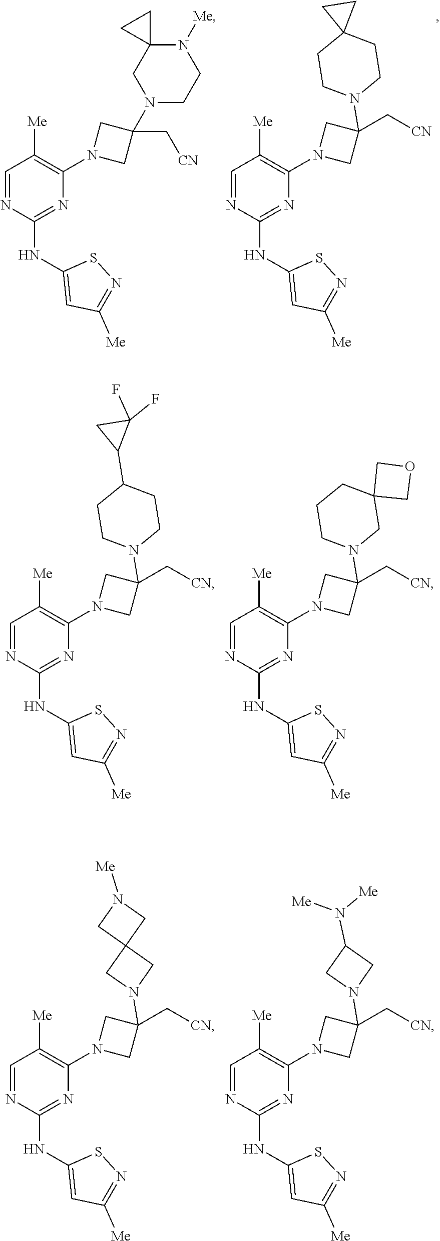 [SMILES: Cc1cc(Nc2ncc(C)c(N3CC(CC#N)(N4CCN(C)C5(CC5)C4)C3)n2)sn1 ; Cc1cc(Nc2ncc(C)c(N3CC(CC#N)(N4CCC5(CC4)CC5)C3)n2)sn1 ; Cc1cc(Nc2ncc(C)c(N3CC(CC#N)(N4CCC(C5CC5(F)F)CC4)C3)n2)sn1 ; Cc1cc(Nc2ncc(C)c(N3CC(CC#N)(N4CCCC5(COC5)C4)C3)n2)sn1 ; Cc1cc(Nc2ncc(C)c(N3CC(CC#N)(N4CC5(CN(C)C5)C4)C3)n2)sn1 ; Cc1cc(Nc2ncc(C)c(N3CC(CC#N)(N4CC(N(C)C)C4)C3)n2)sn1] 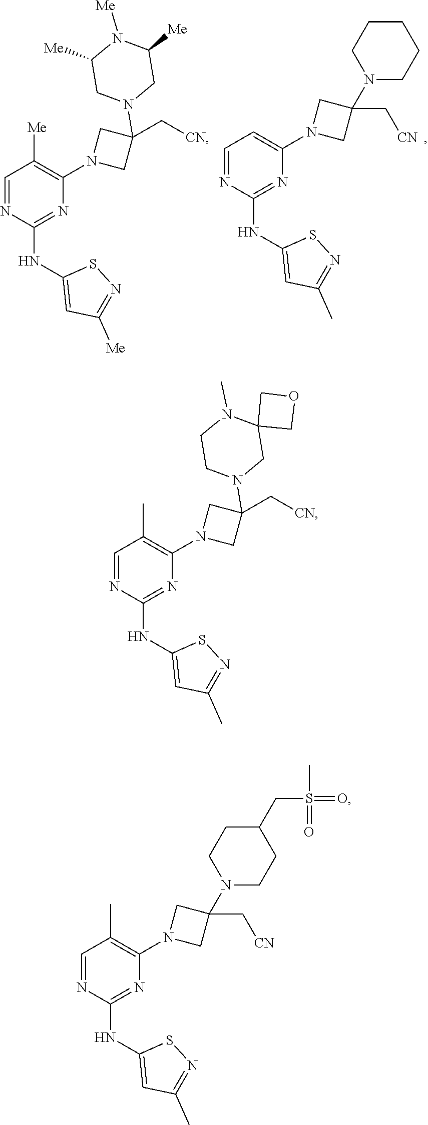 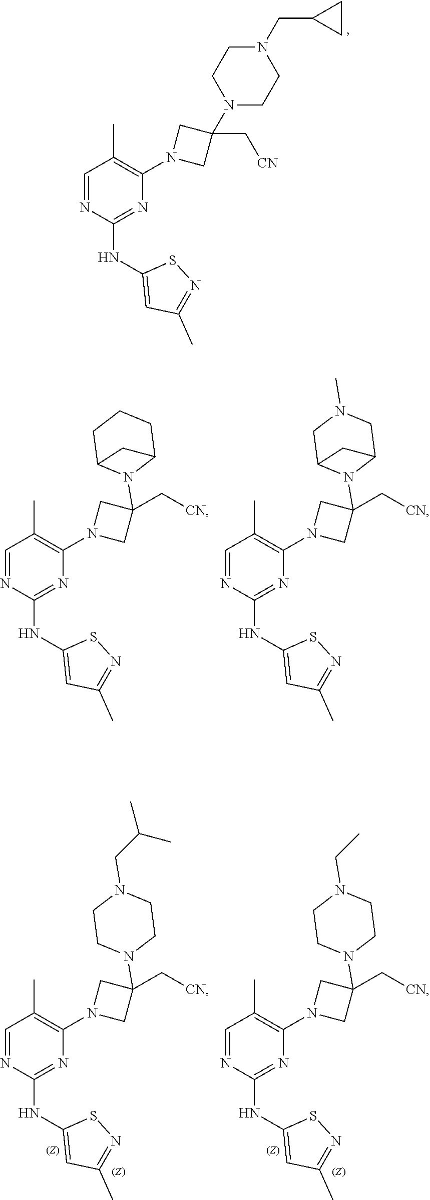 [SMILES: Cc1cc(Nc2ncc(C)c(N3CC(CC#N)(N4CCN(CC5CC5)CC4)C3)n2)sn1 ; Cc1cc(Nc2ncc(C)c(N3CC(CC#N)(N4C5CCCC4C5)C3)n2)sn1 ; Cc1cc(Nc2ncc(C)c(N3CC(CC#N)(N4C5CC4CN(C)C5)C3)n2)sn1 ; Cc1cc(Nc2ncc(C)c(N3CC(CC#N)(N4CCN(CC(C)C)CC4)C3)n2)sn1 ; CCN1CCN(C2(CC#N)CN(c3nc(Nc4cc(C)ns4)ncc3C)C2)CC1] 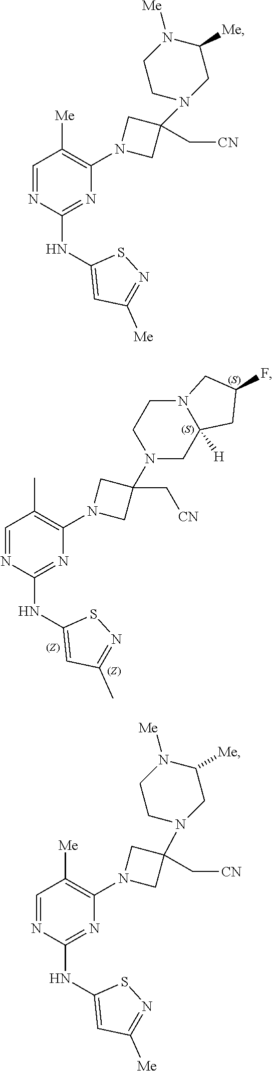 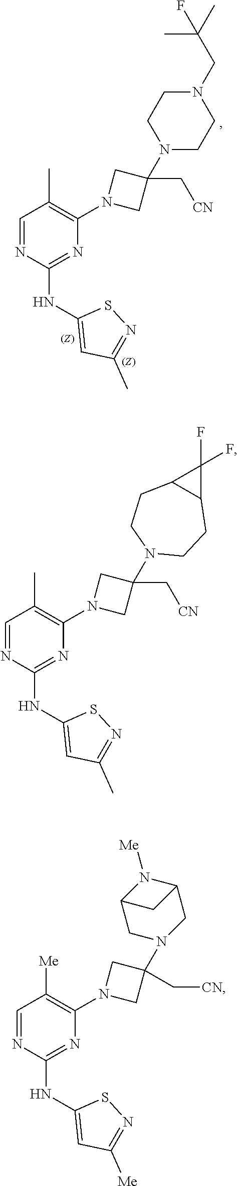 [SMILES: Cc1cc(Nc2ncc(C)c(N3CC(CC#N)(N4CCN(CC(C)(C)F)CC4)C3)n2)sn1 ; Cc1cc(Nc2ncc(C)c(N3CC(CC#N)(N4CCC5C(CC4)C5(F)F)C3)n2)sn1 ; Cc1cc(Nc2ncc(C)c(N3CC(CC#N)(N4CC5CC(C4)N5C)C3)n2)sn1] 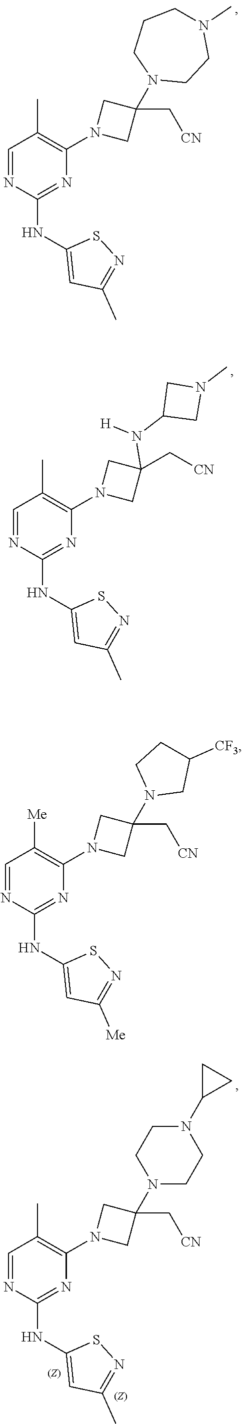 [SMILES: Cc1cc(Nc2ncc(C)c(N3CC(CC#N)(N4CCCN(C)CC4)C3)n2)sn1 ; Cc1cc(Nc2ncc(C)c(N3CC(CC#N)(NC4CN(C)C4)C3)n2)sn1 ; Cc1cc(Nc2ncc(C)c(N3CC(CC#N)(N4CCC(C(F)(F)F)C4)C3)n2)sn1 ; Cc1cc(Nc2ncc(C)c(N3CC(CC#N)(N4CCN(C5CC5)CC4)C3)n2)sn1] 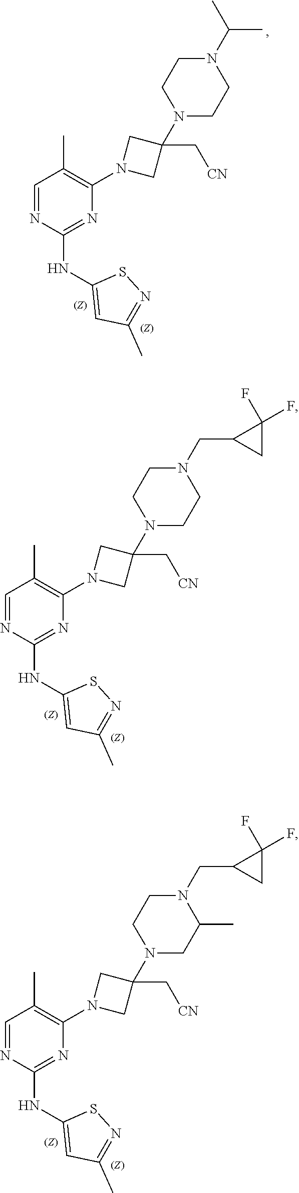 [SMILES: Cc1cc(Nc2ncc(C)c(N3CC(CC#N)(N4CCN(C(C)C)CC4)C3)n2)sn1 ; Cc1cc(Nc2ncc(C)c(N3CC(CC#N)(N4CCN(CC5CC5(F)F)CC4)C3)n2)sn1 ; Cc1cc(Nc2ncc(C)c(N3CC(CC#N)(N4CCN(CC5CC5(F)F)C(C)C4)C3)n2)sn1] 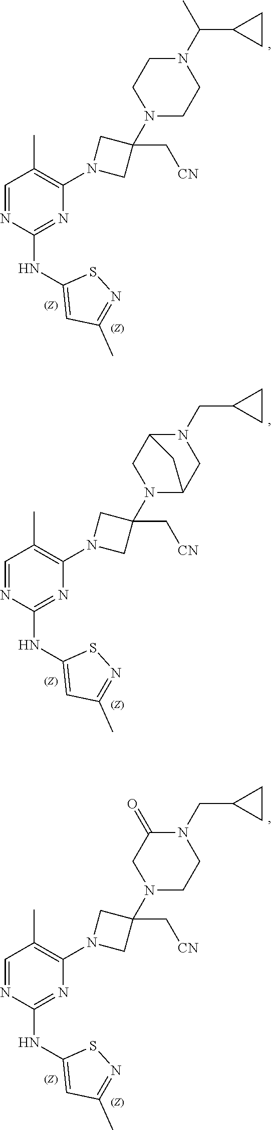 [SMILES: Cc1cc(Nc2ncc(C)c(N3CC(CC#N)(N4CCN(C(C)C5CC5)CC4)C3)n2)sn1 ; Cc1cc(Nc2ncc(C)c(N3CC(CC#N)(N4CC5CC4CN5CC4CC4)C3)n2)sn1 ; Cc1cc(Nc2ncc(C)c(N3CC(CC#N)(N4CCN(CC5CC5)C(=O)C4)C3)n2)sn1] 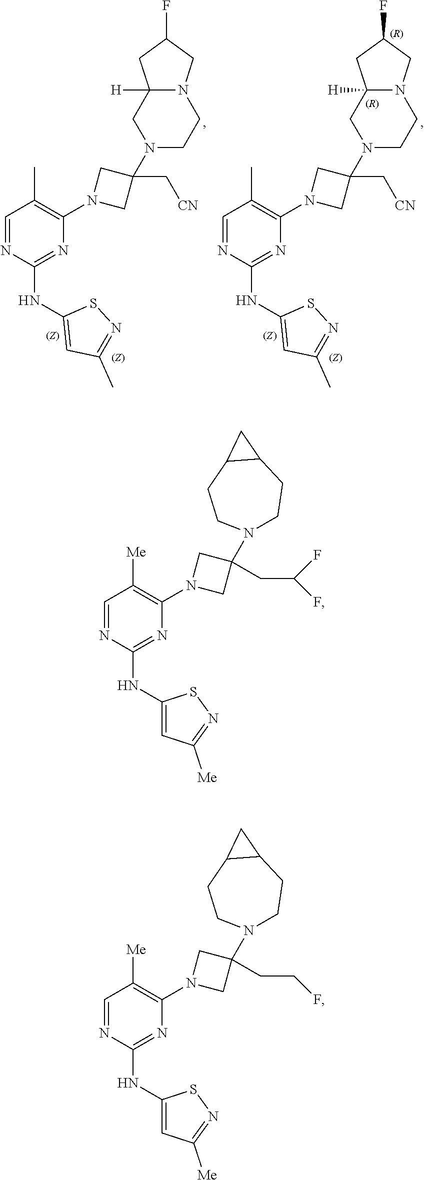 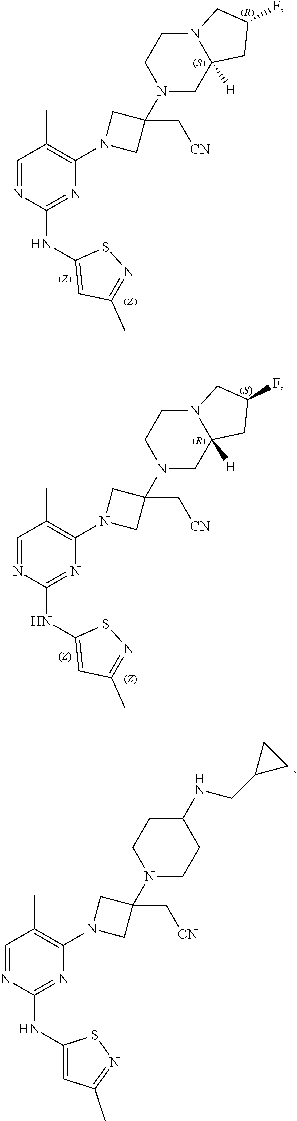 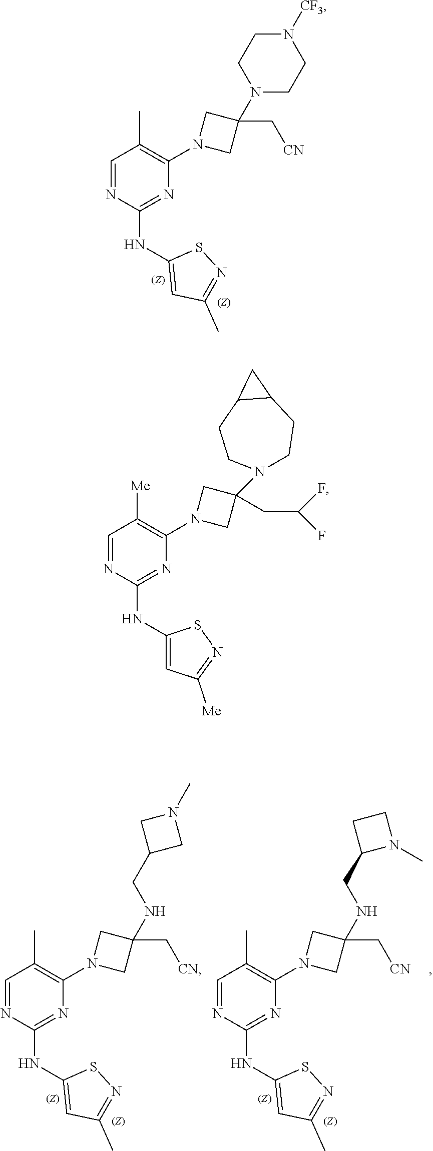 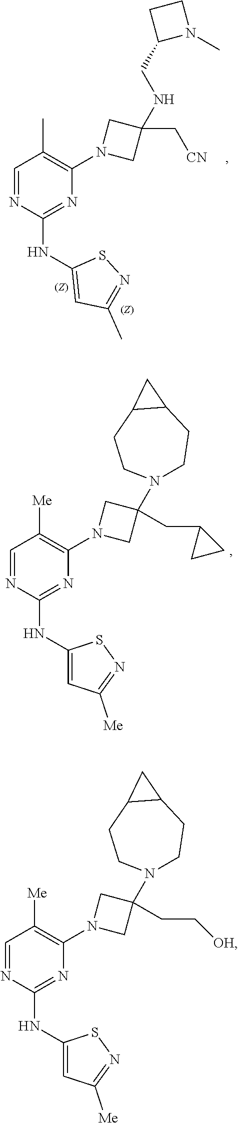 [SMILES: Cc1cc(Nc2ncc(C)c(N3CC(CC#N)(NC[C@@H]4CCN4C)C3)n2)sn1 ; Cc1cc(Nc2ncc(C)c(N3CC(CC4CC4)(N4CCC5CC5CC4)C3)n2)sn1 ; Cc1cc(Nc2ncc(C)c(N3CC(CCO)(N4CCC5CC5CC4)C3)n2)sn1] 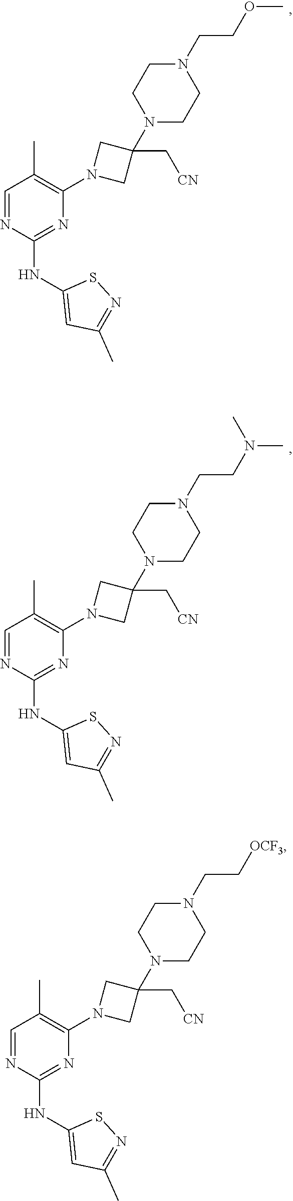 [SMILES: COCCN1CCN(C2(CC#N)CN(c3nc(Nc4cc(C)ns4)ncc3C)C2)CC1 ; Cc1cc(Nc2ncc(C)c(N3CC(CC#N)(N4CCN(CCN(C)C)CC4)C3)n2)sn1 ; Cc1cc(Nc2ncc(C)c(N3CC(CC#N)(N4CCN(CCOC(F)(F)F)CC4)C3)n2)sn1] 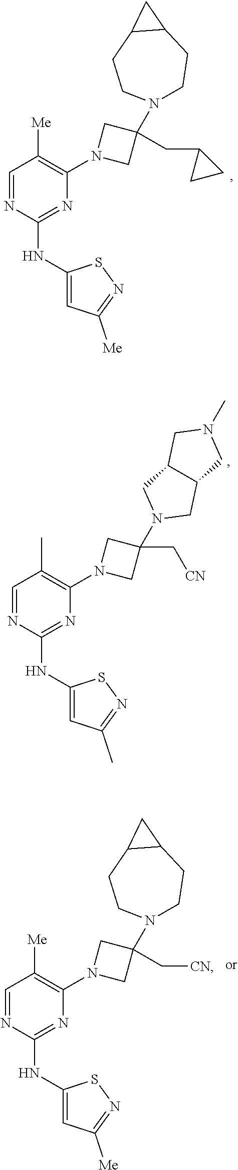 [SMILES: Cc1cc(Nc2ncc(C)c(N3CC(CC4CC4)(N4CCC5CC5CC4)C3)n2)sn1 ; Cc1cc(Nc2ncc(C)c(N3CC(CC#N)(N4C[C@H]5CN(C)C[C@H]5C4)C3)n2)sn1 ; Cc1cc(Nc2ncc(C)c(N3CC(CC#N)(N4CCC5CC5CC4)C3)n2)sn1 ; "or"] 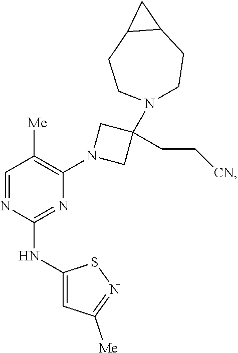 [SMILES: Cc1cc(Nc2ncc(C)c(N3CC(CCC#N)(N4CCC5CC5CC4)C3)n2)sn1]
or a pharmaceutically acceptable salt thereof; 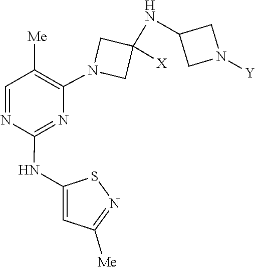
wherein,

| | |
|---|---|
| X is methyl and | Y is methyl, |
| X is ethyl and | Y is methyl, |
| X is cyclobutyl and | Y is methyl, |
| X is 3-fluorocyclobut-1-yl and | Y is methyl, |
| X is cyclopentyl and | Y is methyl, |
| X is tetrahydro-2H-pyran-4-yl and | Y is methyl, |
| X is isopropyl and | Y is methyl, |
| X is cyclohexyl and | Y is methyl, |
| X is cycloheptyl and | Y is methyl, |
| X is methylenecyclopropyl and | Y is methyl, |
| X is 2-fluoroethyl and | Y is methyl, |
| X is 2,2-difluoroethyl and | Y is methyl, |
| X is 2-fluoroethyl and | Y is ethyl, or |
| X is —CH₂CN and | Y is isopropyl, |

or a pharmaceutically acceptable salt thereof; 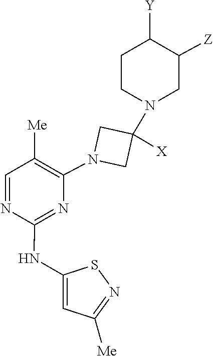
wherein,

| | | |
|---|---|---|
| X is —CH₂CN and | Y is fluoro and | Z is methyl, |
| X is —CH₂CH₂CN and | Y is fluoro and | Z is methyl, |
| X is 2-fluoroethyl and | Y is fluoro and | Z is methyl, |
| X is 3-fluorocyclobut-1-yl and | Y is fluoro and | Z is methyl, |
| X is cyclopentyl and | Y is fluoro and | Z is methyl, |
| X is tetrahydro-2H-pyran-4-yl and | Y is fluoro and | Z is methyl, |
| X is isopropyl and | Y is fluoro and | Z is methyl, |
| X is cyclohexyl and | Y is fluoro and | Z is methyl, |
| X is cycloheptyl and | Y is fluoro and | Z is methyl, |
| X is methylenecyclopropyl and | Y is fluoro and | Z is methyl, |
| X is 2-fluoroethyl and | Y is fluoro and | Z is fluoro, |
| X is 2,2-difluoroethyl and | Y is fluoro and | Z is methyl, |
| X is 2-fluoroethyl and | Y is fluoro and | Z is ethyl, or |
| X is —CH₂CN and | Y is fluoro and | Z is isopropyl, |

or a pharmaceutically acceptable salt thereof; 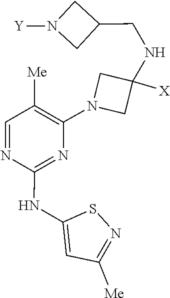
wherein,

| | |
|---|---|
| X is methyl and | Y is methyl, |
| X is ethyl and | Y is methyl, |
| X is cyclobutyl and | Y is methyl, |
| X is 3-fluorocyclobut-1-yl and | Y is methyl, |
| X is cyclopentyl and | Y is methyl, |
| X is tetrahydro-2H-pyran-4-yl and | Y is methyl, |
| X is isopropyl and | Y is methyl, |
| X is cyclohexyl and | Y is methyl, |
| X is cycloheptyl and | Y is methyl, |
| X is methylenecyclopropyl and | Y is methyl, |
| X is 2-fluoroethyl and | Y is methyl, |
| X is 2,2-difluoroethyl and | Y is methyl, |
| X is 2-fluoroethyl and | Y is ethyl, |
| X is —CH₂CN and | Y is isopropyl, or |
| X is —CH₂CN and | Y is methyl, |

or a pharmaceutically acceptable salt thereof; 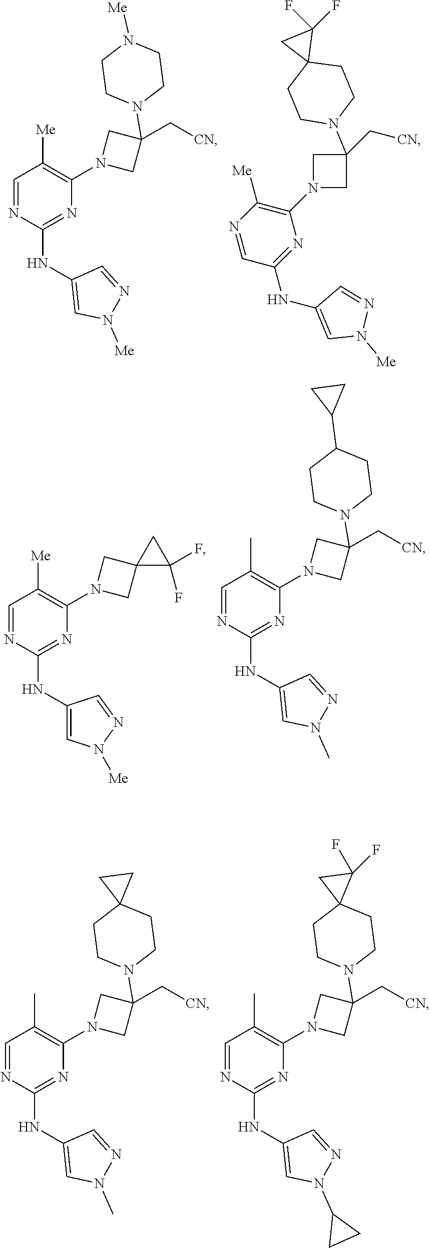 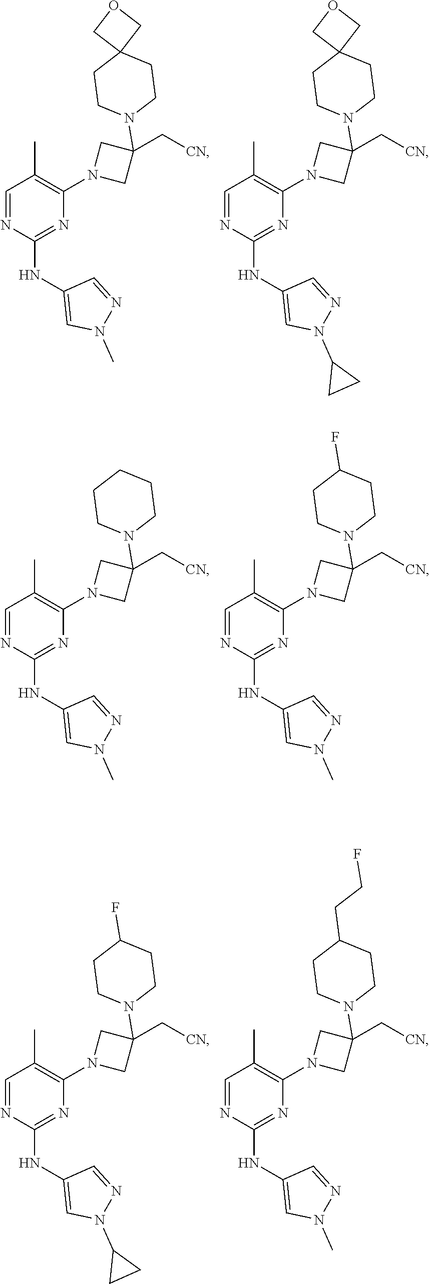 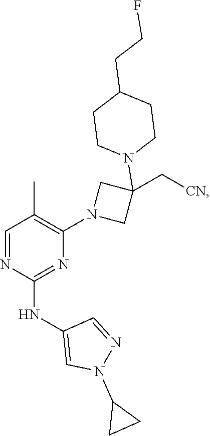 [SMILES: Cc1cnc(Nc2cnn(C3CC3)c2)nc1N1CC(CC#N)(N2CCC(CCF)CC2)C1] 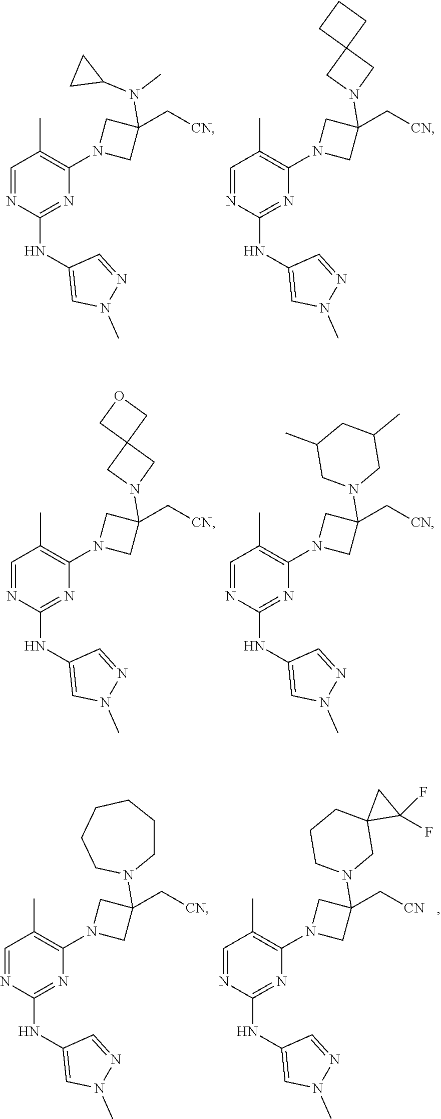 [SMILES: Cc1cnc(Nc2cnn(C)c2)nc1N1CC(CC#N)(N(C)C2CC2)C1 ; Cc1cnc(Nc2cnn(C)c2)nc1N1CC(CC#N)(N2CC3(CCC3)C2)C1 ; Cc1cnc(Nc2cnn(C)c2)nc1N1CC(CC#N)(N2CC3(COC3)C2)C1 ; Cc1cnc(Nc2cnn(C)c2)nc1N1CC(CC#N)(N2CC(C)CC(C)C2)C1 ; Cc1cnc(Nc2cnn(C)c2)nc1N1CC(CC#N)(N2CCCCCC2)C1 ; Cc1cnc(Nc2cnn(C)c2)nc1N1CC(CC#N)(N2CCCC3(C2)CC3(F)F)C1] 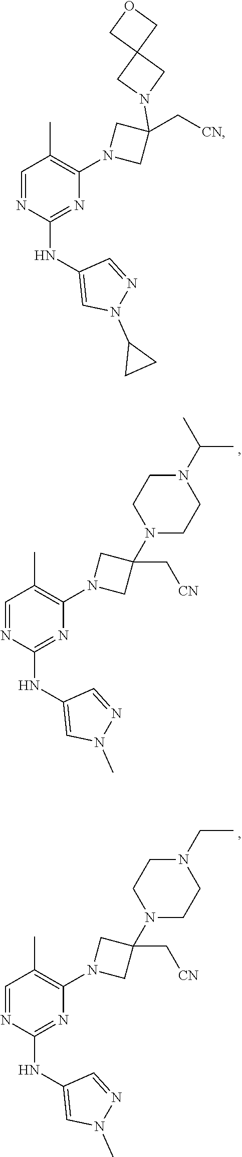 [SMILES: Cc1cnc(Nc2cnn(C3CC3)c2)nc1N1CC(CC#N)(N2CC3(COC3)C2)C1 ; Cc1cnc(Nc2cnn(C)c2)nc1N1CC(CC#N)(N2CCN(C(C)C)CC2)C1 ; CCN1CCN(C2(CC#N)CN(c3nc(Nc4cnn(C)c4)ncc3C)C2)CC1] 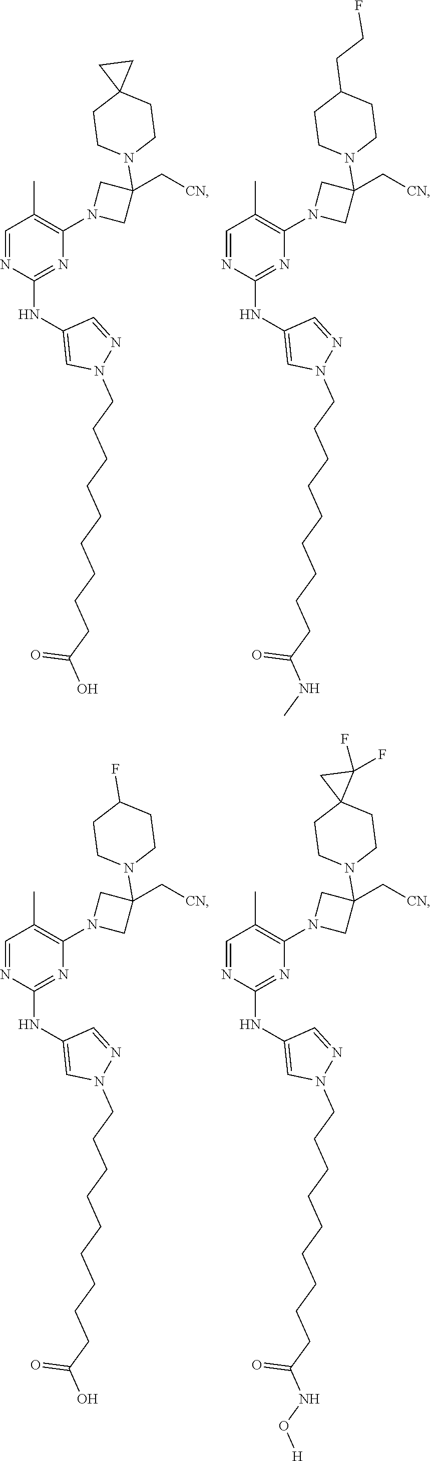 [SMILES: Cc1cnc(Nc2cnn(CCCCCCCCCC(=O)O)c2)nc1N1CC(CC#N)(N2CCC3(CC2)CC3)C1 ; CNC(=O)CCCCCCCCCn1cc(Nc2ncc(C)c(N3CC(CC#N)(N4CCC(CCF)CC4)C3)n2)cn1 ; Cc1cnc(Nc2cnn(CCCCCCCCCC(=O)O)c2)nc1N1CC(CC#N)(N2CCC(F)CC2)C1 ; Cc1cnc(Nc2cnn(CCCCCCCCCC(=O)NO)c2)nc1N1CC(CC#N)(N2CCC3(CC2)CC3(F)F)C1] 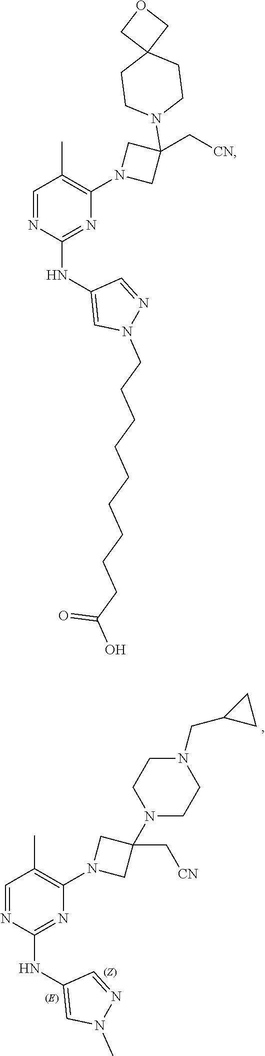 [SMILES: Cc1cnc(Nc2cnn(CCCCCCCCCC(=O)O)c2)nc1N1CC(CC#N)(N2CCC3(CC2)COC3)C1 ; Cc1cnc(Nc2cnn(C)c2)nc1N1CC(CC#N)(N2CCN(CC3CC3)CC2)C1] 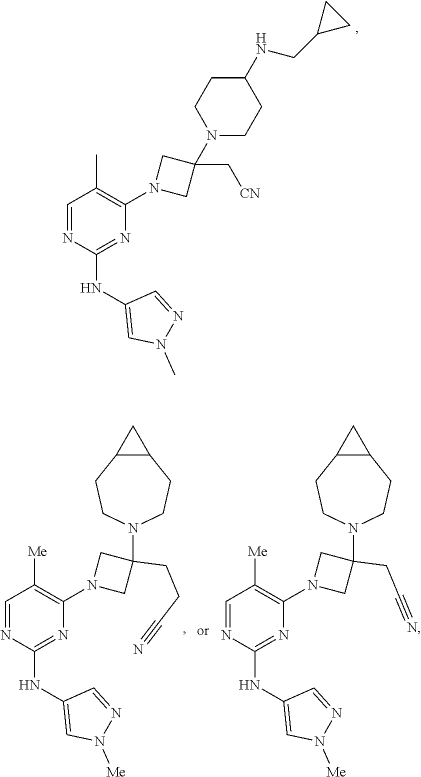 [SMILES: Cc1cnc(Nc2cnn(C)c2)nc1N1CC(CC#N)(N2CCC(NCC3CC3)CC2)C1 ; Cc1cnc(Nc2cnn(C)c2)nc1N1CC(CCC#N)(N2CCC3CC3CC2)C1 ; Cc1cnc(Nc2cnn(C)c2)nc1N1CC(CC#N)(N2CCC3CC3CC2)C1]
or a pharmaceutically acceptable salt thereof; 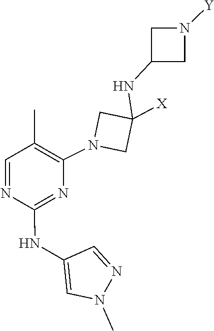
wherein,

| | |
|---|---|
| X is methyl and | Y is methyl, |
| X is ethyl and | Y is methyl, |
| X is cyclobutyl and | Y is methyl, |
| X is 3-fluorocyclobut-1-yl and | Y is methyl, |
| X is cyclopentyl and | Y is methyl, |
| X is tetrahydro-2H-pyran-4-yl and | Y is methyl, |
| X is isopropyl and | Y is methyl, |
| X is cyclohexyl and | Y is methyl, |
| X is cycloheptyl and | Y is methyl, |
| X is methylenecyclopropyl and | Y is methyl, |
| X is 2-fluoroethyl and | Y is methyl, |
| X is 2,2-difluoroethyl and | Y is methyl, |
| X is 2-fluoroethyl and | Y is ethyl, or |
| X is —CH₂CN and | Y is isopropyl, |

or a pharmaceutically acceptable salt thereof; 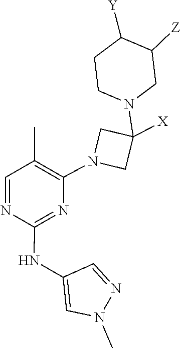
wherein,

| | | |
|---|---|---|
| X is —CH₂CN and | Y is fluoro and | Z is methyl, |
| X is —CH₂CH₂CN and | Y is fluoro and | Z is methyl, |
| X is 2-fluoroethyl and | Y is fluoro and | Z is methyl, |
| X is 3-fluorocyclobut-1-yl and | Y is fluoro and | Z is methyl, |
| X is cyclopentyl and | Y is fluoro and | Z is methyl, |
| X is tetrahydro-2H-pyran-4-yl and | Y is fluoro and | Z is methyl, |
| X is isopropyl and | Y is fluoro and | Z is methyl, |
| X is cyclohexyl and | Y is fluoro and | Z is methyl, |
| X is cycloheptyl and | Y is fluoro and | Z is methyl, |
| X is methylenecyclopropyl and | Y is fluoro and | Z is methyl, |
| X is 2-fluoroethyl and | Y is fluoro and | Z is fluoro, |
| X is 2,2-difluoroethyl and | Y is fluoro and | Z is methyl, |
| X is 2-fluoroethyl and | Y is fluoro and | Z is ethyl, or |
| X is —CH₂CN and | Y is fluoro and | Z is isopropyl, |

or a pharmaceutically acceptable salt thereof; 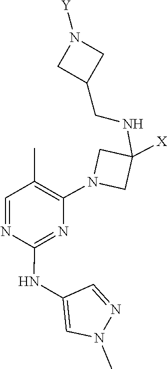
wherein,

| | |
|---|---|
| X is methyl and | Y is methyl, |
| X is ethyl and | Y is methyl, |
| X is cyclobutyl and | Y is methyl, |
| X is 3-fluorocyclobut-1-yl and | Y is methyl, |
| X is cyclopentyl and | Y is methyl, |
| X is tetrahydro-2H-pyran-4-yl and | Y is methyl, |
| X is isopropyl and | Y is methyl, |
| X is cyclohexyl and | Y is methyl, |
| X is cycloheptyl and | Y is methyl, |
| X is methylenecyclopropyl and | Y is methyl, |
| X is 2-fluoroethyl and | Y is methyl, |
| X is 2,2-difluoroethyl and | Y is methyl, |
| X is 2-fluoroethyl and | Y is ethyl, or |
| X is —CH₂CN and | Y is isopropyl, |

or a pharmaceutically acceptable salt thereof; 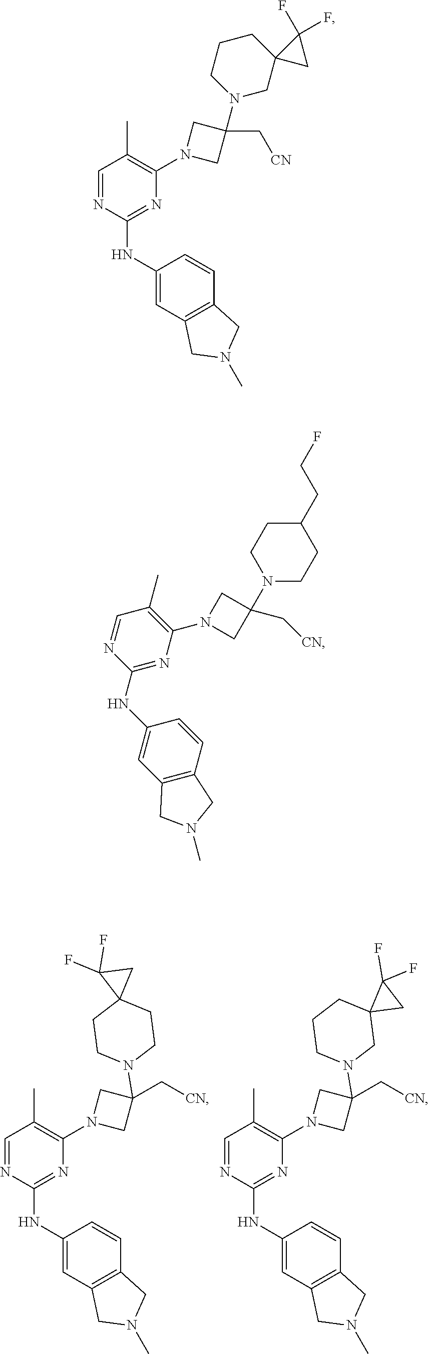 [SMILES: Cc1cnc(Nc2ccc3c(c2)CN(C)C3)nc1N1CC(CC#N)(N2CCCC3(C2)CC3(F)F)C1 ; Cc1cnc(Nc2ccc3c(c2)CN(C)C3)nc1N1CC(CC#N)(N2CCC(CCF)CC2)C1 ; Cc1cnc(Nc2ccc3c(c2)CN(C)C3)nc1N1CC(CC#N)(N2CCC3(CC2)CC3(F)F)C1 ; Cc1cnc(Nc2ccc3c(c2)CN(C)C3)nc1N1CC(CC#N)(N2CCCC3(C2)CC3(F)F)C1] 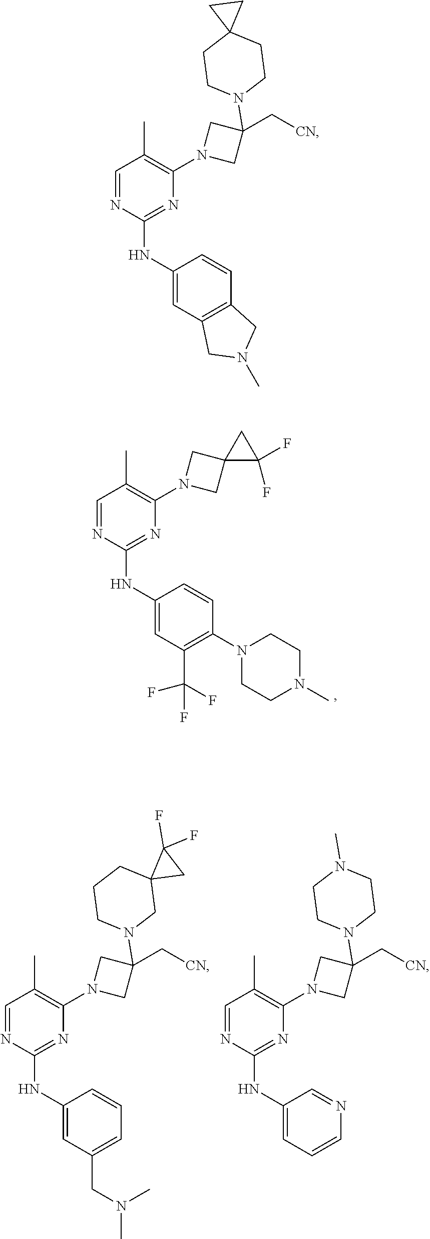 [SMILES: Cc1cnc(Nc2ccc3c(c2)CN(C)C3)nc1N1CC(CC#N)(N2CCC3(CC2)CC3)C1 ; Cc1cnc(Nc2ccc(N3CCN(C)CC3)c(C(F)(F)F)c2)nc1N1CC2(C1)CC2(F)F ; Cc1cnc(Nc2cccc(CN(C)C)c2)nc1N1CC(CC#N)(N2CCCC3(C2)CC3(F)F)C1 ; Cc1cnc(Nc2cccnc2)nc1N1CC(CC#N)(N2CCN(C)CC2)C1] 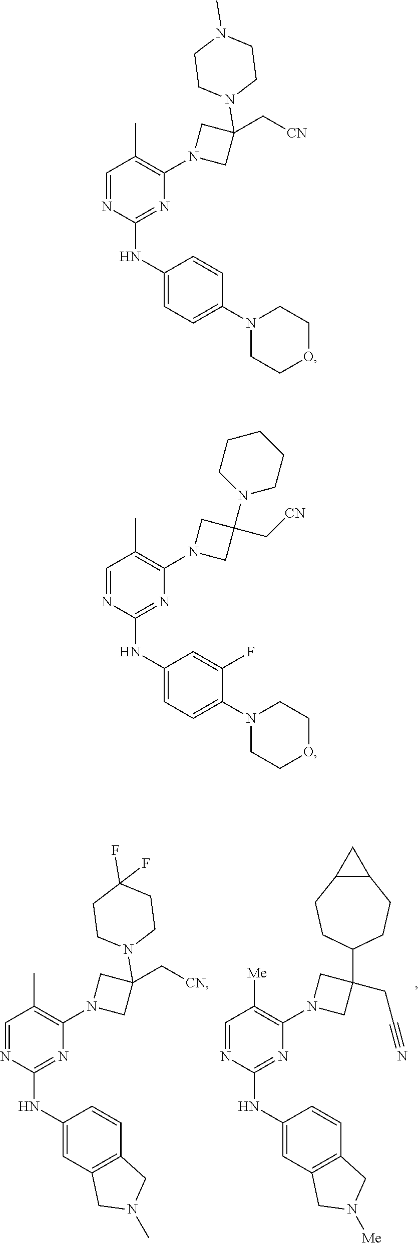 [SMILES: Cc1cnc(Nc2ccc(N3CCOCC3)cc2)nc1N1CC(CC#N)(N2CCN(C)CC2)C1 ; Cc1cnc(Nc2ccc(N3CCOCC3)c(F)c2)nc1N1CC(CC#N)(N2CCCCC2)C1 ; Cc1cnc(Nc2ccc3c(c2)CN(C)C3)nc1N1CC(CC#N)(N2CCC(F)(F)CC2)C1 ; Cc1cnc(Nc2ccc3c(c2)CN(C)C3)nc1N1CC(CC#N)(C2CCC3CC3CC2)C1] 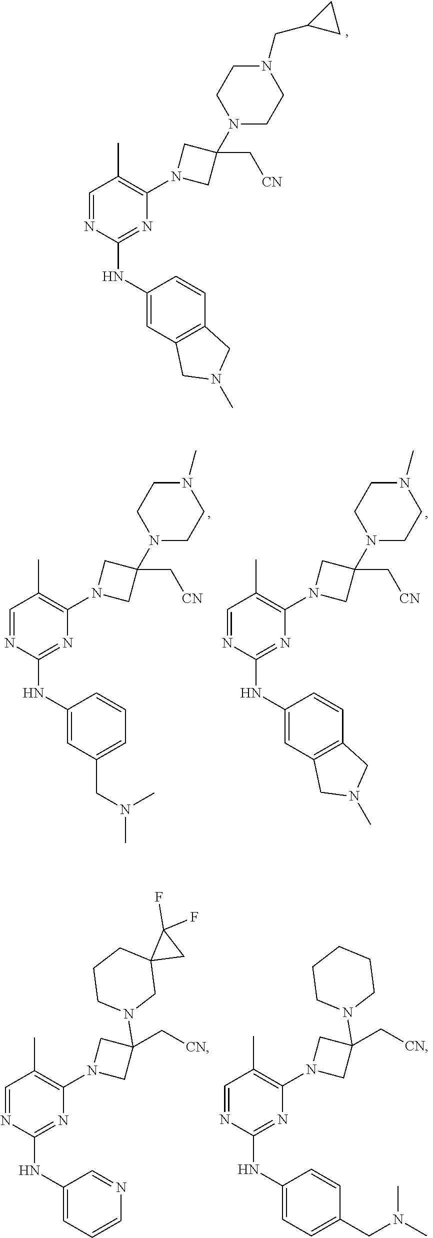 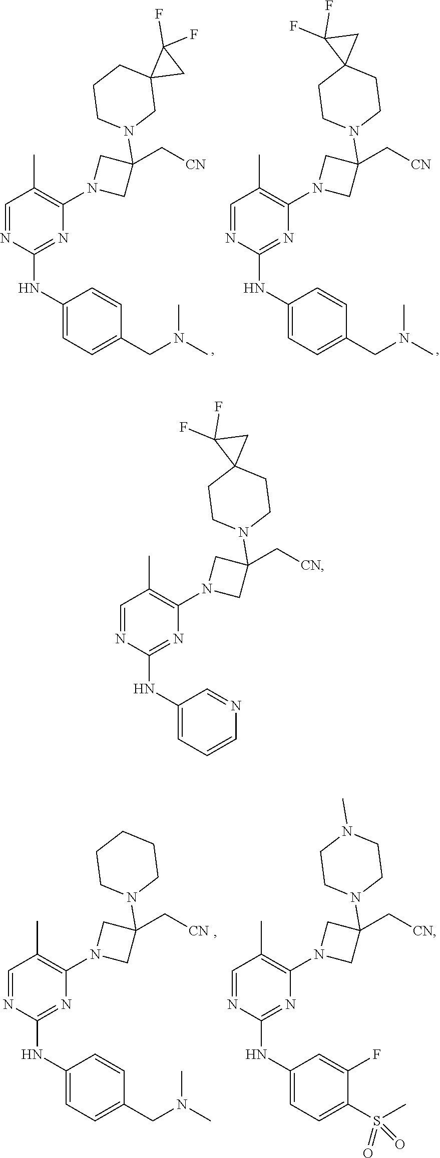 [SMILES: Cc1cnc(Nc2ccc(CN(C)C)cc2)nc1N1CC(CC#N)(N2CCCC3(C2)CC3(F)F)C1 ; Cc1cnc(Nc2ccc(CN(C)C)cc2)nc1N1CC(CC#N)(N2CCC3(CC2)CC3(F)F)C1 ; Cc1cnc(Nc2cccnc2)nc1N1CC(CC#N)(N2CCC3(CC2)CC3(F)F)C1 ; Cc1cnc(Nc2ccc(CN(C)C)cc2)nc1N1CC(CC#N)(N2CCCCC2)C1 ; Cc1cnc(Nc2ccc(S(C)(=O)=O)c(F)c2)nc1N1CC(CC#N)(N2CCN(C)CC2)C1] 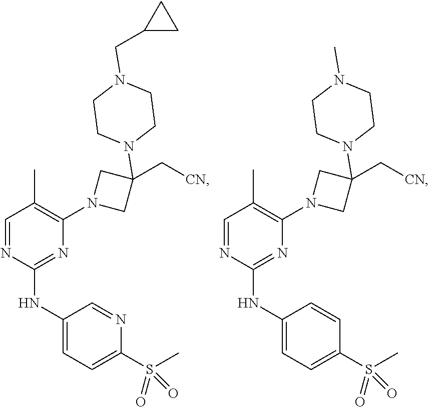 [SMILES: Cc1cnc(Nc2ccc(S(C)(=O)=O)nc2)nc1N1CC(CC#N)(N2CCN(CC3CC3)CC2)C1 ; Cc1cnc(Nc2ccc(S(C)(=O)=O)cc2)nc1N1CC(CC#N)(N2CCN(C)CC2)C1] 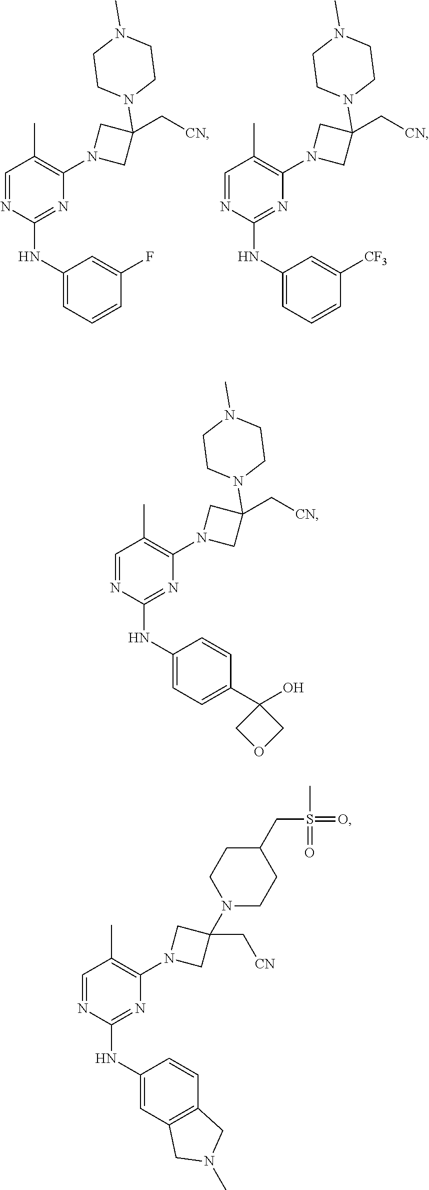 [SMILES: Cc1cnc(Nc2cccc(F)c2)nc1N1CC(CC#N)(N2CCN(C)CC2)C1 ; Cc1cnc(Nc2cccc(C(F)(F)F)c2)nc1N1CC(CC#N)(N2CCN(C)CC2)C1 ; Cc1cnc(Nc2ccc(C3(O)COC3)cc2)nc1N1CC(CC#N)(N2CCN(C)CC2)C1 ; Cc1cnc(Nc2ccc3c(c2)CN(C)C3)nc1N1CC(CC#N)(N2CCC(CS(C)(=O)=O)CC2)C1] 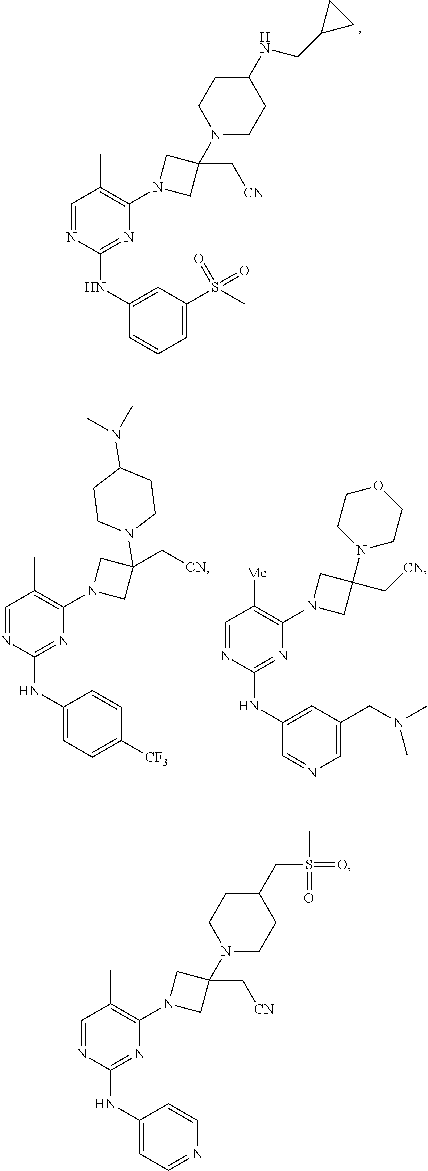 [SMILES: Cc1cnc(Nc2cccc(S(C)(=O)=O)c2)nc1N1CC(CC#N)(N2CCC(NCC3CC3)CC2)C1 ; Cc1cnc(Nc2ccc(C(F)(F)F)cc2)nc1N1CC(CC#N)(N2CCC(N(C)C)CC2)C1 ; Cc1cnc(Nc2cncc(CN(C)C)c2)nc1N1CC(CC#N)(N2CCOCC2)C1 ; Cc1cnc(Nc2ccncc2)nc1N1CC(CC#N)(N2CCC(CS(C)(=O)=O)CC2)C1] 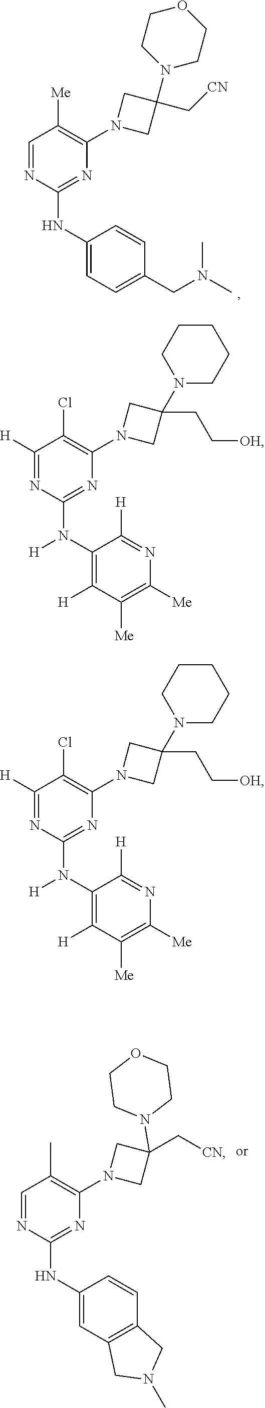 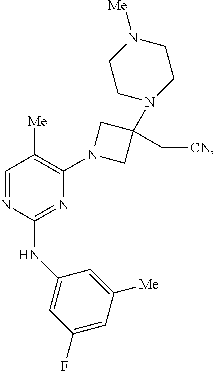 [SMILES: Cc1cc(F)cc(Nc2ncc(C)c(N3CC(CC#N)(N4CCN(C)CC4)C3)n2)c1]
or a pharmaceutically acceptable salt thereof; 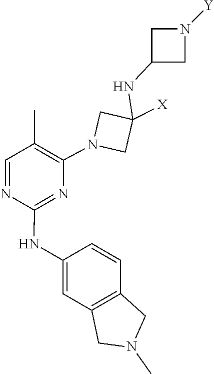
wherein,

| | |
|---|---|
| X is methyl and | Y is methyl, |
| X is ethyl and | Y is methyl, |
| X is cyclobutyl and | Y is methyl, |
| X is 3-fluorocyclobut-1-yl and | Y is methyl, |
| X is cyclopentyl and | Y is methyl, |
| X is tetrahydro-2H-pyran-4-yl and | Y is methyl, |
| X is isopropyl and | Y is methyl, |
| X is cyclohexyl and | Y is methyl, |
| X is cycloheptyl and | Y is methyl, |
| X is methylenecyclopropyl and | Y is methyl, |
| X is 2-fluoroethyl and | Y is methyl, |
| X is 2,2-difluoroethyl and | Y is methyl, |
| X is 2-fluoroethyl and | Y is ethyl, or |
| X is —CH₂CN and | Y is isopropyl, |

or a pharmaceutically acceptable salt thereof; 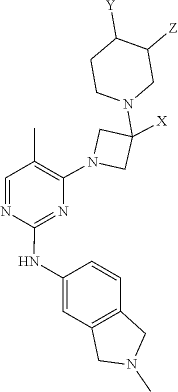
wherein,

| | | |
|---|---|---|
| X is —CH₂CN and | Y is fluoro and | Z is methyl, |
| X is —CH₂CH₂CN and | Y is fluoro and | Z is methyl, |
| X is 2-fluoroethyl and | Y is fluoro and | Z is methyl, |
| X is 3-fluorocyclobut-1-yl and | Y is fluoro and | Z is methyl, |
| X is cyclopentyl and | Y is fluoro and | Z is methyl, |
| X is tetrahydro-2H-pyran-4-yl and | Y is fluoro and | Z is methyl, |
| X is isopropyl and | Y is fluoro and | Z is methyl, |
| X is cyclohexyl and | Y is fluoro and | Z is methyl, |
| X is cycloheptyl and | Y is fluoro and | Z is methyl, |
| X is methylenecyclopropyl and | Y is fluoro and | Z is methyl, |
| X is 2-fluoroethyl and | Y is fluoro and | Z is fluoro, |
| X is 2,2-difluoroethyl and | Y is fluoro and | Z is methyl, |
| X is 2-fluoroethyl and | Y is fluoro and | Z is ethyl, |
| X is —CH₂CN and | Y is fluoro and | Z is isopropyl, or |
| X is —CH₂CN and | Y is chloro and | Z is methyl, |

or a pharmaceutically acceptable salt thereof; 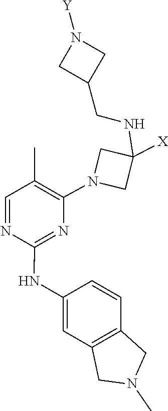
wherein,

| | |
|---|---|
| X is methyl and | Y is methyl, |
| X is ethyl and | Y is methyl, |
| X is cyclobutyl and | Y is methyl, |
| X is 3-fluorocyclobut-1-yl and | Y is methyl, |
| X is cyclopentyl and | Y is methyl, |
| X is tetrahydro-2H-pyran-4-yl and | Y is methyl, |
| X is isopropyl and | Y is methyl, |
| X is cyclohexyl and | Y is methyl, |
| X is cycloheptyl and | Y is methyl, |
| X is methylenecyclopropyl and | Y is methyl, |
| X is 2-fluoroethyl and | Y is methyl, |
| X is 2,2-difluoroethyl and | Y is methyl, |
| X is 2-fluoroethyl and | Y is ethyl, |
| X is —CH₂CN and | Y is isopropyl, or |
| X is —CH₂CN and | Y is methyl, |

or a pharmaceutically acceptable salt thereof; or 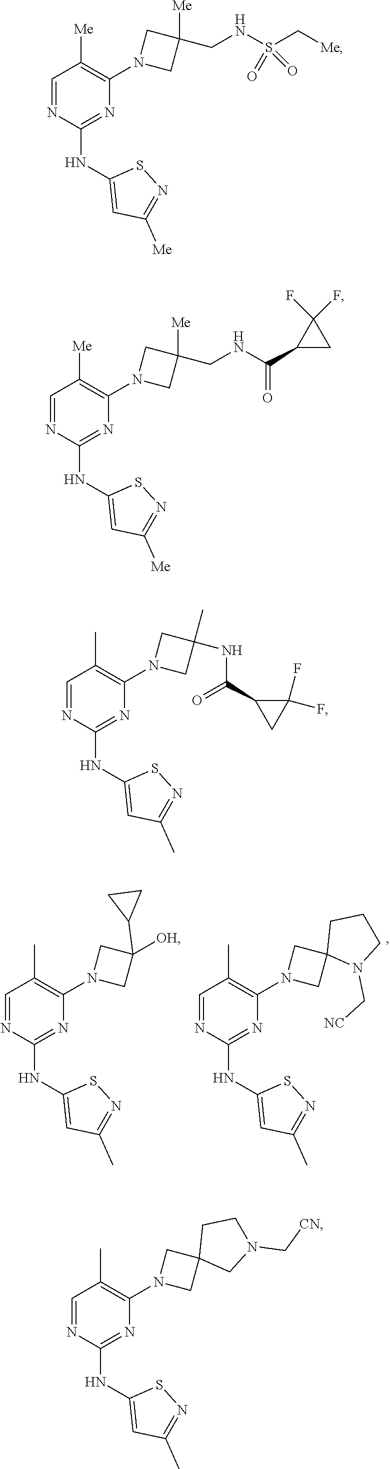 [SMILES: CCS(=O)(=O)NCC1(C)CN(c2nc(Nc3cc(C)ns3)ncc2C)C1 ; Cc1cc(Nc2ncc(C)c(N3CC(C)(CNC(=O)[C@@H]4CC4(F)F)C3)n2)sn1 ; Cc1cc(Nc2ncc(C)c(N3CC(C)(NC(=O)[C@@H]4CC4(F)F)C3)n2)sn1 ; Cc1cc(Nc2ncc(C)c(N3CC(O)(C4CC4)C3)n2)sn1 ; Cc1cc(Nc2ncc(C)c(N3CC4(CCCN4CC#N)C3)n2)sn1 ; Cc1cc(Nc2ncc(C)c(N3CC4(CCN(CC#N)C4)C3)n2)sn1] 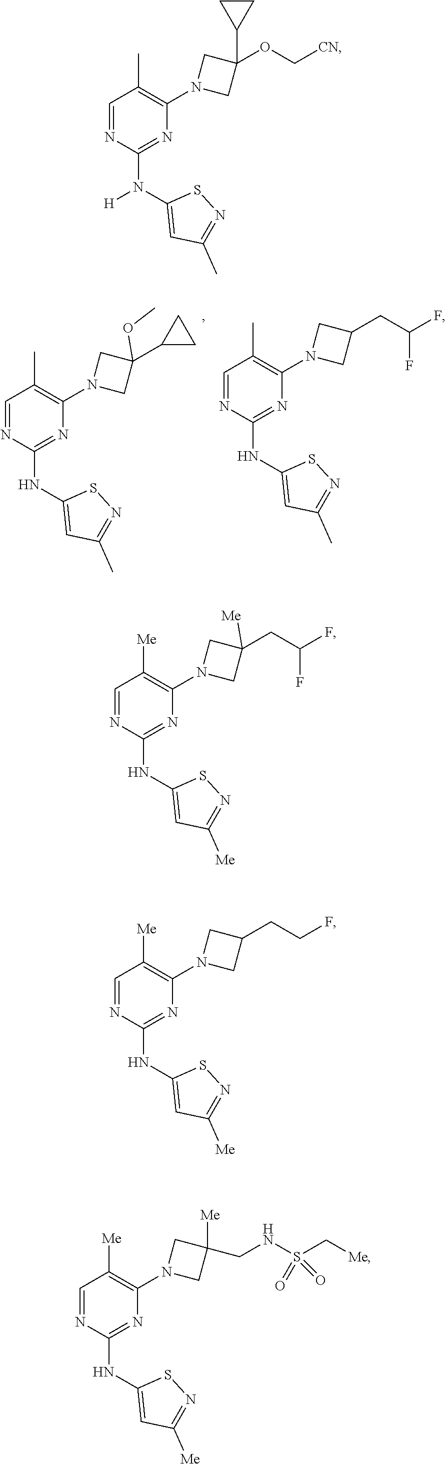 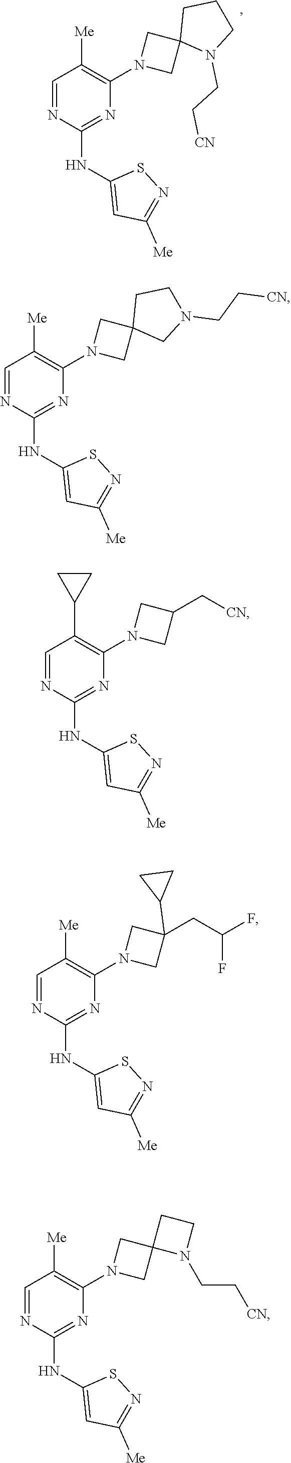 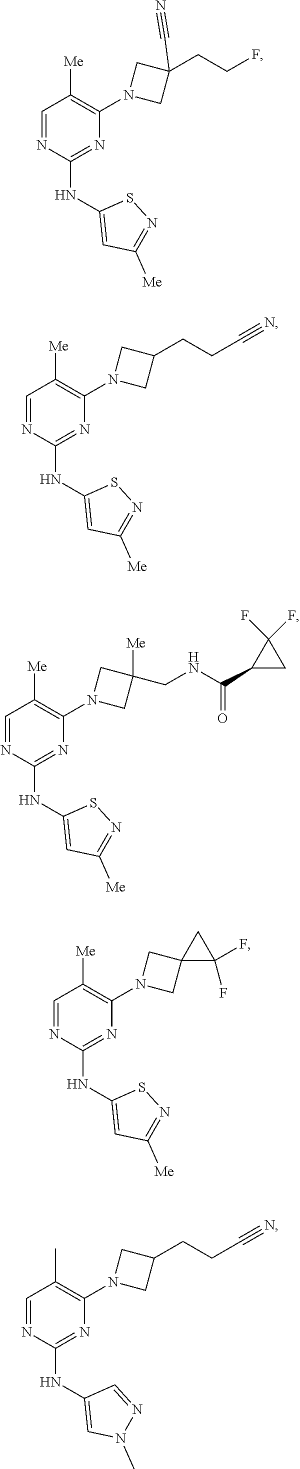 [SMILES: Cc1cc(Nc2ncc(C)c(N3CC(C#N)(CCF)C3)n2)sn1 ; Cc1cc(Nc2ncc(C)c(N3CC(CCC#N)C3)n2)sn1 ; Cc1cc(Nc2ncc(C)c(N3CC(C)(CNC(=O)[C@@H]4CC4(F)F)C3)n2)sn1 ; Cc1cc(Nc2ncc(C)c(N3CC4(C3)CC4(F)F)n2)sn1 ; Cc1cnc(Nc2cnn(C)c2)nc1N1CC(CCC#N)C1] 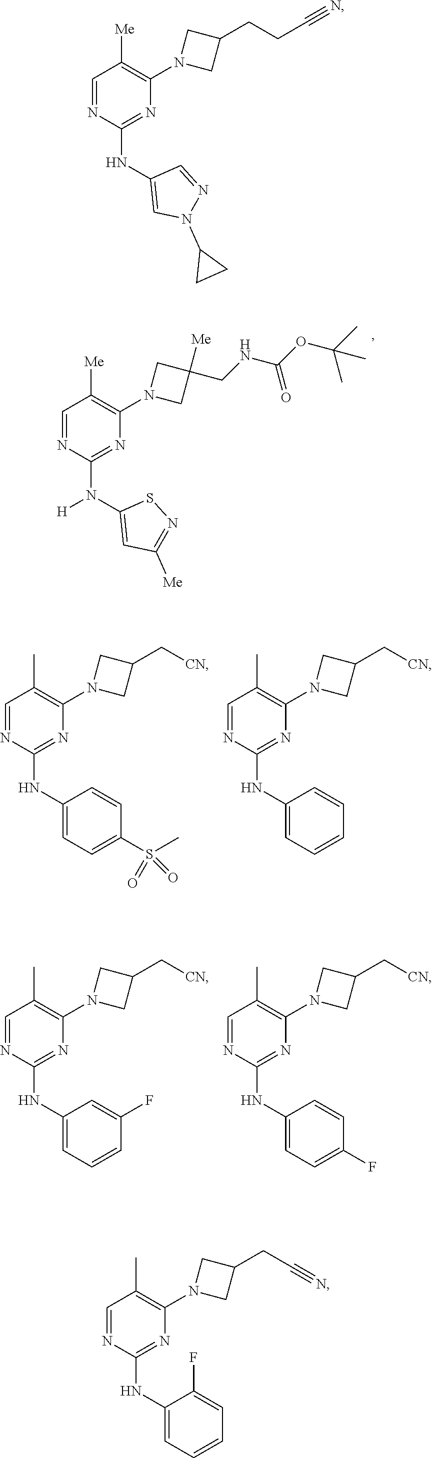 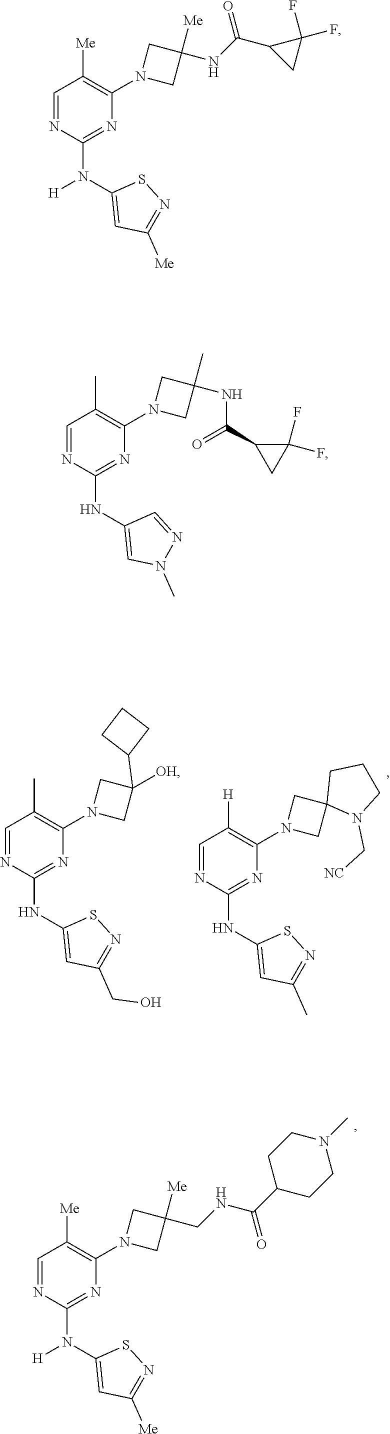 [SMILES: Cc1cc(Nc2ncc(C)c(N3CC(C)(NC(=O)C4CC4(F)F)C3)n2)sn1 ; Cc1cnc(Nc2cnn(C)c2)nc1N1CC(C)(NC(=O)[C@@H]2CC2(F)F)C1 ; Cc1cnc(Nc2cc(CO)ns2)nc1N1CC(O)(C2CCC2)C1 ; Cc1cc(Nc2nccc(N3CC4(CCCN4CC#N)C3)n2)sn1 ; Cc1cc(Nc2ncc(C)c(N3CC(C)(CNC(=O)C4CCN(C)CC4)C3)n2)sn1] 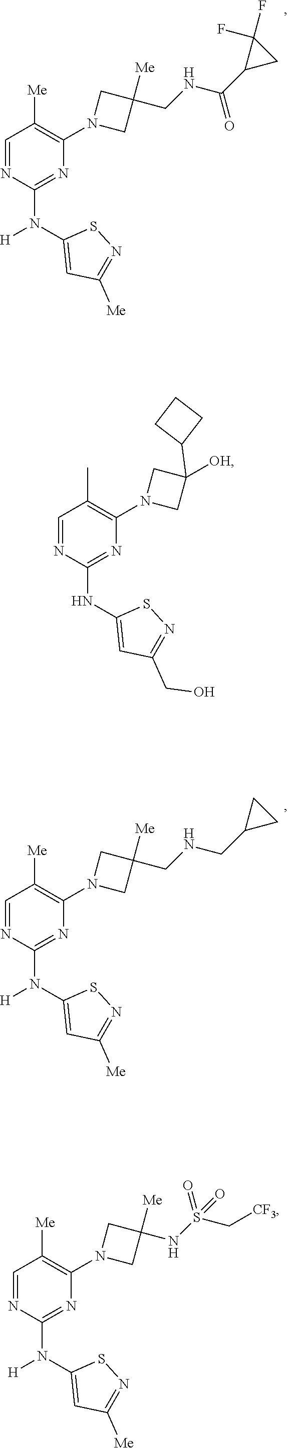 [SMILES: Cc1cc(Nc2ncc(C)c(N3CC(C)(CNC(=O)C4CC4(F)F)C3)n2)sn1 ; Cc1cnc(Nc2cc(CO)ns2)nc1N1CC(O)(C2CCC2)C1 ; Cc1cc(Nc2ncc(C)c(N3CC(C)(CNCC4CC4)C3)n2)sn1 ; Cc1cc(Nc2ncc(C)c(N3CC(C)(NS(=O)(=O)CC(F)(F)F)C3)n2)sn1] 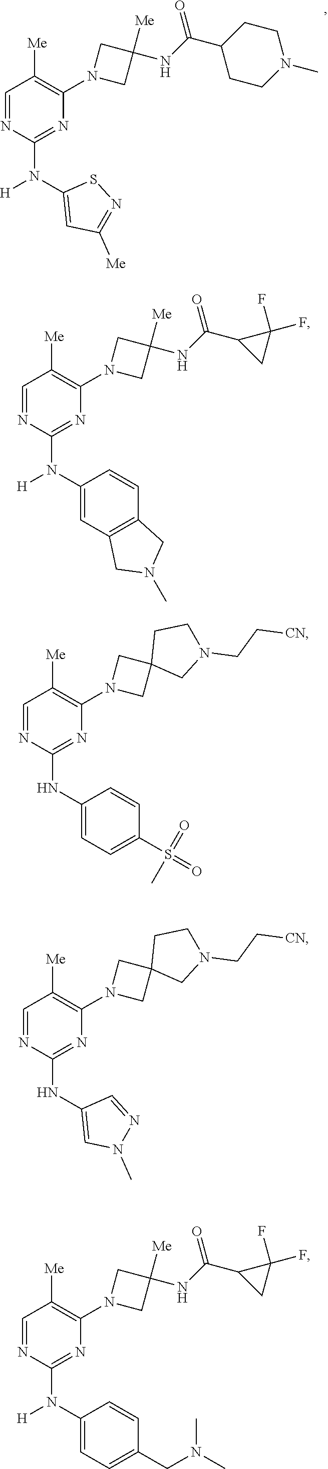 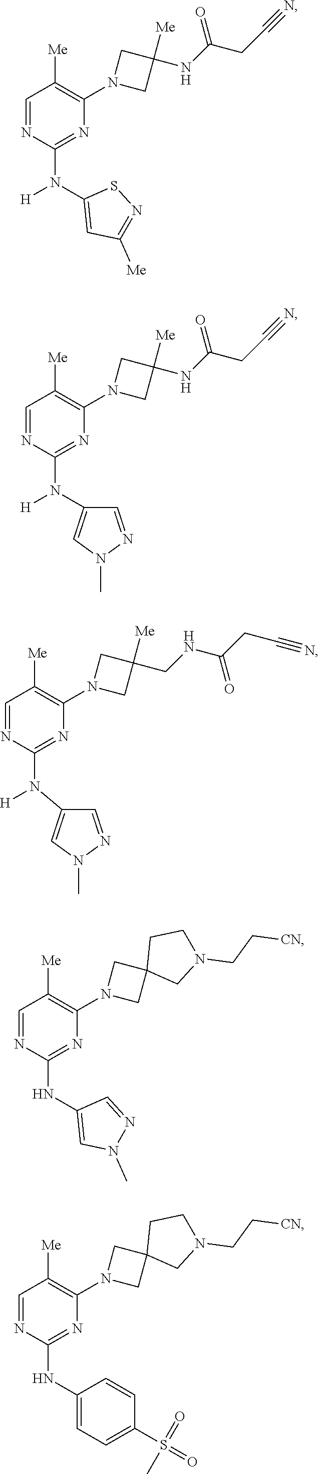 [SMILES: Cc1cc(Nc2ncc(C)c(N3CC(C)(NC(=O)CC#N)C3)n2)sn1 ; Cc1cnc(Nc2cnn(C)c2)nc1N1CC(C)(NC(=O)CC#N)C1 ; Cc1cnc(Nc2cnn(C)c2)nc1N1CC(C)(CNC(=O)CC#N)C1 ; Cc1cnc(Nc2cnn(C)c2)nc1N1CC2(CCN(CCC#N)C2)C1 ; Cc1cnc(Nc2ccc(S(C)(=O)=O)cc2)nc1N1CC2(CCN(CCC#N)C2)C1] 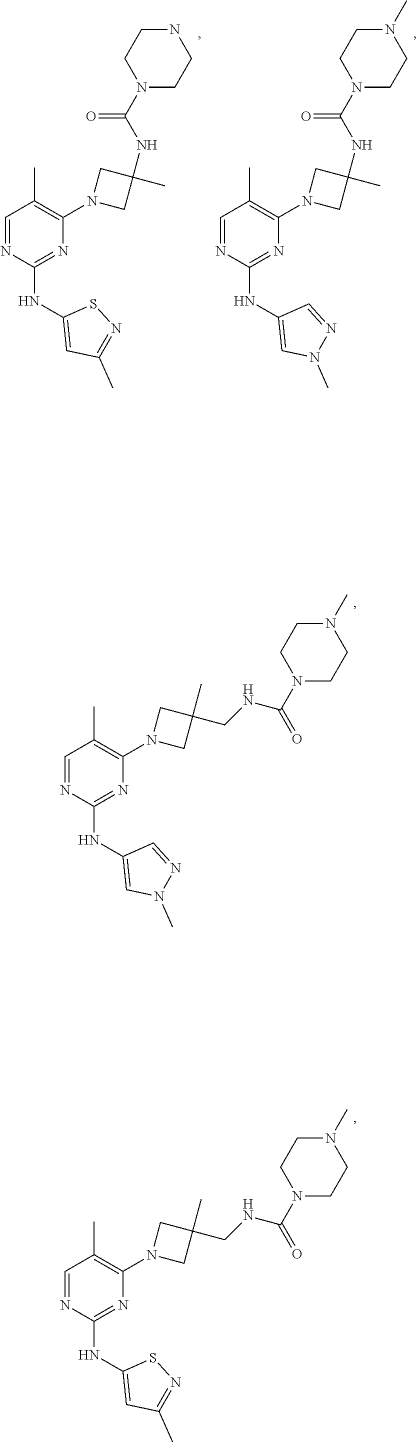 [SMILES: Cc1cc(Nc2ncc(C)c(N3CC(C)(NC(=O)N4CCNCC4)C3)n2)sn1 ; Cc1cnc(Nc2cnn(C)c2)nc1N1CC(C)(NC(=O)N2CCN(C)CC2)C1 ; Cc1cnc(Nc2cnn(C)c2)nc1N1CC(C)(CNC(=O)N2CCN(C)CC2)C1 ; Cc1cc(Nc2ncc(C)c(N3CC(C)(CNC(=O)N4CCN(C)CC4)C3)n2)sn1] 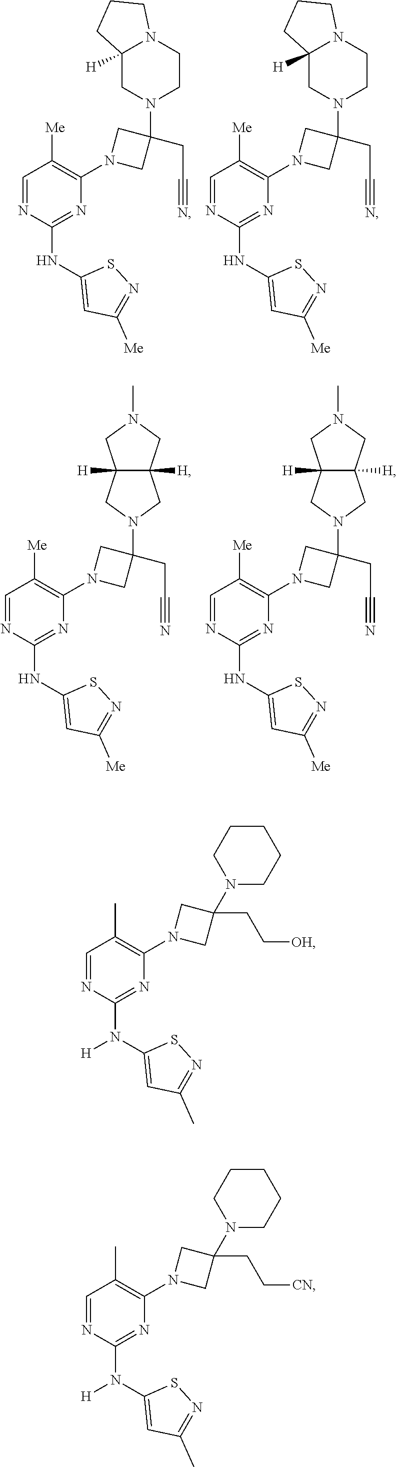 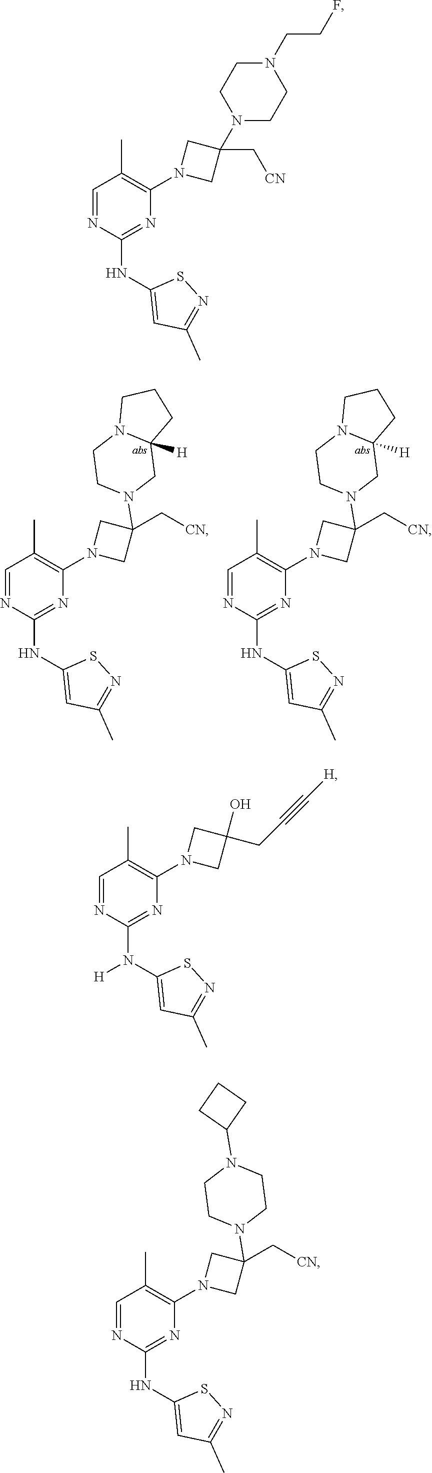 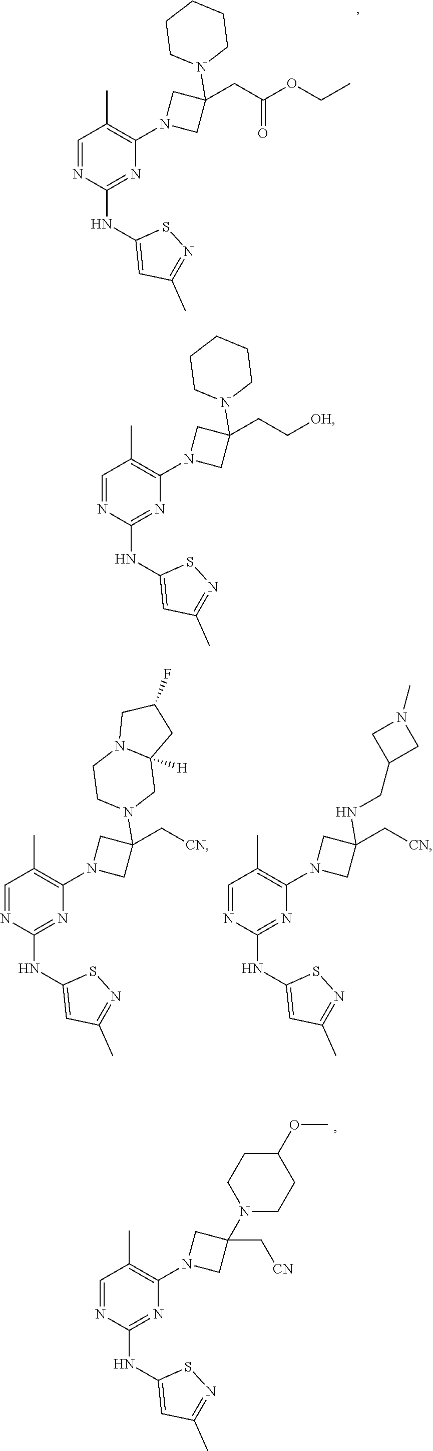 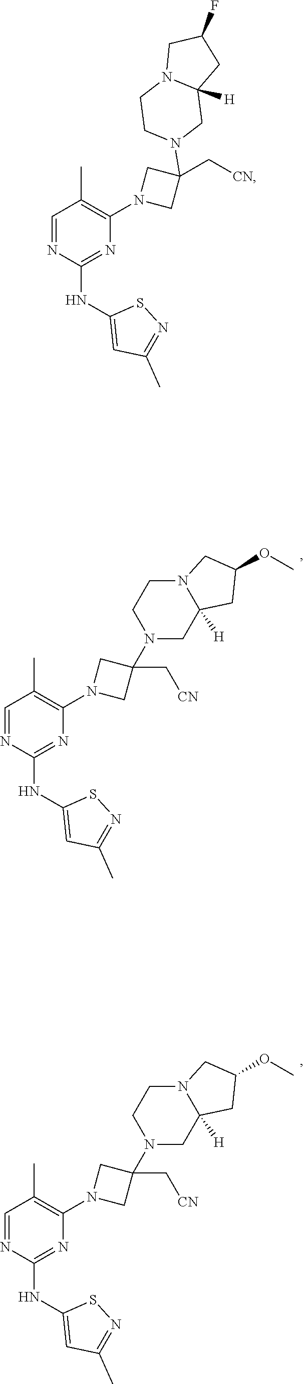 [SMILES: Cc1cc(Nc2ncc(C)c(N3CC(CC#N)(N4CCN5C[C@@H](F)C[C@@H]5C4)C3)n2)sn1 ; CO[C@H]1C[C@H]2CN(C3(CC#N)CN(c4nc(Nc5cc(C)ns5)ncc4C)C3)CCN2C1 ; CO[C@@H]1C[C@H]2CN(C3(CC#N)CN(c4nc(Nc5cc(C)ns5)ncc4C)C3)CCN2C1] 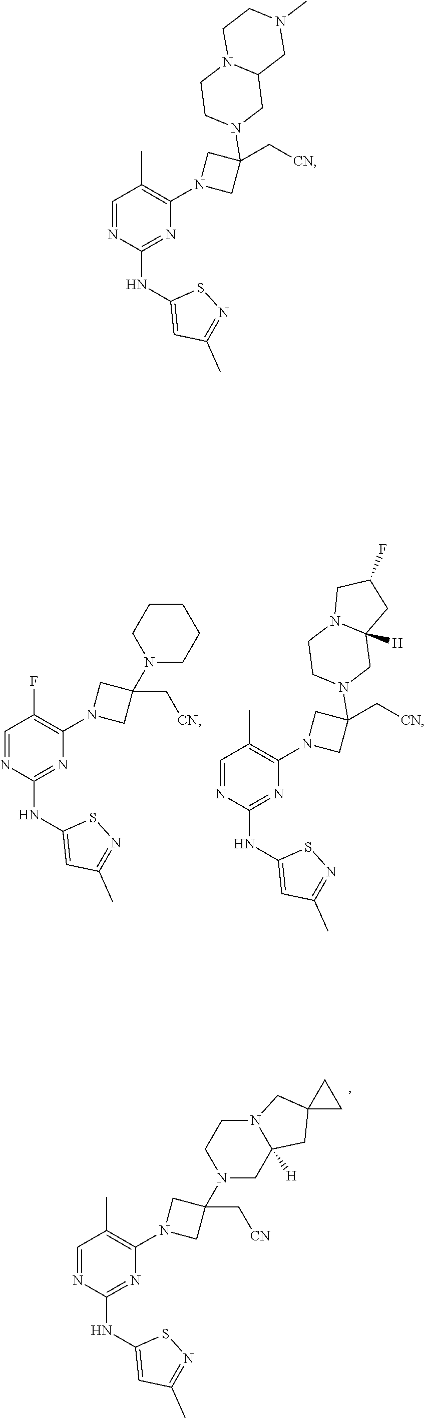 [SMILES: Cc1cc(Nc2ncc(C)c(N3CC(CC#N)(N4CCN5CCN(C)CC5C4)C3)n2)sn1 ; Cc1cc(Nc2ncc(F)c(N3CC(CC#N)(N4CCCCC4)C3)n2)sn1 ; Cc1cc(Nc2ncc(C)c(N3CC(CC#N)(N4CCN5C[C@H](F)C[C@@H]5C4)C3)n2)sn1 ; Cc1cc(Nc2ncc(C)c(N3CC(CC#N)(N4CCN5CC6(CC6)C[C@H]5C4)C3)n2)sn1] 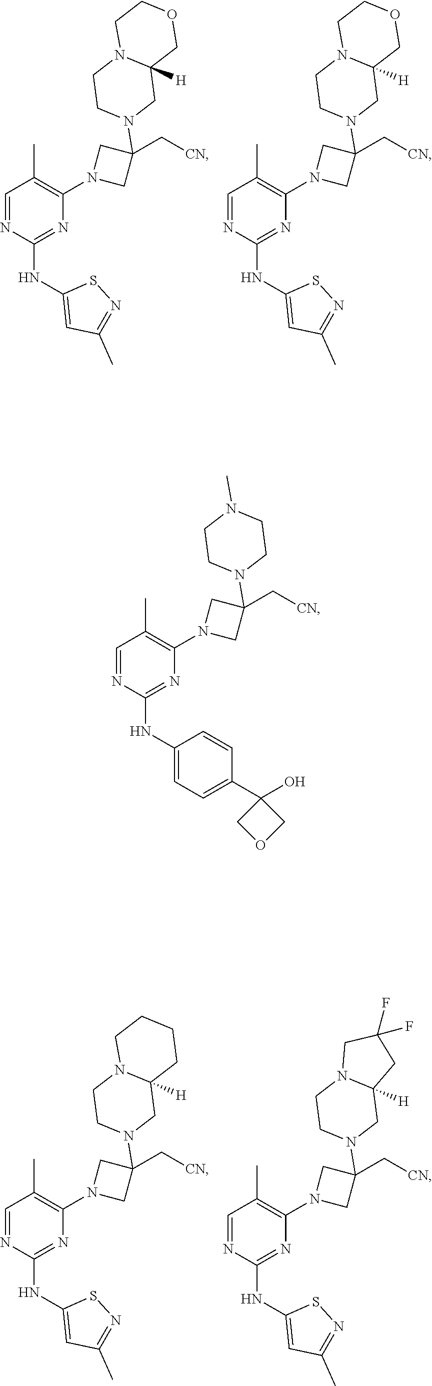 [SMILES: Cc1cc(Nc2ncc(C)c(N3CC(CC#N)(N4CCN5CCOC[C@@H]5C4)C3)n2)sn1 ; Cc1cc(Nc2ncc(C)c(N3CC(CC#N)(N4CCN5CCOC[C@H]5C4)C3)n2)sn1 ; Cc1cnc(Nc2ccc(C3(O)COC3)cc2)nc1N1CC(CC#N)(N2CCN(C)CC2)C1 ; Cc1cc(Nc2ncc(C)c(N3CC(CC#N)(N4CCN5CCCC[C@H]5C4)C3)n2)sn1 ; Cc1cc(Nc2ncc(C)c(N3CC(CC#N)(N4CCN5CC(F)(F)C[C@H]5C4)C3)n2)sn1] 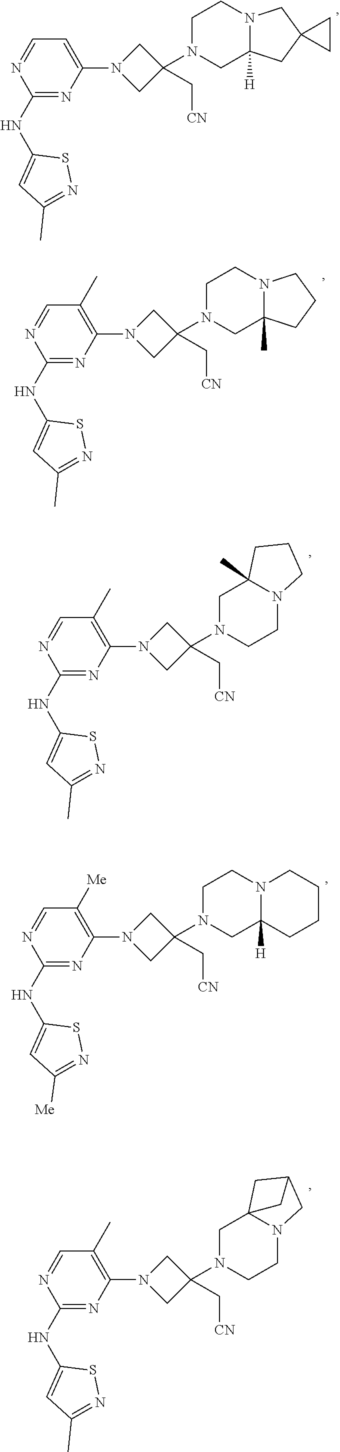 [SMILES: Cc1cc(Nc2nccc(N3CC(CC#N)(N4CCN5CC6(CC6)C[C@H]5C4)C3)n2)sn1 ; Cc1cc(Nc2ncc(C)c(N3CC(CC#N)(N4CCN5CCC[C@]5(C)C4)C3)n2)sn1 ; Cc1cc(Nc2ncc(C)c(N3CC(CC#N)(N4CCN5CCC[C@@]5(C)C4)C3)n2)sn1 ; Cc1cc(Nc2ncc(C)c(N3CC(CC#N)(N4CCN5CCCC[C@@H]5C4)C3)n2)sn1 ; Cc1cc(Nc2ncc(C)c(N3CC(CC#N)(N4CCN5CC6CC5(C6)C4)C3)n2)sn1] 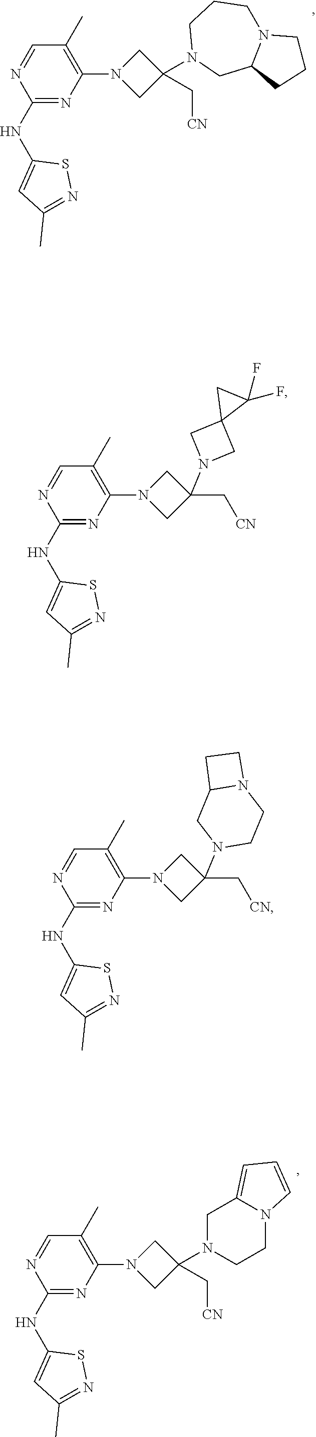 [SMILES: Cc1cc(Nc2ncc(C)c(N3CC(CC#N)(N4CCCN5CCC[C@H]5C4)C3)n2)sn1 ; Cc1cc(Nc2ncc(C)c(N3CC(CC#N)(N4CC5(C4)CC5(F)F)C3)n2)sn1 ; Cc1cc(Nc2ncc(C)c(N3CC(CC#N)(N4CCN5CCC5C4)C3)n2)sn1 ; Cc1cc(Nc2ncc(C)c(N3CC(CC#N)(N4CCn5cccc5C4)C3)n2)sn1] 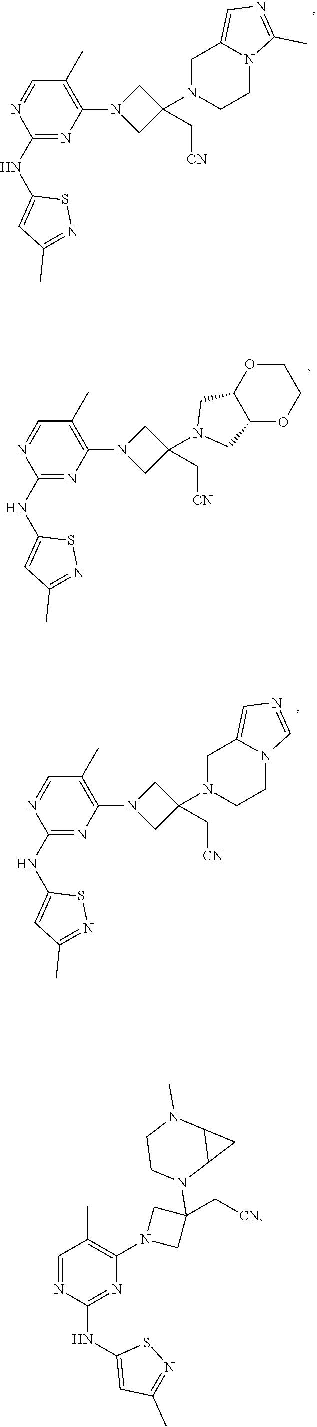 [SMILES: Cc1cc(Nc2ncc(C)c(N3CC(CC#N)(N4CCn5c(cnc5C)C4)C3)n2)sn1 ; Cc1cc(Nc2ncc(C)c(N3CC(CC#N)(N4C[C@@H]5OCCO[C@@H]5C4)C3)n2)sn1 ; Cc1cc(Nc2ncc(C)c(N3CC(CC#N)(N4CCn5cncc5C4)C3)n2)sn1 ; Cc1cc(Nc2ncc(C)c(N3CC(CC#N)(N4CCN(C)C5CC54)C3)n2)sn1] 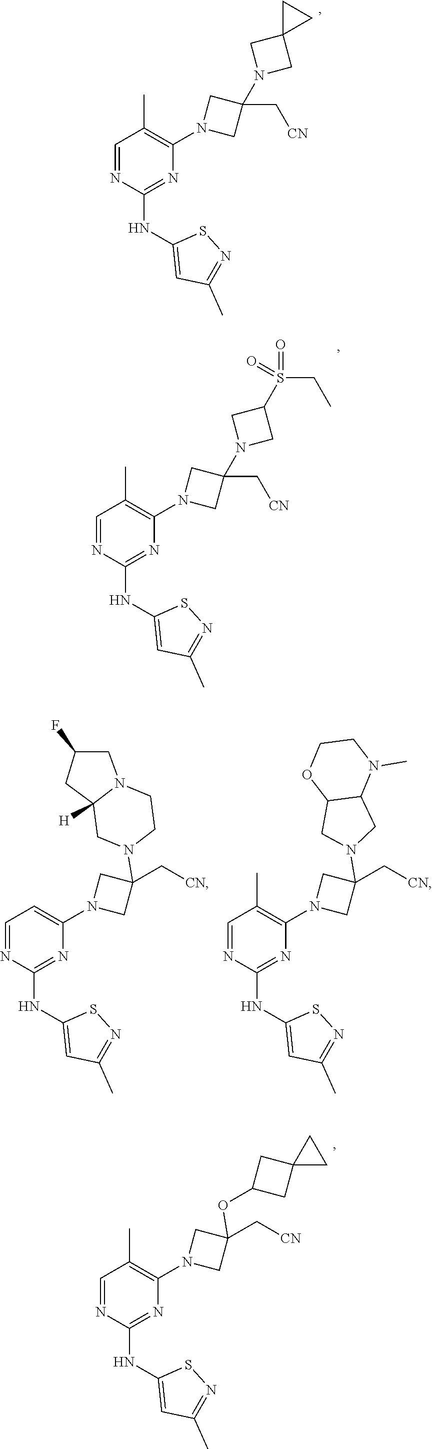 [SMILES: Cc1cc(Nc2ncc(C)c(N3CC(CC#N)(N4CC5(CC5)C4)C3)n2)sn1 ; CCS(=O)(=O)C1CN(C2(CC#N)CN(c3nc(Nc4cc(C)ns4)ncc3C)C2)C1 ; Cc1cc(Nc2nccc(N3CC(CC#N)(N4CCN5C[C@H](F)C[C@H]5C4)C3)n2)sn1 ; Cc1cc(Nc2ncc(C)c(N3CC(CC#N)(N4CC5OCCN(C)C5C4)C3)n2)sn1 ; Cc1cc(Nc2ncc(C)c(N3CC(CC#N)(OC4CC5(CC5)C4)C3)n2)sn1] 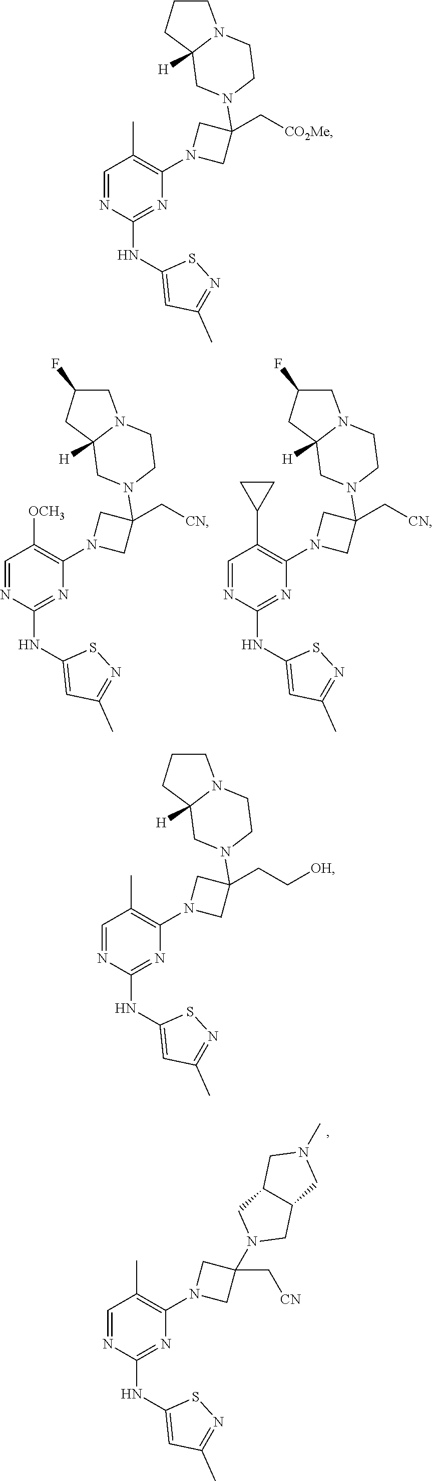 [SMILES: COC(=O)CC1(N2CCN3CCC[C@H]3C2)CN(c2nc(Nc3cc(C)ns3)ncc2C)C1 ; COc1cnc(Nc2cc(C)ns2)nc1N1CC(CC#N)(N2CCN3C[C@H](F)C[C@H]3C2)C1 ; Cc1cc(Nc2ncc(C3CC3)c(N3CC(CC#N)(N4CCN5C[C@H](F)C[C@H]5C4)C3)n2)sn1 ; Cc1cc(Nc2ncc(C)c(N3CC(CCO)(N4CCN5CCC[C@H]5C4)C3)n2)sn1 ; Cc1cc(Nc2ncc(C)c(N3CC(CC#N)(N4C[C@H]5CN(C)C[C@H]5C4)C3)n2)sn1] 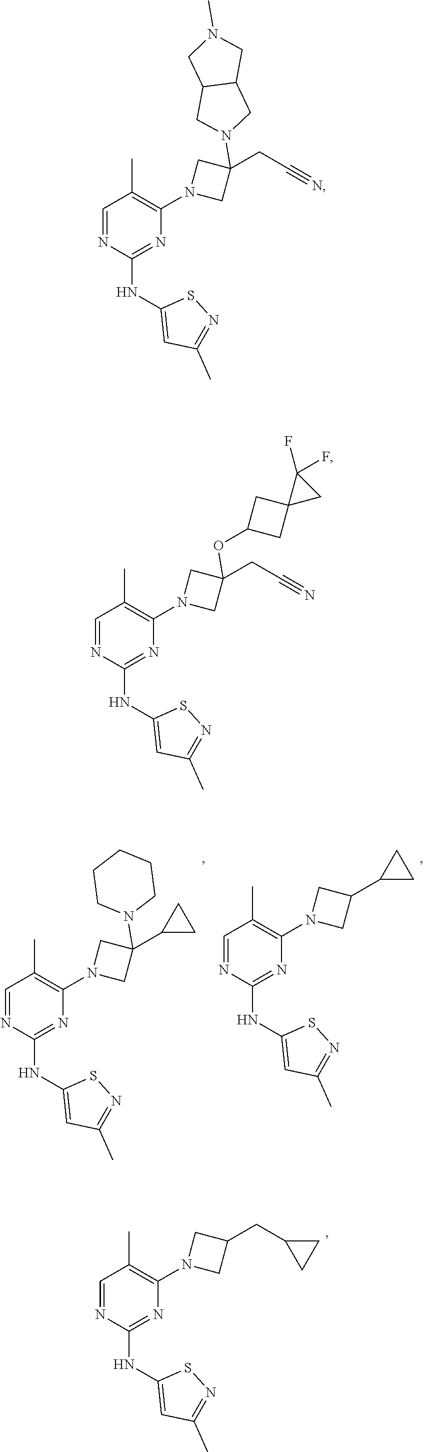 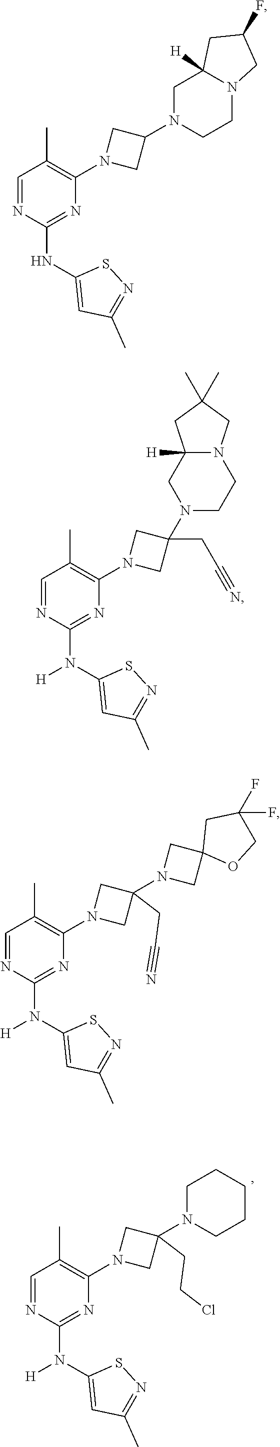 [SMILES: Cc1cc(Nc2ncc(C)c(N3CC(N4CCN5C[C@H](F)C[C@H]5C4)C3)n2)sn1 ; Cc1cc(Nc2ncc(C)c(N3CC(CC#N)(N4CCN5CC(C)(C)C[C@H]5C4)C3)n2)sn1 ; Cc1cc(Nc2ncc(C)c(N3CC(CC#N)(N4CC5(C4)CC(F)(F)CO5)C3)n2)sn1 ; Cc1cc(Nc2ncc(C)c(N3CC(CCCl)(N4CCCCC4)C3)n2)sn1] 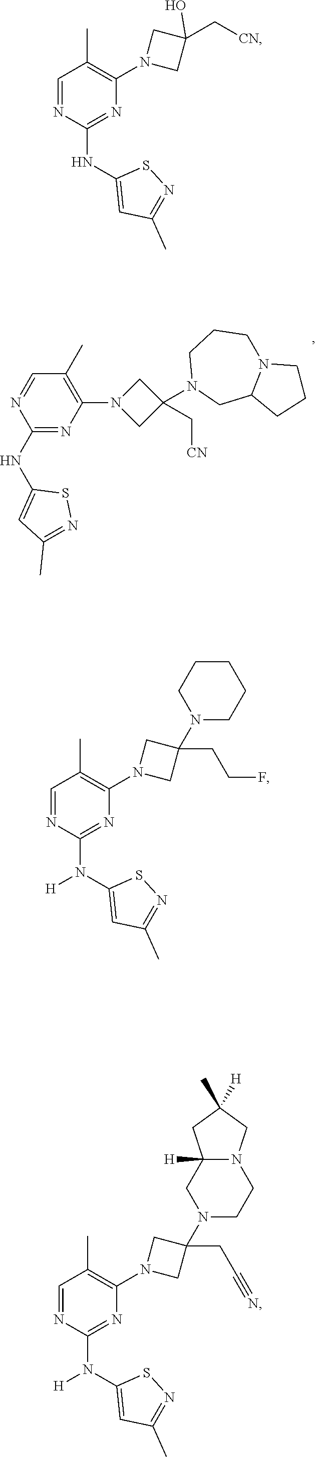 [SMILES: Cc1cc(Nc2ncc(C)c(N3CC(O)(CC#N)C3)n2)sn1 ; Cc1cc(Nc2ncc(C)c(N3CC(CC#N)(N4CCCN5CCCC5C4)C3)n2)sn1 ; Cc1cc(Nc2ncc(C)c(N3CC(CCF)(N4CCCCC4)C3)n2)sn1 ; Cc1cc(Nc2ncc(C)c(N3CC(CC#N)(N4CCN5C[C@H](C)C[C@H]5C4)C3)n2)sn1] 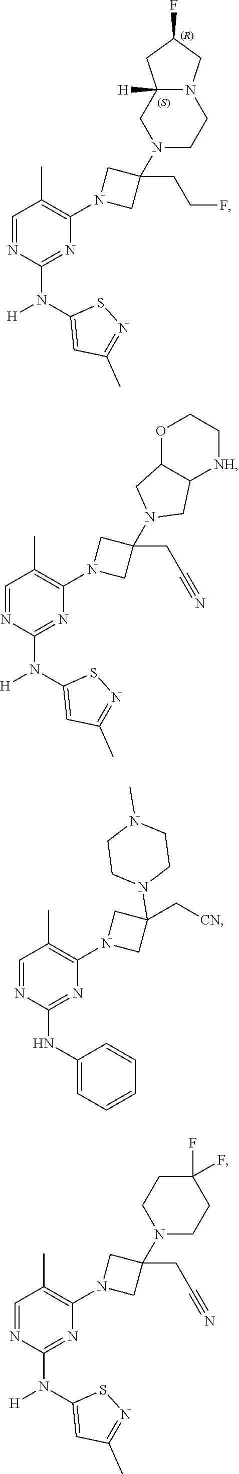 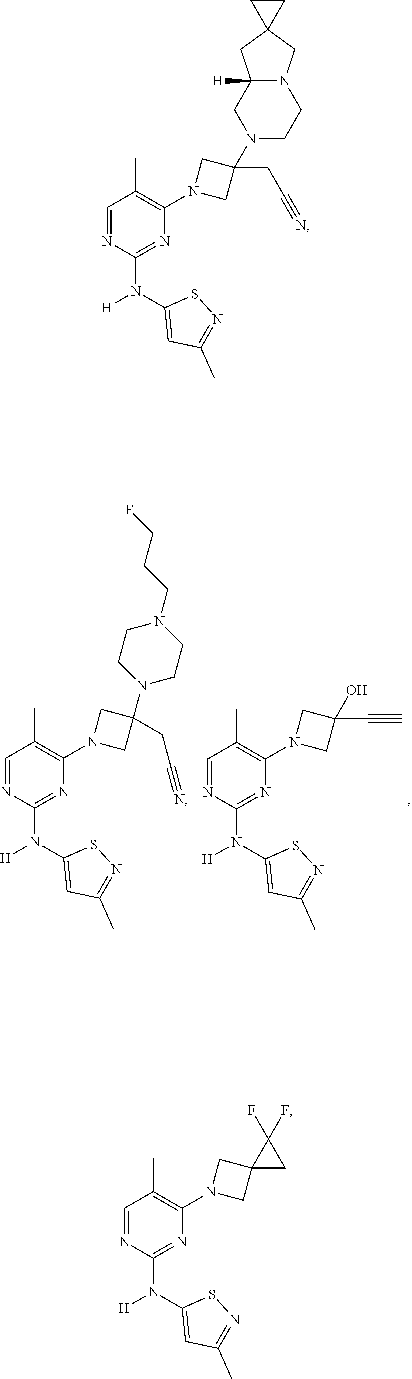 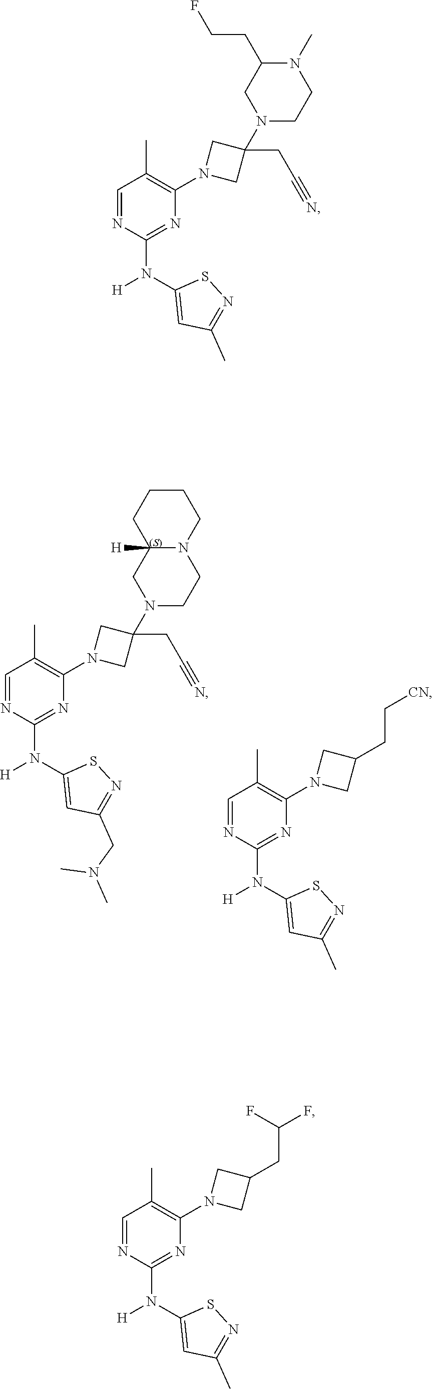 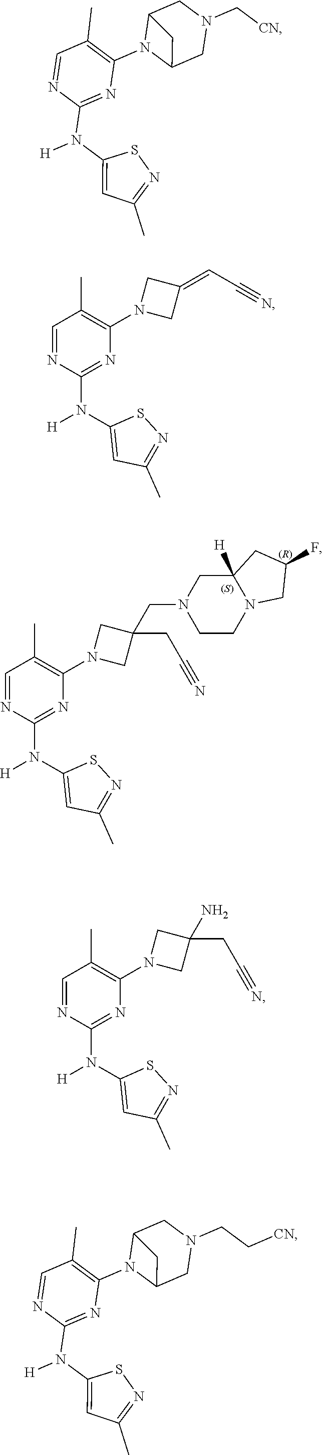 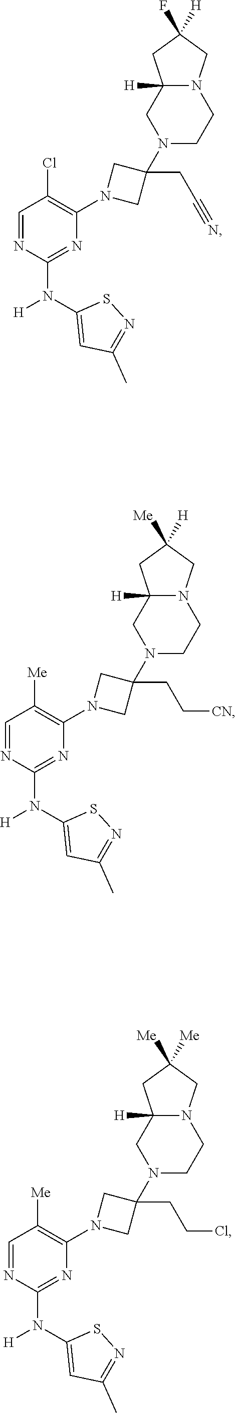 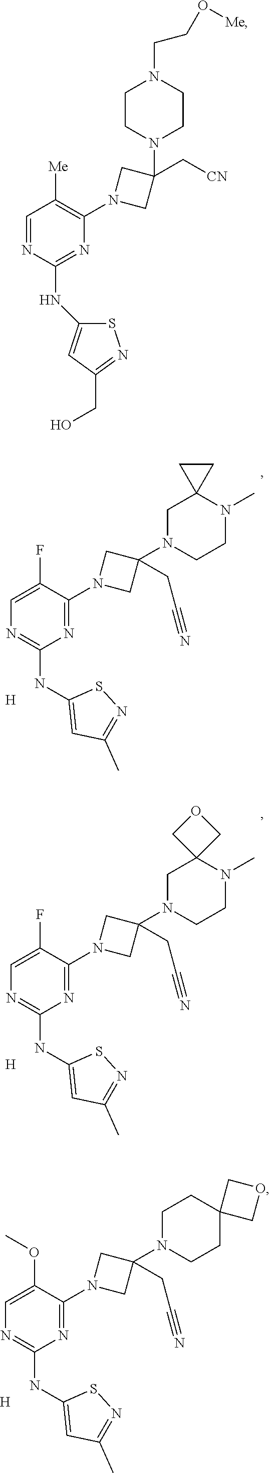 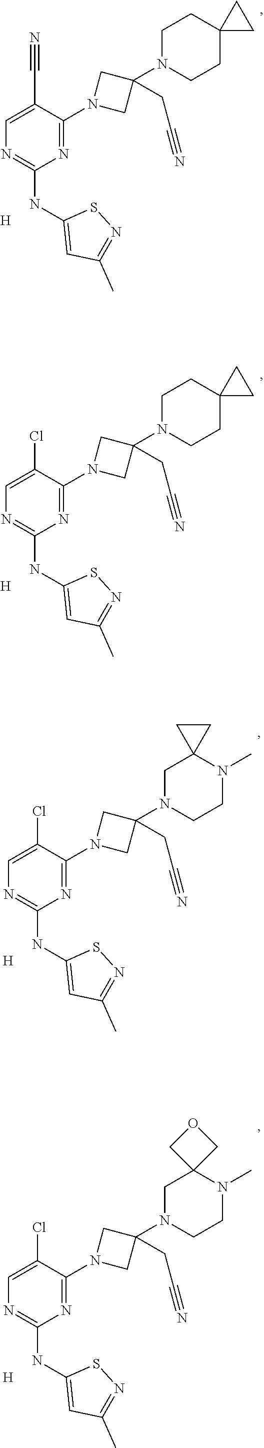 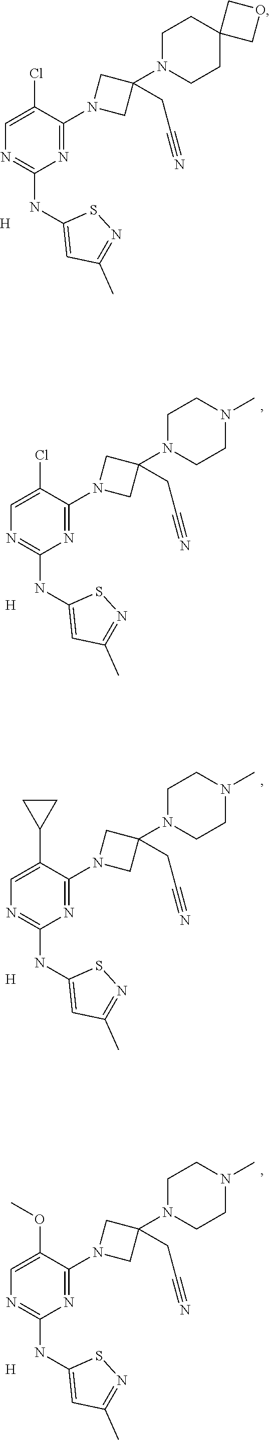 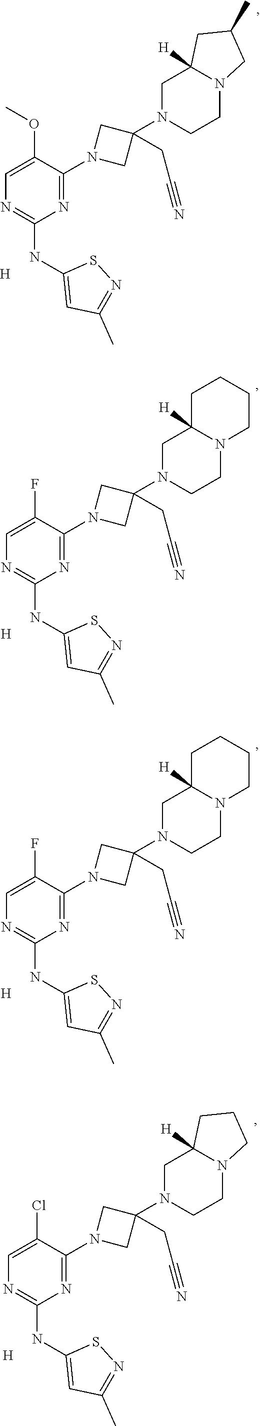 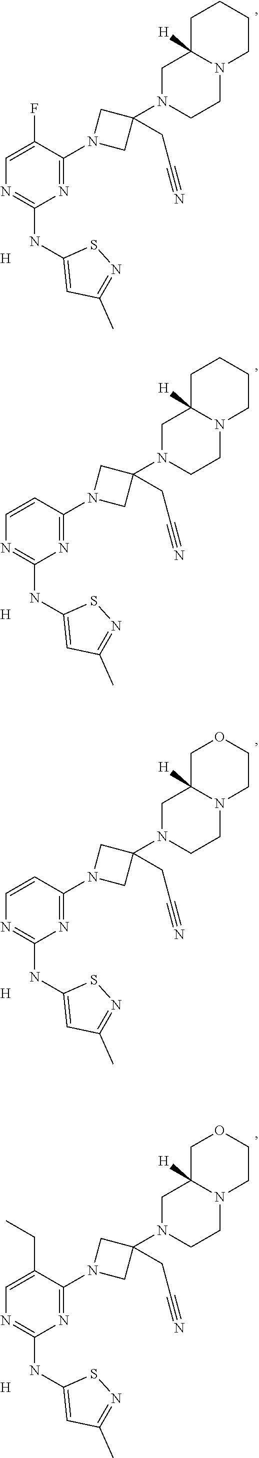 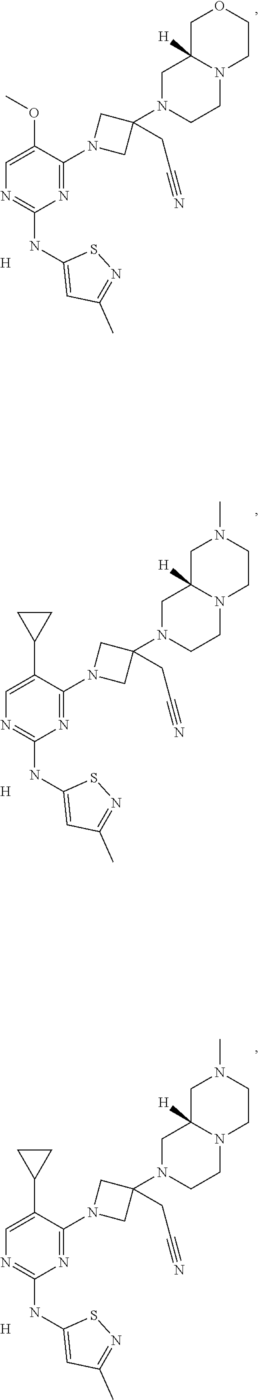 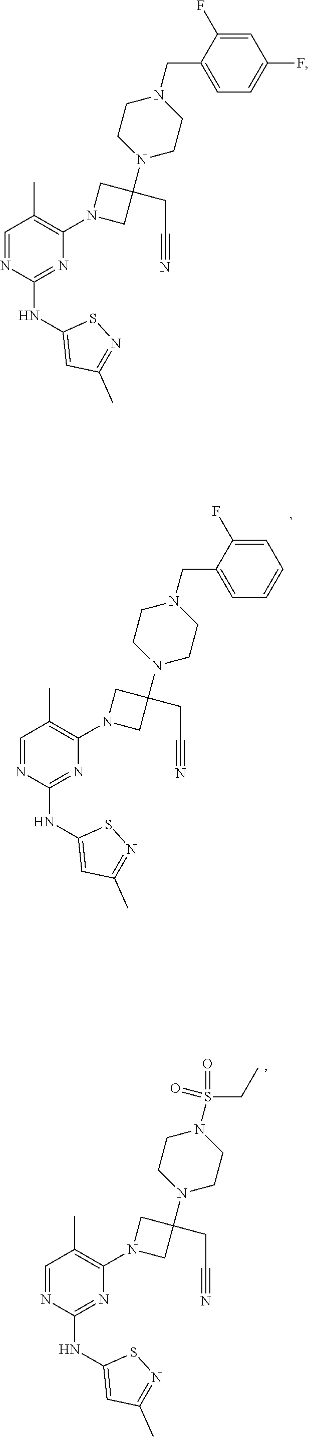 [SMILES: Cc1cc(Nc2ncc(C)c(N3CC(CC#N)(N4CCN(Cc5ccc(F)cc5F)CC4)C3)n2)sn1 ; Cc1cc(Nc2ncc(C)c(N3CC(CC#N)(N4CCN(Cc5ccccc5F)CC4)C3)n2)sn1 ; CCS(=O)(=O)N1CCN(C2(CC#N)CN(c3nc(Nc4cc(C)ns4)ncc3C)C2)CC1] 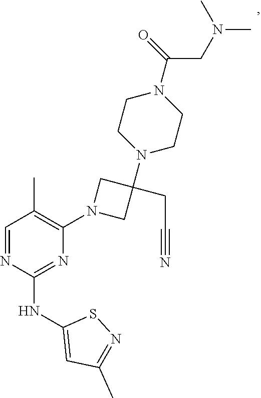 [SMILES: Cc1cc(Nc2ncc(C)c(N3CC(CC#N)(N4CCN(C(=O)CN(C)C)CC4)C3)n2)sn1] 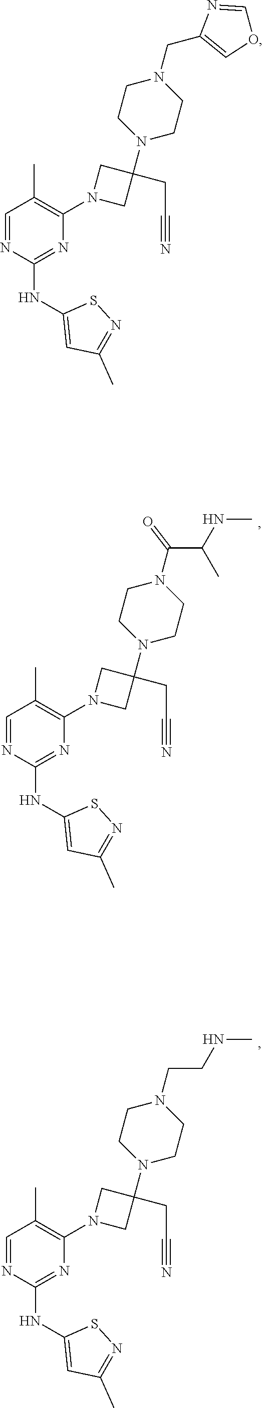 [SMILES: Cc1cc(Nc2ncc(C)c(N3CC(CC#N)(N4CCN(Cc5cocn5)CC4)C3)n2)sn1 ; CNC(C)C(=O)N1CCN(C2(CC#N)CN(c3nc(Nc4cc(C)ns4)ncc3C)C2)CC1 ; CNCCN1CCN(C2(CC#N)CN(c3nc(Nc4cc(C)ns4)ncc3C)C2)CC1] 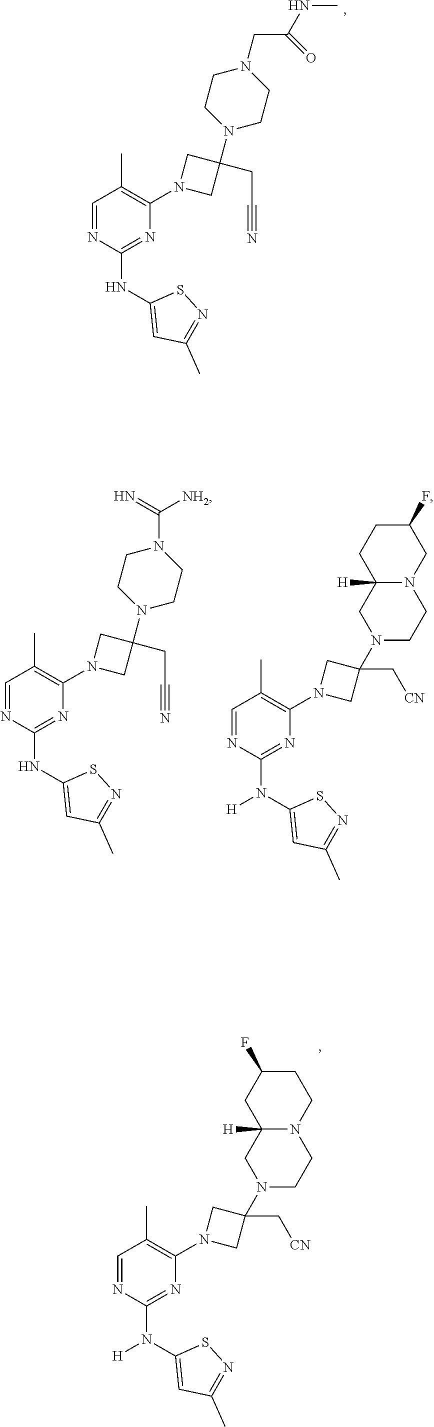 [SMILES: CNC(=O)CN1CCN(C2(CC#N)CN(c3nc(Nc4cc(C)ns4)ncc3C)C2)CC1 ; Cc1cc(Nc2ncc(C)c(N3CC(CC#N)(N4CCN(C(=N)N)CC4)C3)n2)sn1 ; Cc1cc(Nc2ncc(C)c(N3CC(CC#N)(N4CCN5C[C@H](F)CC[C@H]5C4)C3)n2)sn1 ; Cc1cc(Nc2ncc(C)c(N3CC(CC#N)(N4CCN5CC[C@H](F)C[C@H]5C4)C3)n2)sn1] 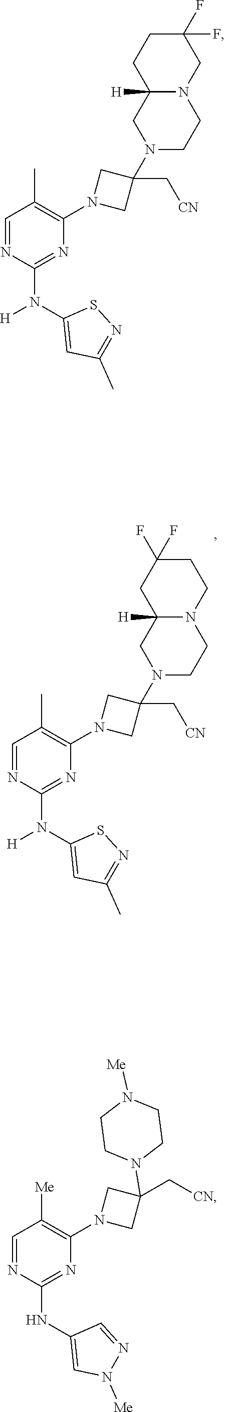 [SMILES: Cc1cc(Nc2ncc(C)c(N3CC(CC#N)(N4CCN5CC(F)(F)CC[C@H]5C4)C3)n2)sn1 ; Cc1cc(Nc2ncc(C)c(N3CC(CC#N)(N4CCN5CCC(F)(F)C[C@H]5C4)C3)n2)sn1 ; Cc1cnc(Nc2cnn(C)c2)nc1N1CC(CC#N)(N2CCN(C)CC2)C1] 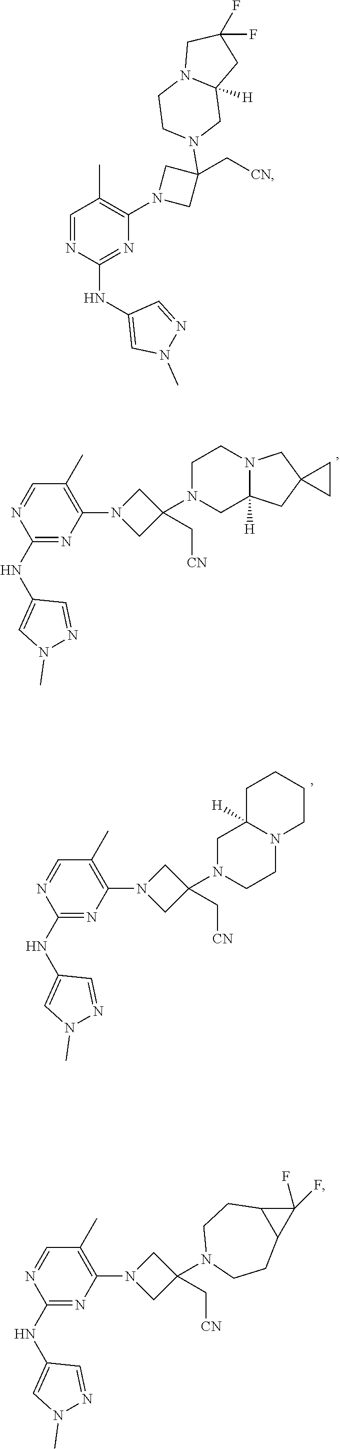 [SMILES: Cc1cnc(Nc2cnn(C)c2)nc1N1CC(CC#N)(N2CCN3CC(F)(F)C[C@H]3C2)C1 ; Cc1cnc(Nc2cnn(C)c2)nc1N1CC(CC#N)(N2CCN3CC4(CC4)C[C@H]3C2)C1 ; Cc1cnc(Nc2cnn(C)c2)nc1N1CC(CC#N)(N2CCN3CCCC[C@@H]3C2)C1 ; Cc1cnc(Nc2cnn(C)c2)nc1N1CC(CC#N)(N2CCC3C(CC2)C3(F)F)C1] 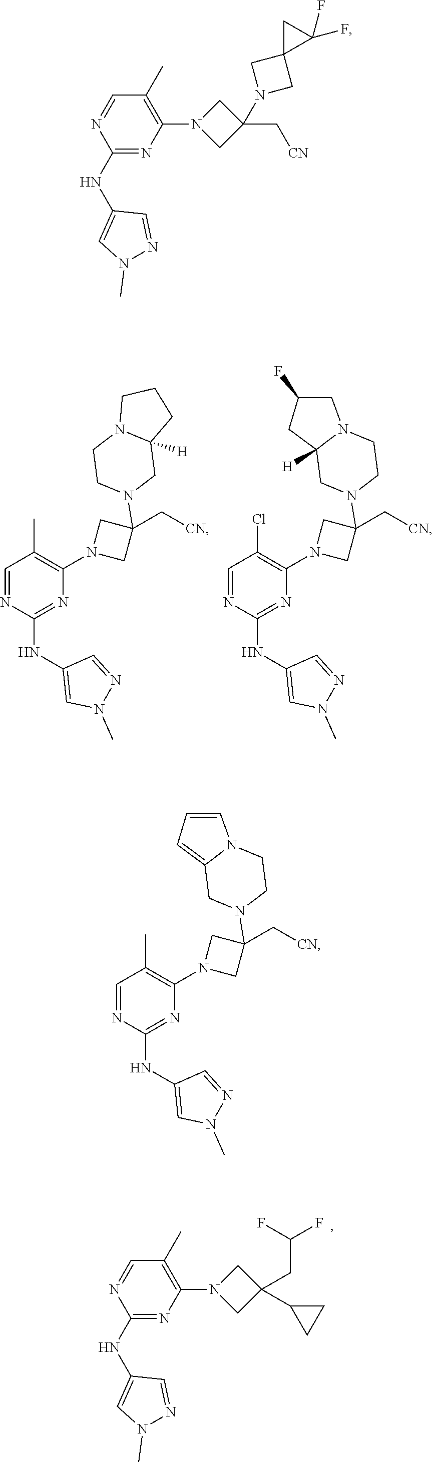 [SMILES: Cc1cnc(Nc2cnn(C)c2)nc1N1CC(CC#N)(N2CC3(C2)CC3(F)F)C1 ; Cc1cnc(Nc2cnn(C)c2)nc1N1CC(CC#N)(N2CCN3CCC[C@H]3C2)C1 ; Cn1cc(Nc2ncc(Cl)c(N3CC(CC#N)(N4CCN5C[C@H](F)C[C@H]5C4)C3)n2)cn1 ; Cc1cnc(Nc2cnn(C)c2)nc1N1CC(CC#N)(N2CCn3cccc3C2)C1 ; Cc1cnc(Nc2cnn(C)c2)nc1N1CC(CC(F)F)(C2CC2)C1] 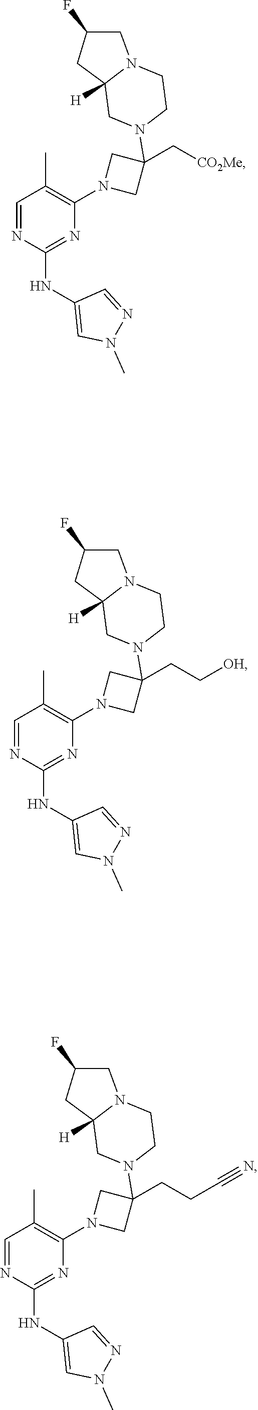 [SMILES: COC(=O)CC1(N2CCN3C[C@H](F)C[C@H]3C2)CN(c2nc(Nc3cnn(C)c3)ncc2C)C1 ; Cc1cnc(Nc2cnn(C)c2)nc1N1CC(CCO)(N2CCN3C[C@H](F)C[C@H]3C2)C1 ; Cc1cnc(Nc2cnn(C)c2)nc1N1CC(CCC#N)(N2CCN3C[C@H](F)C[C@H]3C2)C1] 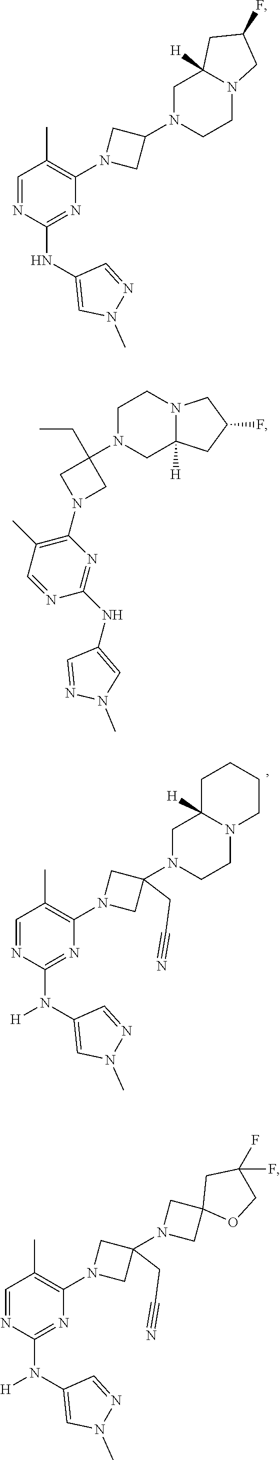 [SMILES: Cc1cnc(Nc2cnn(C)c2)nc1N1CC(N2CCN3C[C@H](F)C[C@H]3C2)C1 ; CCC1(N2CCN3C[C@H](F)C[C@H]3C2)CN(c2nc(Nc3cnn(C)c3)ncc2C)C1 ; Cc1cnc(Nc2cnn(C)c2)nc1N1CC(CC#N)(N2CCN3CCCC[C@H]3C2)C1 ; Cc1cnc(Nc2cnn(C)c2)nc1N1CC(CC#N)(N2CC3(C2)CC(F)(F)CO3)C1] 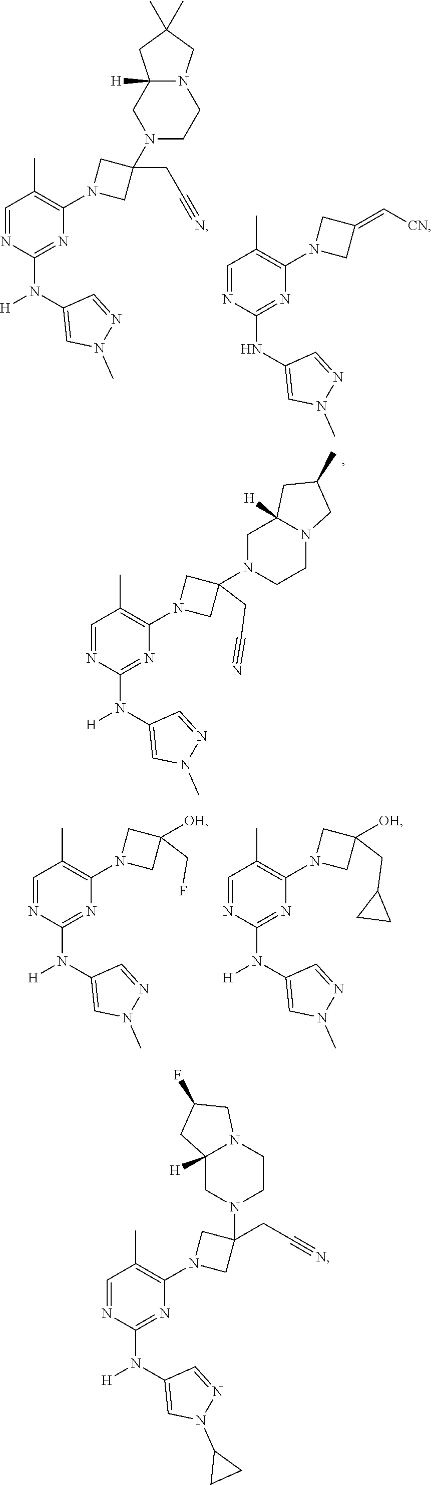 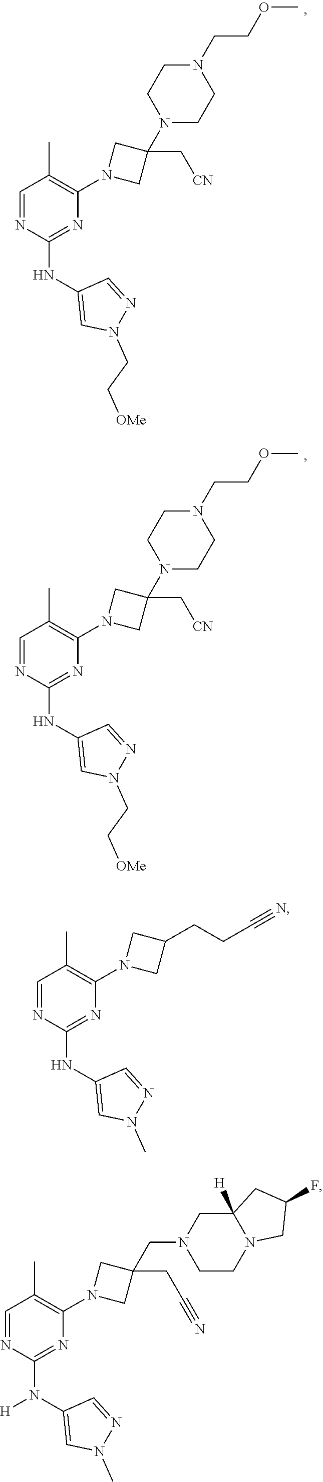 [SMILES: COCCN1CCN(C2(CC#N)CN(c3nc(Nc4cnn(CCOC)c4)ncc3C)C2)CC1 ; COCCN1CCN(C2(CC#N)CN(c3nc(Nc4cnn(CCOC)c4)ncc3C)C2)CC1 ; Cc1cnc(Nc2cnn(C)c2)nc1N1CC(CCC#N)C1 ; Cc1cnc(Nc2cnn(C)c2)nc1N1CC(CC#N)(CN2CCN3C[C@H](F)C[C@H]3C2)C1] 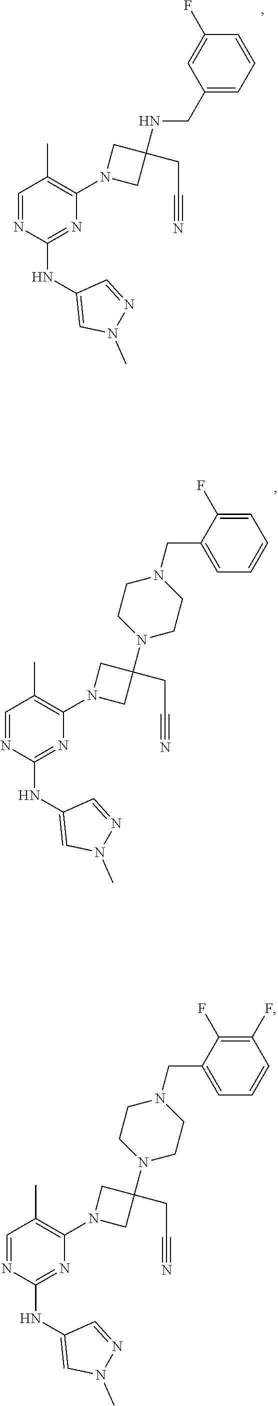 [SMILES: Cc1cnc(Nc2cnn(C)c2)nc1N1CC(CC#N)(NCc2cccc(F)c2)C1 ; Cc1cnc(Nc2cnn(C)c2)nc1N1CC(CC#N)(N2CCN(Cc3ccccc3F)CC2)C1 ; Cc1cnc(Nc2cnn(C)c2)nc1N1CC(CC#N)(N2CCN(Cc3cccc(F)c3F)CC2)C1] 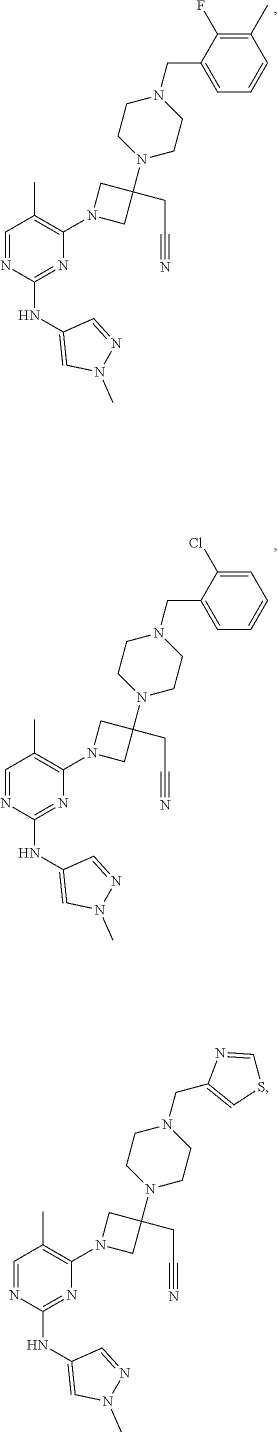 [SMILES: Cc1cnc(Nc2cnn(C)c2)nc1N1CC(CC#N)(N2CCN(Cc3cccc(C)c3F)CC2)C1 ; Cc1cnc(Nc2cnn(C)c2)nc1N1CC(CC#N)(N2CCN(Cc3ccccc3Cl)CC2)C1 ; Cc1cnc(Nc2cnn(C)c2)nc1N1CC(CC#N)(N2CCN(Cc3cscn3)CC2)C1] 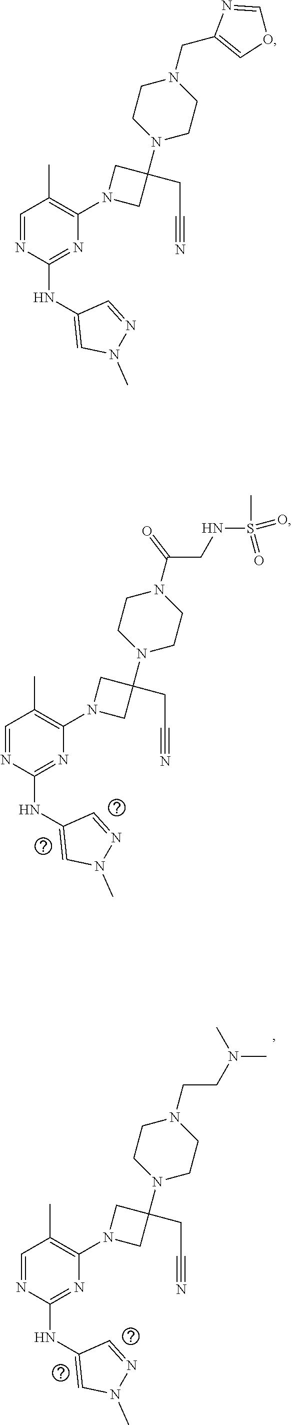 [SMILES: Cc1cnc(Nc2cnn(C)c2)nc1N1CC(CC#N)(N2CCN(Cc3cocn3)CC2)C1 ; Cc1cnc(Nc2cnn(C)c2)nc1N1CC(CC#N)(N2CCN(C(=O)CNS(C)(=O)=O)CC2)C1 ; Cc1cnc(Nc2cnn(C)c2)nc1N1CC(CC#N)(N2CCN(CCN(C)C)CC2)C1] 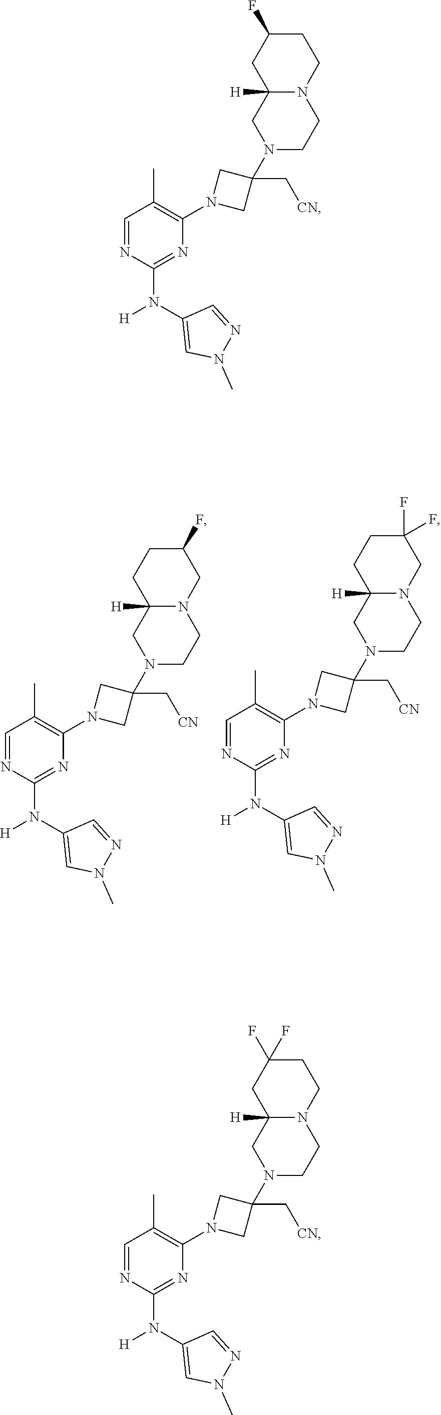 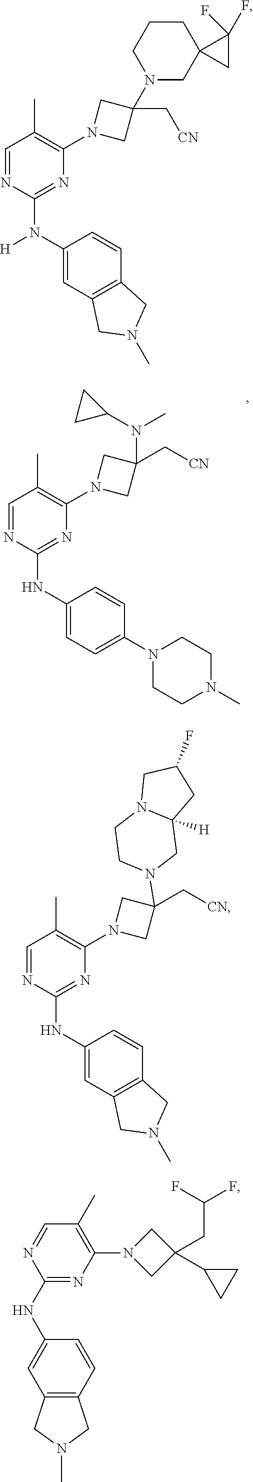 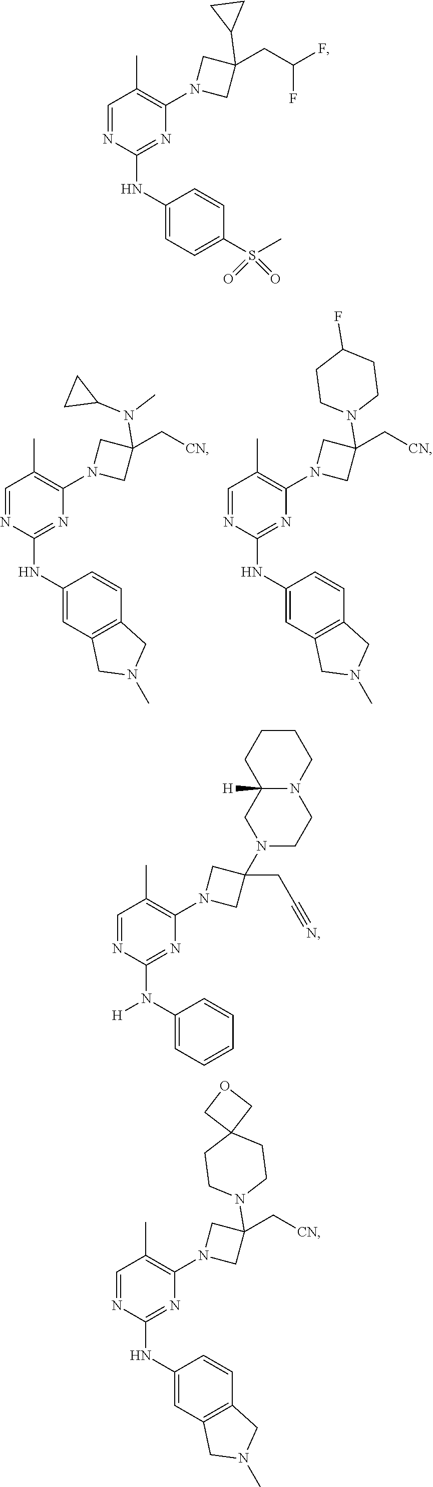 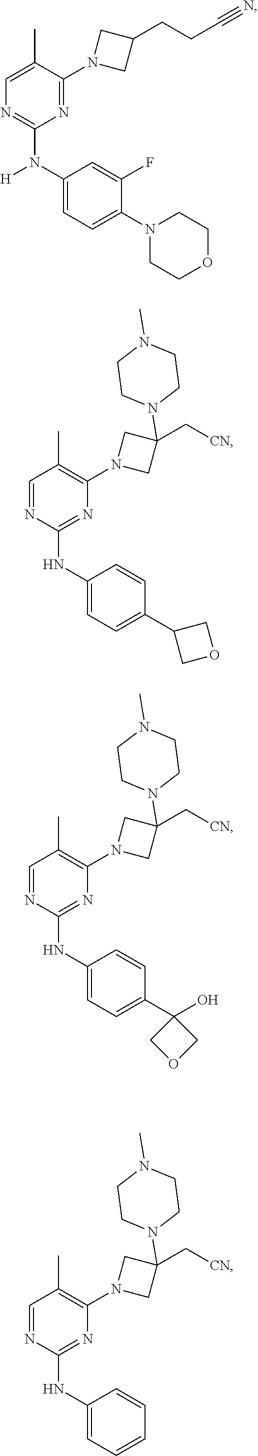 [SMILES: Cc1cnc(Nc2ccc(N3CCOCC3)c(F)c2)nc1N1CC(CCC#N)C1 ; Cc1cnc(Nc2ccc(C3COC3)cc2)nc1N1CC(CC#N)(N2CCN(C)CC2)C1 ; Cc1cnc(Nc2ccc(C3(O)COC3)cc2)nc1N1CC(CC#N)(N2CCN(C)CC2)C1 ; Cc1cnc(Nc2ccccc2)nc1N1CC(CC#N)(N2CCN(C)CC2)C1] 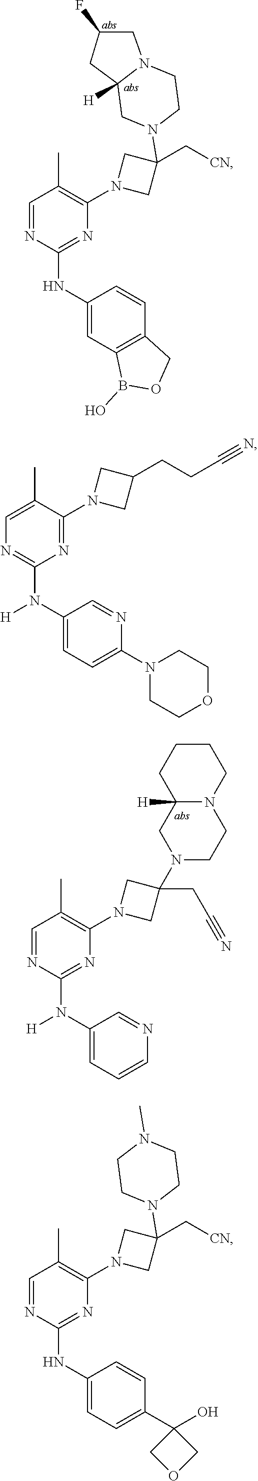 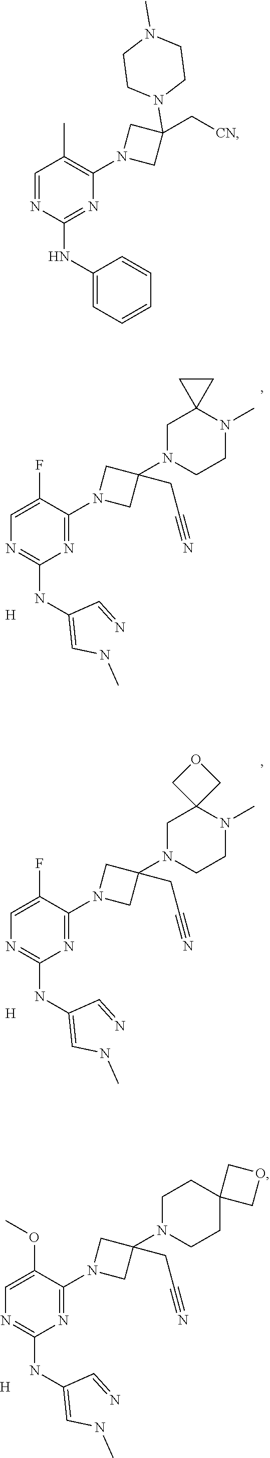 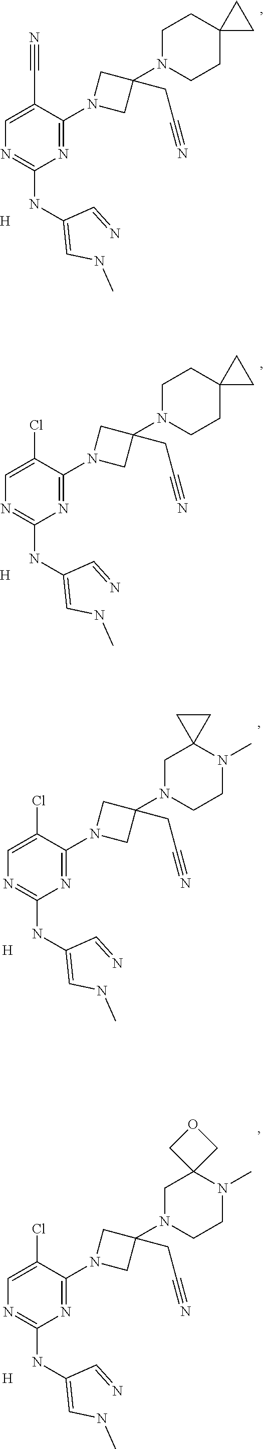 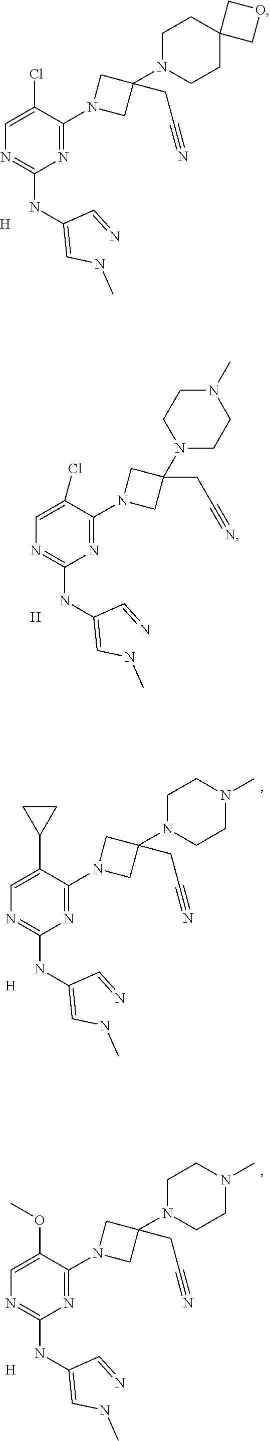 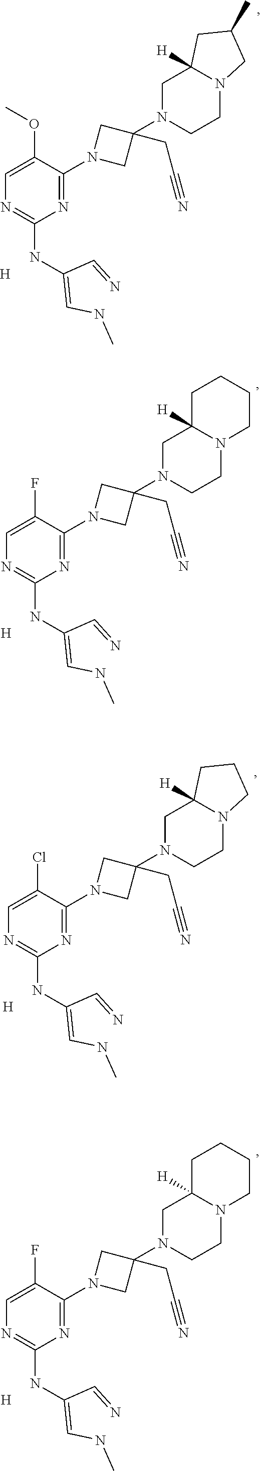 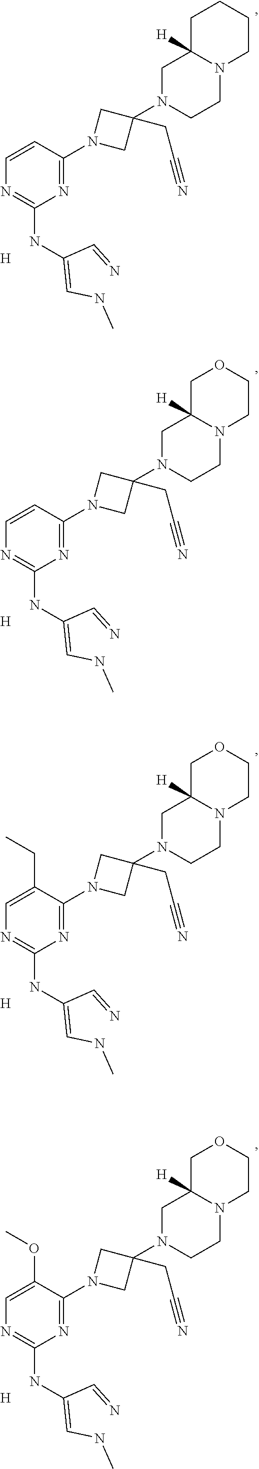 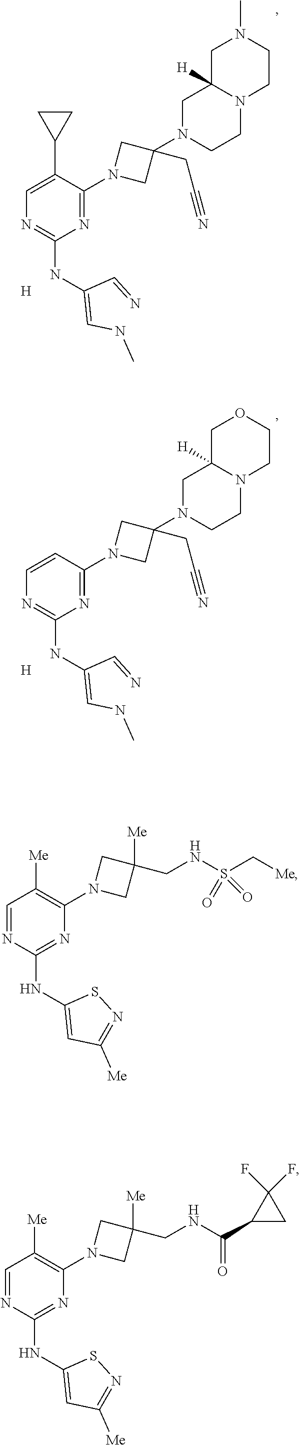 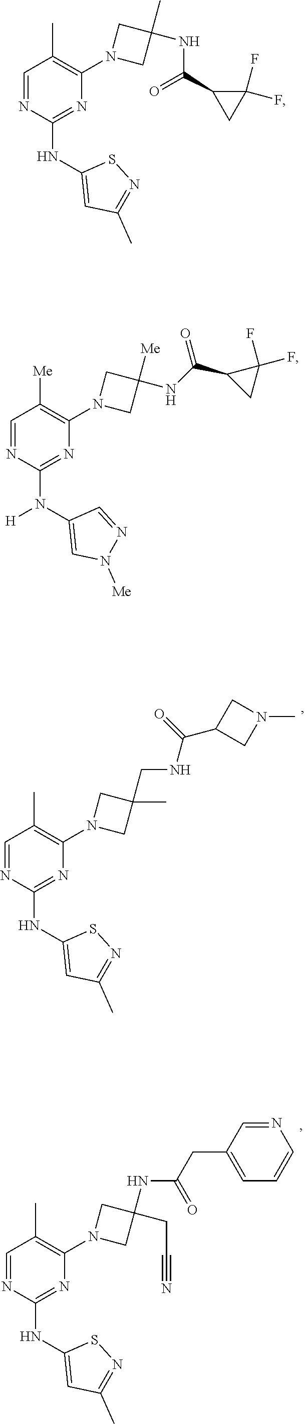 [SMILES: Cc1cc(Nc2ncc(C)c(N3CC(C)(NC(=O)[C@@H]4CC4(F)F)C3)n2)sn1 ; Cc1cnc(Nc2cnn(C)c2)nc1N1CC(C)(NC(=O)[C@@H]2CC2(F)F)C1 ; Cc1cc(Nc2ncc(C)c(N3CC(C)(CNC(=O)C4CN(C)C4)C3)n2)sn1 ; Cc1cc(Nc2ncc(C)c(N3CC(CC#N)(NC(=O)Cc4cccnc4)C3)n2)sn1] 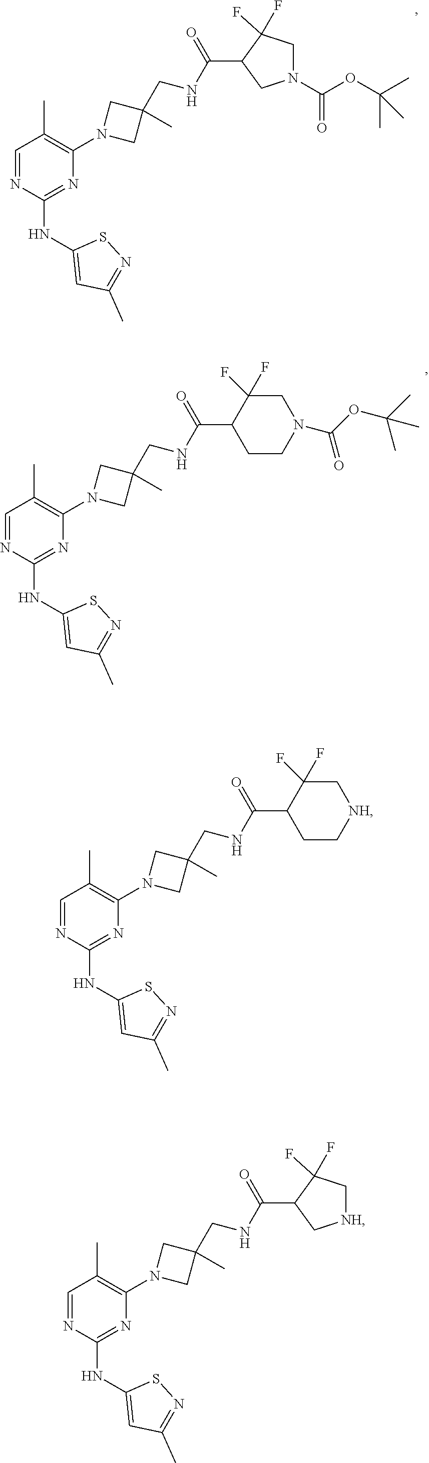 [SMILES: Cc1cc(Nc2ncc(C)c(N3CC(C)(CNC(=O)C4CN(C(=O)OC(C)(C)C)CC4(F)F)C3)n2)sn1 ; Cc1cc(Nc2ncc(C)c(N3CC(C)(CNC(=O)C4CCN(C(=O)OC(C)(C)C)CC4(F)F)C3)n2)sn1 ; Cc1cc(Nc2ncc(C)c(N3CC(C)(CNC(=O)C4CCNCC4(F)F)C3)n2)sn1 ; Cc1cc(Nc2ncc(C)c(N3CC(C)(CNC(=O)C4CNCC4(F)F)C3)n2)sn1] 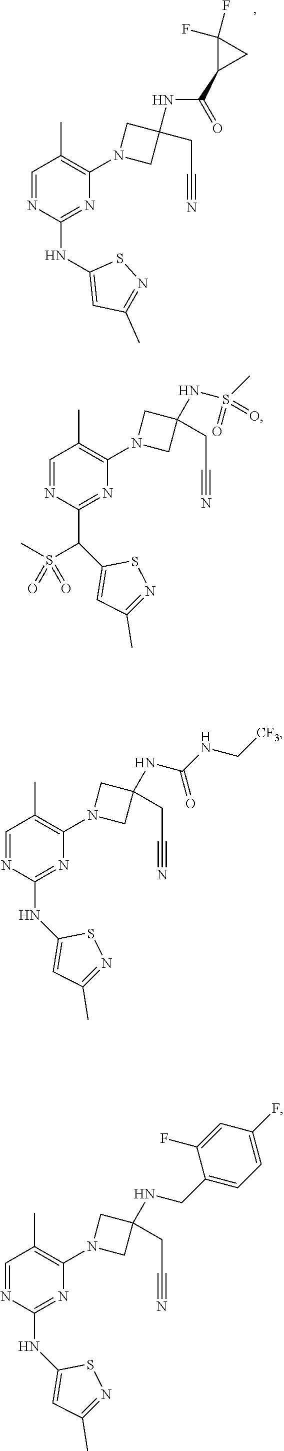 [SMILES: Cc1cc(Nc2ncc(C)c(N3CC(CC#N)(NC(=O)[C@@H]4CC4(F)F)C3)n2)sn1 ; Cc1cc(C(c2ncc(C)c(N3CC(CC#N)(NS(C)(=O)=O)C3)n2)S(C)(=O)=O)sn1 ; Cc1cc(Nc2ncc(C)c(N3CC(CC#N)(NC(=O)NCC(F)(F)F)C3)n2)sn1 ; Cc1cc(Nc2ncc(C)c(N3CC(CC#N)(NCc4ccc(F)cc4F)C3)n2)sn1] 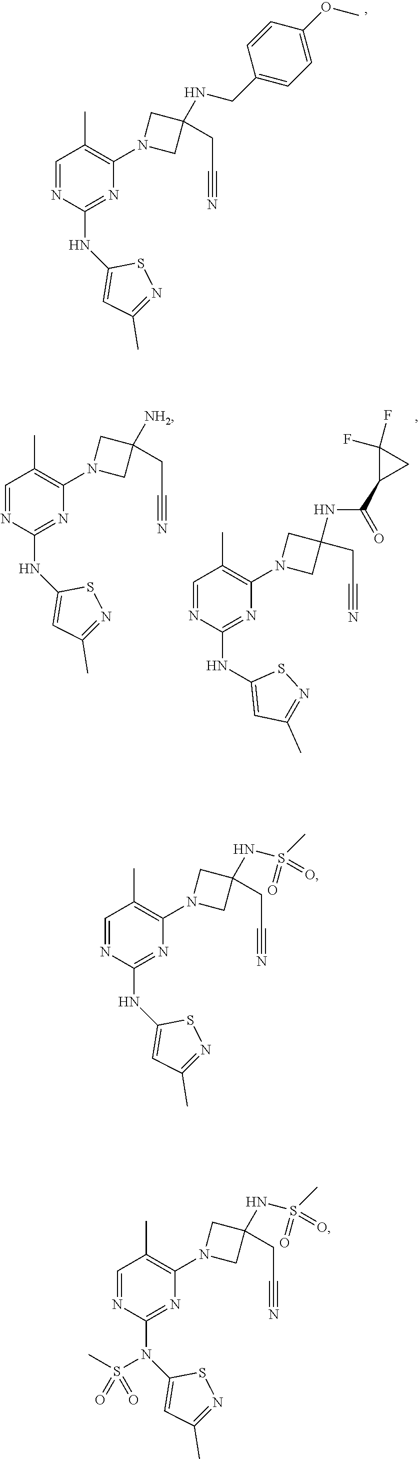 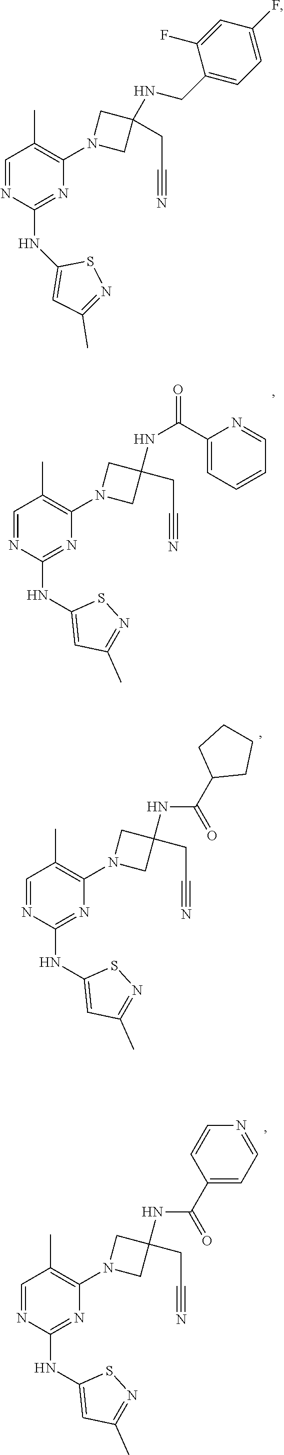 [SMILES: Cc1cc(Nc2ncc(C)c(N3CC(CC#N)(NCc4ccc(F)cc4F)C3)n2)sn1 ; Cc1cc(Nc2ncc(C)c(N3CC(CC#N)(NC(=O)c4ccccn4)C3)n2)sn1 ; Cc1cc(Nc2ncc(C)c(N3CC(CC#N)(NC(=O)C4CCCC4)C3)n2)sn1 ; Cc1cc(Nc2ncc(C)c(N3CC(CC#N)(NC(=O)c4ccncc4)C3)n2)sn1] 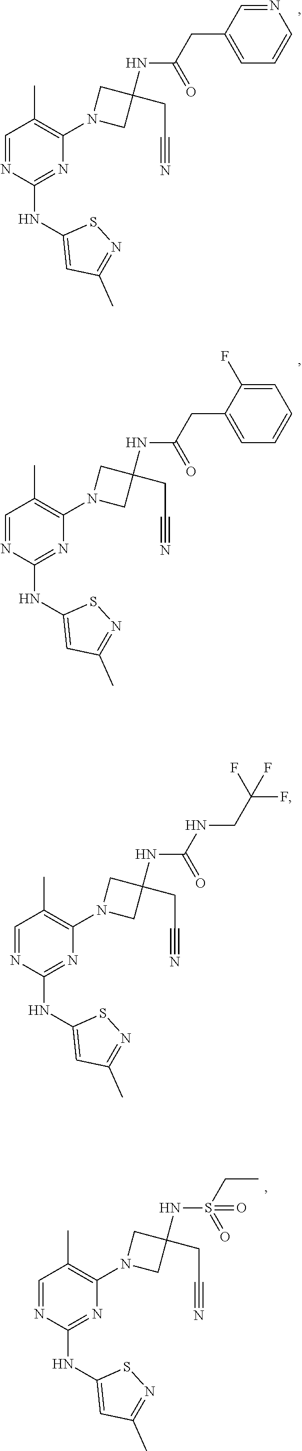 [SMILES: Cc1cc(Nc2ncc(C)c(N3CC(CC#N)(NC(=O)Cc4cccnc4)C3)n2)sn1 ; Cc1cc(Nc2ncc(C)c(N3CC(CC#N)(NC(=O)Cc4ccccc4F)C3)n2)sn1 ; Cc1cc(Nc2ncc(C)c(N3CC(CC#N)(NC(=O)NCC(F)(F)F)C3)n2)sn1 ; CCS(=O)(=O)NC1(CC#N)CN(c2nc(Nc3cc(C)ns3)ncc2C)C1] 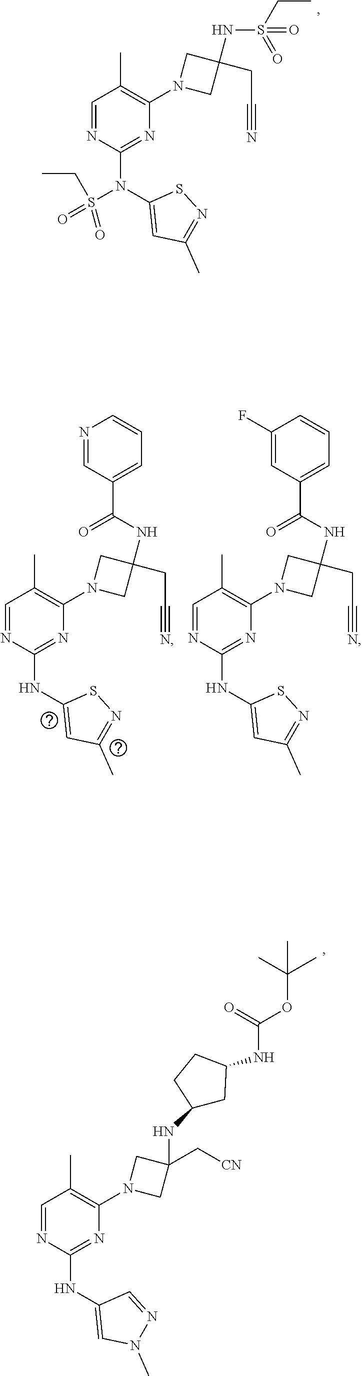 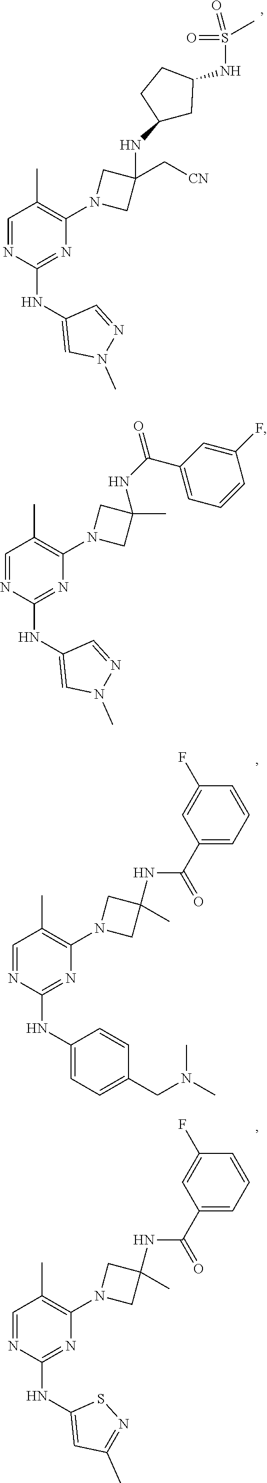 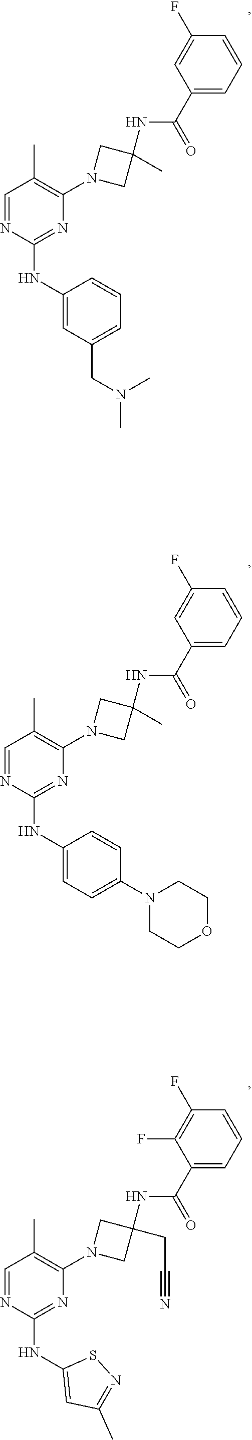 [SMILES: Cc1cnc(Nc2cccc(CN(C)C)c2)nc1N1CC(C)(NC(=O)c2cccc(F)c2)C1 ; Cc1cnc(Nc2ccc(N3CCOCC3)cc2)nc1N1CC(C)(NC(=O)c2cccc(F)c2)C1 ; Cc1cc(Nc2ncc(C)c(N3CC(CC#N)(NC(=O)c4cccc(F)c4F)C3)n2)sn1] 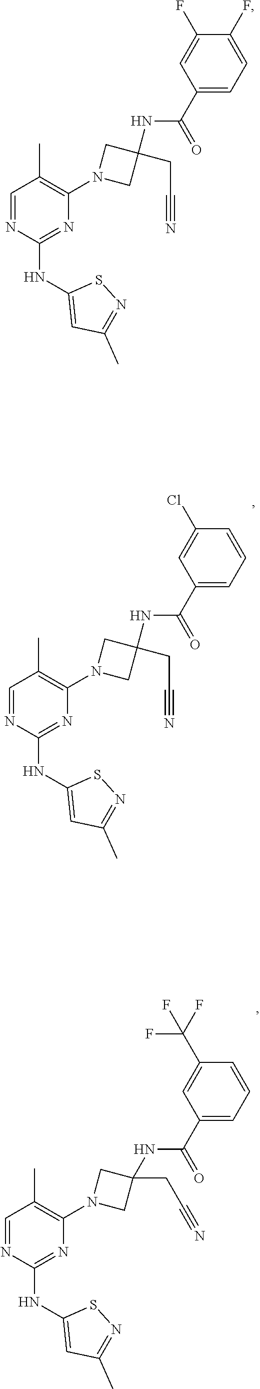 [SMILES: Cc1cc(Nc2ncc(C)c(N3CC(CC#N)(NC(=O)c4ccc(F)c(F)c4)C3)n2)sn1 ; Cc1cc(Nc2ncc(C)c(N3CC(CC#N)(NC(=O)c4cccc(Cl)c4)C3)n2)sn1 ; Cc1cc(Nc2ncc(C)c(N3CC(CC#N)(NC(=O)c4cccc(C(F)(F)F)c4)C3)n2)sn1] 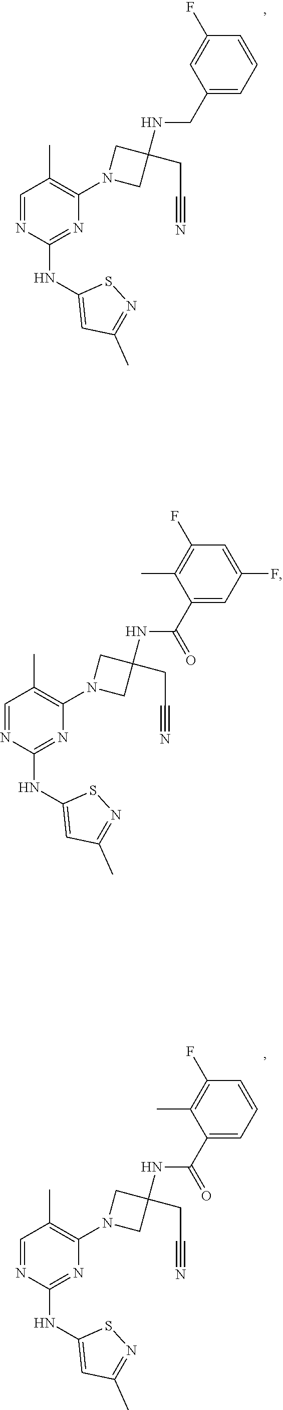 [SMILES: Cc1cc(Nc2ncc(C)c(N3CC(CC#N)(NCc4cccc(F)c4)C3)n2)sn1 ; Cc1cc(Nc2ncc(C)c(N3CC(CC#N)(NC(=O)c4cc(F)cc(F)c4C)C3)n2)sn1 ; Cc1cc(Nc2ncc(C)c(N3CC(CC#N)(NC(=O)c4cccc(F)c4C)C3)n2)sn1] 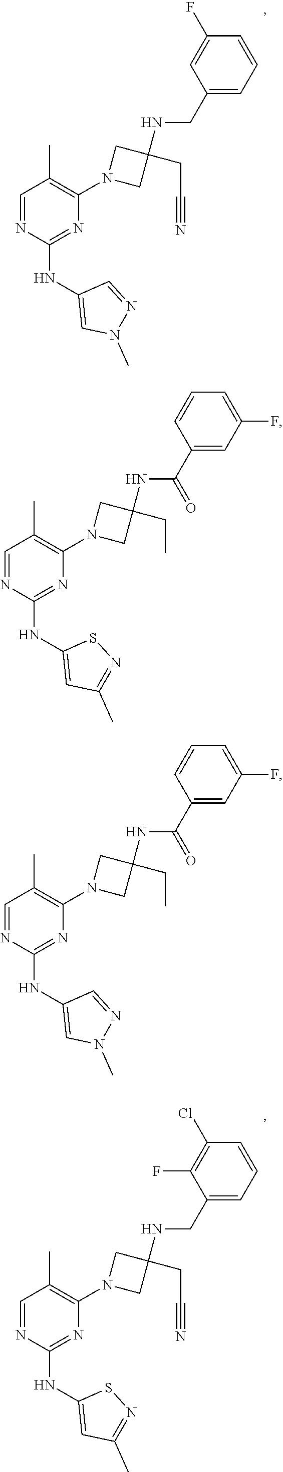 [SMILES: Cc1cnc(Nc2cnn(C)c2)nc1N1CC(CC#N)(NCc2cccc(F)c2)C1 ; CCC1(NC(=O)c2cccc(F)c2)CN(c2nc(Nc3cc(C)ns3)ncc2C)C1 ; CCC1(NC(=O)c2cccc(F)c2)CN(c2nc(Nc3cnn(C)c3)ncc2C)C1 ; Cc1cc(Nc2ncc(C)c(N3CC(CC#N)(NCc4cccc(Cl)c4F)C3)n2)sn1] 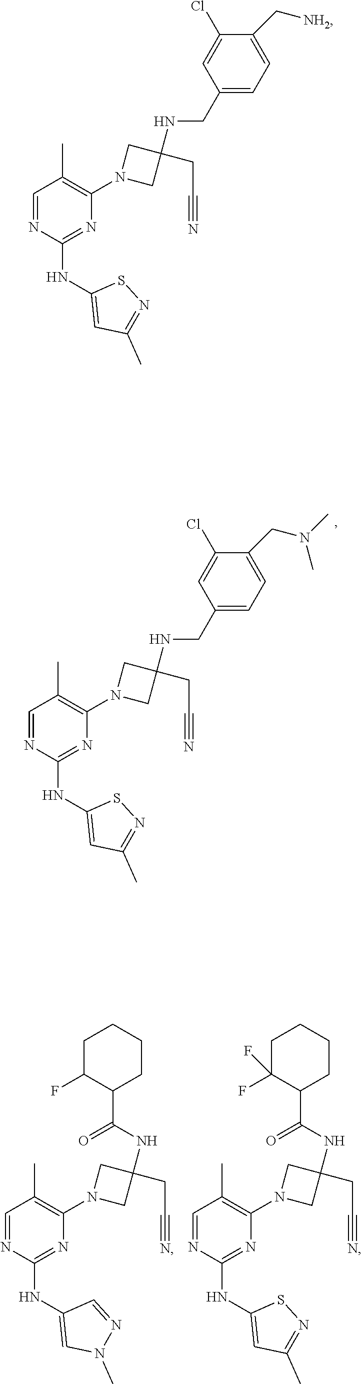 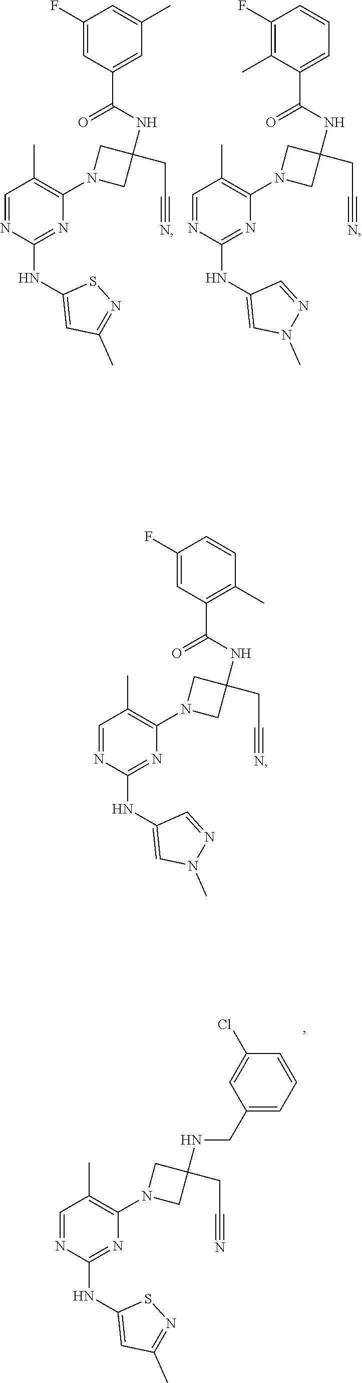 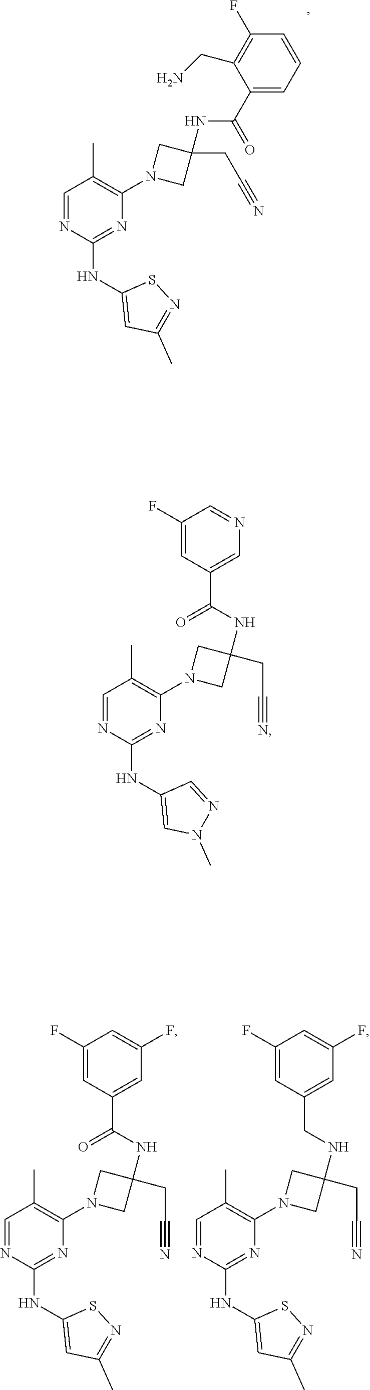 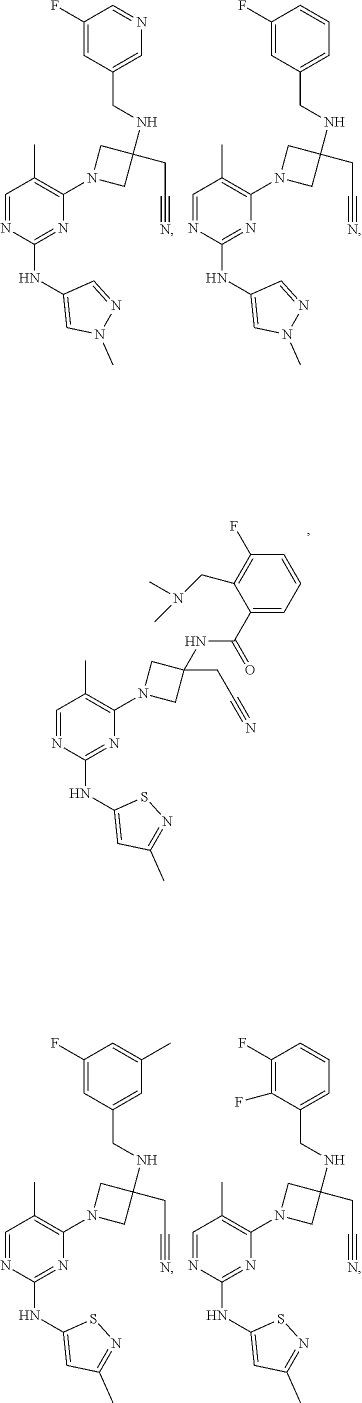 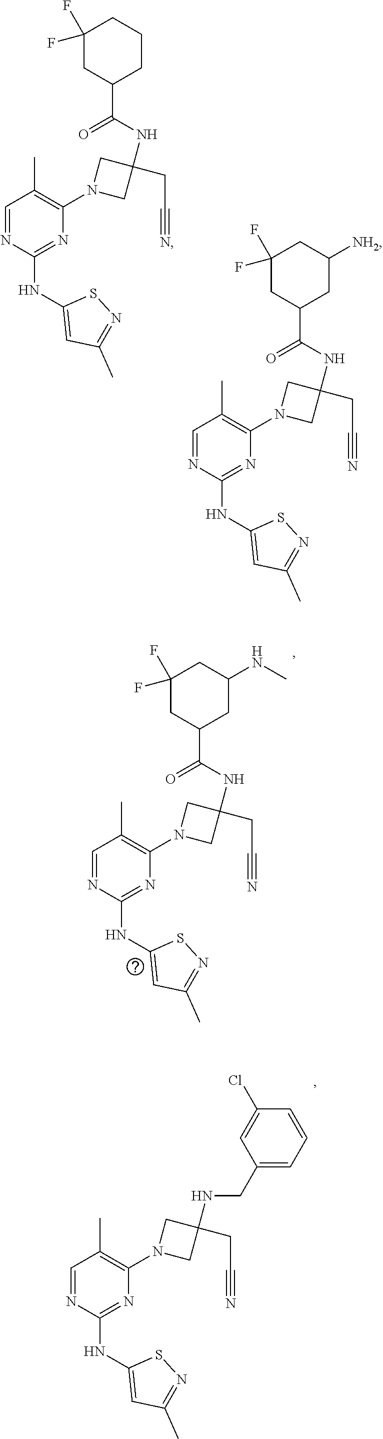 [SMILES: Cc1cc(Nc2ncc(C)c(N3CC(CC#N)(NC(=O)C4CCCC(F)(F)C4)C3)n2)sn1 ; Cc1cc(Nc2ncc(C)c(N3CC(CC#N)(NC(=O)C4CC(N)CC(F)(F)C4)C3)n2)sn1 ; CNC1CC(C(=O)NC2(CC#N)CN(c3nc(Nc4cc(C)ns4)ncc3C)C2)CC(F)(F)C1 ; Cc1cc(Nc2ncc(C)c(N3CC(CC#N)(NCc4cccc(Cl)c4)C3)n2)sn1] 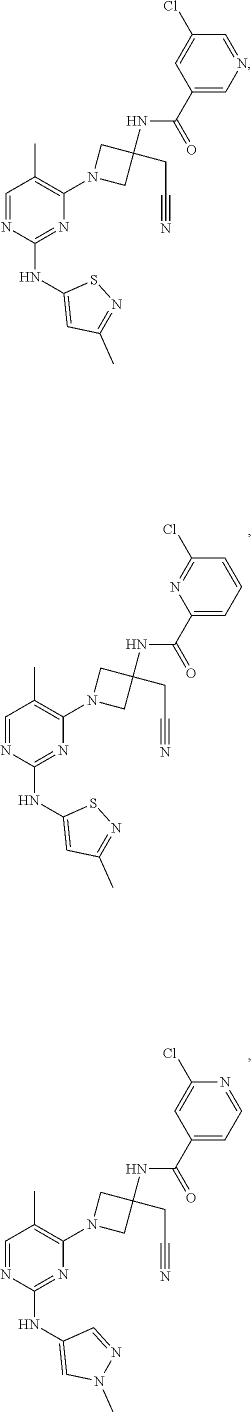 [SMILES: Cc1cc(Nc2ncc(C)c(N3CC(CC#N)(NC(=O)c4cncc(Cl)c4)C3)n2)sn1 ; Cc1cc(Nc2ncc(C)c(N3CC(CC#N)(NC(=O)c4cccc(Cl)n4)C3)n2)sn1 ; Cc1cnc(Nc2cnn(C)c2)nc1N1CC(CC#N)(NC(=O)c2ccnc(Cl)c2)C1] 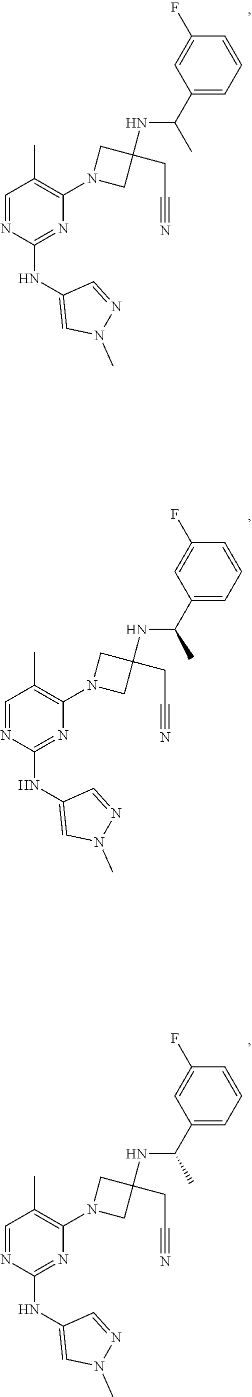 [SMILES: Cc1cnc(Nc2cnn(C)c2)nc1N1CC(CC#N)(NC(C)c2cccc(F)c2)C1 ; Cc1cnc(Nc2cnn(C)c2)nc1N1CC(CC#N)(N[C@H](C)c2cccc(F)c2)C1 ; Cc1cnc(Nc2cnn(C)c2)nc1N1CC(CC#N)(N[C@@H](C)c2cccc(F)c2)C1] 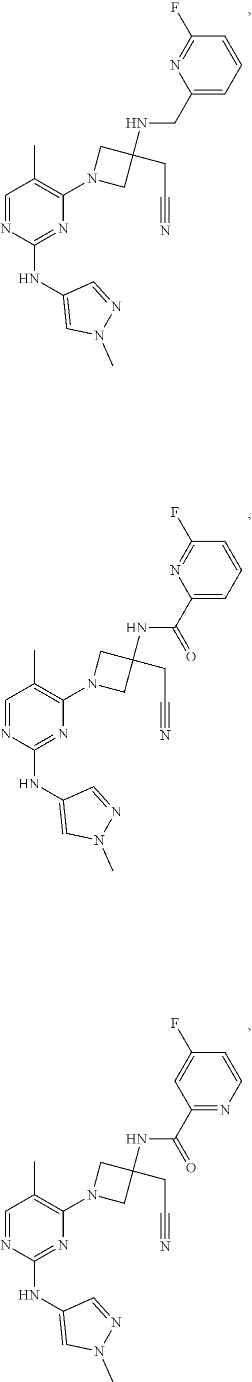 [SMILES: Cc1cnc(Nc2cnn(C)c2)nc1N1CC(CC#N)(NCc2cccc(F)n2)C1 ; Cc1cnc(Nc2cnn(C)c2)nc1N1CC(CC#N)(NC(=O)c2cccc(F)n2)C1 ; Cc1cnc(Nc2cnn(C)c2)nc1N1CC(CC#N)(NC(=O)c2cc(F)ccn2)C1] 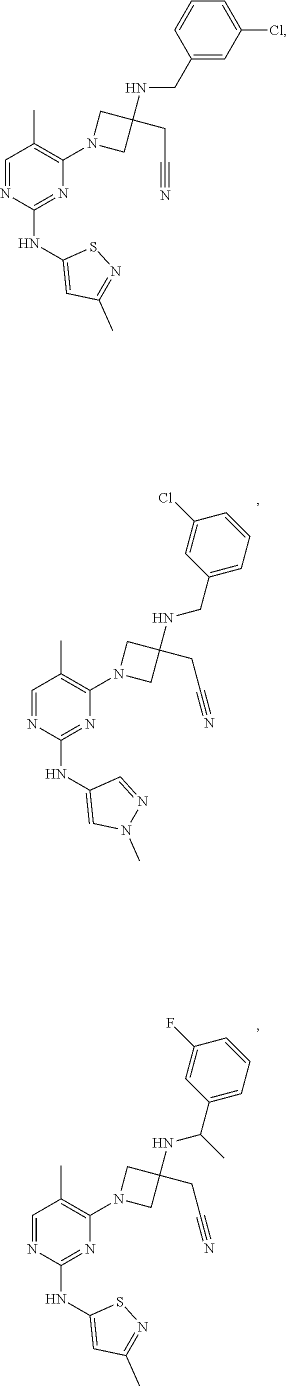 [SMILES: Cc1cc(Nc2ncc(C)c(N3CC(CC#N)(NCc4cccc(Cl)c4)C3)n2)sn1 ; Cc1cnc(Nc2cnn(C)c2)nc1N1CC(CC#N)(NCc2cccc(Cl)c2)C1 ; Cc1cc(Nc2ncc(C)c(N3CC(CC#N)(NC(C)c4cccc(F)c4)C3)n2)sn1] 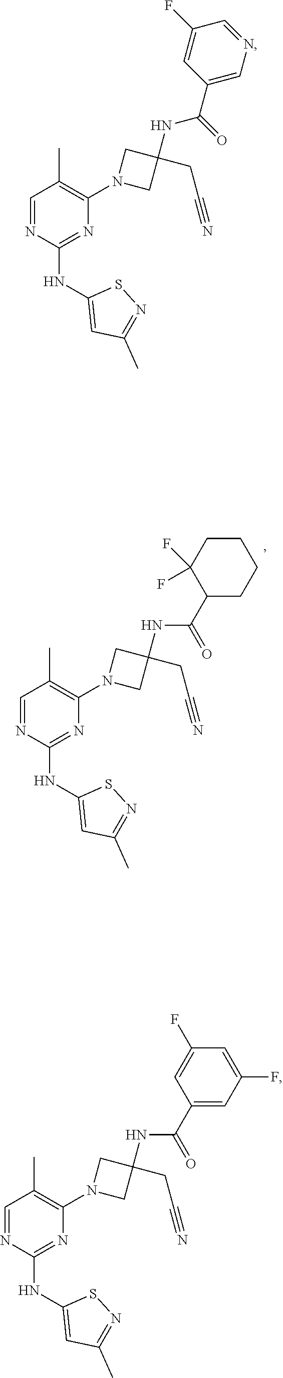 [SMILES: Cc1cc(Nc2ncc(C)c(N3CC(CC#N)(NC(=O)c4cncc(F)c4)C3)n2)sn1 ; Cc1cc(Nc2ncc(C)c(N3CC(CC#N)(NC(=O)C4CCCCC4(F)F)C3)n2)sn1 ; Cc1cc(Nc2ncc(C)c(N3CC(CC#N)(NC(=O)c4cc(F)cc(F)c4)C3)n2)sn1] 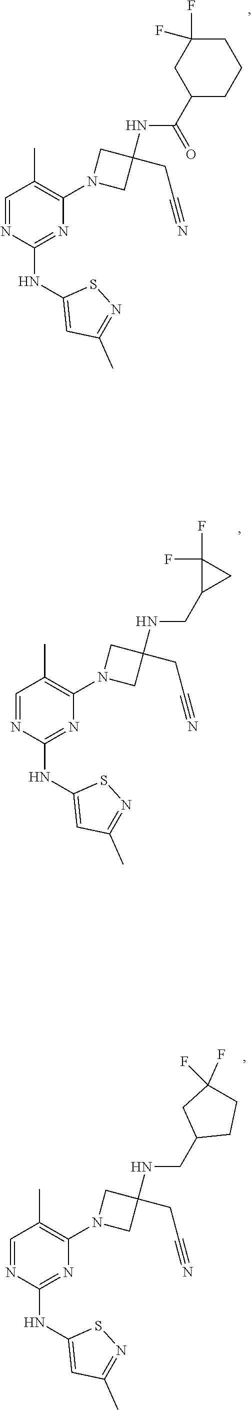 [SMILES: Cc1cc(Nc2ncc(C)c(N3CC(CC#N)(NC(=O)C4CCCC(F)(F)C4)C3)n2)sn1 ; Cc1cc(Nc2ncc(C)c(N3CC(CC#N)(NCC4CC4(F)F)C3)n2)sn1 ; Cc1cc(Nc2ncc(C)c(N3CC(CC#N)(NCC4CCC(F)(F)C4)C3)n2)sn1] 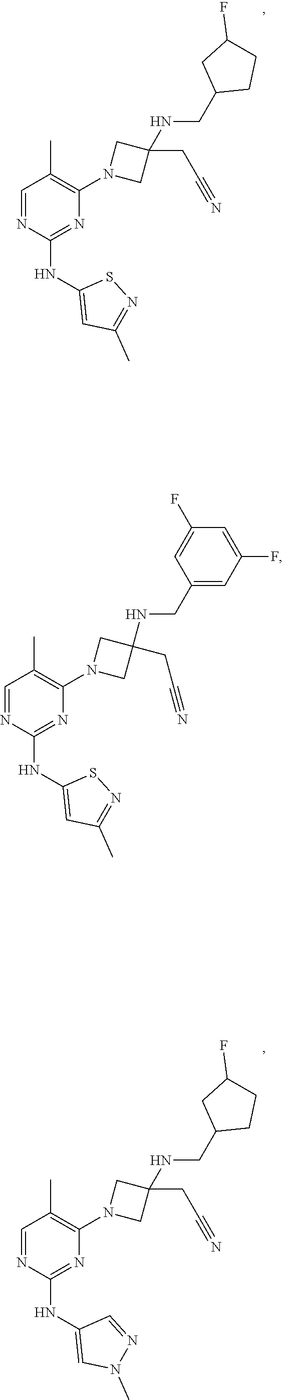 [SMILES: Cc1cc(Nc2ncc(C)c(N3CC(CC#N)(NCC4CCC(F)C4)C3)n2)sn1 ; Cc1cc(Nc2ncc(C)c(N3CC(CC#N)(NCc4cc(F)cc(F)c4)C3)n2)sn1 ; Cc1cnc(Nc2cnn(C)c2)nc1N1CC(CC#N)(NCC2CCC(F)C2)C1] 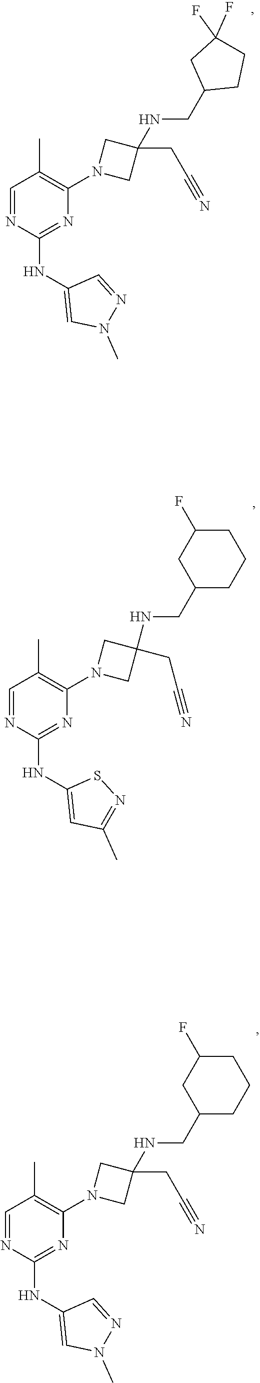 [SMILES: Cc1cnc(Nc2cnn(C)c2)nc1N1CC(CC#N)(NCC2CCC(F)(F)C2)C1 ; Cc1cc(Nc2ncc(C)c(N3CC(CC#N)(NCC4CCCC(F)C4)C3)n2)sn1 ; Cc1cnc(Nc2cnn(C)c2)nc1N1CC(CC#N)(NCC2CCCC(F)C2)C1] 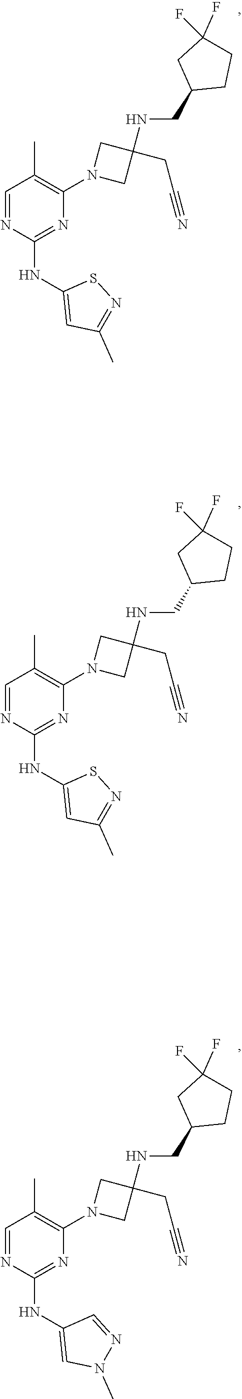 [SMILES: Cc1cc(Nc2ncc(C)c(N3CC(CC#N)(NC[C@@H]4CCC(F)(F)C4)C3)n2)sn1 ; Cc1cc(Nc2ncc(C)c(N3CC(CC#N)(NC[C@H]4CCC(F)(F)C4)C3)n2)sn1 ; Cc1cnc(Nc2cnn(C)c2)nc1N1CC(CC#N)(NC[C@@H]2CCC(F)(F)C2)C1] 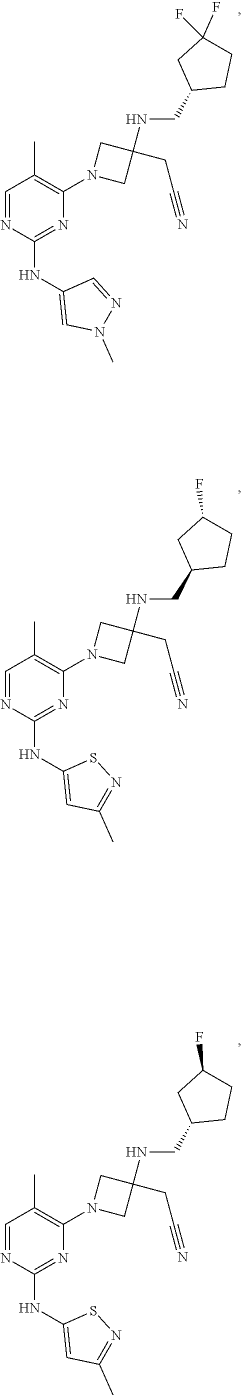 [SMILES: Cc1cnc(Nc2cnn(C)c2)nc1N1CC(CC#N)(NC[C@H]2CCC(F)(F)C2)C1 ; Cc1cc(Nc2ncc(C)c(N3CC(CC#N)(NC[C@@H]4CC[C@@H](F)C4)C3)n2)sn1 ; Cc1cc(Nc2ncc(C)c(N3CC(CC#N)(NC[C@H]4CC[C@H](F)C4)C3)n2)sn1] 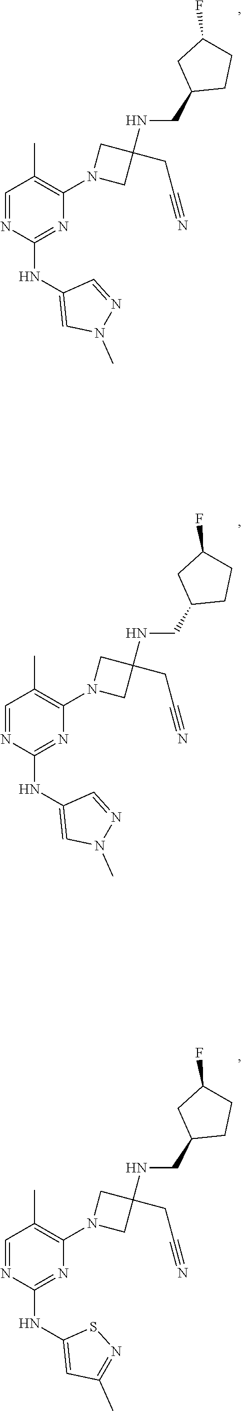 [SMILES: Cc1cnc(Nc2cnn(C)c2)nc1N1CC(CC#N)(NC[C@@H]2CC[C@@H](F)C2)C1 ; Cc1cnc(Nc2cnn(C)c2)nc1N1CC(CC#N)(NC[C@H]2CC[C@H](F)C2)C1 ; Cc1cc(Nc2ncc(C)c(N3CC(CC#N)(NC[C@@H]4CC[C@H](F)C4)C3)n2)sn1] 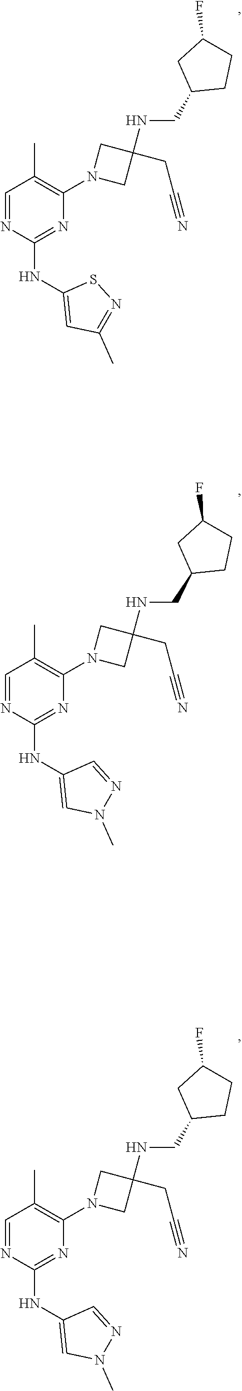 [SMILES: Cc1cc(Nc2ncc(C)c(N3CC(CC#N)(NC[C@H]4CC[C@@H](F)C4)C3)n2)sn1 ; Cc1cnc(Nc2cnn(C)c2)nc1N1CC(CC#N)(NC[C@@H]2CC[C@H](F)C2)C1 ; Cc1cnc(Nc2cnn(C)c2)nc1N1CC(CC#N)(NC[C@H]2CC[C@@H](F)C2)C1] 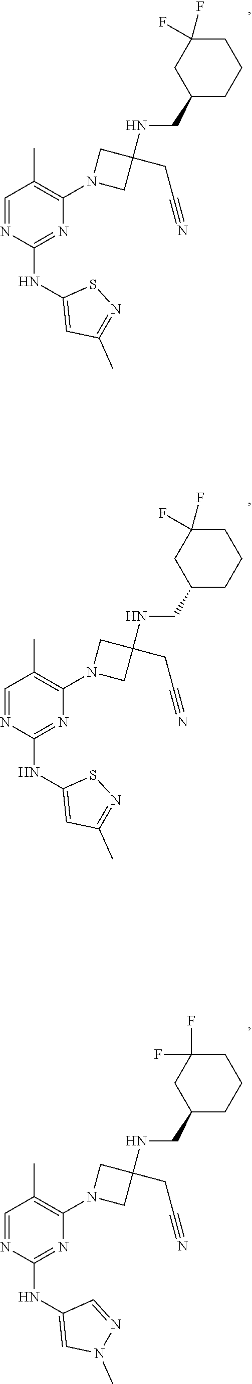 [SMILES: Cc1cc(Nc2ncc(C)c(N3CC(CC#N)(NC[C@@H]4CCCC(F)(F)C4)C3)n2)sn1 ; Cc1cc(Nc2ncc(C)c(N3CC(CC#N)(NC[C@H]4CCCC(F)(F)C4)C3)n2)sn1 ; Cc1cnc(Nc2cnn(C)c2)nc1N1CC(CC#N)(NC[C@@H]2CCCC(F)(F)C2)C1] 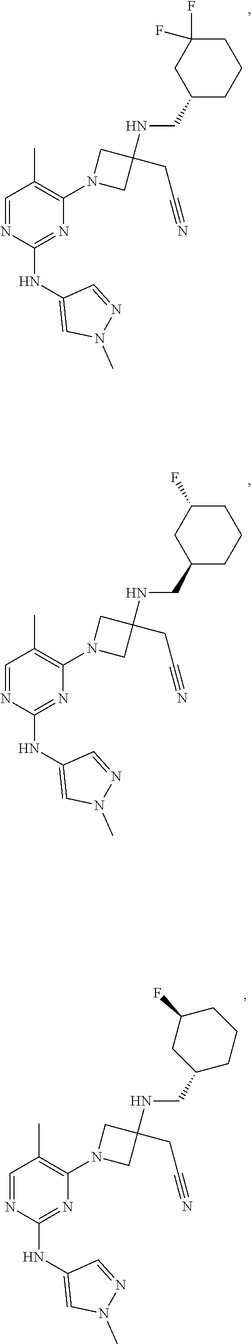 [SMILES: Cc1cnc(Nc2cnn(C)c2)nc1N1CC(CC#N)(NC[C@H]2CCCC(F)(F)C2)C1 ; Cc1cnc(Nc2cnn(C)c2)nc1N1CC(CC#N)(NC[C@@H]2CCC[C@@H](F)C2)C1 ; Cc1cnc(Nc2cnn(C)c2)nc1N1CC(CC#N)(NC[C@H]2CCC[C@H](F)C2)C1] 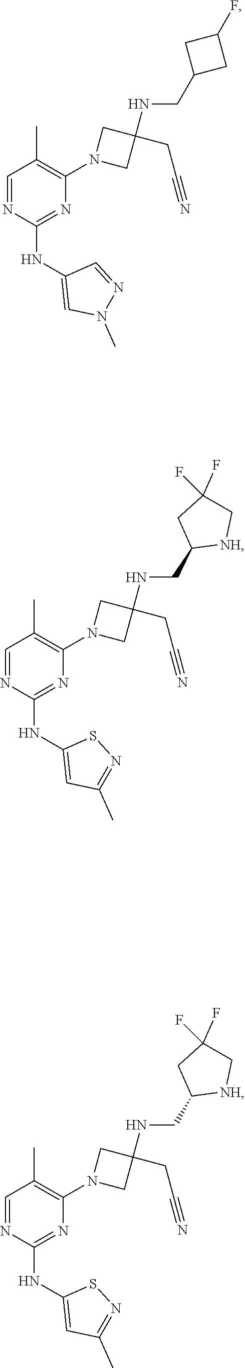 [SMILES: Cc1cnc(Nc2cnn(C)c2)nc1N1CC(CC#N)(NCC2CC(F)C2)C1 ; Cc1cc(Nc2ncc(C)c(N3CC(CC#N)(NC[C@H]4CC(F)(F)CN4)C3)n2)sn1 ; Cc1cc(Nc2ncc(C)c(N3CC(CC#N)(NC[C@@H]4CC(F)(F)CN4)C3)n2)sn1] 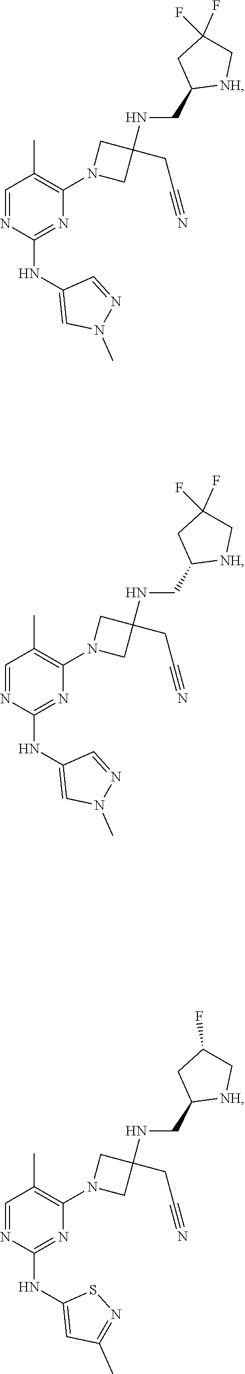 [SMILES: Cc1cnc(Nc2cnn(C)c2)nc1N1CC(CC#N)(NC[C@H]2CC(F)(F)CN2)C1 ; Cc1cnc(Nc2cnn(C)c2)nc1N1CC(CC#N)(NC[C@@H]2CC(F)(F)CN2)C1 ; Cc1cc(Nc2ncc(C)c(N3CC(CC#N)(NC[C@H]4C[C@H](F)CN4)C3)n2)sn1] 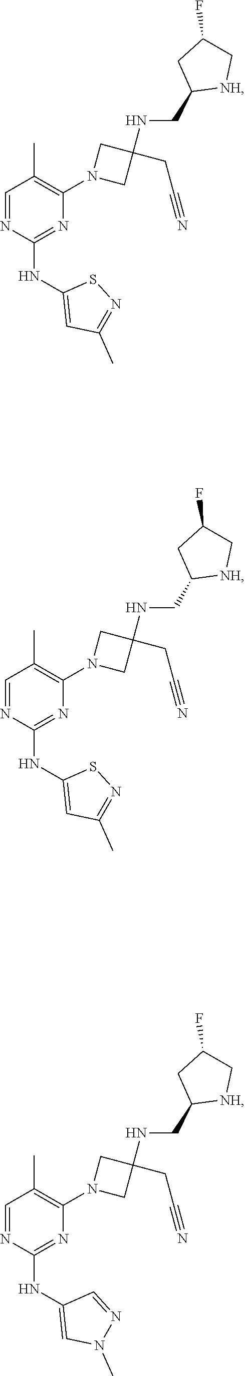 [SMILES: Cc1cc(Nc2ncc(C)c(N3CC(CC#N)(NC[C@H]4C[C@H](F)CN4)C3)n2)sn1 ; Cc1cc(Nc2ncc(C)c(N3CC(CC#N)(NC[C@@H]4C[C@@H](F)CN4)C3)n2)sn1 ; Cc1cnc(Nc2cnn(C)c2)nc1N1CC(CC#N)(NC[C@H]2C[C@H](F)CN2)C1] 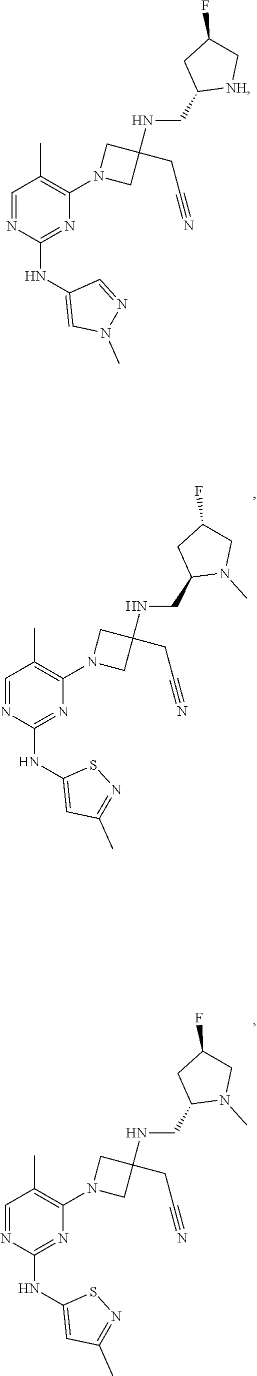 [SMILES: Cc1cnc(Nc2cnn(C)c2)nc1N1CC(CC#N)(NC[C@@H]2C[C@@H](F)CN2)C1 ; Cc1cc(Nc2ncc(C)c(N3CC(CC#N)(NC[C@H]4C[C@H](F)CN4C)C3)n2)sn1 ; Cc1cc(Nc2ncc(C)c(N3CC(CC#N)(NC[C@@H]4C[C@@H](F)CN4C)C3)n2)sn1] 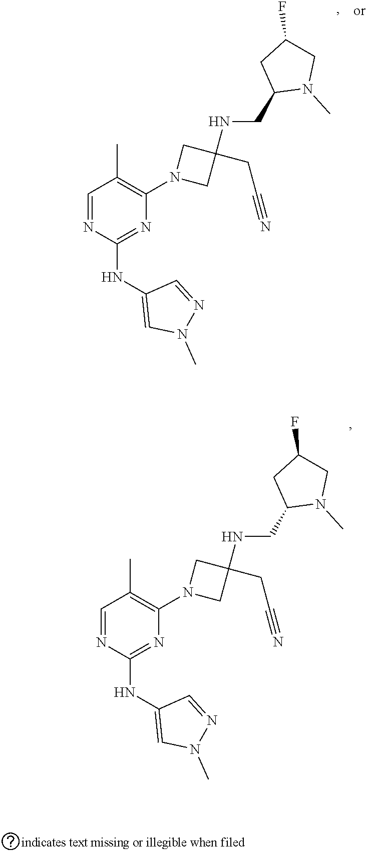 [SMILES: Cc1cnc(Nc2cnn(C)c2)nc1N1CC(CC#N)(NC[C@H]2C[C@H](F)CN2C)C1 ; Cc1cnc(Nc2cnn(C)c2)nc1N1CC(CC#N)(NC[C@@H]2C[C@@H](F)CN2C)C1]
or a pharmaceutically acceptable salt thereof.

33. A method of treating an ocular disease or condition in a subject in need thereof, comprising administering to the subject a therapeutically effective amount of the compound of claim 1.

34. A method of reducing ocular inflammation in a subject in need thereof, comprising administering to the subject a therapeutically effective amount of the compound of claim 1.

35. A method of treating an autoimmune disease or condition in a subject in need thereof, comprising administering to the subject a therapeutically effective amount of the compound of claim 1.

36. A method of treating a disease or condition associated with kinase activity or diseases or conditions affected by kinases, comprising administering to the subject an effective amount of the compound of claim 1.


SEQUENCE LISTING (2 sequences)

SEQ ID NO: 1 - protein, 30 aa, synthetic construct
LYSARGARGARGLEUSERSERLEUARGALA

SEQ ID NO: 2 - protein, 36 aa, synthetic construct
GLUALAILETYRALAALAPROPHEALALYSLYSLYS